(19) United States
(12) Patent Application Publication
(10) Pub. No.: US 2021/0069230 A1
(43) Pub. Date: Mar. 11, 2021

(54) SYNERGISTIC DRUG COMBINATIONS PREDICTED FROM GENOMIC FEATURES AND SINGLE-AGENT RESPONSE PROFILES

(71) Applicant: THE BROAD INSTITUTE, INC., Cambridge, MA (US)

(72) Inventors: Matthew Rees, Cambridge, MA (US); Cory Johannessen, Cambridge, MA (US)

(73) Assignee: THE BROAD INSTITUTE, INC., Cambridge, MA (US)

(21) Appl. No.: 17/046,560

(22) PCT Filed: Apr. 12, 2019
(86) PCT No.: PCT/US19/27170

§ 371 Date: Oct. 9, 2020

Related U.S. Application Data
(60) Provisional application No. 62/657,690, filed on Apr. 13, 2018.

(51) Int. Cl. A61K 31/7088; A61K 31/55; A61K 31/4709; A61K 31/192; A61K 31/4525; A61K 31/325; A61K 31/215; A61K 31/167; A61K 31/381; A61K 31/341; A61K 31/18; A61K 31/343; A61K 31/357; A61K 31/366; A61K 31/695; A61K 31/454; A61K 31/453; A61K 31/506; A61K 31/519; A61K 31/5025; A61K 31/4985; A61K 31/473; A61K 31/7048; A61K 31/496; A61K 31/352; A61K 31/704; A61K 31/4045; A61K 31/138; A61K 31/365; A61K 31/12; A61K 31/5377; A61K 31/52; A61K 31/553; A61K 31/661; A61K 31/4439; A61K 31/4745; A61K 31/437; A61K 31/4453; A61K 31/53; A61K 31/4704; A61K 31/47; A61K 31/416; A61K 31/4725; A61K 31/4402; A61P 35/00

(52) U.S. Cl. CPC A61K 31/7088; A61P 35/00; A61K 31/4709; A61K 31/192; A61K 31/4525; A61K 31/325; A61K 31/215; A61K 31/167; A61K 31/381; A61K 31/341; A61K 31/18; A61K 31/343; A61K 31/357; A61K 31/366; A61K 31/695; A61K 31/454; A61K 31/453; A61K 31/506; A61K 31/519; A61K 31/5025; A61K 31/4985; A61K 31/473; A61K 31/7048; A61K 31/496; A61K 31/352; A61K 31/704; A61K 31/4045; A61K 31/138; A61K 31/365; A61K 31/12; A61K 31/5377; A61K 31/52; A61K 31/553; A61K 31/661; A61K 31/4439; A61K 31/4745; A61K 31/437; A61K 31/4453; A61K 31/53; A61K 31/4704; A61K 31/47; A61K 31/416; A61K 31/4725; A61K 31/4402; A61K 31/55

(57) ABSTRACT

The present disclosure relates to discovery of specific synergistic drug combinations and mechanisms of drug resistance. Compositions involving newly-identified drug combinations as well as diagnostic and therapeutic methods related to such discoveries are provided.

### CROSS-REFERENCE TO RELATED APPLICATION

This application is an International Patent Application which claims the benefit of priority under 35 U.S.C. § 119(e) to U.S. Provisional Application No. 62/657,690, filed on Apr. 13, 2018, entitled, “Synergistic Drug Combinations Predicted from Genomic Features and Single-Agent Response Profiles.” The entire contents of this patent application are hereby incorporated by reference herein.

### FIELD OF THE INVENTION

The invention relates generally to methods and compositions for the diagnosis and treatment of cancers.

### BACKGROUND OF THE INVENTION

Molecular mechanisms that underpin cancer and other diseases are often relevant to disease progression, patient survival and selection of effective therapeutic options. A clear example is the MGMTpromoter, where, depending upon the methylation state of the promoter, dramatic differences in overall survival were observed for glioblastoma subjects receiving temozolomide (Hegi et al. New England Journal of Medicine, 2005, 352: 997-1003, where the presence of methylated MGMT promoter in a sub-population of glioblastoma subjects was associated with longer-term survival (i.e., survival exceeding 24 months in duration) when treated with temozolomide). A need exists for improved compositions, including combination therapeutics, as well as methods for diagnosing and treating cancers, as well as other diseases and disorders.

### BRIEF SUMMARY OF THE INVENTION

The current disclosure relates, at least in part, to compositions and methods for the diagnosis and treatment of cancers. In particular, the instant disclosure has identified combination therapies directed towards sensitizing target cells to agents to which naturally-occurring transcripts would otherwise tend to impart resistance.

In one aspect, the current disclosure provides a pharmaceutical composition for treating a cancer in a subject that includes a KDM6A/KDM6B inhibitor and a MGLL inhibitory agent, and a pharmaceutically acceptable carrier.

In one embodiment, the KDM6A/B inhibitor is GSK-J4, IOX1, GSK-J1 or caffeic acid.

In another embodiment, the MGLL inhibitory agent is JZL 184, URB602, pristimerin, an 0-hexafluorosiopropyl carbamate or an oligonucleotide inhibitor of MGLL.

In an additional embodiment, the cancer is AML, ALL or prostate cancer.

Another aspect of the current disclosure provides a pharmaceutical composition for treating a cancer in a subject that includes an ERK1 inhibitor and an ICMT inhibitory agent, and a pharmaceutically acceptable carrier.

In one embodiment, the ERK1 inhibitor is SCH772984, LY3214996, GDC-0994, FR 180204 or pluripotin.

In another embodiment, the ICMT inhibitory agent is (E,E)-Farnesyl Thiol, ICMT inhibitor 420350, ICMT inhibitor 420351, cysmethynil or an oligonucleotide inhibitor of ICMT.

In one embodiment, the cancer is advanced or metastatic cancer, optionally with an activating mitogen-activated protein kinase pathway alteration, optionally a BRAF mutant metastatic melanoma refractory to or relapsed after treatment with RAF and/or MEK inhibitors, optionally a metastatic melanoma with an NRAS mutation, optionally an advanced, unresectable cancer, optionally an advanced, unresectable, or metastatic non-small cell lung cancer (NSCLC), optionally with a BRAT or RAS mutation; optionally colorectal cancer, optionally with a RAS mutation; optionally in metastatic pancreatic ductal adenocarcinoma.

An additional aspect of the current disclosure provides a pharmaceutical composition for treating a cancer in a subject that includes a STAT3 signaling inhibitor and a UGT1A10 inhibitory agent, and a pharmaceutically acceptable carrier.

In one embodiment, the STAT3 signaling inhibitor is niclosamide (5-Chloro-N-(2-chloro-4-nitrophenyl)-2-hydroxybenzamide), S3I-201, stattic, nifuroxazide, C188-9, SH-4-54, napabucasin, artesunate, BP-1-102, cryptotanshinone, SH5-07 (SH-5-07), ochromycinone (STA-21), APTSTAT3-9R or HO-3867.

In another embodiment, the UGT1A10 inhibitory agent is an oligonucleotide inhibitor of UGT1A10.

In an additional embodiment, the cancer is breast cancer, head and neck squamous cell carcinoma (HNSCC), non-small cell lung cancer (NSCLC), hepatocellular carcinoma (HCC), colorectal cancer (CRC), gastric adenocarcinoma or melanoma.

Another aspect of the current disclosure provides a pharmaceutical composition for treating a cancer in a subject that includes a CDK4/6 inhibitor and a CCNE1 inhibitory agent, and a pharmaceutically acceptable carrier.

In one embodiment, the CDK4/6 inhibitor is flavopiridol, abemaciclib, ribociclib or palbociclib.

In another embodiment, the CCNE1 inhibitory agent is an oligonucleotide inhibitor of CCNE1.

In an additional embodiment, the cancer is breast cancer, non-small cell lung cancer (NSCLC), mantle cell lymphoma, liposarcoma, melanoma, glioblastoma, pancreatic cancer or colorectal cancer.

A further aspect of the current disclosure provides a pharmaceutical composition for treating a cancer in a subject that includes an AKT1/2 inhibitor and an AKT3 inhibitory agent, and a pharmaceutically acceptable carrier.

In one embodiment, the AKT1/2 inhibitor is BAY1125976 or AKT inhibitor VIII.

In another embodiment, the AKT3 inhibitory agent is an oligonucleotide inhibitor of AKT3.

In an additional embodiment, the cancer is breast cancer, head and neck cancer, squamous cell carcinoma, endometrial cancer, non-small cell lung cancer (NSCLC), renal cancer, gastric cancer, ovarian cancer, pancreatic cancer, colon cancer, oesophageal cancer or thyroid cancer.

Another aspect of the current disclosure provides a pharmaceutical composition for treating a cancer in a subject that includes a topoisomerase II inhibitor and a BCL2L1 inhibitory agent, and a pharmaceutically acceptable carrier.

In one embodiment, the topoisomerase II inhibitor is amsacrine, etoposide, etoposide phosphate, teniposide, ICRF-193, genistein or doxorubicin.

In another embodiment, the BCL2L1 inhibitory agent is Z36, 2,3-DCPE hydrochloride, arctigenin, (±)-gossypol, gossypol-acetic acid, R(−)-gossypol or an oligonucleotide inhibitor of BCL2L1.

In an additional embodiment, the cancer is breast cancer, bladder cancer, Kaposi's sarcoma, lymphoma, acute lymphocytic leukemia or colorectal cancer.

An additional aspect of the current disclosure provides a pharmaceutical composition for treating a cancer in a subject that includes a PI3 kinase inhibitor and an IRS2 inhibitory agent, and a pharmaceutically acceptable carrier.

In one embodiment, the PI3 kinase inhibitor is Pictilisib, Idelalisib, Copanlisib, Taselisib, Perifosine, Buparlisib (BKM120), Duvelisib (IPI-145), Alpelisib (BYL719), Umbralisib, (TGR 1202), Copanlisib (BAY 80-6946), PX-866, Dactolisib, CUDC-907, Voxtalisib, CUDC-907, ME-401, IPI-549, SF1126, RP6530, INK1117, XL147 (also known as SAR245408), Palomid 529, GSK1059615, ZSTK474, PWT33597, IC87114, TG100-115, CAL263, RP6503, PI-103, GNE-477 or AEZS-136.

In another embodiment, the IRS2 inhibitory agent is an oligonucleotide inhibitor of IRS2. In an additional embodiment, the cancer is leukemia, breast cancer, lung cancer, colorectal cancer, hematologic malignancies, thyroid cancer, inflammatory conditions, multiple myeloma or lymphoma, optionally a B-cell lymphoma, optionally CLL or follicular lymphoma.

A further aspect of the current disclosure provides a pharmaceutical composition for treating a cancer in a subject that includes a GPX4 inhibitor and an AIFM2 inhibitory agent, and a pharmaceutically acceptable carrier.

In one embodiment, the GPX4 inhibitor is ML210, racemic RSL3, (1S,3R)-RSL3, ML162, CIL56, DPI19, DPI18, DPI17, DPI13, DPI12, altretamine or FIN56.

In another embodiment, the AIFM2 inhibitory agent is an oligonucleotide inhibitor of AIFM2.

In an additional embodiment, the cancer is a diffuse large B cell lymphoma (DLBCL) or a renal cell carcinoma.

Another aspect of the current disclosure provides a pharmaceutical composition for treating a cancer in a subject that includes an inducer of reactive oxygen species (ROS) and an ABCC1 inhibitory agent, and a pharmaceutically acceptable carrier.

In one embodiment, the inducer of reactive oxygen species (ROS) is BRD1378, BRD5459, BRD56491 or BRD9092.

In another embodiment, the ABCC1 inhibitory agent is MK-571, Reversan or an oligonucleotide inhibitor of ABCC1.

In an additional embodiment, the cancer is colon, pancreatic, breast, glioma, glioblastoma, non-small cell lung cancer, multiple myeloma, prostate cancer, hepatoma or leukemia.

An additional aspect of the current disclosure provides a pharmaceutical composition for treating a cancer in a subject that includes a JNK1 inhibitor and an ABCG2 inhibitory agent, and a pharmaceutically acceptable carrier.

In one embodiment, the JNK1 inhibitor is ZG-10, tanzisertib (CC-930), SP600125, JNK Inhibitor V, INK Inhibitor XIV, L-JNKi 1 trifluoroacetate salt, INK Inhibitor XV, JNK Inhibitor XI or INK-IN-8.

In another embodiment, the ABCG2 inhibitory agent is elacridar, vismodegib, fumitremorgin C, Ko 143, novobiocin sodium salt or an oligonucleotide inhibitor of ABCG2.

In an additional embodiment, the cancer is liver cancer, breast cancer, skin cancer, brain cancer, leukaemia, multiple myeloma or lymphoma.

A further aspect of the current disclosure provides a pharmaceutical composition for treating a cancer in a subject that includes an E3-ubiquitin ligase inhibitor and a UGT1A6 inhibitory agent, and a pharmaceutically acceptable carrier.

In one embodiment, the E3-ubiquitin ligase inhibitor is SMER-3, Heclin or SZL P1-41.

In another embodiment, the UGT1A6 inhibitory agent is an oligonucleotide inhibitor of UGT1A6.

In an additional embodiment, the cancer is malignant melanoma or multiple myeloma.

In another aspect, the current disclosure provides a method for reducing resistance of a cell, tissue and/or subject to a KDM6A/KDM6B inhibitor that involves administering a MGLL inhibitory agent to the cell, tissue and/or subject, thereby reducing resistance of the cell, tissue and/or subject to the KDM6A/KDM6B inhibitor.

In an additional aspect, the current disclosure provides a method for reducing resistance of a cell, tissue and/or subject to an ERK1 inhibitor that involves administering an ICMT inhibitory agent to the cell, tissue and/or subject, thereby reducing resistance of the cell, tissue and/or subject to the ERK1 inhibitor.

Another aspect of the current disclosure provides a method for reducing resistance of a cell, tissue and/or subject to a STAT3 signaling inhibitor that involves administering an UGT1A10 inhibitory agent to the cell, tissue and/or subject, thereby reducing resistance of the cell, tissue and/or subject to the STAT3 signaling inhibitor.

A further aspect of the current disclosure provides a method for reducing resistance of a cell, tissue and/or subject to a CDK4/6 inhibitor that involves administering a CCNE1 inhibitory agent to the cell, tissue and/or subject, thereby reducing resistance of the cell, tissue and/or subject to the CDK4/6 inhibitor.

Another aspect of the current disclosure provides a method for reducing resistance of a cell, tissue and/or subject to an AKT1/2 inhibitor that involves administering an AKT3 inhibitory agent to the cell, tissue and/or subject, thereby reducing resistance of the cell, tissue and/or subject to the AKT1/2 inhibitor.

An additional aspect of the current disclosure provides a method for reducing resistance of a cell, tissue and/or subject to a topoisomerase II inhibitor that involves administering a BCL2L1 inhibitory agent to the cell, tissue and/or subject, thereby reducing resistance of the cell, tissue and/or subject to the topoisomerase II inhibitor.

In another aspect, the current disclosure provides a method for reducing resistance of a cell, tissue and/or subject to a PI3 kinase inhibitor that involves administering an IRS2 inhibitory agent to the cell, tissue and/or subject, thereby reducing resistance of the cell, tissue and/or subject to the PI3 kinase inhibitor.

In an additional aspect, the current disclosure provides a method for reducing resistance of a cell, tissue and/or subject to a GPX4 inhibitor that involves administering an AIFM2 inhibitory agent to the cell, tissue and/or subject, thereby reducing resistance of the cell, tissue and/or subject to the GPX4 inhibitor.

Another aspect of the current disclosure provides a method for reducing resistance of a cell, tissue and/or subject to an inducer of reactive oxygen species (ROS) that involves administering an ABCC1 inhibitory agent to the cell, tissue and/or subject, thereby reducing resistance of the cell, tissue and/or subject to the ROS inducer.

An additional aspect of the current disclosure provides a method for reducing resistance of a cell, tissue and/or subject to a JNK1 inhibitor that involves administering an ABCG2 inhibitory agent to the cell, tissue and/or subject, thereby reducing resistance of the cell, tissue and/or subject to the JNK1 inhibitor.

In a further aspect, the current disclosure provides a method for reducing resistance of a cell, tissue and/or subject to an E3-ubiquitin ligase inhibitor that involves administering an UGT1A6 inhibitory agent to the cell, tissue and/or subject, thereby reducing resistance of the cell, tissue and/or subject to the E3-ubiquitin ligase inhibitor.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, lung cancer (e.g., bronchogenic carcinoma, small cell lung cancer (SCLC), non-small cell lung cancer (NSCLC), adenocarcinoma of the lung); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CIVIL), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; vulvar cancer (e.g., Paget's disease of the vulva); diffuse large B-cell lymphoma (DLBCL), as well as the broader class of lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; hematopoietic cancers (e.g., myeloid malignancies (e.g., acute myeloid leukemia (AML) (e.g., B-cell AML, T-cell AML), myelodysplastic syndrome, myeloproliferative neoplasm, chronic myelomonocytic leukemia (CMML) and chronic myelogenous leukemia (CIVIL) (e.g., B-cell CML, T-cell CML)) and lymphocytic leukemia such as acute lymphocytic leukemia (ALL) (e.g., B-cell ALL, T-cell ALL) and chronic lymphocytic leukemia (CLL) (e.g., B-cell CLL, T-cell CLL)); and brain cancer (e.g., meningioma, glioblastomas, glioma (e.g., astrocytoma, oligodendroglioma), medulloblastoma).

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable salts, esters, amides, and prodrugs” as used herein refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference.).

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A-1E depict the systematic discovery, as disclosed herein, of combination targets that improve initial responses (as compared to single-agent effects). FIG. 1A shows the process of relating whole transcriptomes of up to 824 (CTRP) or 985 (GDSC) cancer cell lines to response profiles of 481 (CTRP) or 266 (GDSC) small molecules to identify transcripts correlated with drug resistance. FIG. 1B shows a graph representing the number of transcripts (of 20,327) passing a Bonferroni-adjusted- and z-score significance cutoff for at least one small molecule in at least one of 45 cellular sub-contexts (lineage, histology, growth mode, mutation) in the CTRP dataset, FIG. 1C shows the number of compounds associated with each gene showing a significant correlation in both the CTRP and GDSC datasets: FIG. JD depicts a heatmap showing co-expression (Pearson correlation) across cell lines of most frequently associated genes. Heatmaps were generated using Morpheus (software.broadinstitute.orgimorpheus), with rows and columns clustered (metric: one minus Pearson correlation; linkage method: average). FIG. JE shows results from FIG. 1B after filtration for potential confounding factors such as gene co-expression and non-specific associations.

FIGS. 2A and 2B depict the prioritization of gene-drug pairs. FIG. 2A presents compound-centric box-whisker plots of Fisher's-z-transformed Pearson correlation coefficients between basal gene expression for up to 20,037 transcripts and small-molecule sensitivity (AUC) data. Putative resistance-associated transcripts are labeled. Tukey outliers (1.5× interquartile range) are shown. Correlations from CTRP are shown in black, and from GDSC in blue. FIG. 2B shows gene-centric scatter plots of z-transformed correlation coefficients against per-compound z-scores for 413 (CTRP; black dots)+267 (GDSC; blue dots) small molecules for nine highlighted genes.

FIGS. 3A to 3L show how ectopic expression of candidate co-targets is sufficient to drive drug resistance. FIG. 3A shows a key for resistance gene: index compound profiling, which demonstrated the effects of lentiviral overexpression of nominated transcripts (red) on compound sensitivity after 72 hours (or 120 hours, for MGMT in FIG. 3E below), as measured by CellTiter-Glo (or by live-cell imaging, for MGMT in FIG. 3E below). Parental cell lines are shown in black, with control ORFs in blue. Cell viability values (GR) are normalized to DMSO treatment and to initial (D0) cell number. Each point represents mean±SD (n=2-4 technical replicates) and is representative of at least two independent infections. FIG. 3B shows the effects of lentiviral overexpression of MGLL (red) on GSK-J4 sensitivity after 72 hours. FIG. 3C shows the effects of lentiviral overexpression of AIFM2 (red) on ML210 sensitivity after 72 hours, with impact of the AIFM2 D285N mutant (AIFM2′) also shown. FIG. 3D shows the effects of lentiviral overexpression of MCL1 (red) on navitoclax sensitivity after 72 hours. FIG. 3E shows the effects of lentiviral overexpression of MGMT (red) on temozolomide sensitivity after 120 hours. FIG. 3F shows the effects of lentiviral overexpression of SLC7A11 (red) on PRIMA-1 sensitivity after 72 hours. FIG. 3G shows the effects of lentiviral overexpression of ABCB1 (red) on paclitaxel sensitivity after 72 hours. FIG. 3H shows the effects of lentiviral overexpression of BCL2L1 (red) on doxorubicin sensitivity after 72 hours. FIG. 3I shows the effects of lentiviral overexpression of IRS2 (red) on pictilisib sensitivity after 72 hours. FIG. 3J depicts the log (2)-fold change (in sequencing reads per million) values (adjusted for vehicle treatment) relative to early timepoint for 17,255 ORFs after treatment with GSK-J4 for 9 days in G402 cells. The median value of three replicates per concentration is shown. FIG. 3K depicts the log (2)-fold change (in sequencing reads per million) values (adjusted for vehicle treatment) relative to early timepoint for 17,255 ORFs after treatment with ML210 for 9 days in G402 cells. Comparison between two replicates is shown. FIG. 3L depicts the log (2)-fold change (in sequencing reads per million) values (adjusted for vehicle treatment) relative to early timepoint for 17,255 ORFs after treatment with PRIMA-1 for 9 days in G402 cells. Comparison between two replicates is shown. ORFs showing consistent enrichment across replicates are labeled in red in each of FIGS. 3J-3L.

FIGS. 4A to 4T depict how co-inhibition of candidate co-targets and index-therapeutic targets engenders synergistic responses. FIG. 4A shows results of the 72-hour co-treatment of index compound GSK-J4 with vehicle (black) and increasing concentrations of co-target inhibitor JZL184 (blue to red), in COLO800 cells. FIG. 4B shows results of the 72-hour co-treatment of index compound GSK-J4 with vehicle (black) and increasing concentrations of co-target inhibitor JZL184 (blue to red), in A498 cells. FIG. 4C shows results of the 72-hour co-treatment of index compound temozolomide with vehicle (black) and increasing concentrations of co-target inhibitor 06-benzylguanine (O6BG, blue to red), in SIMA cells. FIG. 4D shows results of the 72-hour co-treatment of index compound temozolomide with vehicle (black) and increasing concentrations of co-target inhibitor O6BG (blue to red), in G402 cells. FIG. 4E shows results of the 72-hour co-treatment of index compound paclitaxel with vehicle (black) and increasing concentrations of co-target inhibitor elacridar (blue to red), in SIMA cells. FIG. 4F shows results of the 72-hour co-treatment of index compound paclitaxel with vehicle (black) and increasing concentrations of co-target inhibitor elacridar (blue to red), in CCK81 cells. FIG. 4G shows results of the 72-hour co-treatment of index compound navitoclax with vehicle (black) and increasing concentrations of co-target inhibitor S63845 (blue to red), in RKO cells. FIG. 4H shows results of the 72-hour co-treatment of index compound navitoclax with vehicle (black) and increasing concentrations of co-target inhibitor S63845 (blue to red), in VMCUB1 cells. FIG. 4I shows results of the 72-hour co-treatment of index compound doxorubicin with vehicle (black) and increasing concentrations of co-target inhibitor WEHI-539 (blue to red), in SKLU1 cells. FIG. 4J shows results of the 72-hour co-treatment of index compound doxorubicin with vehicle (black) and increasing concentrations of co-target inhibitor WEHI-539 (blue to red), in HEC1A cells. FIG. 4K shows results of the 72-hour co-treatment of index compound PRIMA-1 with vehicle (black) and increasing concentrations of co-target inhibitor erastin (blue to red), in NCIH1944 cells. FIG. 4L shows results of the 72-hour co-treatment of index compound PRIMA-1 with vehicle (black) and increasing concentrations of co-target inhibitor erastin (blue to red), in SKLU1 cells. FIG. 4M shows results of the 72-hour co-treatment of index compound nicosamide with vehicle (black) and increasing concentrations of co-target inhibitor canagliflozin (blue to red), in SUIT2 cells. FIG. 4N shows results of the 72-hour co-treatment of index compound nicosamide with vehicle (black) and increasing concentrations of co-target inhibitor emodin (blue to red), in SUIT2 cells. FIG. 4O shows results of the 72-hour co-treatment of index compound nicosamide with vehicle (black) and increasing concentrations of co-target inhibitor canagliflozin (blue to red), in SW948 cells. FIG. 4P shows results of the 72-hour co-treatment of index compound nicosamide with vehicle (black) and increasing concentrations of co-target inhibitor emodin (blue to red), in SW948 cells. FIG. 4Q shows the effects of infection of MeWo cells with control or AIFM2-targeting sgRNAs on response to ML210. FIG. 4R shows effects of these sgRNAs on AIFM2 protein levels (by western). GAPDH was used as a loading control. FIG. 4S shows the effects of infection of WM2664 cells with control or IRS2-targeting sgRNAs on response to pictilisib. FIG. 4T shows effects of these sgRNAs on IRS2 protein levels (by western). Beta actin was used as a loading control.

FIGS. 5A to 5G show that combinational drug treatment specifically induces synergy in cell lines with outlier synergistic target expression. FIG. 5A shows the combination of the inhibitor of MGLL with each of 8 compounds across 12 cell lines. FIG. 5B shows the combination of the inhibitor of MGMT with each of 8 compounds across 12 cell lines. FIG. 5C shows the combination of the inhibitor of ABCB1 with each of 8 compounds across 12 cell lines. FIG. 5D shows the combination of the inhibitor of UGT1A10 with each of 8 compounds across 12 cell lines. FIG. 5E shows the combination of the inhibitor of BCL2L1 with each of 8 compounds across 12 cell lines. FIG. 5F shows the combination of the inhibitor of MCL1 with each of 8 compounds across 12 cell lines. FIG. 5G shows the combination of the inhibitor of SLC7A11 with each of 8 compounds across 12 cell lines. For each of FIGS. 5A-5G, degree of sensitization for each drug combination is shown by color. Degree of sensitization is defined as the magnitude of GR₅₀ shift for combination versus single-agent treatment, normalized to the overall range of single-agent GR₅₀ values across the entire cell-line panel. Basal mRNA levels for the named transcript in each panel across each cell line are shown as gray bars.

FIGS. 6A to 6E demonstrate that MGLL activity and expression are sufficient for resistance to GSK-J4. FIG. 6A shows a waterfall plot of GR₅₀ values for GSK-J4 treatment alone and in combination with 1 μM JZL184 across 27 cell lines. FIG. 6B presents basal MGLL expression as a function of relative GR₅₀ values. FIG. 6C shows a comparison of adjusted ICso values of GSK-J4 with and without 1 μM JZL184 across 370 pooled cell lines using PRISM. FIG. 6D depicts the effects on GSK-J4 sensitivity of overexpression of HcRed, MGLL, or the kinase-dead MGLL^{s132A} mutant in G402 cells in the presence or absence of JZL184. FIG. 6E shows the effects of sgRNA and ORF perturbations on GSK-J4 sensitivity in HEC1A cells in the presence or absence of JZL184. M=MGLL, A=MGLL^{S132A}.

FIGS. 7A to 7M show how MGLL modifies GSK-J4 and reduces its anti-proliferative efficacy. FIG. 7A depicts the chemical structures of the canonical MGLL substrate 2-arachidonoylglycerol and products arachidonic acid and glycerol. FIG. 7B depicts the chemical structures of GSK-J4, GSK-J5, and KDOBA67. The ethyl esters required for cleavage (into GSK-J1 and GSK-J2) are highlighted in red. FIG. 7C shows the effect of JZL184 co-treatment on sensitivity to GSK-J4 in MGLL-proficient KMRC3 cells. FIG. 7D shows the effect of JZL184 co-treatment on sensitivity to GSK-J1 (GSK-J4 analog) in MGLL-proficient KMRC3 cells. FIG. 7E shows the effect of JZL184 co-treatment on sensitivity to GSK-J2 (GSK-J4 analog) in MGLL-proficient KMRC3 cells. FIG. 7F shows the effect of JZL184 co-treatment on sensitivity to GSK-J5 (GSK-J4 analog) in MGLL-proficient KMRC3 cells. FIG. 7G shows the effect of JZL184 co-treatment on sensitivity to KDOBA67 (GSK-J4 analog) in MGLL-proficient KMRC3 cells. FIG. 7H shows the effect of GSK-J4 on MGLL-deficient parental G402 cells or MGLL-deficient G402 cells overexpressing HcRed, MGLL, or two kinase-dead MGLL mutants (S132A and D249N). FIG. 7I shows the effect of KDOBA67 (GSK-J4 analog) on MGLL-deficient parental G402 cells or MGLL-deficient G402 cells overexpressing HcRed, MGLL, or two kinase-dead MGLL mutants (S132A and D249N). FIG. 7J shows the IncuCyte time-dependent effects on cell growth for GSK-J4 in KMRC3 cells, as measured by change in phase percent confluence from time=0 values. Increasing concentrations are shown as a blue-to-red scale. FIG. 7K shows the IncuCyte time- and JZL184-dependent effects on cell growth for GSK-J4 and JZL184 in combination in KMRC3 cells, as measured by change in phase percent confluence from time=0 values. FIG. 7L shows the IncuCyte time-dependent effects on cell growth for KDOBA67 in KMRC3 cells, as measured by change in phase percent confluence from time=0 values. FIG. 7M shows the IncuCyte time- and JZL184-dependent effects on cell growth for KDOBA67 and JZL184 in combination in KMRC3 cells, as measured by change in phase percent confluence from time=0 values.

FIGS. 8A to 8C depict identification of resistance-associated transcripts in the GDSC dataset. FIG. 8A shows a pie chart of the number of transcripts (of 17,737) passing a Bonferroni-adjusted- and z-score significance cutoff for at least one small molecule in at least one of 51 cellular sub-contexts (lineage, histology, growth mode, mutation) in the GDSC dataset. FIG. 8B depicts a heatmap showing scaled relative expression across cell lines of most frequently associated genes. Heatmaps were generated using Morpheus oftware.broadinstitute.org/morpheus) with rows and columns clustered (metric: one minus Pearson correlation; linkage method: average). FIG. 8C shows the results from FIG. 8A after filtration for potential confounding factors such as gene co-expression and non-specific associations.

FIGS. 9A and 9B depict prioritized resistance-associated transcripts. FIG. 9A presents x-y scatterplots of sensitivity (AUC) versus gene expression (TPM) for nominated relationships in the CTRP dataset. FIG. 9B shows x-y scatterplots of sensitivity (AUC) versus gene expression (RMA normalization) for nominated relationships in the GDSC dataset.

FIG. 10 shows the validation of single-agent response and protein levels. Sensitivity after 72 hours, as measured by CellTiter-Glo, of a panel of cell lines to each nominated index compound is shown, as are western blots that assessed protein levels. Cell viability values (GR) are normalized to DMSO treatment and to initial (D0) cell number. Expression of each nominated co-target is shown by color (red to blue) and below the panel as bar graphs. For western blots, beta actin (ACTB) or vinculin (VCL) was used as a loading control as indicated and samples were ordered by their relative compound sensitivity.

FIG. 11 presents supporting data for ORF overexpression experiments across additional compounds and cellular models. Effects of lentiviral overexpression of nominated transcripts (red) on compound sensitivity after 72 hours (or 120 hours, for MGMT), as measured by CellTiter-Glo (or live-cell imaging, for MGMT) are shown, and western blots were performed to assess protein levels after overexpression. Parental cell lines are shown in black, with control ORFs in blue. Cell viability values (GR) are normalized to DMSO treatment and to initial (D0) cell number. Each point represents mean±SD (n=2-4 technical replicates) and is representative of at least two independent infections. For western blots, glyceraldehyde-3-phosphate dehydrogenase (GAPDH) or vinculin (VCL) was used as a loading control as indicated.

FIG. 12 presents supporting data for ORF overexpression experiments across additional compounds and cellular models. Effects of lentiviral overexpression of nominated transcripts (red) on compound sensitivity after 72 hours, as measured by CellTiter-Glo are shown, and western blots were performed to assess protein levels after overexpression. Parental cell lines are shown in black, with control ORFs in blue. Cell viability values (GR) are normalized to DMSO treatment and to initial (D0) cell number. Each point represents mean±SD (n=2-4 technical replicates) and is representative of at least two independent infections. For Western blots, vinculin (VCL) was used as a loading control.

FIG. 13 presents supporting data for ORF overexpression experiments across additional compounds and cellular models. Effects of lentiviral overexpression of nominated transcripts (red) on compound sensitivity after 72 hours, as measured by CellTiter-Glo are shown, and western blots were performed to assess protein levels after overexpression. Parental cell lines are shown in black, with control ORFs in blue. Cell viability values (GR) are normalized to DMSO treatment and to initial (D0) cell number. Each point represents mean±SD (n=2-4 technical replicates) and is representative of at least two independent infections. For Western blots, beta actin (ACTB) or vinculin (VCL) was used as a loading control as indicated.

FIGS. 14A to 14S present supporting data for co-inhibition experiments across additional compounds and cellular models, further to FIG. 3 above. FIG. 14A shows 72-hour co-treatment of omacetaxine with vehicle (black) and increasing concentrations of co-target BCL2L1 inhibitor WEHI-539 (blue to red) in the endogenously-proficient SKLU1 cell line. FIG. 14B shows 72-hour co-treatment of omacetaxine with vehicle (black) and increasing concentrations of co-targetBCL2L1 inhibitor WEHI-539 (blue to red) in the endogenously-proficient HEC1A cell line. FIG. 14C shows 72-hour co-treatment of PI-103 with vehicle (black) and increasing concentrations of co-target UGT1A10 inhibitor canagliflozin (blue to red) in the endogenously-proficient SW498 cell line. FIG. 14D shows 72-hour co-treatment of PI-103 with vehicle (black) and increasing concentrations of co-target UGT1A10 inhibitor emodin (blue to red) in the endogenously-proficient SW498 cell line. FIG. 14E shows 72-hour co-treatment of KX2-391 with vehicle (black) and increasing concentrations of co-target ABCB1 inhibitor elacridar (blue to red) in the endogenously-proficient SIMA cell line. FIG. 14F shows 72-hour co-treatment of GSK-J4 with vehicle (black) and increasing concentrations of co-target MGLL inhibitor JZL184 (blue to red) in the ORF-overexpressing G402 cell line. FIG. 14G shows 72-hour co-treatment of PRIMA-1 with vehicle (black) and increasing concentrations of co-target SLC7A11 inhibitor erastin (blue to red) in the ORF-overexpressing G402 cell line. FIG. 14H shows 72-hour co-treatment of carmustine with vehicle (black) and increasing concentrations of co-target MGMT inhibitor O6BG (blue to red) in the ORF-overexpressing WM2664 cell line. FIG. 14I shows 72-hour co-treatment of doxorubicin with vehicle (black) and increasing concentrations of co-target BCL2L1 inhibitor WEHI-539 (blue to red) in the ORF-overexpressing CJM cell line. FIG. 14J shows 72-hour co-treatment of paclitaxel with vehicle (black) and increasing concentrations of co-target ABCB1 inhibitor elacridar (blue to red) in the ORF-overexpressing WM2664 cell line. FIG. 14K shows 72-hour co-treatment of navitoclax with vehicle (black) and increasing concentrations of co-target MCL1 inhibitor S63845 (blue to red) in the ORF-overexpressing HCC1500 cell line. FIG. 14L shows the effects of infection of MeWo cells with control or AIFM2-targeting sgRNAs on response to GSK-J4. FIG. 14M shows the effects of infection of MeWo cells with control or AIFM2-targeting sgRNAs on response to paclitaxel. FIG. 14L shows the effects of infection of MeWo cells with control or A/FM2-targeting sgRNAs on response to doxorubicin. FIG. 14O shows the effects of infection of WM2664 cells with control or IRS2-targeting sgRNAs on response to AZD8055. FIG. 14P shows the effects of infection of WM2664 cells with control or IRS2-targeting sgRNAs on response to dactolisib. FIG. 14O shows the effects of infection of WM2664 cells with control or IRS2-targeting sgRNAs on response to ZSTK474. FIG. 14R presents dependency data from the DepMap portal (www.depmap.org) for BCL2L1 as a function of BCL2L1 expression (blue to red) and measured sensitivity (GR₅₀) to the BCL2L1 inhibitor WEHI-539. FIG. 14S presents dependency data from the DepMap portal (www.depmap.org) for MCL1 as a function of BCL2L1 expression (blue to red) and measured sensitivity (GR₅₀) to the MCL1 inhibitor S63845.

FIGS. 15A to 15D show synergy calculations for co-inhibition experiments where inhibition of the co-target showed significant dose-dependent toxicity. FIG. 15A shows the legend for subsequent FIGS. 15B-15D, where combination effect (deviation from null model) is shown by color. FIG. 15B presents the deviation from Loewe additivity (synergy) for the combination navitoclax×S63845 across a panel of cell lines. The overall F-statistic and p-value (from meanR test) are shown for each cell line. FIG. 15C presents the deviation from Loewe additivity (synergy) for the combination doxorubicin×WEHI-539 across a panel of cell lines. The overall F-statistic and p-value (from meanR test) are shown for each cell line. FIG. 15D presents the deviation from Loewe additivity (synergy) for the combination PRIMA-1×erastin across a panel of cell lines. The overall F-statistic and p-value (from meanR test) are shown for each cell line.

FIG. 16 presents the underlying data for FIG. 5 above. Degree of sensitization for each drug combination is shown relative to the cell line most sensitive to that index compound.

FIGS. 17A to 17P show that MGLL activity and expression are sufficient for resistance to GSK-J4. FIG. 17A presents data of sensitivity after 72 hours, as measured by CellTiter-Glo, of a panel of cell lines to GSK-J4. Cell viability values (GR) are normalized to DMSO treatment and to initial (D0) cell number. FIG. 17B presents data of sensitivity after 72 hours, as measured by CellTiter-Glo, of a panel of cell lines to GSK-J4+1 μM JZL184. Cell viability values (GR) are normalized to DMSO treatment and to initial (D0) cell number. FIG. 17C presents data of sensitivity after 72 hours, as measured by CellTiter-Glo, of a panel of cell lines to JZL184 alone. Cell viability values (GR) are normalized to DMSO treatment and to initial (D0) cell number. FIG. 17D shows copy number (log 2 relative to ploidy) and expression (TPM) values for KDM6A across cell lines from the DepMap portal. FIG. 17E shows copy number (log 2 relative to ploidy) and expression (TPM) values for KDM6B across cell lines from the DepMap portal. FIG. 17F shows median viability values for 370 cell lines from PRISM pooled-cell-line experiment for GSK-J4, GSK-J4+1 μM JZL184 (“combo”), or JZL184 alone as a function of MGLL expression. Low, TPM<2; medium, 2<TPM<4; high, TPM>4. FIG. 17G shows expression (TPM) values for RNAi (DEMETER2) and CRISPR (CERES) for MGLL across cell lines from the DepMap portal. Cell lines are subdivided by CCLE lineage annotation. FIG. 17H shows dependency scores for RNAi (DEMETER2) and CRISPR (CERES) for RNAi (DEMETER2) and CRISPR (CERES) for MGLL across cell lines from the DepMap portal. FIG. 17I depicts concentration-dependent viability values for JZL184 across 242 cell lines from CTRPv1. FIG. 17J shows western blots probing MGLL expression in SUIT2 cells, G402 cells and HEC1A cells, across indicated conditions/point mutant forms of MGLL. FIG. 17K shows the effect on GSK-J4 sensitivity of overexpression of MGLL in MIAPACA2 cells, with or without JZL184 co-treatment. FIG. 17L shows the effect on GSK-J4 sensitivity of infection with sgRNAs targeting MGLL (or control guides) in SUIT2 cells. FIG. 17M shows the effect on GSK-J4 sensitivity of infection with sgRNAs targeting MGLL (or control guides) in HEC1A cells. FIG. 17N further shows the effect on GSK-J4 sensitivity of infection with sgRNAs targeting MGLL (or control guides) in HEC1A cells, without JZL184 co-treatment. FIG. 17O shows the effect on GSK-J4 sensitivity of infection with sgRNAs targeting MGLL (or control guides) in HEC1A cells, with JZL184 co-treatment. FIG. 17P shows western blots probing MGLL expression. Beta actin (ACTB) or vinculin (VCL) was used as a loading control as indicated in FIGS. 17J and 17P.

FIGS. 18A to 18R demonstrate that the effects of MGLL are specific to GSK-J4, and were not observed with other chromatin-modifying compounds or GSK-J4 analogues. FIG. 18A shows the effects of JZL184 co-treatment (blue) on sensitivity to chromatin-modifying compound CPI-455 (annotated target: KDM5) in MGLL-proficient KMRC3 cells. FIG. 18B shows the effects of JZL184 co-treatment (blue) on sensitivity to chromatin-modifying compound KDOAM25 (annotated target: KDM5) in MGLL-proficient KMRC3 cells. FIG. 18C shows the effects of JZL184 co-treatment (blue) on sensitivity to chromatin-modifying compound GSK2879552 (annotated target: KDM1A) in MGLL-proficient KMRC3 cells. FIG. 18D shows the effects of JZL184 co-treatment (blue) on sensitivity to chromatin-modifying compound ORY-1001 (annotated target: KDM1A) in MGLL-proficient KMRC3 cells. FIG. 18E shows the effects of JZL184 co-treatment (blue) on sensitivity to chromatin-modifying compound JIB-04 (annotated target: pan-KDM) in MGLL-proficient KMRC3 cells. FIG. 18F shows the effects of JZL184 co-treatment (blue) on sensitivity to chromatin-modifying compound IOX1 (annotated target: pan-KDM) in MGLL-proficient KMRC3 cells. FIG. 18G shows the effects of JZL184 co-treatment (blue) on sensitivity to chromatin-modifying compound EPZ004777 (annotated target: DOT1L) in MGLL-proficient KMRC3 cells. FIG. 18H shows the effects of JZL184 co-treatment (blue) on sensitivity to chromatin-modifying compound tazemetostat (annotated target: EZH2) in MGLL-proficient KMRC3 cells. FIG. 18I shows the effects of JZL184 co-treatment (blue) on sensitivity to chromatin-modifying compound panobinostat (annotated target: pan-HDAC) in MGLL-proficient KMRC3 cells. FIG. 18J shows the effects of JZL184 co-treatment (blue) on sensitivity to chromatin-modifying compound vorinostat (annotated target: pan-HDAC) in MGLL-proficient KMRC3 cells. FIG. 18K shows the IncuCyte time-dependent effects on cell growth for GSK-J4 in G402 HcRed-overexpressing cells, as measured by change in phase percent confluence from time=0 values. Increasing concentrations are shown as a blue-to-red scale. FIG. 18L shows the IncuCyte time- and JZL184-dependent effects on cell growth for GSK-J4 in G402 HcRed-overexpressing cells, as measured by change in phase percent confluence from time=0 values. Increasing concentrations are shown as a blue-to-red scale. FIG. 18M shows the IncuCyte time-dependent effects on cell growth for GSK-J4 in G402 MGLL-overexpressing cells, as measured by change in phase percent confluence from time=0 values. Increasing concentrations are shown as a blue-to-red scale. FIG. 18N shows the IncuCyte time- and JZL184-dependent effects on cell growth for GSK-J4 in G402 MGLL-overexpressing cells, as measured by change in phase percent confluence from time=0 values. Increasing concentrations are shown as a blue-to-red scale. FIG. 18O shows the IncuCyte time-dependent effects on cell growth for KDOBA67 in G402 HcRed-overexpressing cells, as measured by change in phase percent confluence from time=0 values. Increasing concentrations are shown as a blue-to-red scale. FIG. 18P shows the IncuCyte time- and JZL184-dependent effects on cell growth for KDOBA67 in G402 HcRed-overexpressing cells, as measured by change in phase percent confluence from time=0 values. Increasing concentrations are shown as a blue-to-red scale. FIG. 18Q shows the IncuCyte time-dependent effects on cell growth for KDOBA67 in G402 MGLL-overexpressing cells, as measured by change in phase percent confluence from time=0 values. Increasing concentrations are shown as a blue-to-red scale. FIG. 18R shows the IncuCyte time- and JZL184-dependent effects on cell growth for KDOBA67 in G402 MGLL-overexpressing cells, as measured by change in phase percent confluence from time=0 values. Increasing concentrations are shown as a blue-to-red scale.

FIGS. 19A and 19B present listings relevant to filtered small molecule/mRNA relationships. FIG. 19A presents a key. FIG. 19B specifically presents 768 filtered small molecule/mRNA relationships across 348 unique small molecules and 488 unique transcripts.

FIG. 20 presents transcripts associated with the same compound across two independent datasets (CTRPv2.0 and GDSC).

FIG. 21 depicts consideration of additional genomic features associated with small-molecule response, specifically depicting summarized results from decision-tree-based analyses of the 768 connections in FIG. 19B above.

FIG. 22 presents a listing of prioritized resistance-associated transcripts.

FIG. 23 presents a listing of sgRNA sequences employed in the instant disclosure.

### DETAILED DESCRIPTION OF THE INVENTION

The instant disclosure is based, at least in part, upon the discovery that the basal gene-transcription state of cancer cell lines can be assessed in concert with the cell-viability profiles of single-agent small molecules, to identify specific synergistic drug combinations and identify mechanisms of drug resistance. Compositions involving such newly-identified drug combinations as well as discovery and therapeutic methods related to such discovery are described in additional detail below.

Understanding and predicting variability in initial responses to therapeutic agents is expected to improve cancer patient outcomes. The instant disclosure is based, at least in part, upon identification of a means for leveraging both data presenting the basal gene-transcription state of cancer cell lines with cell-viability profiles of small molecules, to thereby nominate specific mechanisms of, and combinations to overcome, intrinsic resistance. As described in additional detail below, >500,000 sensitivity profiles were analyzed herein to identify >500 candidate compound—gene pairs where outlier expression of a transcript was correlated with resistance to a small molecule. Eight such relationships were validated herein, including certain that are established clinically relevant interactions. One heretofore unappreciated relationship was assessed in particular detail: it was identified herein that endogenous or exogenous expression of MGLL, encoding monoglyceride lipase, conferred resistance to the lysine demethylase inhibitor GSK-J4 and that MGLL-proficient cell lines could be sensitized to GSK-J4 by MGLL inhibitor co-treatment. These initial studies have thereby highlighted the value of integrating gene-expression features with small-molecule response profiles to identify patient populations most likely to benefit from treatment, and to nominate rational candidates for combinations.

As noted above, the genomic causes of variability in initial responses to therapeutic agents are often unclear. The instant disclosure has employed systematic pharmacogenomic analyses to identify transcripts that mark drug-resistant cellular models, are sufficient to confer resistance, and can be inhibited to improve initial sensitivity, thereby not only providing an assortment of combination therapies for further development, but also laying a framework for future drug-combination discovery efforts in cancer.

Despite incredible progress in cancer therapeutics, curing metastatic disease remains an elusive goal. Not all patients will respond to treatment, and those that do will often become drug resistant and will progress on therapy (1). For example, BRAF^{V600E}-mutant melanoma is one of the indications where inhibitors of RAF, MEK and ERK are most effective. However, patient response rates remain in the 60-70% range (2, 3). Although the reasons for incomplete responses in vivo are multifactorial, this general pattern of variable initial response is also observed in cellular models of BRAF^{V600E}-mutant melanoma, which has indicated that at least a fraction of limited response is cancer-cell intrinsic (4). For patients that do initially respond, resistance is nearly inevitable (1). Thus, improving the depth, breadth, and duration of initial responses is critical for improving outcomes in cancer patients with advanced disease.

Drug combinations promise to improve treatment outcomes by increasing initial clinical responses and/or forestalling drug resistance. However, an understanding of which drugs should be combined, and which patients would benefit from a given combination, has heretofore remained largely unknown. Comprehensive empirical drug combination studies remain infeasible: for example, to test all possible combinations of agents from the largest single-agent dataset released to date would require 7×10⁹ individual measurements (496 small molecules across 887 cellular models over 16 concentrations in duplicate). Although recent efforts have made cellular multiplexing a possibility, the combinatorial space remains enormous (5, 6).

Many combination efforts have attempted to alleviate the challenges of scale by reducing the number of concentrations and/or cell lines tested, and/or by using “anchor” therapeutics to test one drug against many, using metrics such as combinatorial synergy to evaluate results (7-10). However, such experiments naturally limit the ability to identify interactions that extend beyond current drug/indication contexts. In addition, such studies are often underpowered for the discovery of genomic features that may one day be useful as clinical biomarkers, informing specific contexts in which drug combinations are likely to be maximally effective. As a consequence, when synergistic combinations have been described, the mechanistic underpinnings often remain elusive, and critical considerations such as relevant concentration(s), selectivity across models or other compounds, and magnitude of effect are not always immediately clear.

It was initially examined herein whether the basal gene-transcription state of cancer cell lines, in concert with the response profiles of hundreds of single-agent small molecules, could be leveraged to nominate therapeutic targets whose expression and function limit initial responses to small molecule inhibitors. It was envisioned that such an approach would identify causal resistance genes and provide a genomic feature that was indicative of primary drug resistance. Moreover, inhibiting the function of the protein products of identified drug-resistance genes would also be predicted to enhance initial drug responses. If successful, such an approach would eliminate the need to test all possible drug/drug combinations across cellular models while concurrently identifying the genetic contexts in which such combinations are likely to be maximally effective. The instant disclosure describes the conception and successful implementation of such an approach. Collectively, beyond the studies and specific combination therapies presented herein, it is contemplated that the instant approach can be leveraged to generate pre-clinical evidence that can be utilized to guide drug-combination trials.

Details regarding various individual components of the combination therapies identified herein include the following.

KDM6A/B Inhibitors

Known KDM6A/B inhibitors include GSK-J4, as well as IOX1, GSK-J1 and Caffeic acid. Such agents can be used to treat, e.g., AML, ALL (including T-ALL), prostate cancer, among other types of cancer.

GSK-J4 is an inhibitor of H3K27me3/me2-demethylases and has the following structure:

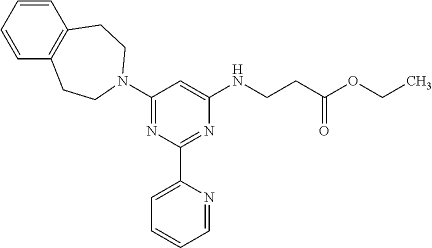 [SMILES: CCOC(=O)CCNc1cc(N2CCc3ccccc3CC2)nc(-c2ccccn2)n1]

Exemplary administration concentrations of GSK-J4 include 10 nM and 25 nM, with an exemplary stock solution of 42 mg/mL (100 nM) for GSK-J4.

IOX1 has the following structure:

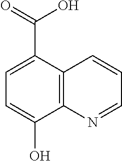 [SMILES: O=C(O)c1ccc(O)c2ncccc12]

GSK-J1 has the following structure:

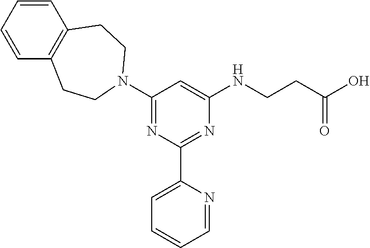 [SMILES: O=C(O)CCNc1cc(N2CCc3ccccc3CC2)nc(-c2ccccn2)n1]

Caffeic acid has the following structure:

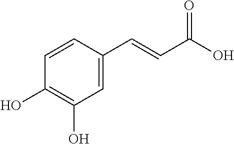 [SMILES: O=C(O)/C=C/c1ccc(O)c(O)c1]

MGLL Inhibitors

Monoglyceride lipase (MGL) is the main enzyme responsible for the hydrolytic deactivation of the endocannabinoid 2-arachidonoyl-sn-glycerol (2-AG), and is an intracellular serine hydrolase that plays critical roles in many physiological and pathological processes, such as pain, inflammation, neuroprotection and cancer. Several classes of MGL inhibitors have been developed, from early reversible ones, such as URB602 and pristimerin, to carbamoylating agents that react with the catalytic serine, such as JZL184 and more recent 0-hexafluoroisopropyl carbamates. Other inhibitors that modulate MGL activity by interacting with conserved regulatory cysteines act through unknown mechanisms. Inhibition of MGLL can therefore be performed in a cell, tissue and/or subject using agents known in the art, including, e.g., JZL 184, URB602, pristimerin, 0-hexafluorosiopropyl carbamates, etc., as well as via administration of targeted oligonucleotide inhibitors (e.g., antisense and/or RNAi moieties, including siRNA, DsiRNA, shRNA, etc.).

JZL 184 (4-nitrophenyl 4-(dibenzo[d][1,3]dioxol-5-yl(hydroxy)methyl)piperidine-1-carboxylate) has the following structure:

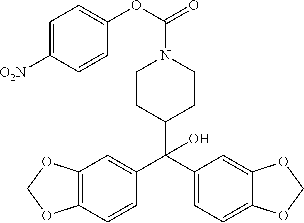 [SMILES: O=C(Oc1ccc([N+](=O)[O-])cc1)N1CCC(C(O)(c2ccc3c(c2)OCO3)c2ccc3c(c2)OCO3)CC1]

An exemplary dosage for JZL 184 is 12 mg/kg, i.p. JZL184 inhibits Monoacylglycerol lipase (MAGL) by irreversible active-site carbamoylation and 75% of MAGL is inhibited at a dose of 4 mg/kg and a near-complete blockade of MAGL occurs at a dose of 16 mg/kg (Long et al.).

URB602 has the following structure:

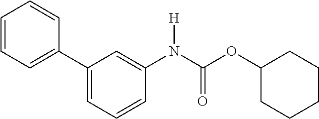 [SMILES: O=C(Nc1cccc(-c2ccccc2)c1)OC1CCCCC1]

Pristimerin has the following structure:

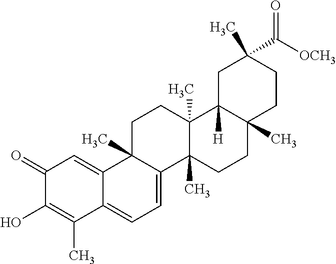

ERK1 Inhibitors

Known ERK1 inhibitors include SCH772984, LY3214996, GDC-0994, FR 180204 and pluripotin.

SCH772984 is an ERK1 inhibitor having the following structure:

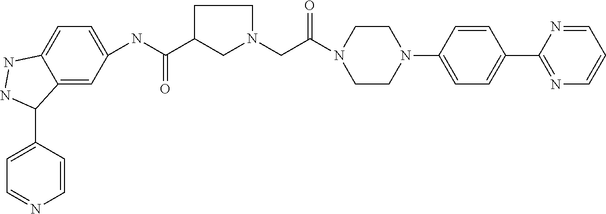 [SMILES: O=C(Nc1ccc2c(c1)C(c1ccncc1)NN2)C1CCN(CC(=O)N2CCN(c3ccc(-c4ncccn4)cc3)CC2)C1]

LY3214996 is an ERK1 inhibitor having the following structure:

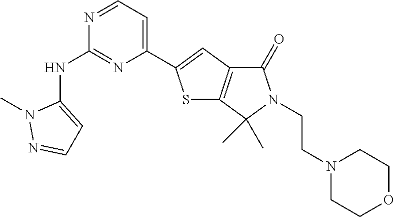 [SMILES: Cn1nccc1Nc1nccc(-c2cc3c(s2)C(C)(C)N(CCN2CCOCC2)C3=O)n1]

GDC-0994 is an ERK1 inhibitor having the following structure:

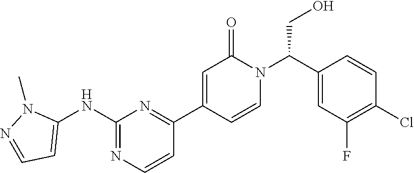 [SMILES: Cn1nccc1Nc1nccc(-c2ccn([C@H](CO)c3ccc(Cl)c(F)c3)c(=O)c2)n1]

FR 180204 is an ERK1 inhibitor having the following structure:

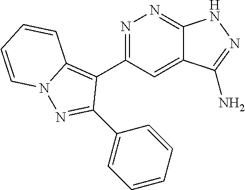 [SMILES: Nc1n[nH]c2nnc(-c3c(-c4ccccc4)nn4ccccc34)cc12]

Pluripotin is an ERK1 inhibitor having the following structure:

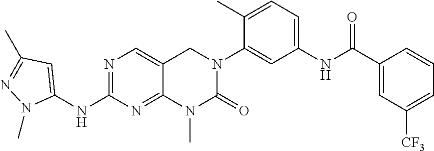 [SMILES: Cc1cc(Nc2ncc3c(n2)N(C)C(=O)N(c2cc(NC(=O)c4cccc(C(F)(F)F)c4)ccc2C)C3)n(C)n1]

ERK1/2 inhibitors are currently being used, e.g., in clinical trials for treatment of advanced or metastatic cancer (solid tumors), including advanced or metastatic cancer with an activating mitogen-activated protein kinase pathway alteration, BRAF mutant metastatic melanoma refractory to or relapsed after treatment with RAF and/or MEK inhibitors, Metastatic melanoma with an NRAS mutation; advanced, unresectable cancer and advanced, unresectable, or metastatic non-small cell lung cancer (NSCLC) with a BRAF or RAS mutation; colorectal cancer with a RAS mutation; and metastatic pancreatic ductal adenocarcinoma, among other forms of cancer.

ICMT Inhibitors

Inhibition of ICMT can be performed in a cell, tissue and/or subject using agents known in the art, including, e.g., (E,E)-Farnesyl Thiol, ICMT inhibitor 420350, ICMT inhibitor 420351 and cysmethynil, as well as via administration of targeted oligonucleotide inhibitors (e.g., antisense and/or RNAi moieties, including siRNA, DsiRNA, shRNA, etc.).

(E,E)-Farnesyl Thiol has the following structure:

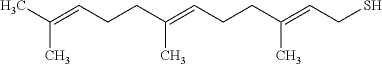 [SMILES: CC(C)=CCC/C(C)=C/CC/C(C)=C/CS]

ICMT inhibitor 420350 has the following structure:

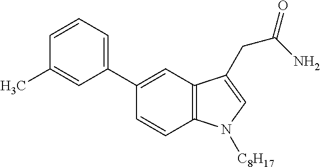

ICMT inhibitor 420351 has the following structure:

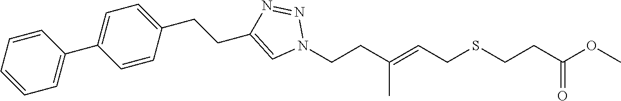 [SMILES: COC(=O)CCSC/C=C(\C)CCn1cc(CCc2ccc(-c3ccccc3)cc2)nn1]

Cysmethynil has the following structure:

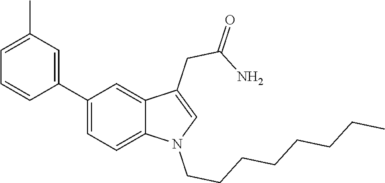 [SMILES: CCCCCCCCn1cc(CC(N)=O)c2cc(-c3cccc(C)c3)ccc21]

CDK4/6 Inhibitors

CDK4 and CDK6 are cyclin-dependent kinases that control the transition between the G1 and S phases of the cell cycle. The S phase is the period during which the cell synthesizes new DNA and prepares itself to divide during the process of mitosis. CDK4/6 activity is typically deregulated and overactive in cancer cells. There can be amplification or overexpression of the genes encoding cyclins or of the genes encoding the CDKs themselves. Additionally, loss of endogenous INK4 inhibitors, by gene deletion, mutation, or promoter hypermethylation, can also lead to overactivity of CDK4 and CDK6. Known CDK4/6 inhibitors include flavopiridol, abemaciclib, ribociclib and palbociclib.

Flavopiridol has the following structure:

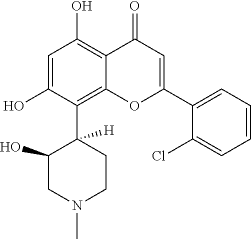 [SMILES: CN1CC[C@H](c2c(O)cc(O)c3c(=O)cc(-c4ccccc4Cl)oc23)[C@H](O)C1]

Abemaciclib has the following structure:

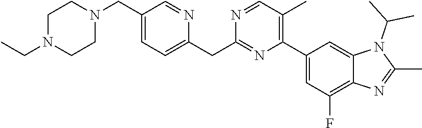 [SMILES: CCN1CCN(Cc2ccc(Cc3ncc(C)c(-c4cc(F)c5nc(C)n(C(C)C)c5c4)n3)nc2)CC1]

Ribociclib has the following structure:

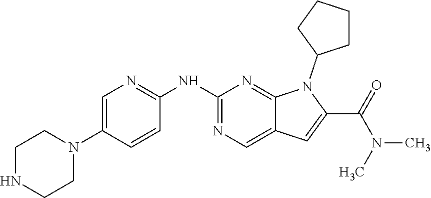 [SMILES: CN(C)C(=O)c1cc2cnc(Nc3ccc(N4CCNCC4)cn3)nc2n1C1CCCC1]

Palbociclib has the following structure:

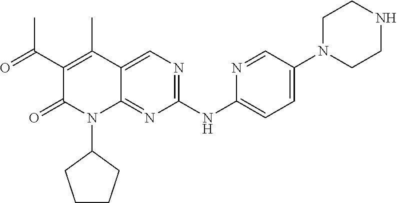 [SMILES: CC(=O)c1c(C)c2cnc(Nc3ccc(N4CCNCC4)cn3)nc2n(C2CCCC2)c1=O]

Abemaciclib, ribociclib and palbociclib have been FDA approved for the treatment of hormone receptor (HR)-positive, human epidermal growth factor receptor 2 (HER2)-negative advanced or metastatic breast cancer that has progressed after endocrine therapy.

CDK4/6 inhibitors have been described for use in a number of cancers, including breast cancer, non-small cell lung cancer (NSCLC) (especially the KRAS-mutant subset, as KRAS-driven lung cancer has been identified as highly dependent on CDK4, and genetic or pharmacologic ablation of CDK4 activity has effects on both the establishment and maintenance of these tumors), mantle cell lymphoma (a malignancy defined by a translocation involving CCND1 resulting in cyclin D1 overexpression), liposarcoma (in which CDK4 is often amplified), melanoma, glioblastoma, pancreatic cancer, and colorectal cancer, among others.

CCNE1 Inhibitors

Inhibition of CCNE1 can be performed in a cell, tissue and/or subject using agents known in the art, including, e.g., via administration of CCNE1-targeted oligonucleotide inhibitors (e.g., antisense and/or RNAi moieties, including siRNA, DsiRNA, shRNA, etc.).

AKT1/2 Inhibitors

Known AKT1/2 inhibitors include BAY1125976 and AKT inhibitor VIII (isozyme-selective), among other Akt inhibitors.

BAY1125976 has the following structure:

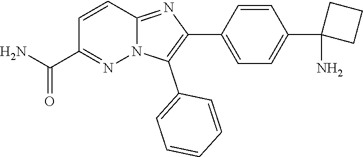 [SMILES: NC(=O)c1ccc2nc(-c3ccc(C4(N)CCC4)cc3)c(-c3ccccc3)n2n1]

AKT inhibitor VIII (isozyme-selective) has the following structure:

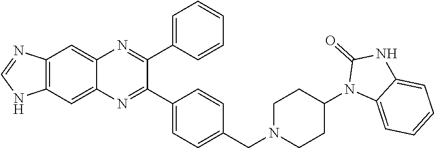 [SMILES: O=c1[nH]c2ccccc2n1C1CCN(Cc2ccc(-c3nc4cc5[nH]cnc5cc4nc3-c3ccccc3)cc2)CC1]

AKT inhibitor VIII has been noted as preferentially inhibiting Akt1 and Akt2 over Akt3, with IC50 values of 3.5, 40 and 1900 nM, respectively (Bilodeau M T et al.).

AKT1/2 inhibitors have been described for use in a number of cancers, including in breast cancers, head and neck squamous cell carcinomas, endometrial cancer, non-small cell lung cancer (NSCLC), renal cancers, gastric cancers, ovarian cancers, pancreatic cancers, colon cancers, oesophageal cancers and thyroid cancers.

AKT3 Inhibitors

Inhibition of AKT3 can be performed in a cell, tissue and/or subject using agents known in the art, including, e.g., via administration of targeted oligonucleotide inhibitors (e.g., antisense and/or RNAi moieties, including siRNA, DsiRNA, shRNA, etc.).

Topoisomerase II Inhibitors

Topoisomerase inhibitors are chemical compounds that block the action of topoisomerase (topoisomerase I and II), which are enzymes that control the changes in DNA structure (Dorlands Medical Dictionary: topoisomerase inhibitor) by catalyzing the breaking and rejoining of the phosphodiester backbone of DNA strands during the normal cell cycle.

Topoisomerases have become popular targets for cancer chemotherapy treatments. Without wishing to be bound by theory, it is thought that topoisomerase inhibitors block the ligation step of the cell cycle, generating single and double stranded breaks that harm the integrity of the genome. Introduction of these breaks subsequently leads to apoptosis and cell death.

Topoisomerase inhibitors can also function as antibacterial agents (Mitscher. Chemical Reviews 105: 559-92). Quinolones (including nalidixic acid and ciprofloxacin) have this function (Fisher and Pan. New Antibiotic Targets, Methods in Molecular Medicine 142: 11-23). Quinolones bind to these enzymes and prevent them from decatenation replicating DNA.

Topoisomerase II inhibitors include etoposide (VP-16), teniposide, doxorubicin, daunorubicin, mitoxantrone, amsacrine, ellipticines, aurintricarboxylic acid (Benchokroun et al. Biochemical Pharmacology. 49: 305-13), and HU-331, a quinolone synthesized from cannabidiol.

Topoisomerase II inhibitors are split into two main classes: topoisomerase poisons, which target the topoisomerase-DNA complex, and topoisomerase inhibitors, which disrupt catalytic turnover. Examples of topoisomerase poisons include:
Eukaryotic type II topoisomerase inhibitors (topo II): amsacrine, etoposide, etoposide phosphate, teniposide and doxorubicin. These drugs are anti-cancer therapies.
Bacterial type II topoisomerase inhibitors (gyrase and topo IV): fluoroquinolones. These are antibacterials and include fluoroquinolones such as ciprofloxacin.

Some of these poisons encourage the forward cleavage reaction (fluoroquinolones), while other poisons prevent the re-ligation of DNA (etoposide and teniposide). Interestingly, poisons of type IIA topoisomerases can target prokaryotic and eukaryotic enzymes preferentially, making them attractive drug candidates. Ciprofloxacin targets prokaryotes in excess of a thousandfold more than it targets eukaryotic topo IIs. Despite this, Ciprofloxacin is a potent dose-dependent type II poison, which is why it causes mass destruction of tissue cells (Mukherjee et al. Mutation Research Letters. 301: 87-92). This poor safety profile is one reason the FDA has advised fluoroquinolones only be used as a treatment of last resort.

Topo II inhibitors that target the N-terminal ATPase domain of topo II and prevent topo II from turning over include ICRF-193 (Robinson et al. Cell Cycle. 6: 1265-7) and genistein. The structure of ICRF-193 bound to the ATPase domain was solved by Classen (Proceedings of the National Academy of Sciences, 2004) showing that the drug binds in a non-competitive manner and locks down the dimerization of the ATPase domain (Baird et al. Journal of Biological Chemistry. 276: 27893-8).

Amsacrine, etoposide, teniposide and doxorubicin structures include the following:

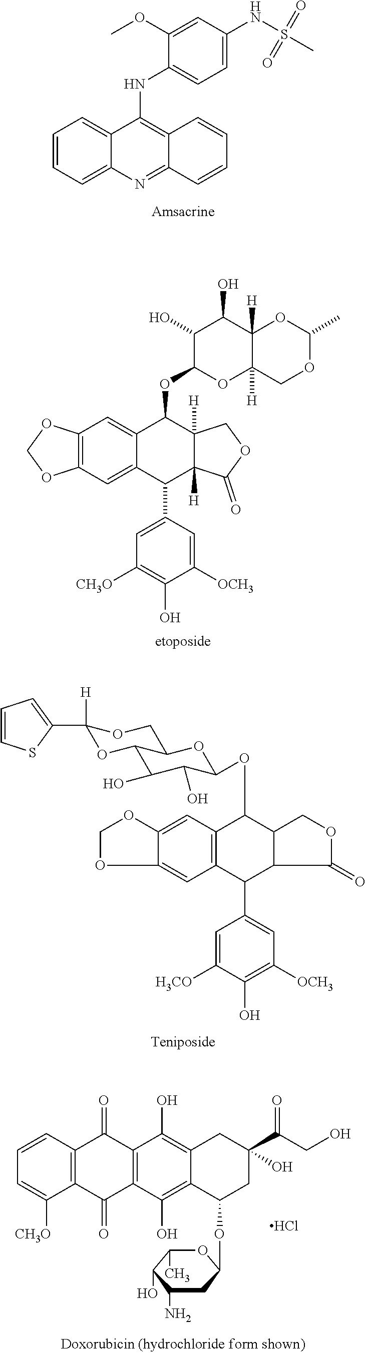

ICRF-193 has the following structure:

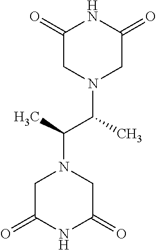 [SMILES: C[C@H]([C@H](C)N1CC(=O)NC(=O)C1)N1CC(=O)NC(=O)C1]

Genistein has the following structure:

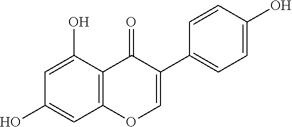 [SMILES: O=c1c(-c2ccc(O)cc2)coc2cc(O)cc(O)c12]

Cancers commonly treated with topoisomerase II inhibitors include breast cancer, bladder cancer, Kaposi's sarcoma, lymphoma, acute lymphocytic leukemia and colorectal cancer, among others.

BCL2L1 Inhibitors

Inhibition of BCL2L1 can be performed in a cell, tissue and/or subject using agents known in the art, including, e.g., Z36, 2,3-DCPE hydrochloride, arctigenin, (±)-gossypol, gossypol-acetic acid and R(−)-gossypol, as well as via administration of targeted oligonucleotide inhibitors (e.g., antisense and/or RNAi moieties, including siRNA, DsiRNA, shRNA, etc.).

Z36 has the following structure:

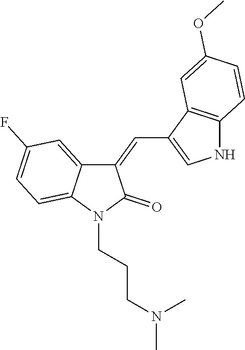 [SMILES: COc1ccc2[nH]cc(/C=C3\C(=O)N(CCCN(C)C)c4ccc(F)cc43)c2c1]

2,3-DCPE hydrochloride has the following structure:

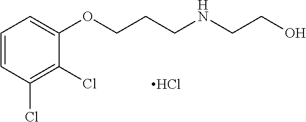 [SMILES: OCCNCCCOc1cccc(Cl)c1Cl ; Cl]

Arctigenin has the following structure:

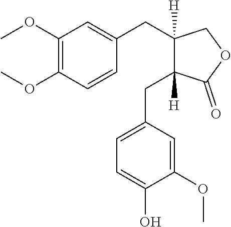 [SMILES: COc1cc(C[C@H]2C(=O)OC[C@@H]2Cc2ccc(OC)c(OC)c2)ccc1O]

(±)-Gossypol has the following structure:

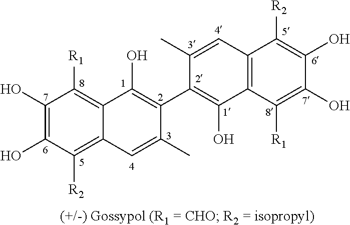

gossypol-acetic acid has the following structure:

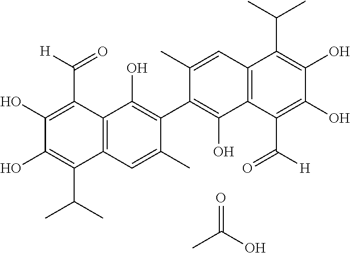

R(−)-gossypol has the following structure:

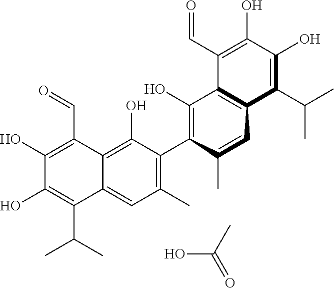

STAT3 Signaling Inhibitors

Known STAT3 signaling inhibitors include niclosamide (5-Chloro-N-(2-chloro-4-nitrophenyl)-2-hydroxybenzamide), S31-201, stattic, nifuroxazide, C188-9, SH-4-54, napabucasin, artesunate, BP-1-102, cryptotanshinone, SH5-07 (SH-5-07), ochromycinone (STA-21), APTSTAT3-9R and HO-3867.

Niclosamide has the following structure:

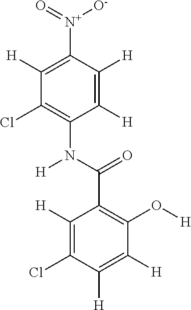 [SMILES: O=C(Nc1ccc([N+](=O)[O-])cc1Cl)c1cc(Cl)ccc1O]

S31-201 has the following structure:

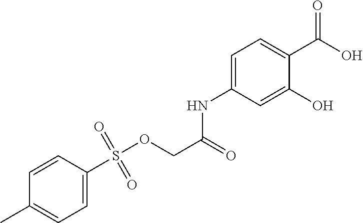 [SMILES: Cc1ccc(S(=O)(=O)OCC(=O)Nc2ccc(C(=O)O)c(O)c2)cc1]

Stattic has the following structure:

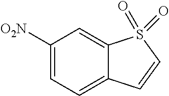 [SMILES: O=[N+]([O-])c1ccc2c(c1)S(=O)(=O)C=C2]

Nifuroxazide has the following structure:

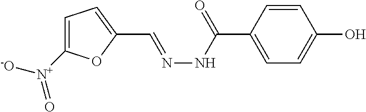

C188-9 has the following structure:

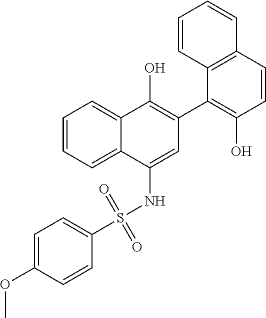 [SMILES: COc1ccc(S(=O)(=O)Nc2cc(-c3c(O)ccc4ccccc34)c(O)c3ccccc23)cc1]

SH-4-54 has the following structure:

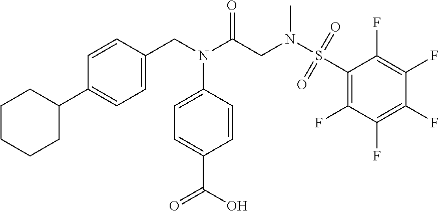 [SMILES: CN(CC(=O)N(Cc1ccc(C2CCCCC2)cc1)c1ccc(C(=O)O)cc1)S(=O)(=O)c1c(F)c(F)c(F)c(F)c1F]

Napabucasin has the following structure:

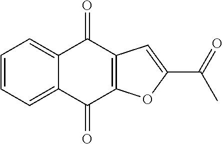 [SMILES: CC(=O)c1cc2c(o1)C(=O)c1ccccc1C2=O]

Artesunate has the following structure:

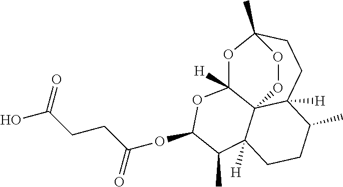 [SMILES: C[C@H]1[C@@H](OC(=O)CCC(=O)O)O[C@@H]2O[C@]3(C)CC[C@H]4[C@H](C)CC[C@@H]1[C@@]24OO3]

BP-1-102 has the following structure:

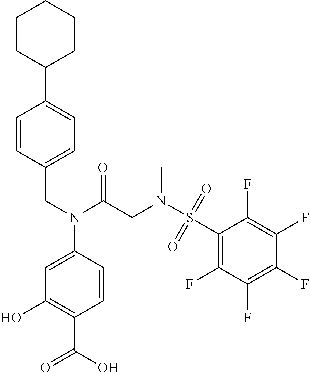 [SMILES: CN(CC(=O)N(Cc1ccc(C2CCCCC2)cc1)c1ccc(C(=O)O)c(O)c1)S(=O)(=O)c1c(F)c(F)c(F)c(F)c1F]

Cryptotanshinone has the following structure:

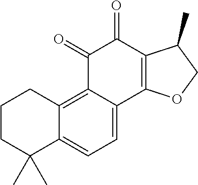 [SMILES: C[C@H]1COC2=C1C(=O)C(=O)c1c2ccc2c1CCCC2(C)C]

SH5-07 (SH-5-07) has the following structure:

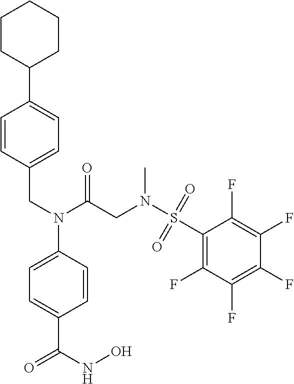 [SMILES: CN(CC(=O)N(Cc1ccc(C2CCCCC2)cc1)c1ccc(C(=O)NO)cc1)S(=O)(=O)c1c(F)c(F)c(F)c(F)c1F]

Ochromycinone (STA-21) has the following structure:

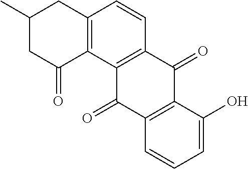 [SMILES: CC1CC(=O)c2c(ccc3c2C(=O)c2cccc(O)c2C3=O)C1]

HO-3867 has the following structure:

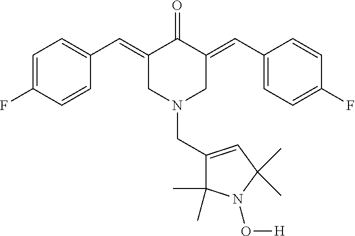 [SMILES: CC1(C)C=C(CN2C/C(=C\c3ccc(F)cc3)C(=O)/C(=C/c3ccc(F)cc3)C2)C(C)(C)N1O]

STAT3 has been described as an attractive target for drug development to treat many types of cancer including breast cancer, head and neck squamous cell carcinoma (HNSCC), non-small cell lung cancer (NSCLC), hepatocellular carcinoma (HCC), colorectal cancer (CRC), gastric adenocarcinoma and melanoma, among other forms of cancer.

UGT1A10 Inhibitors

Inhibition of UGT1A10 can be performed in a cell, tissue and/or subject using agents known in the art, including, e.g., via administration of targeted oligonucleotide inhibitors (e.g., antisense and/or RNAi moieties, including siRNA, DsiRNA, shRNA, etc.).

PI3 Kinase Inhibitors

A large variety of PI3 kinase inhibitors are known in the art. Exemplary PI3 kinase inhibitors include Pictilisib, Idelalisib (FDA approved for leukemia and two types of lymphoma), Copanlisib (FDA approved for the treatment of adult patients with relapsed follicular lymphoma (FL) who have received at least two prior systemic therapies), Taselisib, Perifosine, Buparlisib (BKM120), Duvelisib (IPI-145), Alpelisib (BYL719), Umbralisib, (TGR 1202), Copanlisib (BAY 80-6946), PX-866, Dactolisib, CUDC-907, Voxtalisib, CUDC-907, ME-401, IPI-549, SF1126, RP6530, INK1117, XL147 (also known as SAR245408), Palomid 529, GSK1059615, ZSTK474, PWT33597, IC87114, TG100-115, CAL263, RP6503, PI-103, GNE-477 and AEZS-136.

Pictilisib has the following structure:

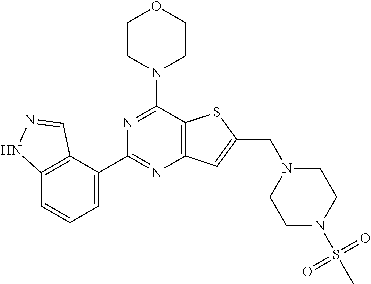 [SMILES: CS(=O)(=O)N1CCN(Cc2cc3nc(-c4cccc5[nH]ncc45)nc(N4CCOCC4)c3s2)CC1]

Idelalisib (FDA approved for leukemia and two types of lymphoma) has the following structure:

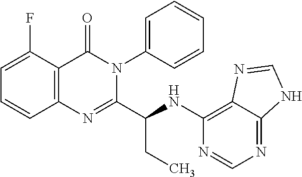 [SMILES: CC[C@H](Nc1ncnc2[nH]cnc12)c1nc2cccc(F)c2c(=O)n1-c1ccccc1]

Copanlisib (FDA approved for the treatment of adult patients with relapsed follicular lymphoma (FL) who have received at least two prior systemic therapies) has the following structure:

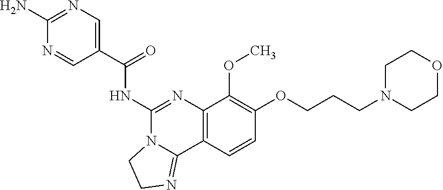 [SMILES: COc1c(OCCCN2CCOCC2)ccc2c1N=C(NC(=O)c1cnc(N)nc1)N1CCN=C21]

Taselisib has the following structure:

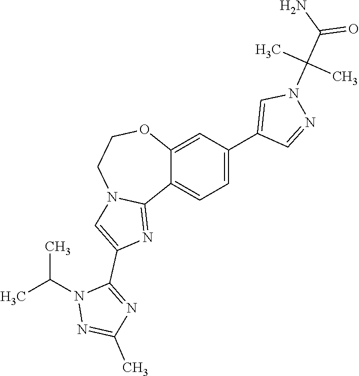 [SMILES: Cc1nc(-c2cn3c(n2)-c2ccc(-c4cnn(C(C)(C)C(N)=O)c4)cc2OCC3)n(C(C)C)n1]

Perifosine has the following structure:

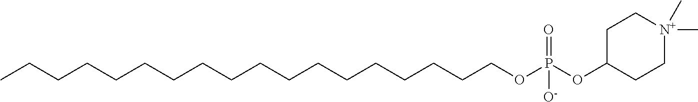 [SMILES: CCCCCCCCCCCCCCCCCCOP(=O)([O-])OC1CC[N+](C)(C)CC1]

Buparlisib (BKM120) has the following structure:

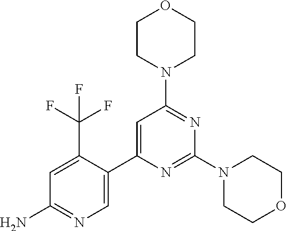 [SMILES: Nc1cc(C(F)(F)F)c(-c2cc(N3CCOCC3)nc(N3CCOCC3)n2)cn1]

Duvelisib (IPI-145) has the following structure:

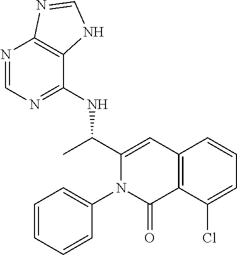 [SMILES: C[C@H](Nc1ncnc2nc[nH]c12)c1cc2cccc(Cl)c2c(=O)n1-c1ccccc1]

Alpelisib (BYL719) has the following structure:

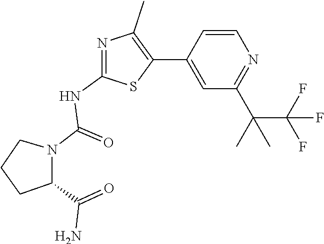 [SMILES: Cc1nc(NC(=O)N2CCC[C@H]2C(N)=O)sc1-c1ccnc(C(C)(C)C(F)(F)F)c1]

Umbralisib, (TGR 1202) has the following structure:

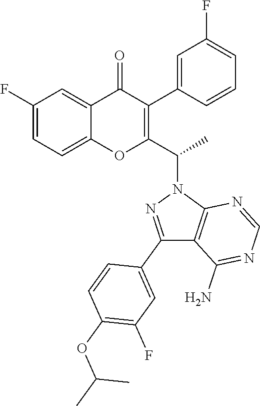 [SMILES: CC(C)Oc1ccc(-c2nn([C@@H](C)c3oc4ccc(F)cc4c(=O)c3-c3cccc(F)c3)c3ncnc(N)c23)cc1F]

PX-866 has the following structure:

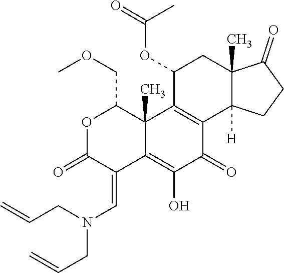 [SMILES: C=CCN(/C=C1\C(=O)O[C@H](COC)[C@@]2(C)C1=C(O)C(=O)C1=C2[C@H](OC(C)=O)C[C@]2(C)C(=O)CC[C@@H]12)CC=C]

Dactolisib has the following structure:

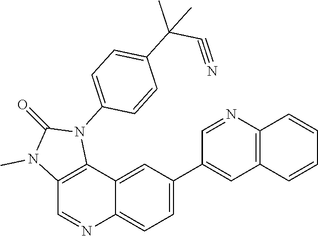 [SMILES: Cn1c(=O)n(-c2ccc(C(C)(C)C#N)cc2)c2c3cc(-c4cnc5ccccc5c4)ccc3ncc21]

CUDC-907 has the following structure:

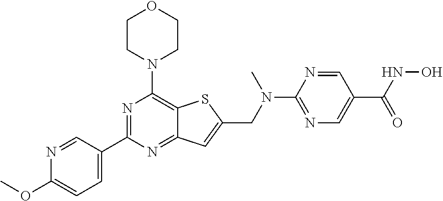 [SMILES: COc1ccc(-c2nc(N3CCOCC3)c3sc(CN(C)c4ncc(C(=O)NO)cn4)cc3n2)cn1]

Voxtalisib has the following structure:

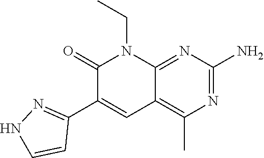 [SMILES: CCn1c(=O)c(-c2cc[nH]n2)cc2c(C)nc(N)nc21]

CUDC-907 has the following structure:

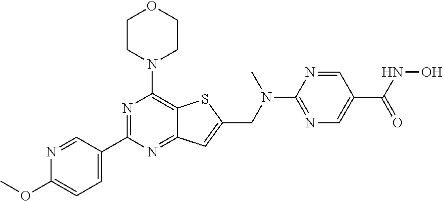 [SMILES: COc1ccc(-c2nc(N3CCOCC3)c3sc(CN(C)c4ncc(C(=O)NO)cn4)cc3n2)cn1]

ME-401 has the following structure:

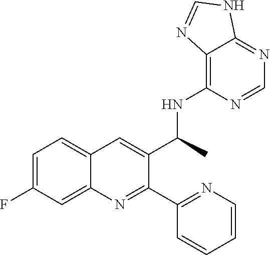 [SMILES: C[C@H](Nc1ncnc2[nH]cnc12)c1cc2ccc(F)cc2nc1-c1ccccn1]

IPI-549 has the following structure:

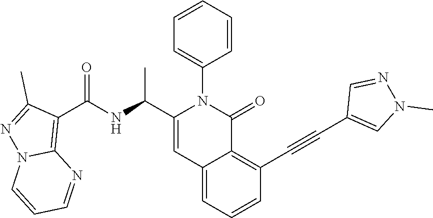 [SMILES: Cc1nn2cccnc2c1C(=O)N[C@@H](C)c1cc2cccc(C#Cc3cnn(C)c3)c2c(=O)n1-c1ccccc1]

SF1126 has the following structure:

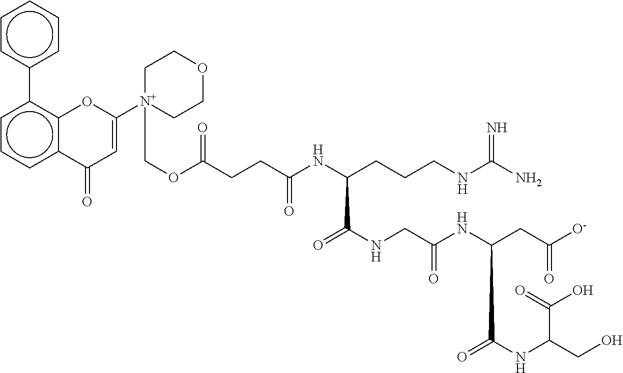

RP6530 has the following structure:

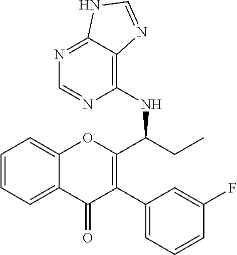 [SMILES: CC[C@H](Nc1ncnc2[nH]cnc12)c1oc2ccccc2c(=O)c1-c1cccc(F)c1]

INK1117 has the following structure:

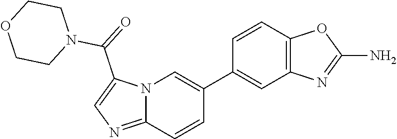 [SMILES: Nc1nc2cc(-c3ccc4ncc(C(=O)N5CCOCC5)n4c3)ccc2o1]

XL147 (also known as SAR245408) has the following structure:

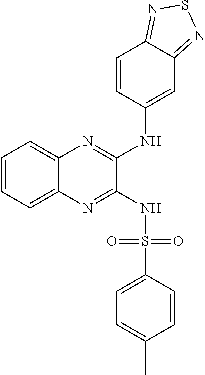 [SMILES: Cc1ccc(S(=O)(=O)Nc2nc3ccccc3nc2Nc2ccc3nsnc3c2)cc1]

Palomid 529 has the following structure:

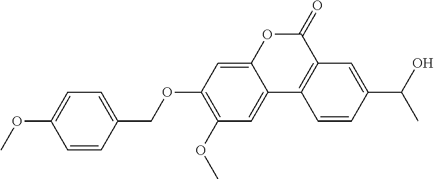 [SMILES: COc1ccc(COc2cc3oc(=O)c4cc(C(C)O)ccc4c3cc2OC)cc1]

GSK1059615 has the following structure:

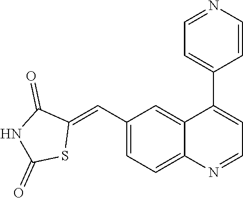 [SMILES: O=C1NC(=O)/C(=C/c2ccc3nccc(-c4ccncc4)c3c2)S1]

ZSTK474 has the following structure:

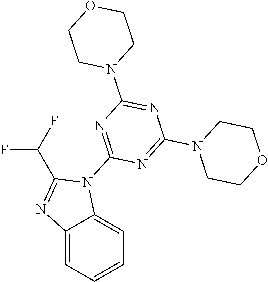 [SMILES: FC(F)c1nc2ccccc2n1-c1nc(N2CCOCC2)nc(N2CCOCC2)n1]

PWT33597 has the following structure:

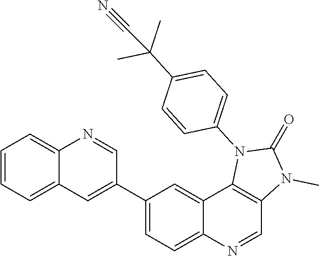 [SMILES: Cn1c(=O)n(-c2ccc(C(C)(C)C#N)cc2)c2c3cc(-c4cnc5ccccc5c4)ccc3ncc21]

IC87114 has the following structure:

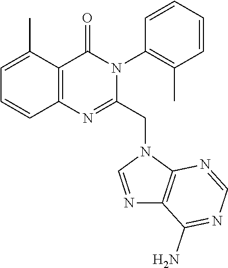 [SMILES: Cc1ccccc1-n1c(Cn2cnc3c(N)ncnc32)nc2cccc(C)c2c1=O]

TG100-115 has the following structure:

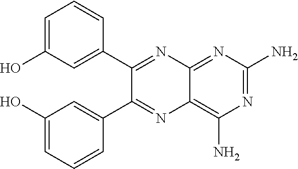 [SMILES: Nc1nc(N)c2nc(-c3cccc(O)c3)c(-c3cccc(O)c3)nc2n1]

RP6503 has the following structure:

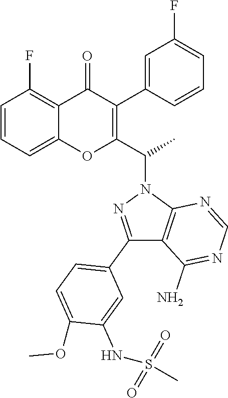

PI-103 has the following structure:

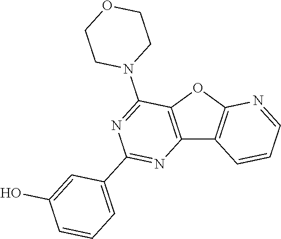 [SMILES: Oc1cccc(-c2nc(N3CCOCC3)c3oc4ncccc4c3n2)c1]

GNE-477 has the following structure:

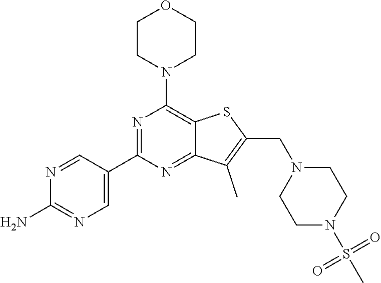 [SMILES: Cc1c(CN2CCN(S(C)(=O)=O)CC2)sc2c(N3CCOCC3)nc(-c3cnc(N)nc3)nc12]

PI3 kinase inhibitors have been described and tested for therapeutic benefit against a variety of cancers, including leukemia, breast cancer, lung cancer, CLL, colorectal cancer, hematologic malignancies, thyroid cancer, inflammatory conditions, multiple myeloma, and lymphoma, including B-cell lymphomas, e.g., CLL and follicular lymphoma.

IRS2 Inhibitors

Inhibition of IRS2 can be performed in a cell, tissue and/or subject using agents known in the art, including, e.g., via administration of targeted oligonucleotide inhibitors (e.g., antisense and/or RNAi moieties, including siRNA, DsiRNA, shRNA, etc.).

GPX4 Inhibitors

Known GPX4 inhibitors include ML210, racemic RSL3, (1S,3R)-RSL3, ML162, CIL56, DPI19, DPI18, DPI17, DPI13, DPI12, altretamine and FIN56.

ML210 has the following structure:

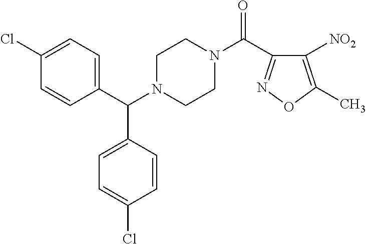 [SMILES: Cc1onc(C(=O)N2CCN(C(c3ccc(Cl)cc3)c3ccc(Cl)cc3)CC2)c1[N+](=O)[O-]]

(1S,3R)-RSL3 has the following structure:

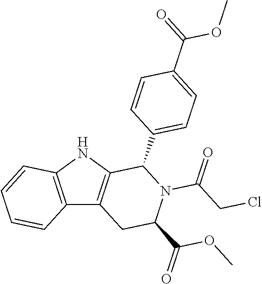 [SMILES: COC(=O)c1ccc([C@H]2c3[nH]c4ccccc4c3C[C@H](C(=O)OC)N2C(=O)CCl)cc1]

ML162 has the following structure:

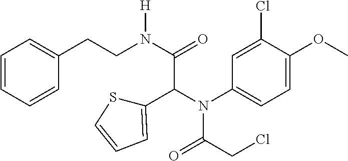 [SMILES: COc1ccc(N(C(=O)CCl)C(C(=O)NCCc2ccccc2)c2cccs2)cc1Cl]

CIL56 has the following structure:

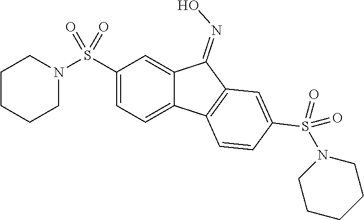 [SMILES: O=S(=O)(c1ccc2c(c1)C(=NO)c1cc(S(=O)(=O)N3CCCCC3)ccc1-2)N1CCCCC1]

DPI19 has the following structure:

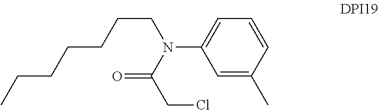 [SMILES: CCCCCCCN(C(=O)CCl)c1cccc(C)c1]

DPI18 has the following structure:

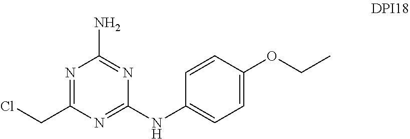 [SMILES: CCOc1ccc(Nc2nc(N)nc(CCl)n2)cc1]

DPI17 has the following structure:

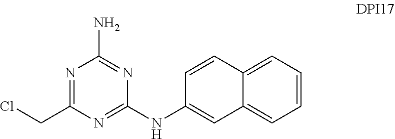 [SMILES: Nc1nc(CCl)nc(Nc2ccc3ccccc3c2)n1]

DPI13 has the following structure:

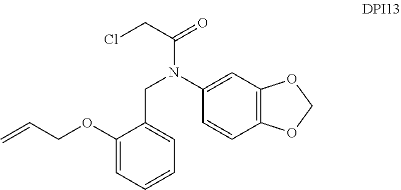 [SMILES: C=CCOc1ccccc1CN(C(=O)CCl)c1ccc2c(c1)OCO2]

DPI12 has the following structure:

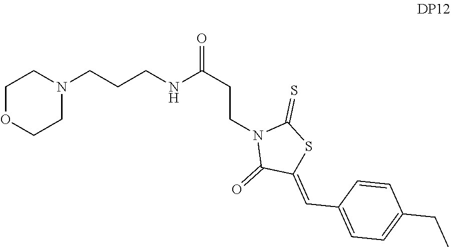 [SMILES: CCc1ccc(/C=C2\SC(=S)N(CCC(=O)NCCCN3CCOCC3)C2=O)cc1]

Altretamine has the following structure:

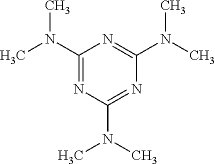

FIN56 has the following structure:

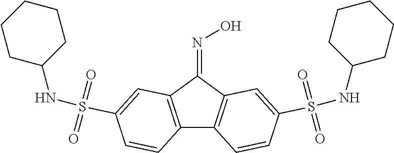 [SMILES: O=S(=O)(NC1CCCCC1)c1ccc2c(c1)C(=NO)c1cc(S(=O)(=O)NC3CCCCC3)ccc1-2]

GPX4 inhibitors have been described for therapeutic use in diffuse large B cell lymphomas (DLBCLs) and renal cell carcinomas, among other forms of cancer.

AIFM2 Inhibitors

Inhibition of AIFM2 can be performed in a cell, tissue and/or subject using agents known in the art, including, e.g., via administration of targeted oligonucleotide inhibitors (e.g., antisense and/or RNAi moieties, including siRNA, DsiRNA, shRNA, etc.).

Inducer of ROS

Elevation of reactive oxygen species (ROS) levels has been observed in many cancer cells relative to nontransformed cells, and recent reports have suggested that small-molecule enhancers of ROS may selectively kill cancer cells in various in vitro and in vivo models (Adams et al. ACS Chem Biol. 8: 923-929). Exemplary known inducers of ROS include BRD1378, BRD5459, BRD56491 and BRD9092.

BRD 1378 has the following structure:

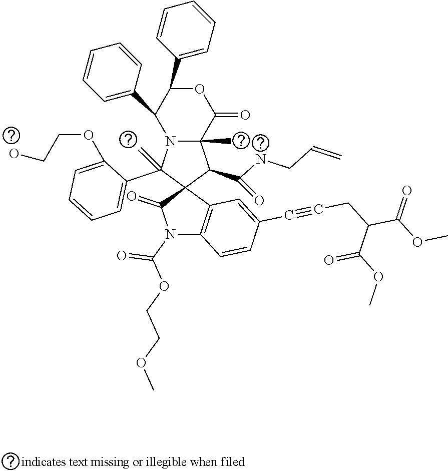

BRD5459 has the following structure:

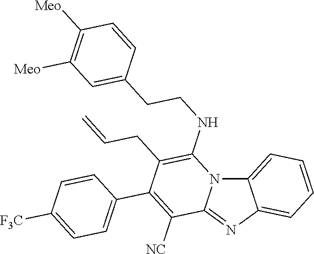

BRD56491 has the following structure:

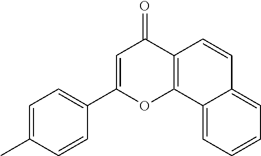 [SMILES: Cc1ccc(-c2cc(=O)c3ccc4ccccc4c3o2)cc1]

BRD9092 has the following structure:

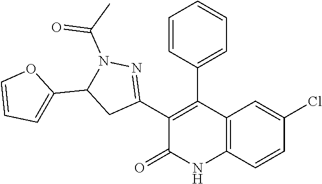 [SMILES: CC(=O)N1N=C(c2c(-c3ccccc3)c3cc(Cl)ccc3[nH]c2=O)CC1c1ccco1]

Induction of ROS as a cancer therapy has been suggested for a variety of cancers, including colon, pancreatic, breast, glioma, glioblastoma, non-small cell lung cancer, multiple myeloma, prostate cancer, hepatoma and leukemia. Without wishing to be bound by theory, induction of apoptosis and/or autophagy/autophagic cell death has been implicated in such posited anti-cancer effects.

ABCC1 Inhibitors

Inhibition of ABCC1 can be performed in a cell, tissue and/or subject using agents known in the art, including, e.g., MK-571 and Reversan, as well as via administration of targeted oligonucleotide inhibitors (e.g., antisense and/or RNAi moieties, including siRNA, DsiRNA, shRNA, etc.).

MK-571 has the following structure:

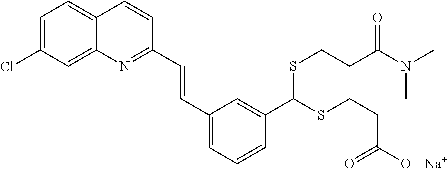 [SMILES: CN(C)C(=O)CCSC(SCCC(=O)O)c1cccc(/C=C/c2ccc3ccc(Cl)cc3n2)c1 ; [Na+]]

Reversan has the following structure:

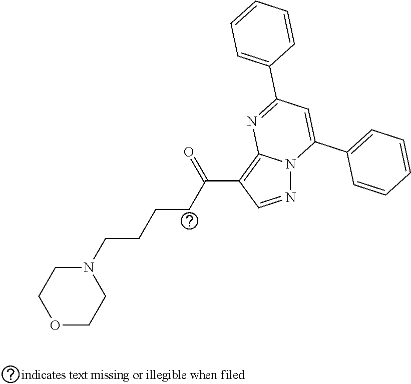 [SMILES: O=C(CCCCN1CCOCC1)c1cnn2c(-c3ccccc3)cc(-c3ccccc3)nc12]

JNK1 Inhibitors

Inhibition of JNK1 has been identified as a therapy for a variety of cancers, including, e.g., liver cancer (e.g., hepatocellular carcinoma), breast cancer, skin cancer, brain cancer (glioblastoma), leukaemia, multiple myeloma and lymphoma, among other forms of cancer.

Known JNK1 inhibitors include ZG-10, tanzisertib (CC-930), SP600125, JNK Inhibitor V, JNK Inhibitor XIV, L-JNKi 1 trifluoroacetate salt, INK Inhibitor XV, INK Inhibitor XI and JNK-IN-8.

ZG-10 has the following structure:

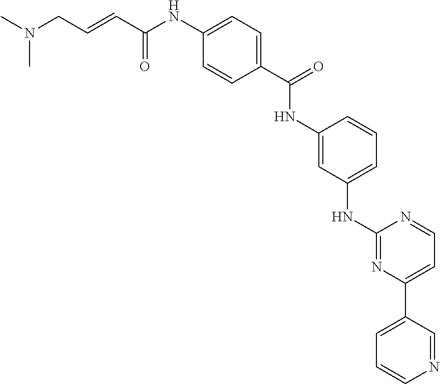 [SMILES: CN(C)C/C=C/C(=O)Nc1ccc(C(=O)Nc2cccc(Nc3nccc(-c4cccnc4)n3)c2)cc1]

Tanzisertib (CC-930) has the following structure:

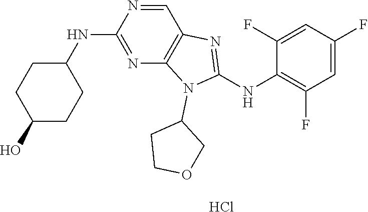

SP600125 has the following structure:

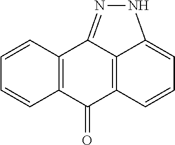 [SMILES: O=C1c2ccccc2-c2n[nH]c3cccc1c23]

JNK Inhibitor V has the following structure:

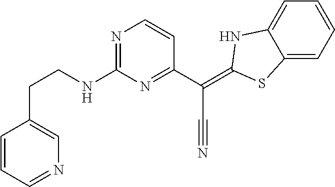 [SMILES: N#C/C(=C1/Nc2ccccc2S1)c1ccnc(NCCc2cccnc2)n1]

JNK Inhibitor XIV has the following structure:

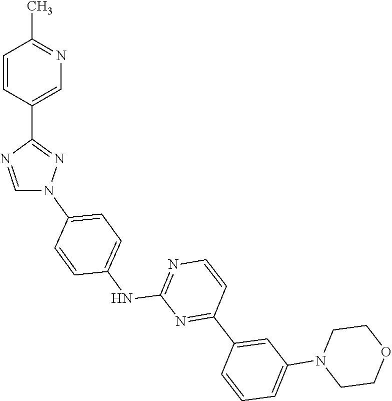 [SMILES: Cc1ccc(-c2ncn(-c3ccc(Nc4nccc(-c5cccc(N6CCOCC6)c5)n4)cc3)n2)cn1]

L-JNKi 1 trifluoroacetate salt has the following structure:

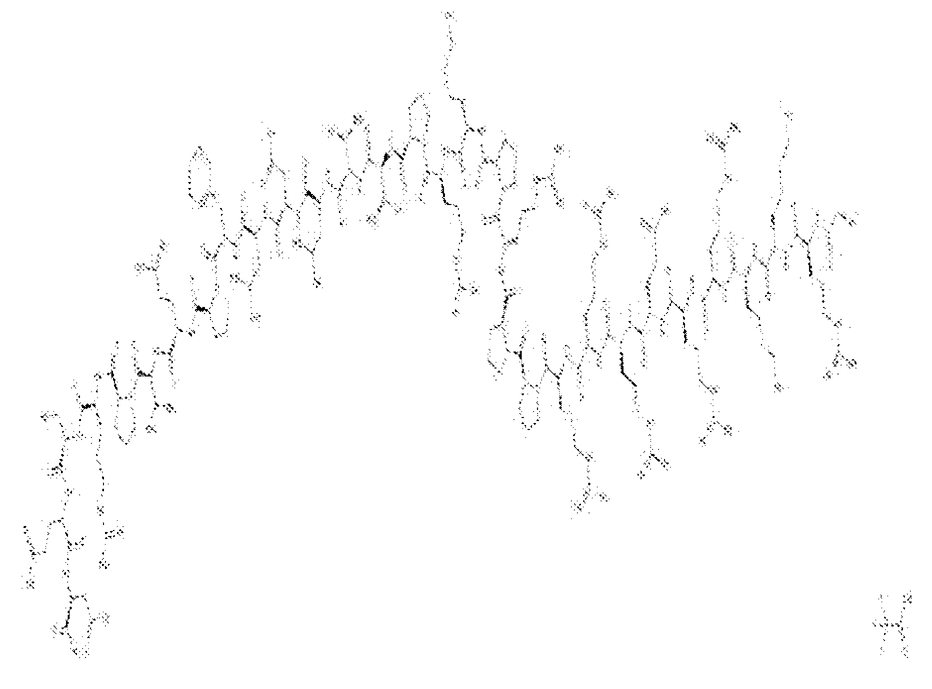

JNK Inhibitor XV has the following structure:

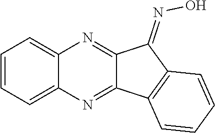 [SMILES: O/N=C1\c2ccccc2-c2nc3ccccc3nc21]

JNK Inhibitor XI has the following structure:

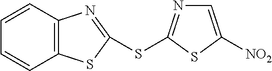 [SMILES: O=[N+]([O-])c1cnc(Sc2nc3ccccc3s2)s1]

JNK-IN-8 has the following structure:

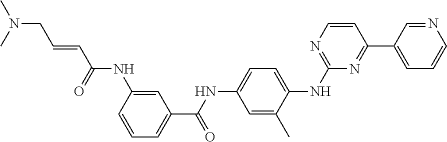 [SMILES: Cc1cc(NC(=O)c2cccc(NC(=O)/C=C/CN(C)C)c2)ccc1Nc1nccc(-c2cccnc2)n1]

ABCG2 Inhibitors

Inhibition of ABCG2 can be performed in a cell, tissue and/or subject using agents known in the art, including, e.g., elacridar, vismodegib, fumitremorgin C, Ko 143 and novobiocin sodium salt, as well as via administration of targeted oligonucleotide inhibitors (e.g., antisense and/or RNAi moieties, including siRNA, DsiRNA, shRNA, etc.).

Elacridar has the following structure:

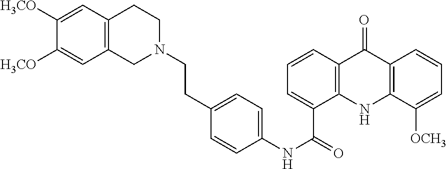 [SMILES: COc1cc2c(cc1OC)CN(CCc1ccc(NC(=O)c3cccc4c(=O)c5cccc(OC)c5[nH]c34)cc1)CC2]

Vismodegib has the following structure:

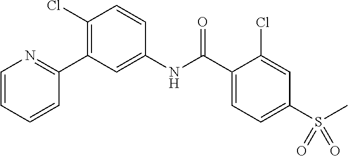 [SMILES: CS(=O)(=O)c1ccc(C(=O)Nc2ccc(Cl)c(-c3ccccn3)c2)c(Cl)c1]

Fumitremorgin C has the following structure:

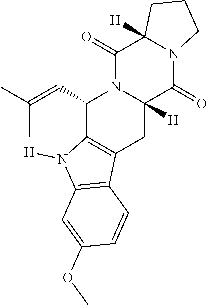 [SMILES: COc1ccc2c3c([nH]c2c1)[C@H](C=C(C)C)N1C(=O)[C@@H]2CCCN2C(=O)[C@@H]1C3]

Ko 143 has the following structure:

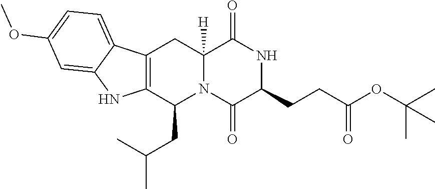 [SMILES: COc1ccc2c3c([nH]c2c1)[C@H](CC(C)C)N1C(=O)[C@H](CCC(=O)OC(C)(C)C)NC(=O)[C@@H]1C3]

Novobiocin sodium salt has the following structure:

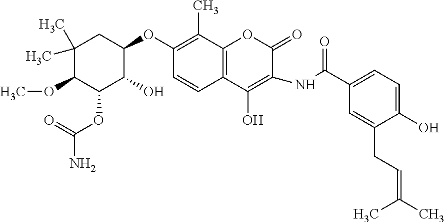 [SMILES: CO[C@@H]1[C@@H](OC(N)=O)[C@@H](O)[C@H](Oc2ccc3c(O)c(NC(=O)c4ccc(O)c(CC=C(C)C)c4)c(=O)oc3c2C)CC1(C)C]

E3-Ubiquitin Ligase Inhibitors

Exemplary known E3-ubiquitin ligase inhibitors include SMER-3, Heclin and SZL P1-41. SMER-3 has the following structure:

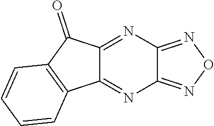 [SMILES: O=C1c2ccccc2-c2nc3nonc3nc21]

Heclin has the following structure:

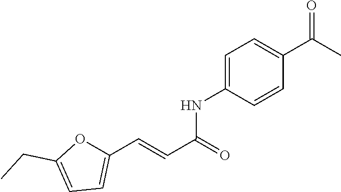 [SMILES: CCc1ccc(/C=C/C(=O)Nc2ccc(C(C)=O)cc2)o1]

SZL P1-41 has the following structure:

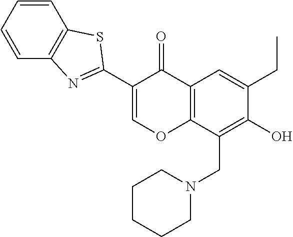 [SMILES: CCc1cc2c(=O)c(-c3nc4ccccc4s3)coc2c(CN2CCCCC2)c1O]

Inhibition of E3-ubiquitin ligase has been proposed as a therapy for malignant melanoma and multiple myeloma, as well as other forms of cancer.

UGT1A6 Inhibitors

Inhibition of UGT1A6 can be performed in a cell, tissue and/or subject using agents known in the art, including, e.g., via administration of targeted oligonucleotide inhibitors (e.g., antisense and/or RNAi moieties, including siRNA, DsiRNA, shRNA, etc.).

Treatment Selection

The methods described herein can be used for selecting, and then optionally administering, an optimal treatment for a subject. Thus the methods described herein include methods for the treatment of a disease or disorder, particularly cancer. Generally, the methods include administering a therapeutically effective amount of a treatment as described herein, to a subject who is in need of, or who has been determined to be in need of, such treatment.

As used in this context, to “treat” means to ameliorate at least one symptom of the cancer. For example, a treatment can result in a reduction in tumor size, tumor growth, cancer cell number, cancer cell growth, or metastasis or risk of metastasis.

For example, the methods can include selecting and/or administering a treatment that includes a therapeutically effective amount of a KDM6A/KDM6B inhibitor and a MGLL inhibitory agent to a subject having a cancer.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Combination Treatments

The compositions and methods of the present disclosure may be used in the context of a number of therapeutic or prophylactic applications. In order to increase the effectiveness of a treatment with the compositions of the present disclosure, e.g., a combined KDM6A/KDM6B inhibitor and a MGLL inhibitory agent, or to augment the protection of another therapy (second therapy), it may be desirable to further combine these compositions and methods with one another, or with other agents and methods effective in the treatment, amelioration, or prevention of diseases and pathologic conditions, for example, cancers.

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

Pharmaceutical Compositions

Agents of the present disclosure can be incorporated into a variety of formulations for therapeutic use (e.g., by administration) or in the manufacture of a medicament (e.g., for treating or preventing a cancer) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds (e.g., FDA-approved compounds) of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethyl succinates.

Furthermore, the term “pharmaceutically acceptable prodrugs” as used herein refers to those prodrugs of the certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Prodrugs as Novel Delivery Systems, Vol. 14 of the A.C. S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, such as the combination of a KDM6A/KDM6B inhibitor and a MGLL inhibitory agent, etc., in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

Pharmaceutical Dosages

Pharmaceutical compositions of the present disclosure containing an agent described herein may be used (e.g., administered to an individual, such as a human individual, in need of treatment with a KDM6A/KDM6B inhibitor and a MGLL inhibitory agent, etc.) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chl orhexi dine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent or combination of agents (e.g., a KDM6A/KDM6B inhibitor and a MGLL inhibitory agent, etc.) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

FDA-approved drugs provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent or combined agents (e.g., a KDM6A/KDM6B inhibitor and a MGLL inhibitory agent, etc.) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The FDA-approved drug or other therapy is administered to the subject in an amount sufficient to achieve a desired effect at a desired site (e.g., reduction of cancer size, cancer cell abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent (or combination of agents) of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent or combination of agents (e.g., a KDM6A/KDM6B inhibitor and a MGLL inhibitory agent, etc.) described herein. In certain embodiments, a dose described herein includes independently between 1 mg and 3 mg, inclusive, of an agent or combination of agents (e.g., a KDM6A/KDM6B inhibitor and a MGLL inhibitory agent, etc.) described herein. In certain embodiments, a dose described herein includes independently between 3 mg and 10 mg, inclusive, of an agent or combination of agents (e.g., a KDM6A/KDM6B inhibitor and a MGLL inhibitory agent, etc.) described herein. In certain embodiments, a dose described herein includes independently between 10 mg and 30 mg, inclusive, of an agent or combination of agents (e.g., a KDM6A/KDM6B inhibitor and a MGLL inhibitory agent, etc.) described herein. In certain embodiments, a dose described herein includes independently between 30 mg and 100 mg, inclusive, of an agent or combination of agents (e.g., a KDM6A/KDM6B inhibitor and a MGLL inhibitory agent, etc.) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent or combination of agents (e.g., a KDM6A/KDM6B inhibitor and aMGLL inhibitory agent, etc.) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent or combination of agents (e.g., a KDM6A/KDM6B inhibitor and a MGLL inhibitory agent, etc.) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent, or first combination of therapeutic agents, of the disclosure is administered prior to, in combination with, at the same time, or after administration of the agent of the disclosure. In some embodiments, the second therapeutic agent is selected from the group consisting of a chemotherapeutic, an antioxidant, an antiinflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, additional KDM6A/KDM6B inhibitors and/or MGLL inhibitory agents, etc., epigenetic modifier inhibitors, etc., other anti-cancer agents, immunomodulatory agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

Kits

The instant disclosure also provides kits containing agents of this disclosure for use in the methods of the present disclosure. Kits of the instant disclosure may include one or more containers comprising an agent or combination of agents (e.g., a KDM6A/KDM6B inhibitor and a MGLL inhibitory agent, etc.) of this disclosure and/or may contain agents (e.g., oligonucleotide primers, probes, etc.) for identifying a cancer or subject as possessing one or more variant sequences. In some embodiments, the kits further include instructions for use in accordance with the methods of this disclosure. In some embodiments, these instructions comprise a description of administration of the agent to treat or diagnose, e.g., a cancer, according to any of the methods of this disclosure. In some embodiments, the instructions comprise a description of how to detect a certain class of cancer, for example in an individual, in a tissue sample, or in a cell. The kit may further comprise a description of selecting an individual suitable for treatment based on identifying whether that subject has a specific type of cancer.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a class of cancer, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, at least one active agent, or combination of active agents in the composition is a KDM6A/KDM6B inhibitor and a MGLL inhibitory agent, etc. The container may further comprise a second and/or additional pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

Identification of small-molecule/transcript pairs

Area-under-concentration-response-curve data from the Cancer Therapeutics Response Portal were downloaded from the NCI CTD² Data Portal (ocg.cancer.gov/programs/ctd2/data-portal; with the designated given filename CTRPv2.0 2015 ctd2 ExpandedDataset.zip). All AUCs that resulted from fits through fewer than 12 concentrations or that had a value greater than 16 were removed. Additionally, cell lines that had data for fewer than 20% of compounds were removed. RNA sequencing data (expressed as log 2(transcripts per million+1)) for shared cell lines was downloaded from the Broad Institute Dependency Map Portal (depmap.org/portal/; filename CCLE_depMap_19Q1_TPM.csv). Transcripts were restricted to protein-coding transcripts using the biomaRt R package (37) and all transcripts with a maximum value of 0 across all cell lines tested were removed.

Genomics of Drugs Sensitivity in Cancer area-under-concentration-response-curve and RMA-normalized basal expression data were downloaded from the GDSC portal (www.cancerrxgene.org/downloads; filenames v17.3_fitted_dose_response.xlsx and sanger1018_brainarray_ensemblgene_rma.txt.gz). Cell lines that had data for fewer than 20% of compounds were removed.

Pearson correlation coefficients were calculated between AUC values and basal gene expression and normalized for cell number using Fisher's Z-transformation across all cell lines and subsets containing at least 30 cell lines defined by each dataset (by lineage, histology, microsatellite stability status, growth mode, and mutation). Mutation-based subsets (known cancer hotspot mutations) were derived from DepMap portal hotspot mutation data (for CTRP) or Cancer Functional Events information (for GDSC) (11).

Correlations were filtered using the following criteria, while excluding the most and least sensitive cell line in each subset (to reduce the likelihood of erroneous outlier data points dominating correlations): 1) the absolute magnitude of correlation, with significance corresponding to a Bonferroni-corrected, two-tailed distribution adjusted for the number of transcripts tested; 2) degree of “outlierness”, requiring at least 3 standard deviations from the distribution mean; 3) number of cell lines (n>9); 4) differential transcript expression of at least 3 units (log 2 scale) in the data subset; 5) removal of confounding genes, defined as those that were significantly associated with at least 50% (CTRP) or 25% (GDSC) of all small molecules in the dataset; 6) gene co-expression. For gene co-expression, an iterative semi-partial correlation approach was used to capture orthogonal correlated genes that, upon adjustment, remained significant as described by Kim (38) (with Bonferroni correction for the number of genes tested) and were at least 3 standard deviations from the mean. In an attempt to not exclude very highly co-expressed genes that are essentially indistinguishable, all genes were retained with pairwise r>0.8 and sensitivity-expression correlations within 0.25 standard deviations.

For analysis of GSK-J4 sensitivity across 638 cell lines, AUC data was downloaded from the Lochmann et al. manuscript and correlated sensitivity with expression using the GDSC expression file and procedure as described above (23).

Cellular Assays

Cell lines were taken from viably frozen stocks of the same original provenance as for CTRP and DepMap data, with identity confirmed by SNP-based DNA fingerprinting as described previously (12, 13, 39). Cell lines were propagated using the same media and culture conditions as for CTRPv2.0 experiments. For drug-treatment assays, 1000 cells per well were plated into 30 or 504, of media in 384-well plates. For live-cell imaging, cells were plated into clear-bottom plates (Corning) and imaged on an IncuCyte S3 throughout the course of the experiment. For CellTiter-Glo experiments, the initial viability of a subset of plated cells was established at the same time as compound addition using CellTiter-Glo as described previously (24). Remaining cells were exposed to compounds and/or vehicle (constant DMSO volume) using a Tecan D300e Digital Dispenser, and CellTiter-Glo was added 72 hours later. DMSO- and D0-normalized concentration-response curves were generated using four-parameter nonlinear regression using the drc package in R (40). Biphasic curves were also fit and prioritized relative to standard four-parameter curves where appropriate using the BIC (Bayesian information criteria) metric. For calculation of drug combination effects, curves were fit and deviation from the null model (Loewe additivity) assessed using the BIGL package in R (reference: PMID 29263342) (41). General-purpose optimization (Nelder-Mean algorithm) was used for single-agent fits, and the “model” option was used to predict variance. Overall significance was assessed using the bootstrapped meanR test (n=1000 iterations), and per-concentration significance using maxR, as described by the package authors.

Small Molecules

Small molecules were purchased from the following vendors: ABT-737 (Selleck S1002), alpelisib (Selleck S2814), AZD8055 (Selleck S1555), BI-2536 (Selleck S1109), BMS-754807 (Active Biochem A-1013), canagliflozin (Selleck S2760), carmustine (Selleck S3669), CP-100356 (Sigma-Aldrich PZ0171), CPI-455 (Selleck S8287), cysmethynil (Cayman 14745), dactolisib (Selleck S1009), doxorubicin (Selleck S1208), elacridar (Sigma-Aldrich SML0486), emodin (Selleck S2295), EPZ004777 (Tocris 5567), erastin (Tocris 5449), GSK-J1 (Selleck S7581), GSK-J2 (Tocris 4688), GSK-J4 (Selleck S7070), GSK-J5 (Tocris 4689), GSK2879552 (Selleck S7796), IOX1 (Tocris 4464), JIB04 (Tocris 4972), JZL184 (Selleck S4904), KDOAM25 (Sigma-Aldrich SML1588), KX2-391 (Selleck S2700), linsitinib (Selleck S1091), ML210 (Sigma-Aldrich SML0521), navitoclax (Selleck S1001), niclosamide (Sigma-Aldrich N3510), nutlin-3a (Selleck S8059), NVP-ADW742 (Selleck S1088), 06-benzylguanine (Sigma-Aldrich B2292), omacetaxine (Tocris 1416), ORY-1001 (Selleck S7795), paclitaxel (Selleck S1150), panobinostat (Selleck S1030), PI-103 (Selleck S1038), pictilisib (Selleck S1065), pluripotin (Sigma-Aldrich SML0444), PRIMA-1 (Selleck S7723), S63845 (Active Biochem A-6044), shikonin (Selleck S8279), sirolimus (Selleck S1039), SMER-3 (Tocris 4375), SNS-032 (Selleck S1145), tazemetostat (Selleck S7128), temozolomide (Tocris 2706), tivozanib (Selleck S1207), trametinib (Selleck S2673), venetoclax (Selleck S8048), verapamil (Cayman 14288), vorinostat (Sigma-Aldrich SML0061), WEHI-539 (MedChem Express HY-15607A), YM-155 (Selleck S1130), and ZSTK474 (Selleck S1072). KDOBA67 was synthesized from GSK-J4 as described previously (26).

Western Blots

Cells were washed with cold PBS, scraped, and pellets were collected for lysis in 1% NP-40 buffer (150 mM NaCl, 50 mM Tris pH 7.5, 2 mM EDTA pH 8, 25 mM NaF and 1% NP-40) containing 2× protease inhibitors (Roche) and 1× Phosphatase Inhibitor Cocktails I and II (CalBioChem). Lysates were quantitated using BCA Assay (Pierce) and normalized. Following denaturation (95° C.) in LDS sample buffer (Invitrogen), proteins were resolved by electrophoresis in NuPage Novex 4-12% gels (Invitrogen). Protein was transferred to nitrocellulose membranes using the iBlot2 system (Invitrogen), blocked in LI-COR blocking buffer, then probed with primary antibodies. All primary antibodies were used at a 1:1000 dilution, and secondary antibodies (LI-COR) used at a 1:10000 dilution. Following antibody incubation, proteins were detected using the LI-COR Odyssey CLx Infrared Imaging System. Primary antibodies used were: MGLL (Santa Cruz sc-134789 and Abcam ab152002), MCL1 (Cell Signaling 5453), SLC7A11 (Cell Signaling 12691), BCL2L1 (Cell Signaling 2764), MGMT (Cell Signaling 2739), AIFM2 (Santa Cruz sc-377120), IRS2 (Cell Signaling 4502), ABCB1 (Cell Signaling 12683), beta actin (Cell Signaling 3700), and vinculin (Cell Signaling 4650).

Lentiviral Overexpression

For individual ORF overexpression experiments, ORF DNA in the pLX-317 expression vector was obtained from the Broad Institute's Genetic Perturbation Platform (GPP) for MGLL, ABCB1, MCL1, BCL2L1, AIFM2, IRS2, LacZ, eGFP, and HcRed. MGMT (NM 002412) and SLC7A11 (NM_014331) were synthesized (GenScript) and subcloned into the pLX-307 vector for expression. Lentiviral production in 293T cells was carried out according to the protocol “shRNA/sgRNA/ORF Low Throughput Viral Production (10 cm dish/6 well)” from the Broad Institute's Genetic Perturbation Platform portal (GPP; portals.broadinstitute.org/gpp/public/resources/protocols). Infection and selection were carried out according to GPP Viral Infection protocols, followed by cell seeding in 384-well plates for compound treatment or harvesting for Western blotting as described above.

Site-directed mutagenesis was used to introduce the S132A and D249N mutations into MGLL in the pDONR223 vector. Sequence-verified clones were then recombined into lentiviral expression vectors pLX-313 or pLX-307.

For near-genome-scale screening of the ORFeome library, G402 cells were infected with virus generated by the GPP using the published protocol with the following optimized conditions: 2004, virus and 1.5e6 cells/well, with 4 μg/mL polybrene (19). Cells were selected with 1 μg/mL puromycin beginning 2 days after infection. Cells were split into arms after selection completion (5 days post-infection), and then drug was added the following day or cells were harvested (for early timepoint collection). Cell collection, gDNA extraction, PCR, and sequencing to measure barcode abundance were carried out as described previously (19). Relative sequencing abundance was used to calculate median log(2) fold-change of sequencing reads per million for drug (or vehicle) conditions relative to early timepoint (three replicates per condition). To account for ORFs that had drug-independent effects on proliferation, results were adjusted to those from vehicle-treated conditions using linear regression.

CRISPR-Cas9-Mediated Gene Knockout

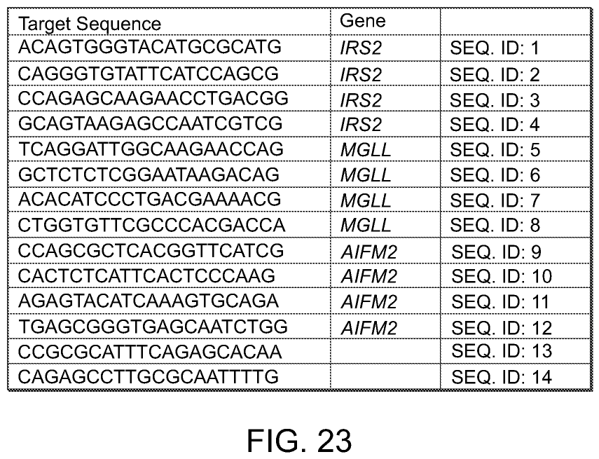

Cas9 stably-infected HEC1A, MEWO, SUIT2, and WM2664 cells and sgRNAs (pXPR_BRD003 vector) targeting AIFM2, IRS2, MGLL, or control guides (non-cutting) were acquired from the GPP (FIG. 23). Cell line identities were confirmed to match parental cells using SNP fingerprinting. Virus was generated and infection carried out as described above. After puromycin selection (3 μg/mL for MEWO, 1.5 μg/mL for WM2664, 2 μg/mL for SUIT2 and HEC1A), cells were seeded in 384-well plates for compound treatment or harvested for Western blotting as described above.

PRISM Pooled Cell-Line Combination Screen

A pool of 549 cell lines were treated for 5 days with GSK-J4 (20 μM top dose, eight concentrations, twofold dilution) in the presence or absence of JZL184 (1 μM). JZL184 alone was tested at 1 μM and 10 μM. All conditions were tested in triplicate.

### Example 2: Identification of Transcripts Associated with Drug Resistance from Cancer Cell

Line Data

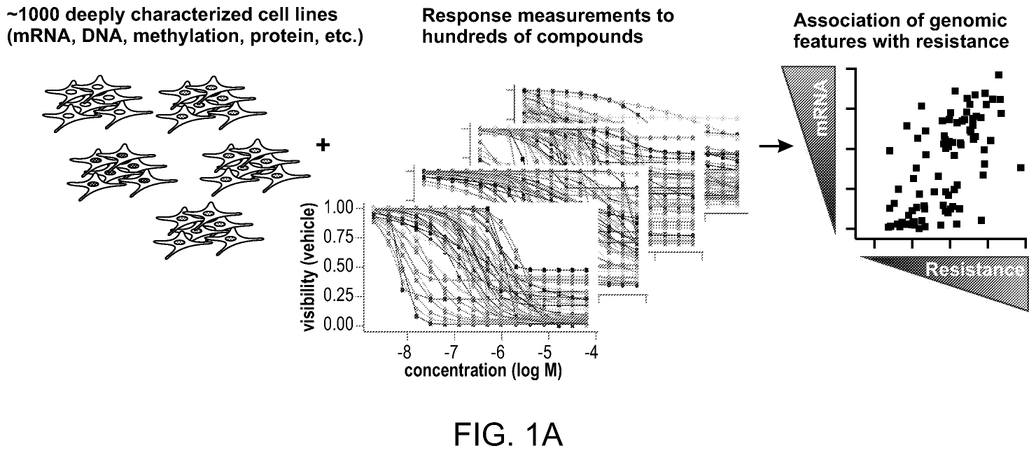

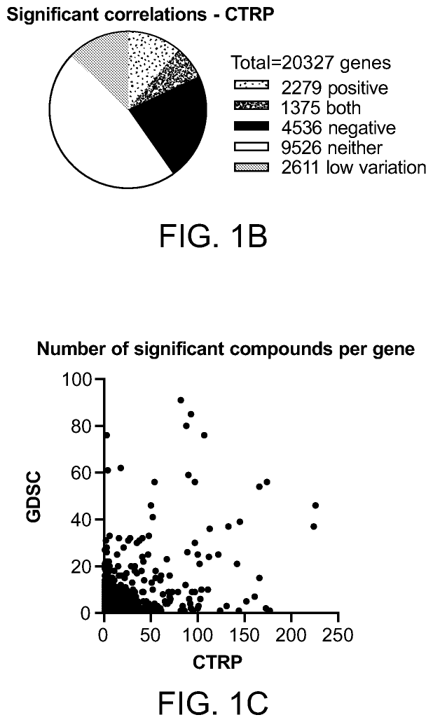

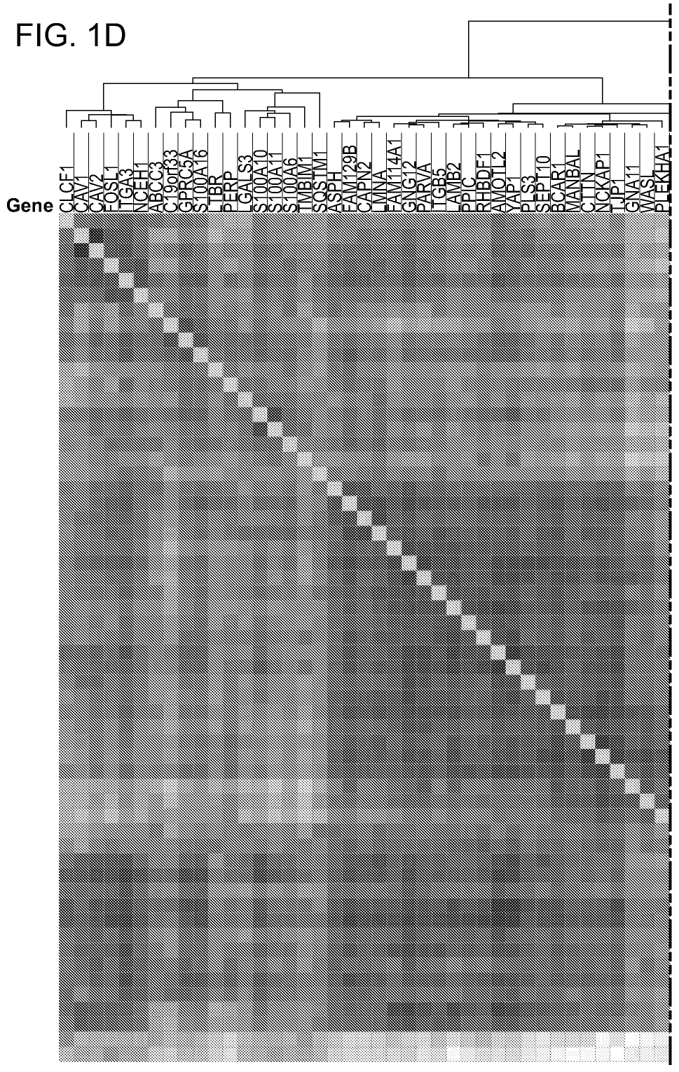

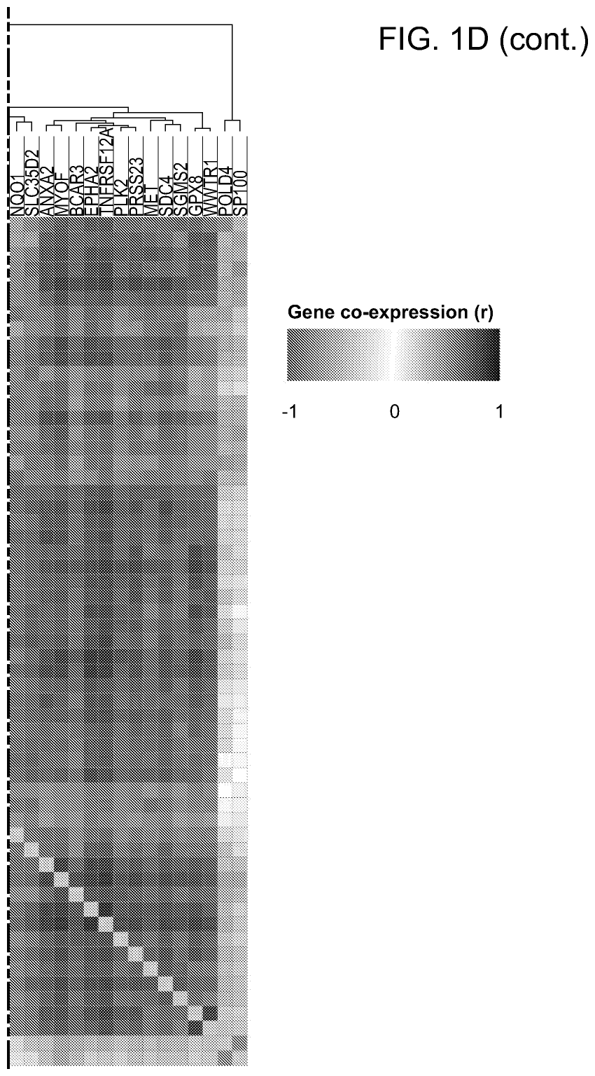

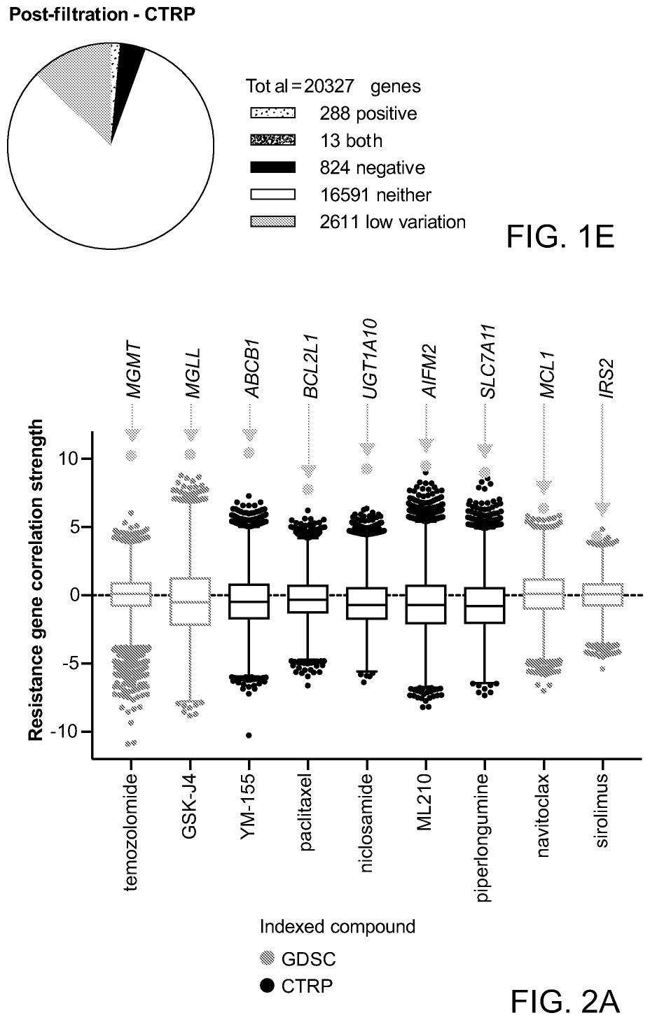

To begin to associate basal transcript levels with drug response, public small-molecule sensitivity data were downloaded and expressed as area under concentration-response curves (AUCs), from the Cancer Therapeutics Response Portal (CTRP) v2.0, RNA sequencing data from the Cancer Dependency Map (DepMap) portal, and AUC data and microarray expression data from the Genomics of Drug Sensitivity in Cancer (GDSC) portal (11-14). For both datasets, the response was correlated to each small molecule with basal gene expression for all protein-coding, differentially-expressed transcripts across all cell lines and annotated cell-line subsets defined by each project (e.g., lineage, growth mode, hotspot mutation) (FIG. 1A).

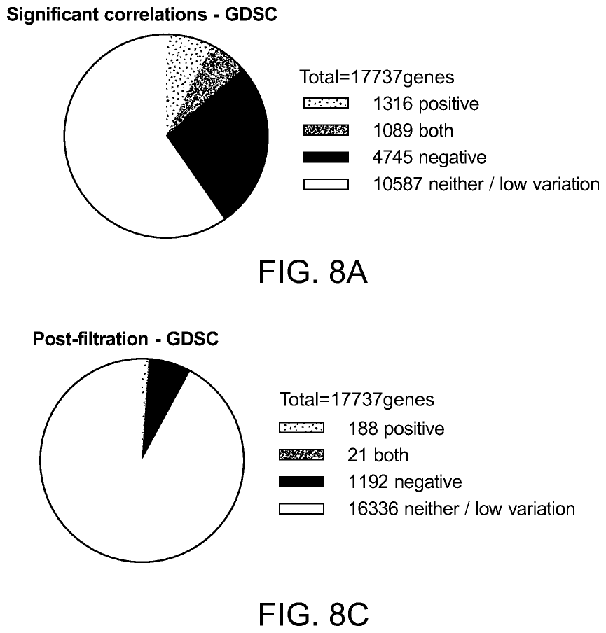

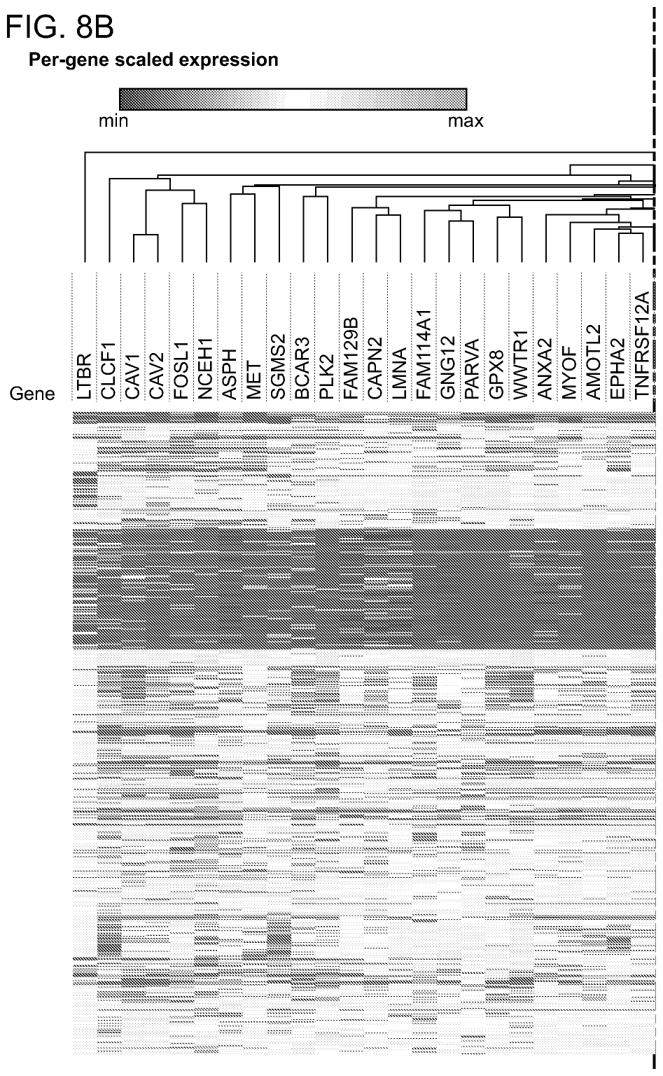

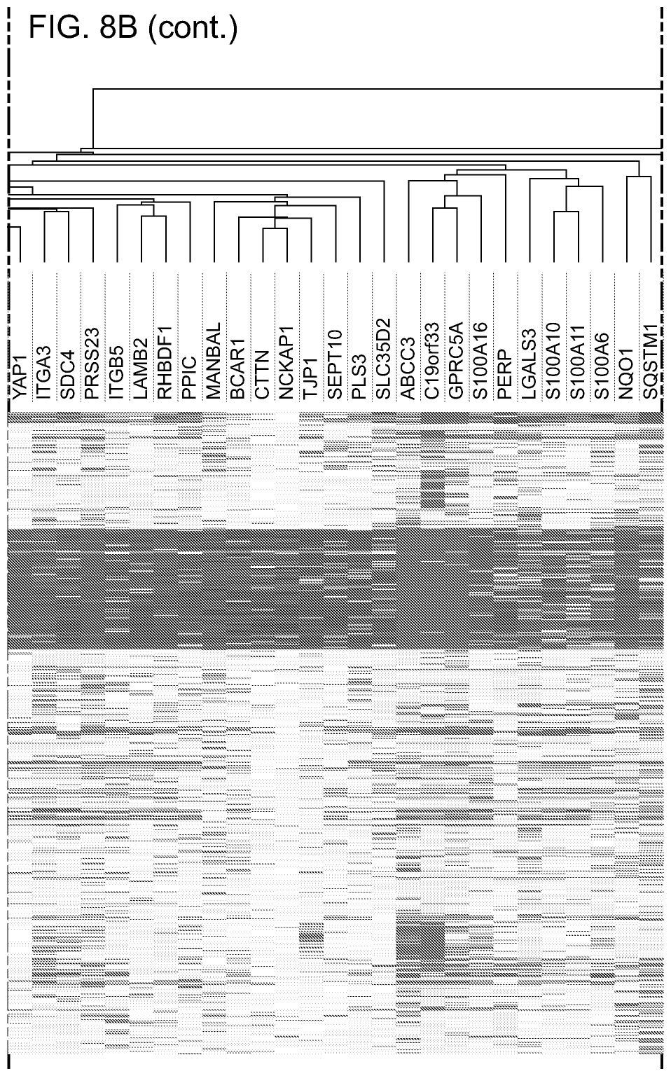

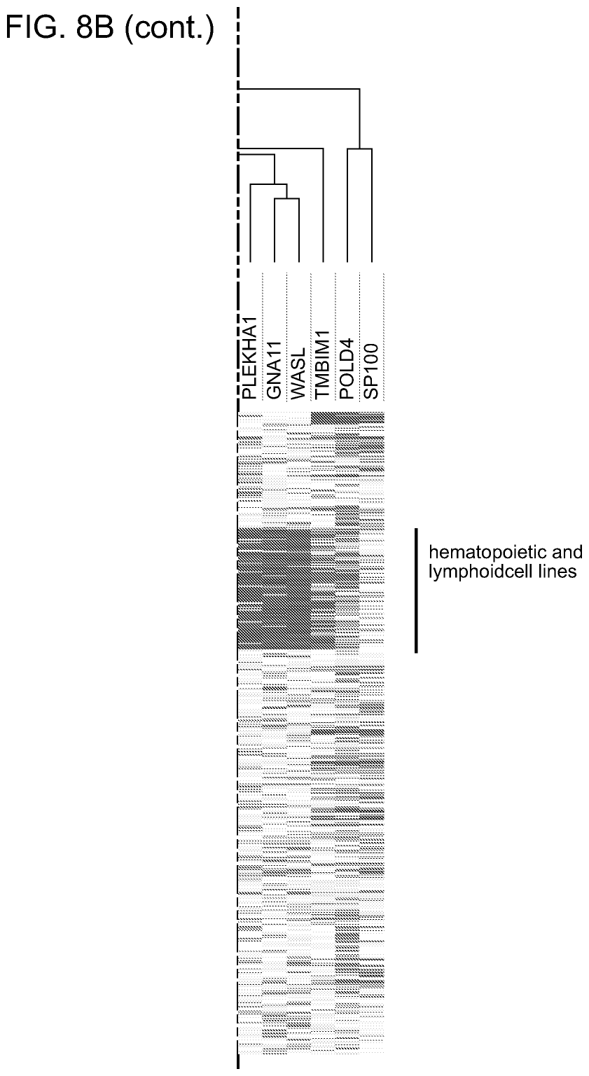

As an initial filter, further assessment was restricted to correlations that were significant by both their absolute magnitude (adjusted for cell-line number) and the associated z-score for each compound, which provides a measure of selectivity for each correlation. Associated genes that were likely proxies of co-expressed genes with a much stronger association were excluded using a regression-based approach (FIGS. 1B and 8A). Broadly, remaining significantly correlated transcripts could be classified into three groups: 1) transcripts that were correlated selectively with individual small molecules (or groups of mechanistically-related small molecules), 2) transcripts associated across multiple small molecules of diverse annotated mechanisms (e.g., drug efflux pumps) and 3) transcripts that tended to be correlated with tens to hundreds of small molecules that appeared to mark differences in global expression across subsets of cell lines (e.g., by lineage). To enrich for the discovery of candidate resistance genes that were exquisitely specific to individual therapies (or groups of mechanistically-related therapies), transcripts that were reflective of global transcriptional differences across cell lines were excluded, as they appeared to merely reflect subsets of cell lines that were in general less responsive to drug treatment (e.g., hematopoietic and lymphoid lines; FIGS. 1C, 1D, and 8B).

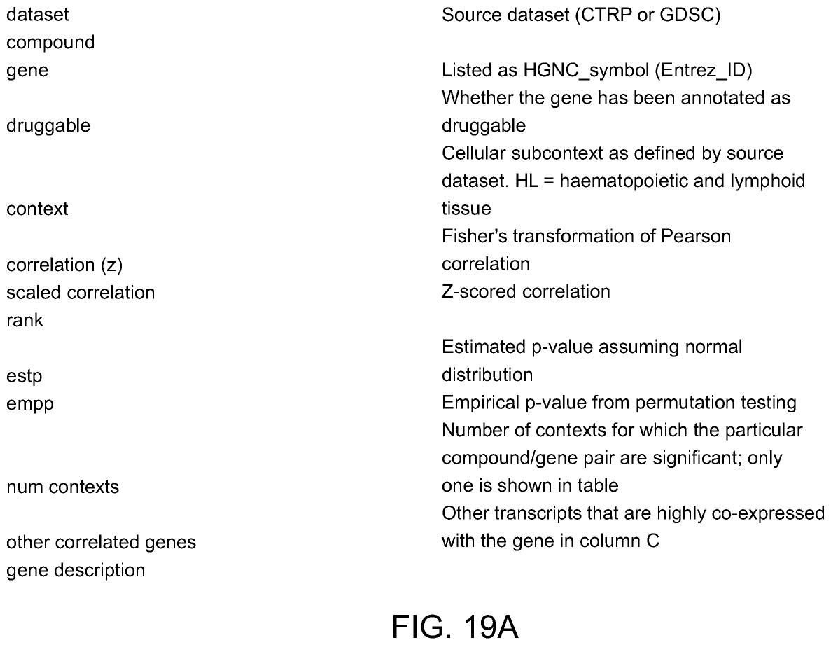

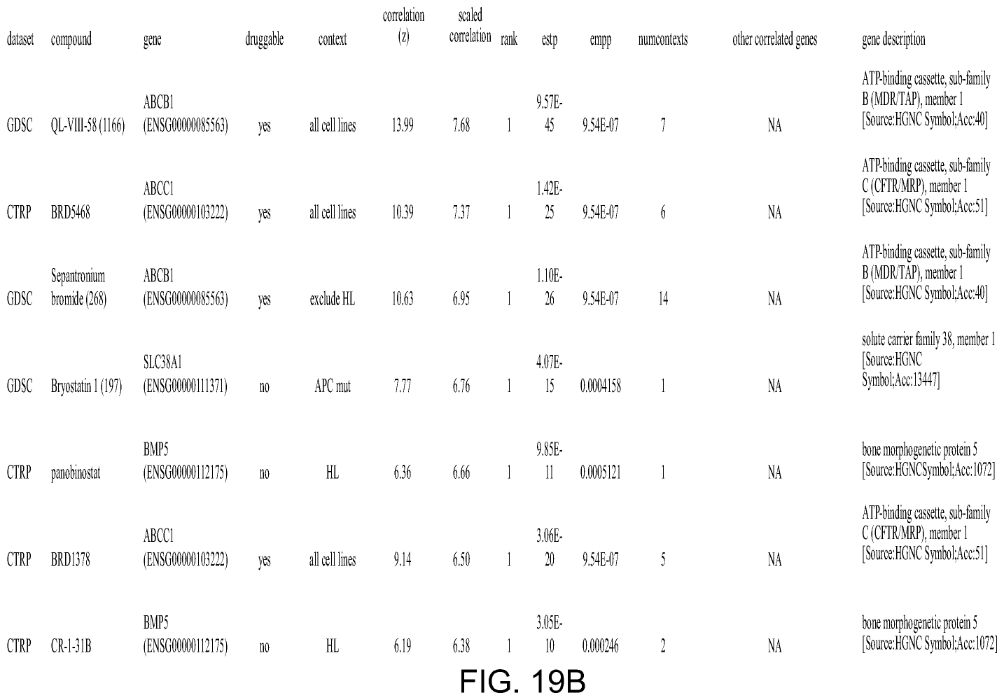

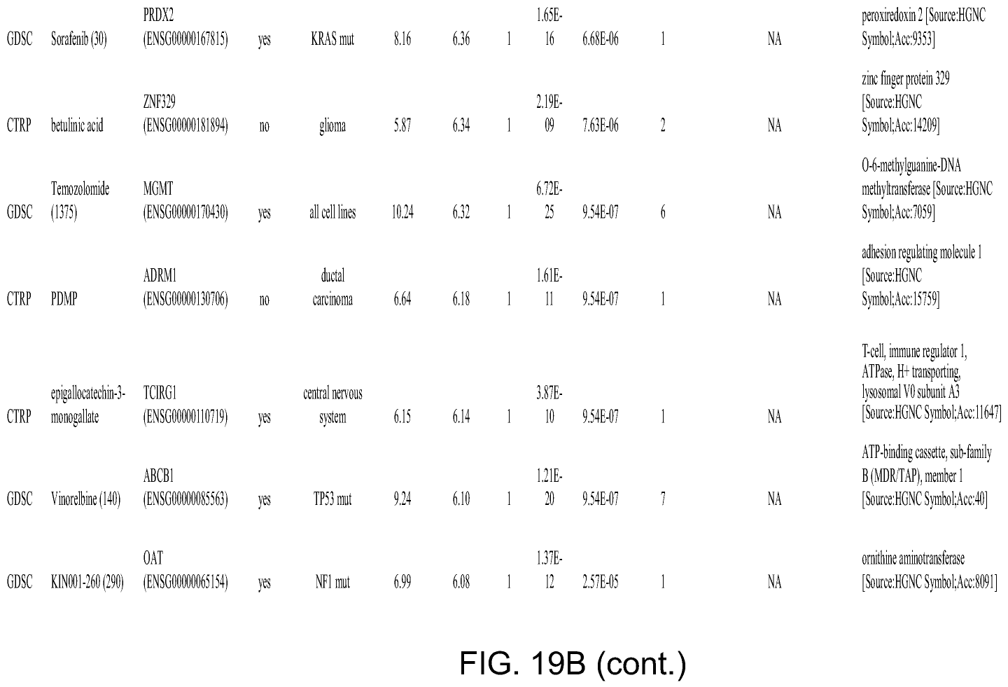

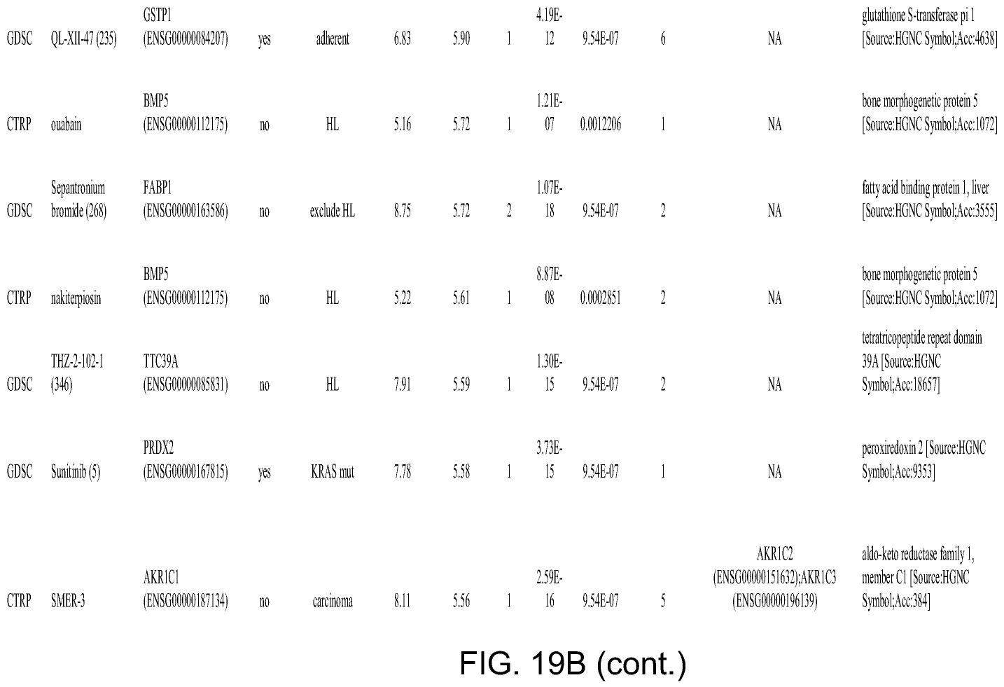

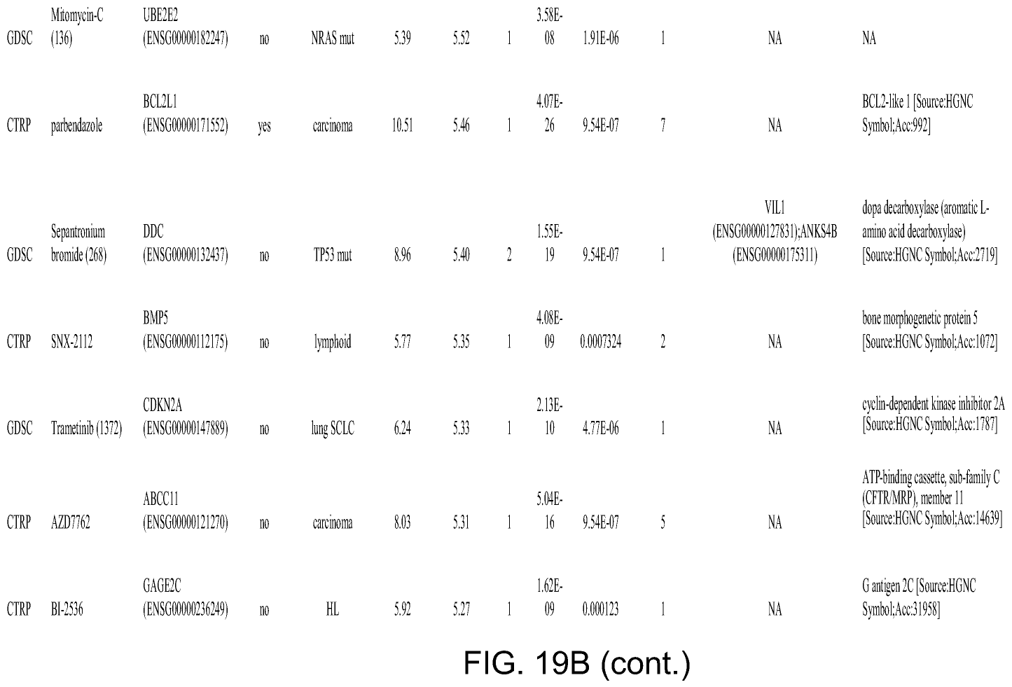

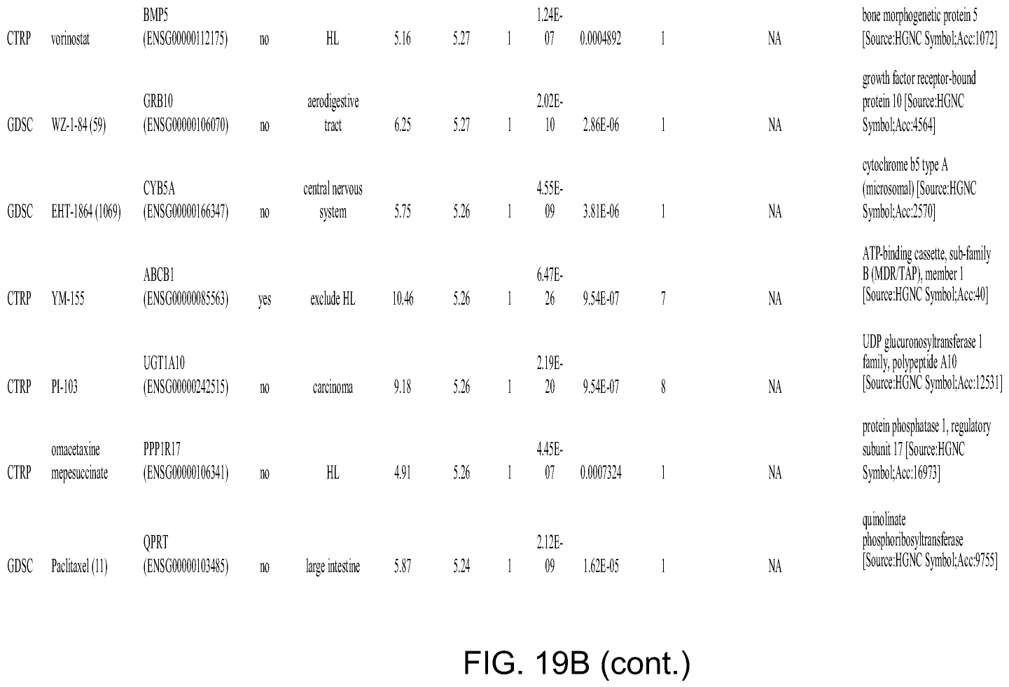

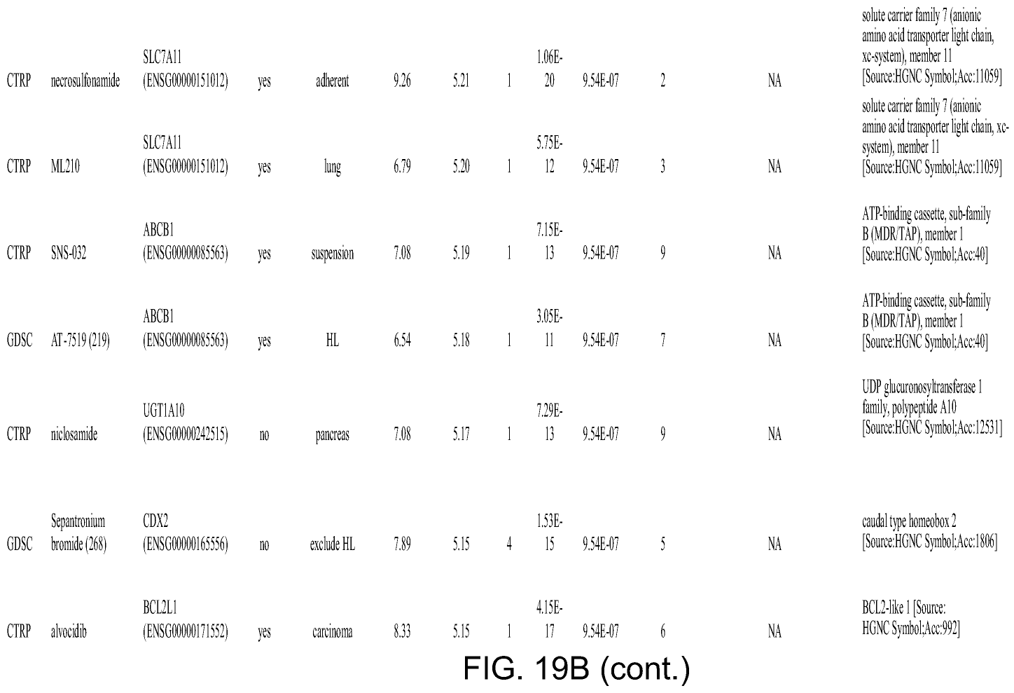

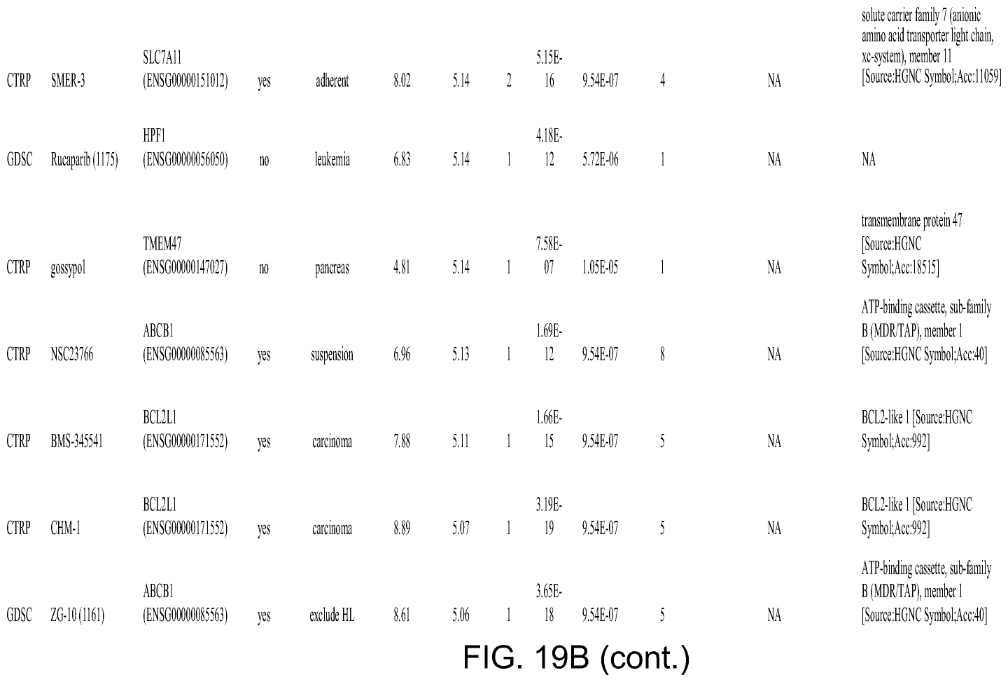

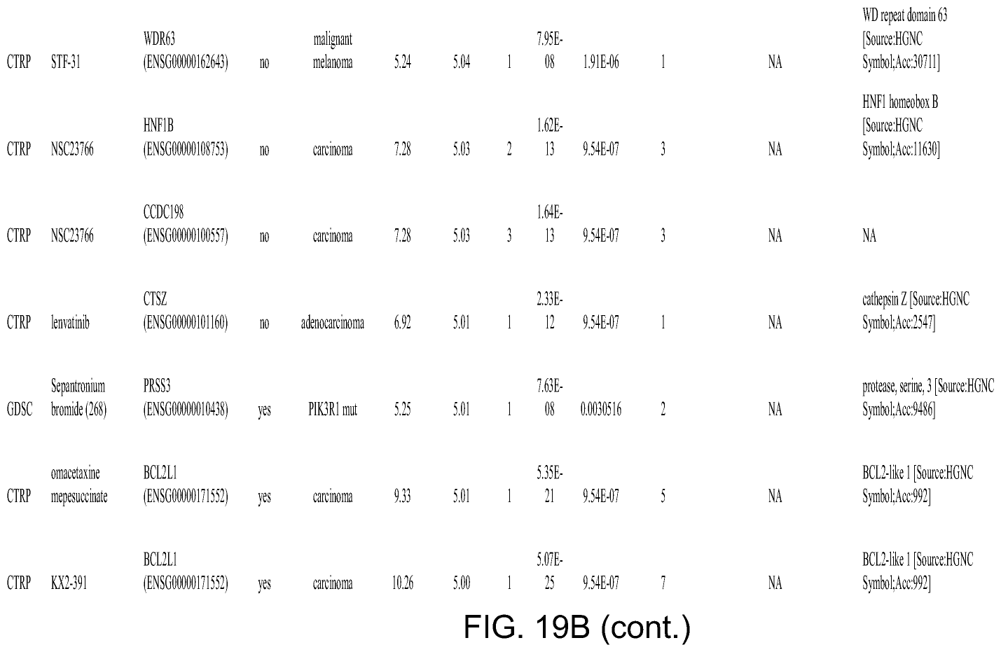

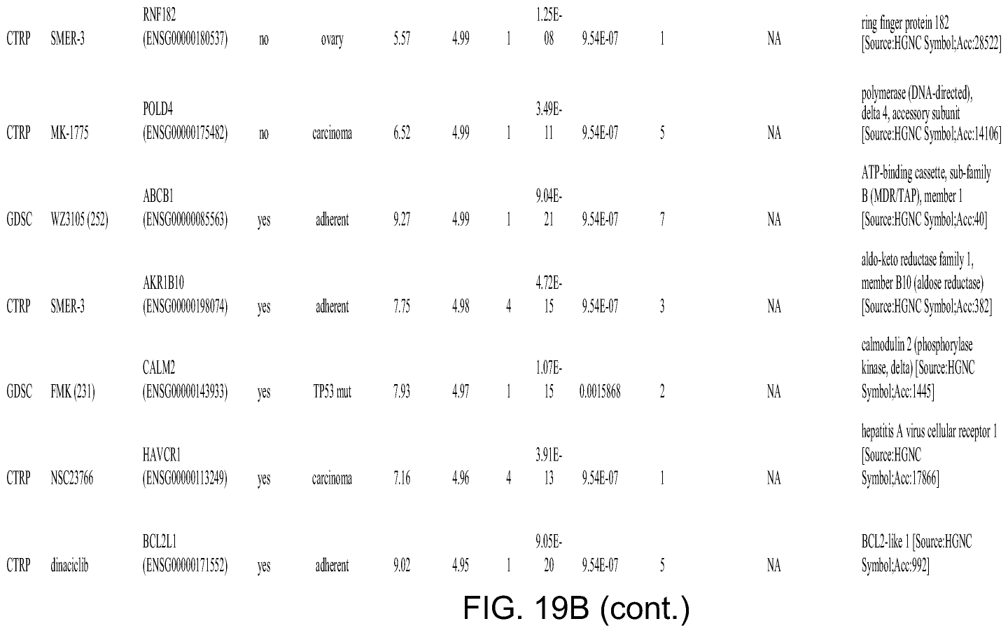

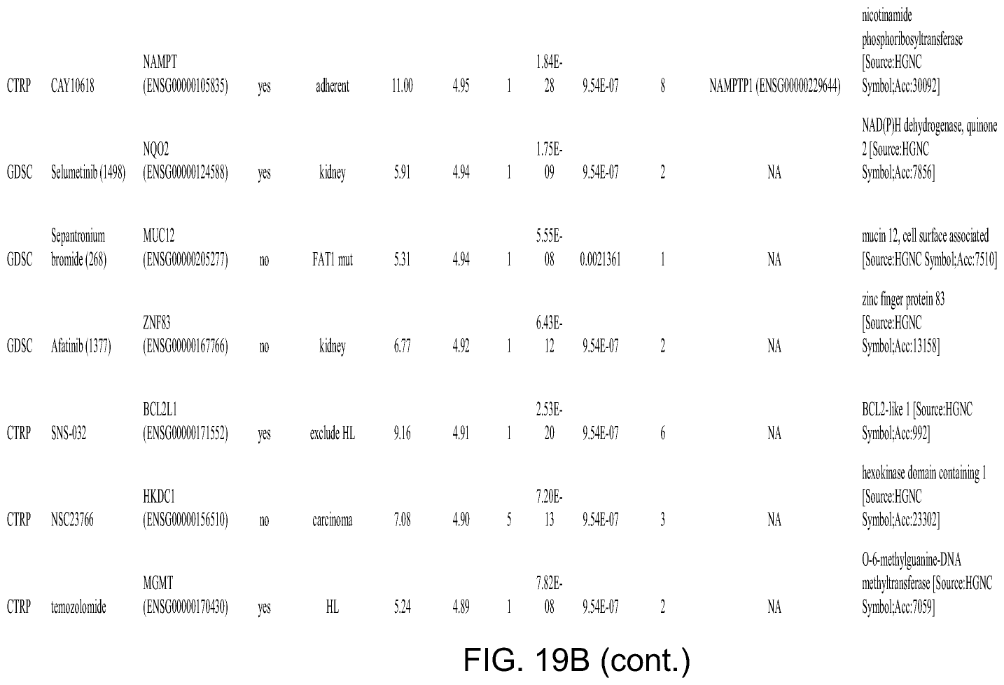

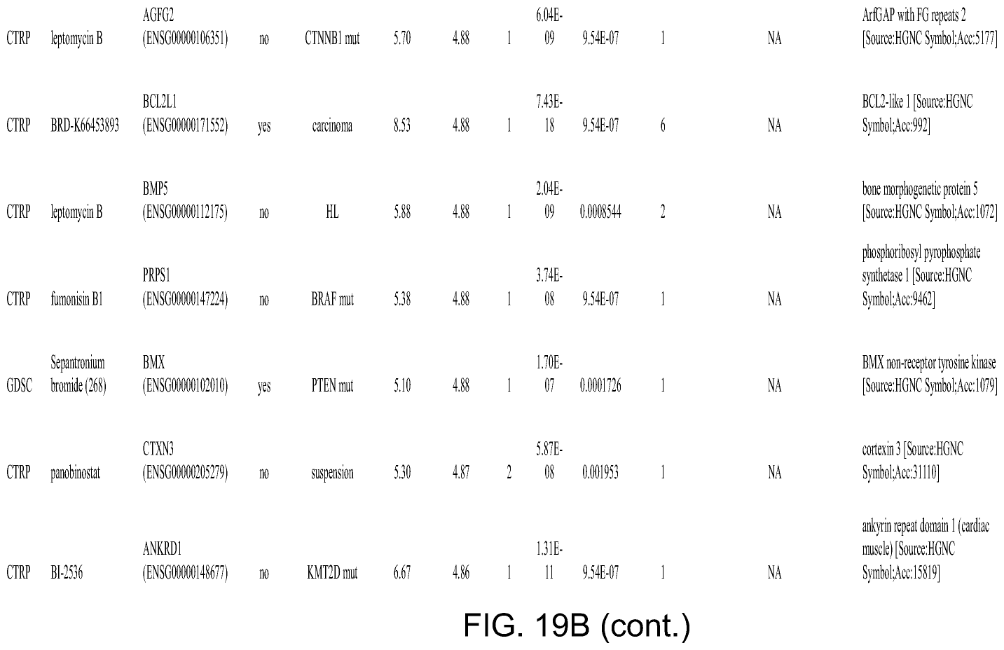

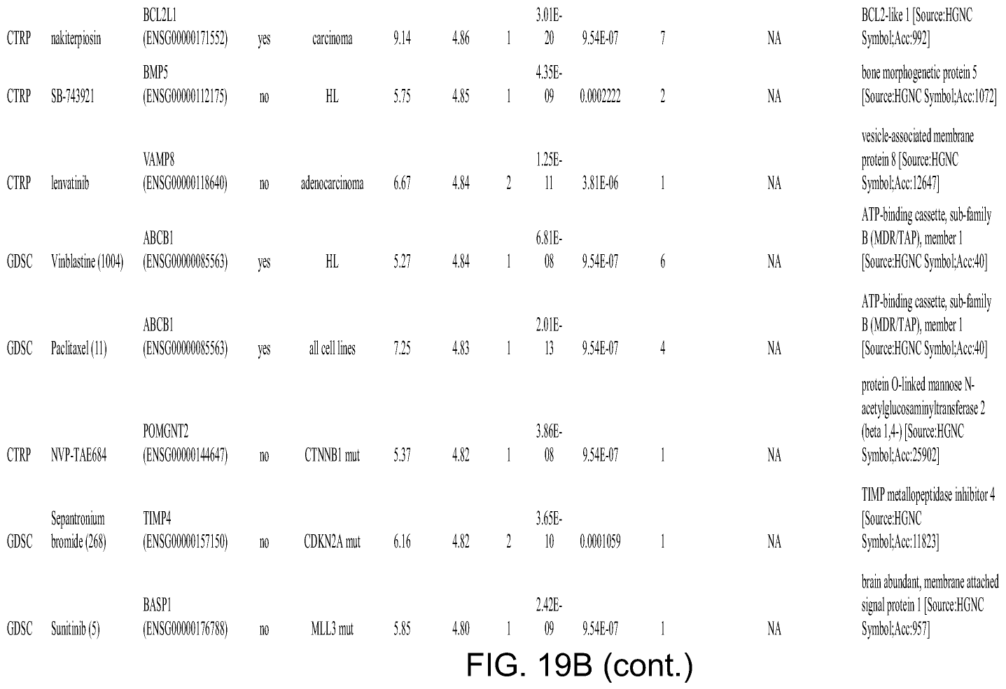

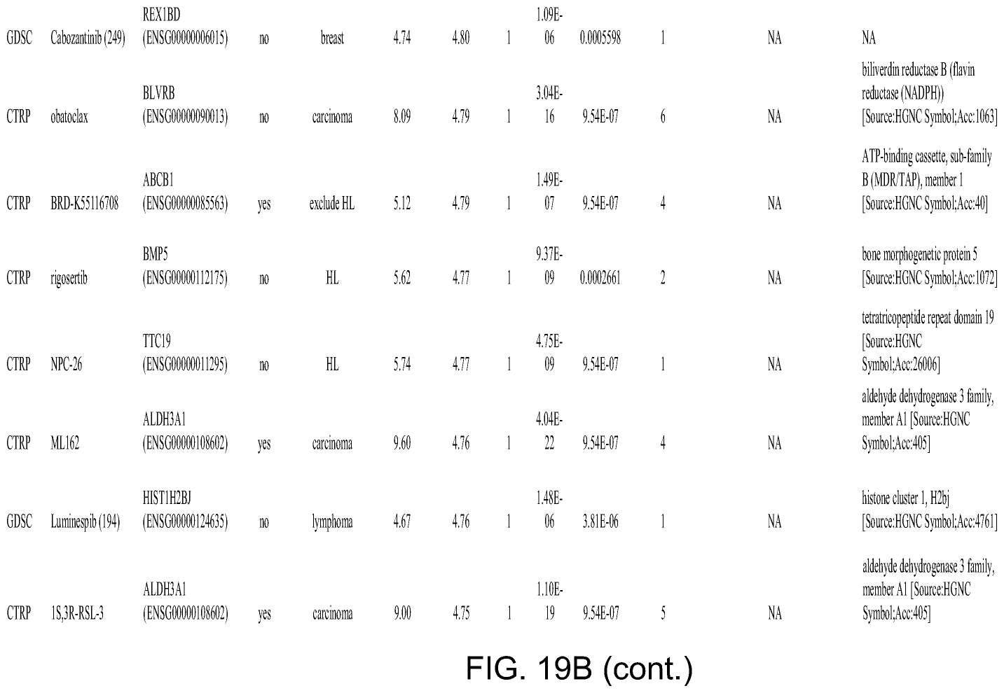

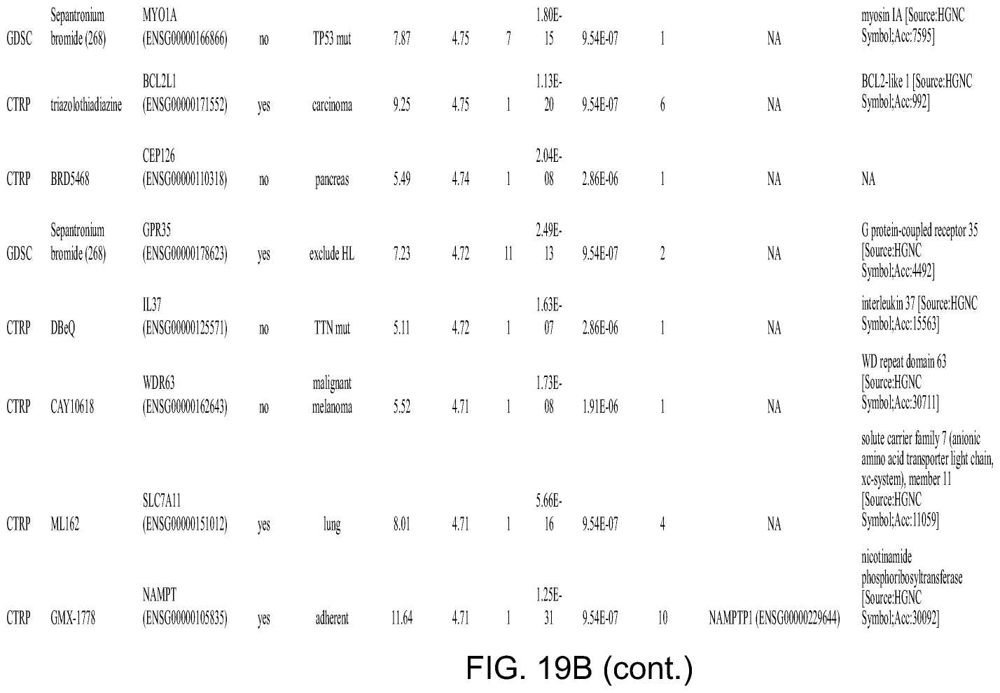

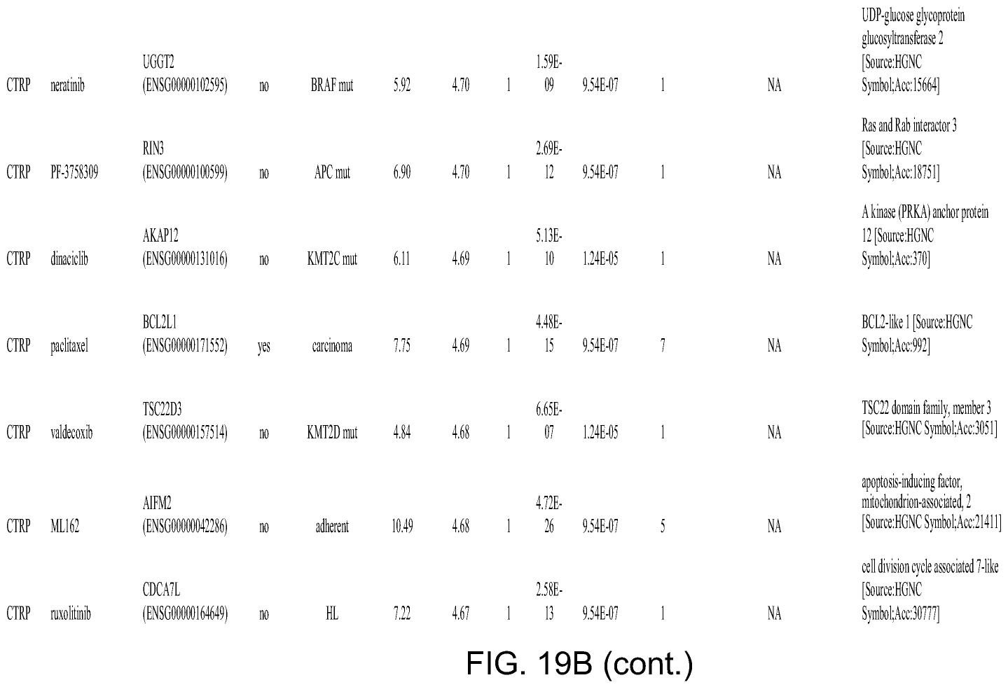

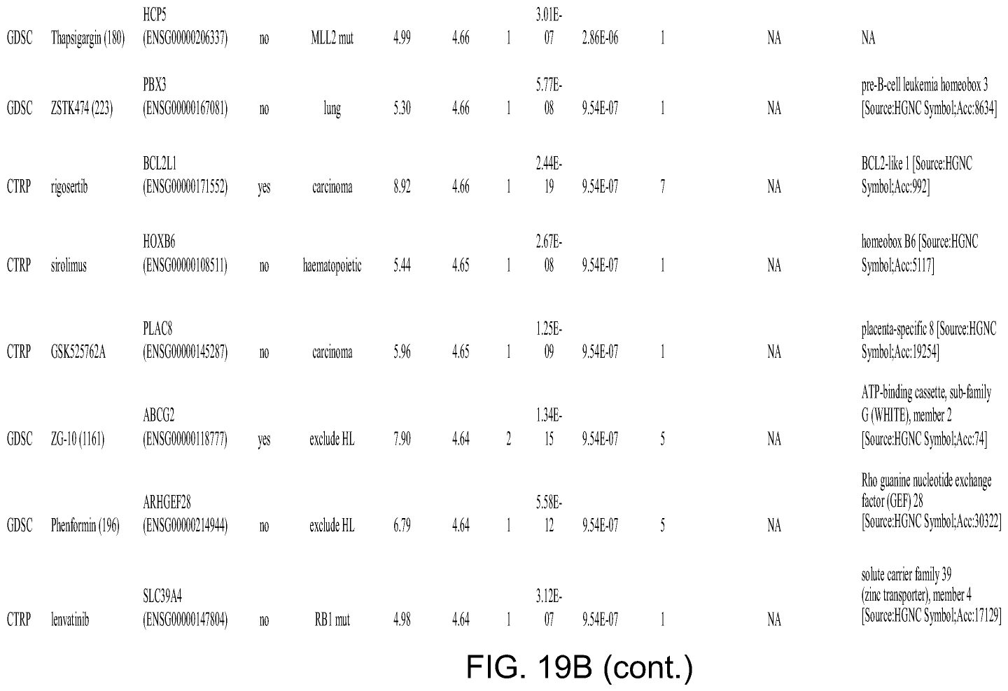

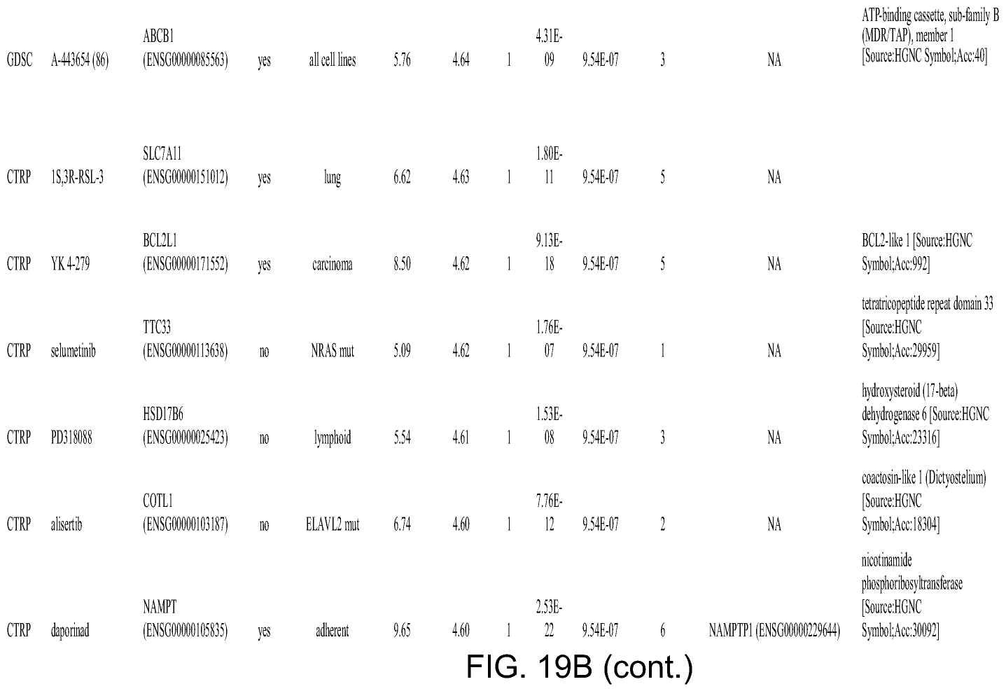

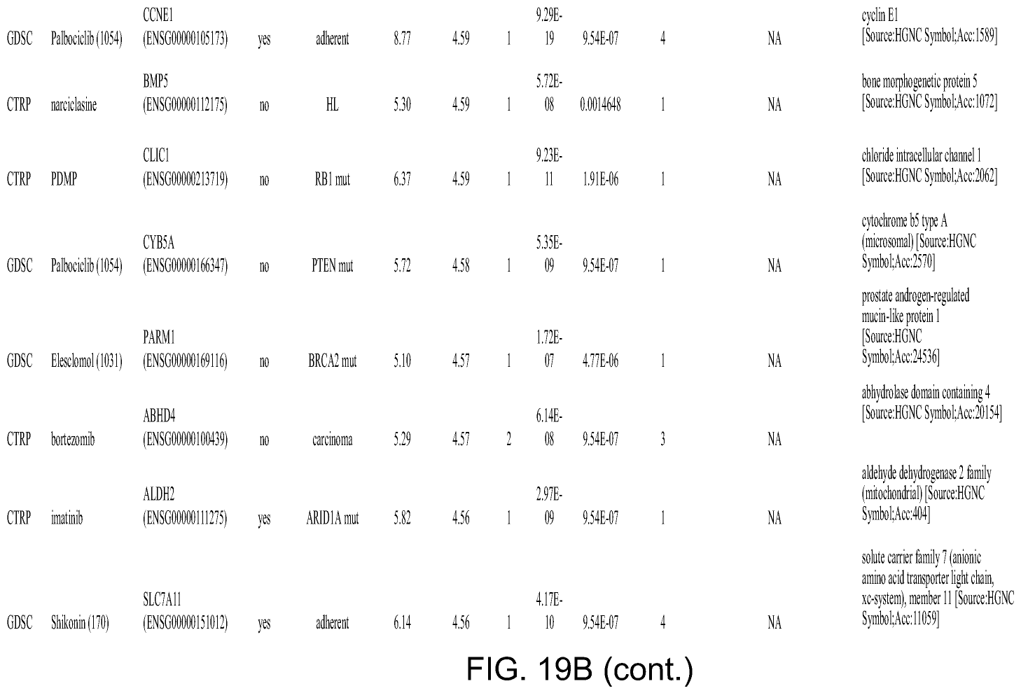

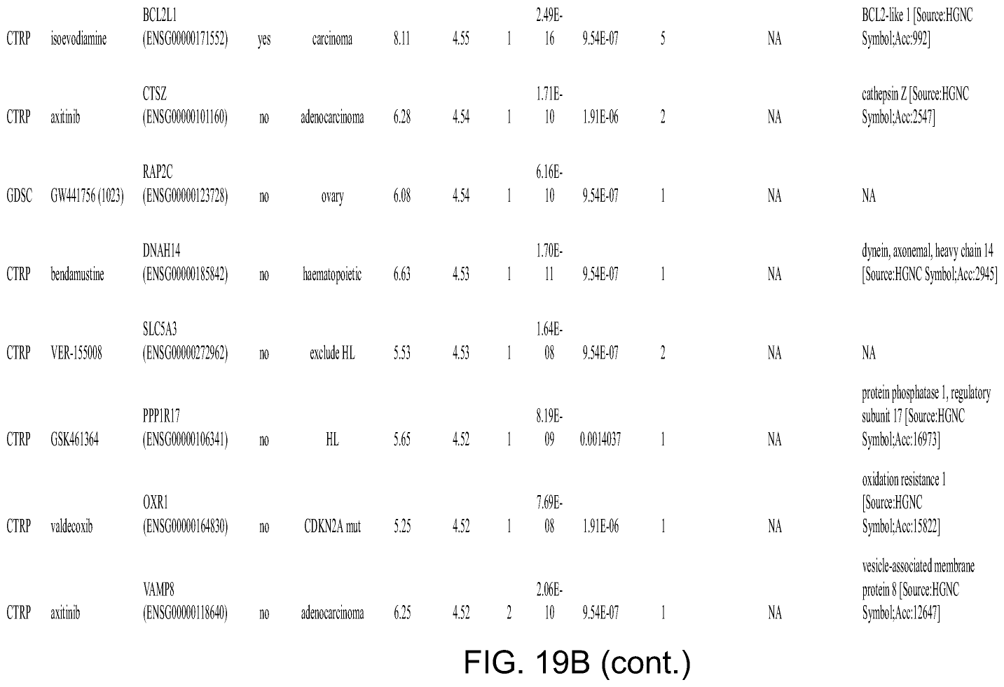

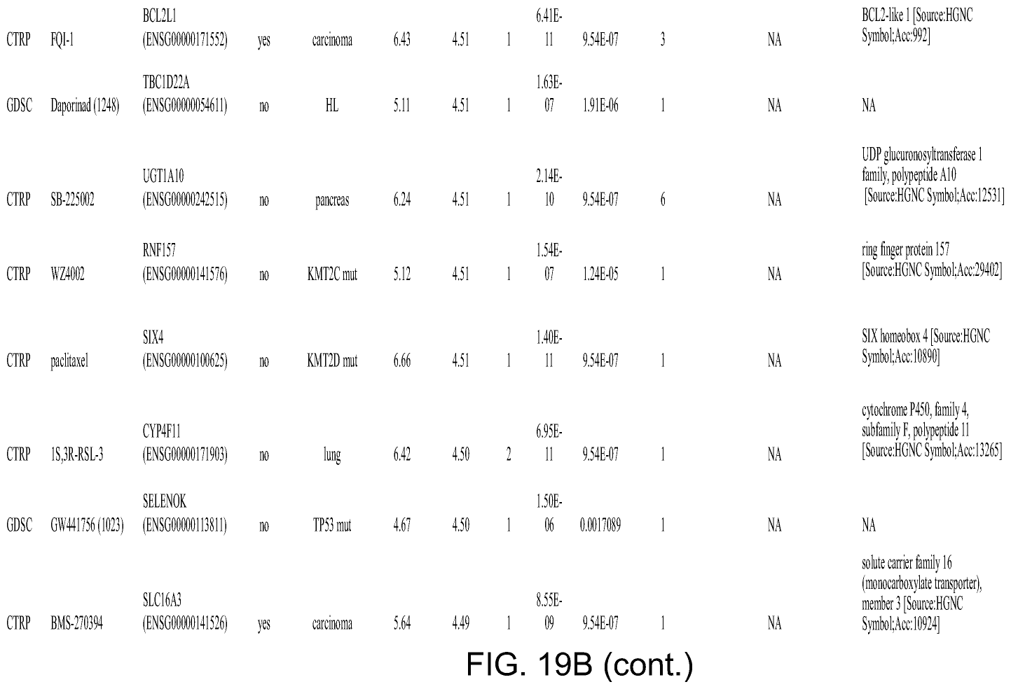

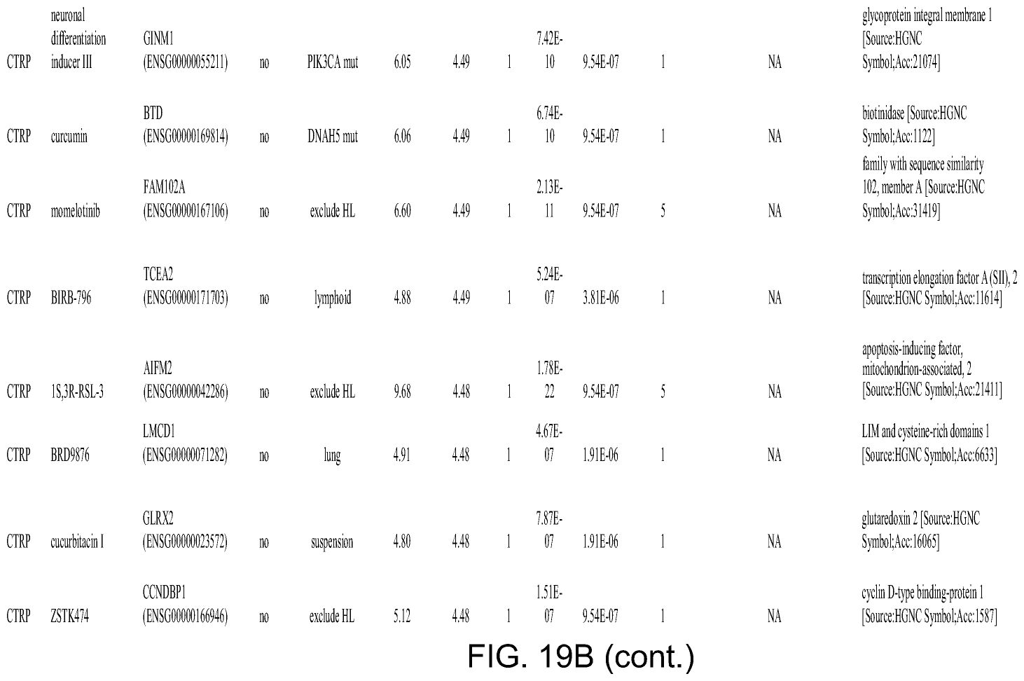

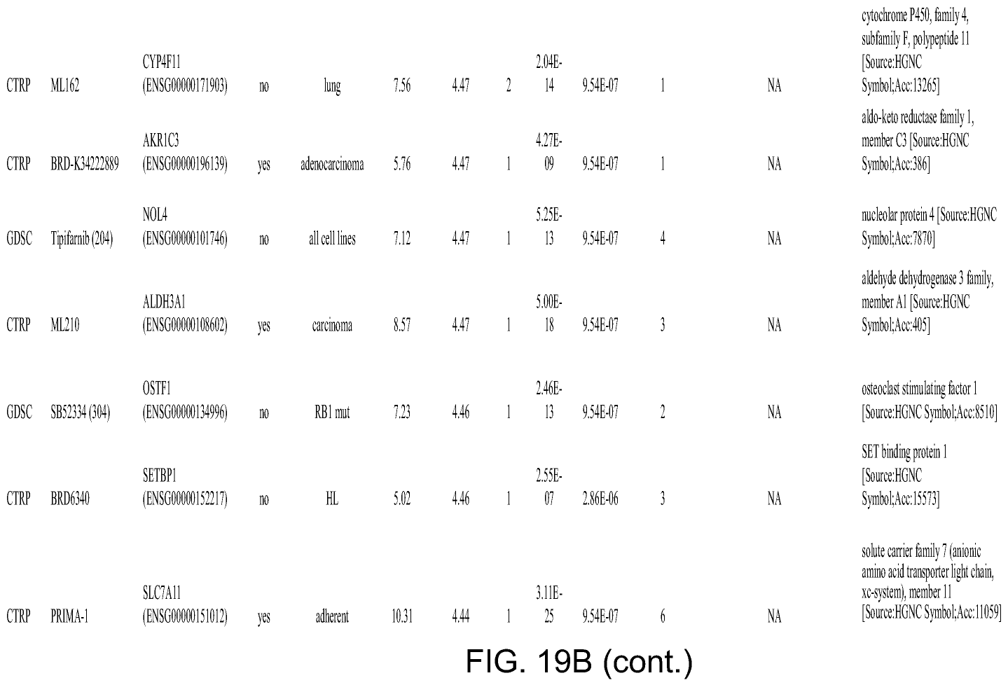

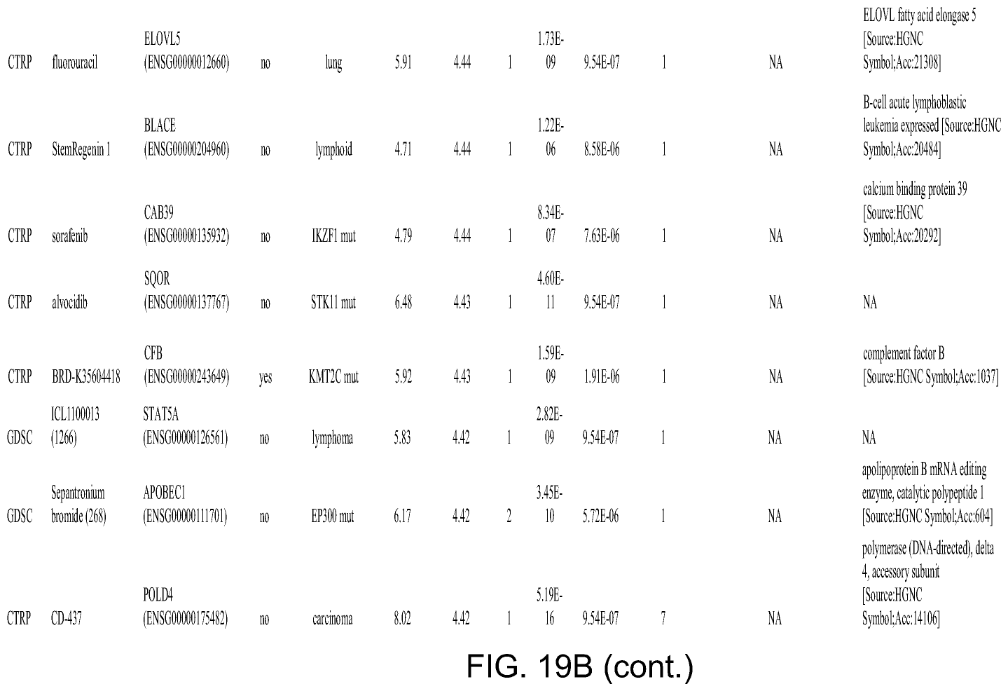

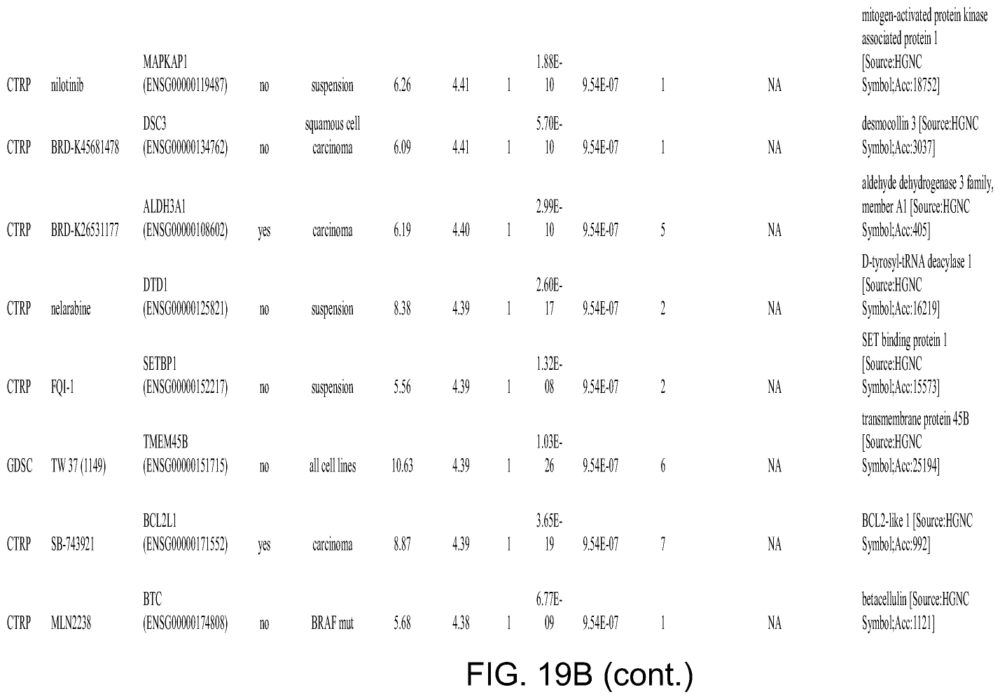

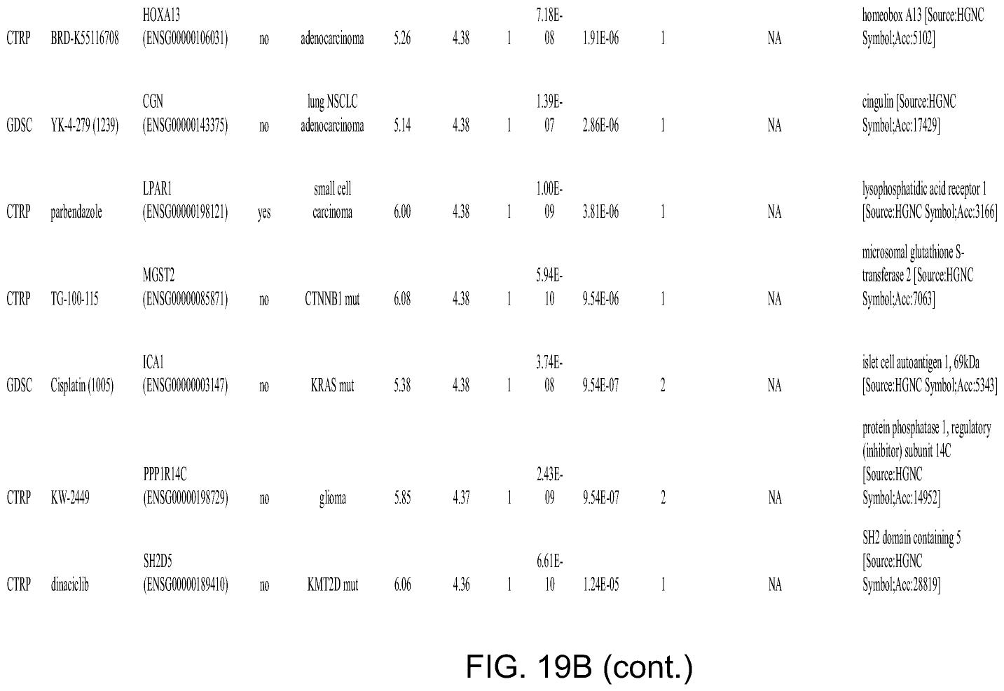

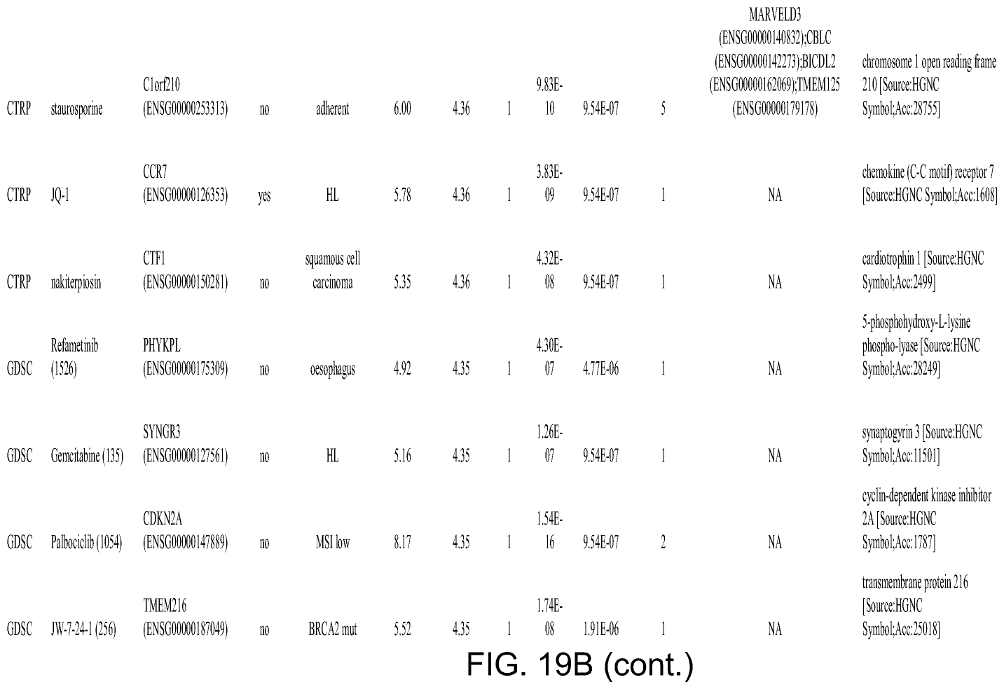

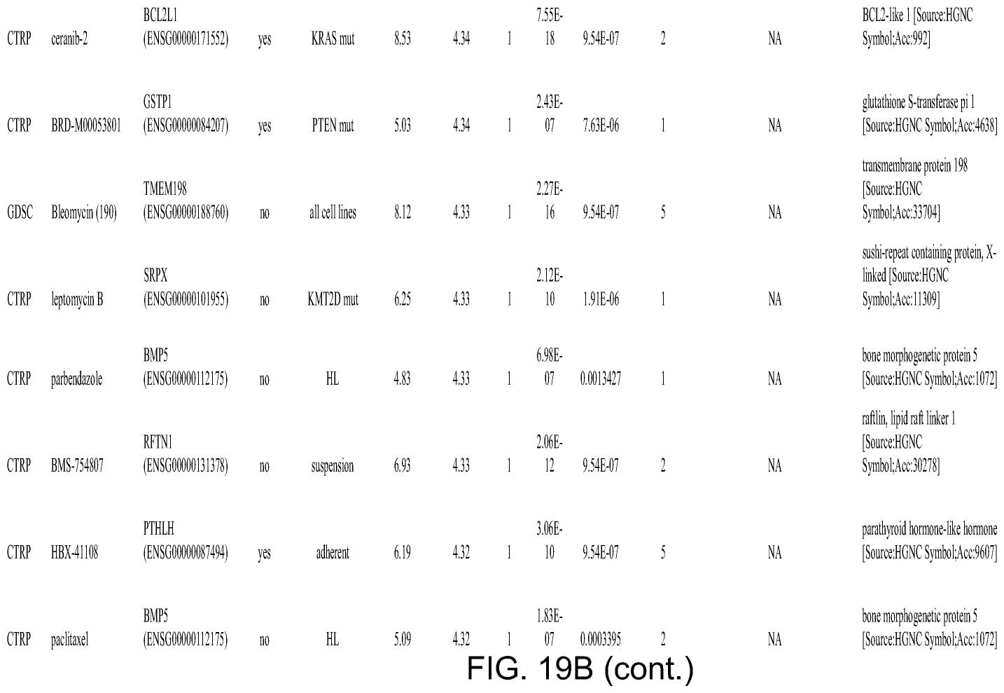

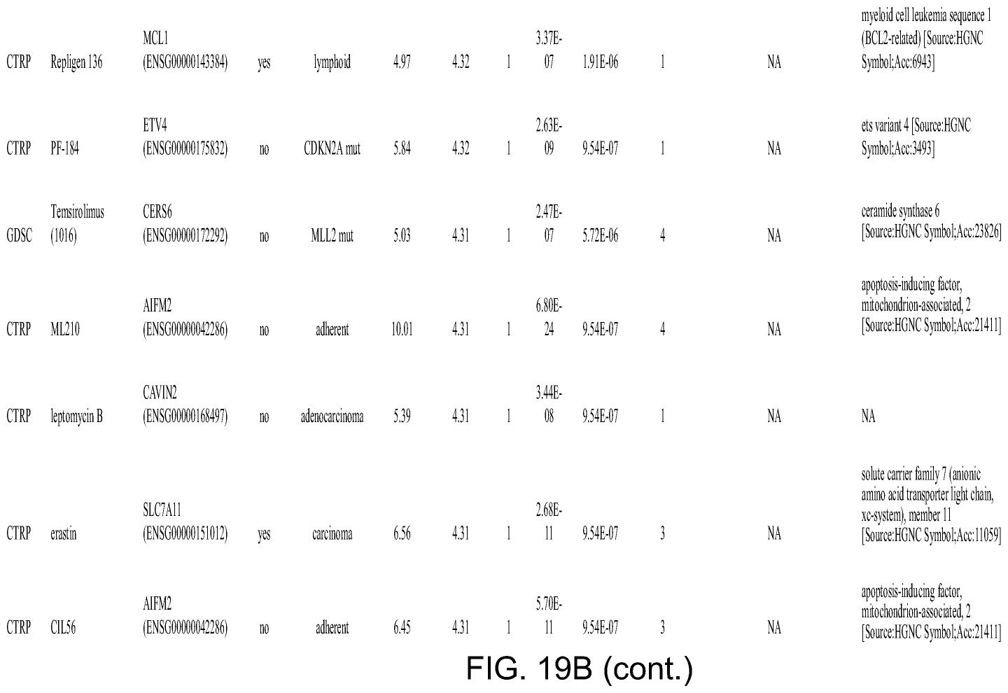

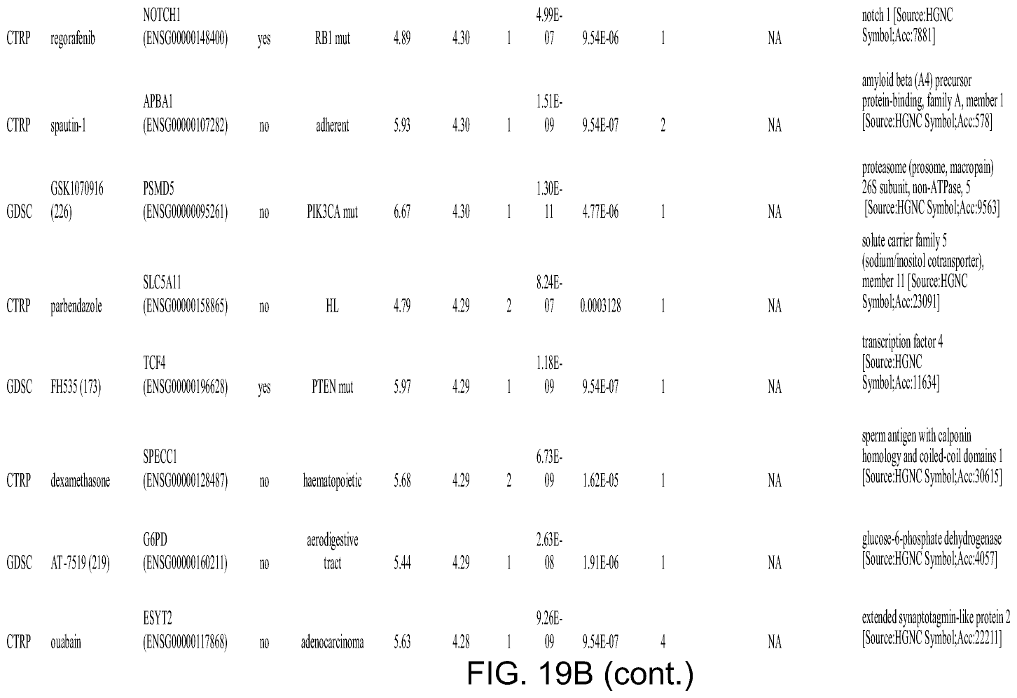

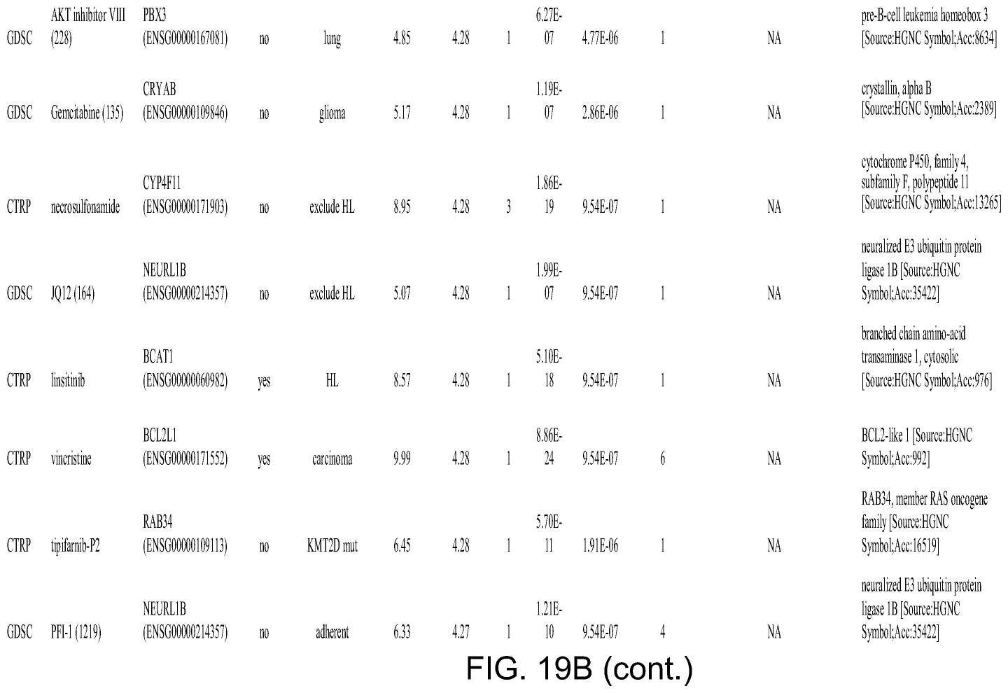

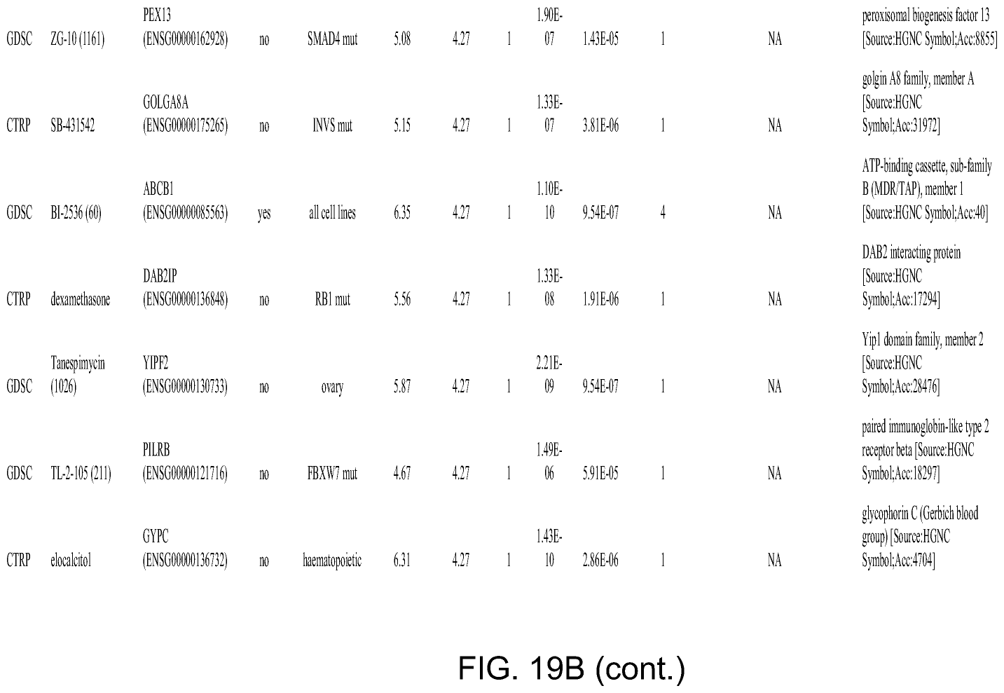

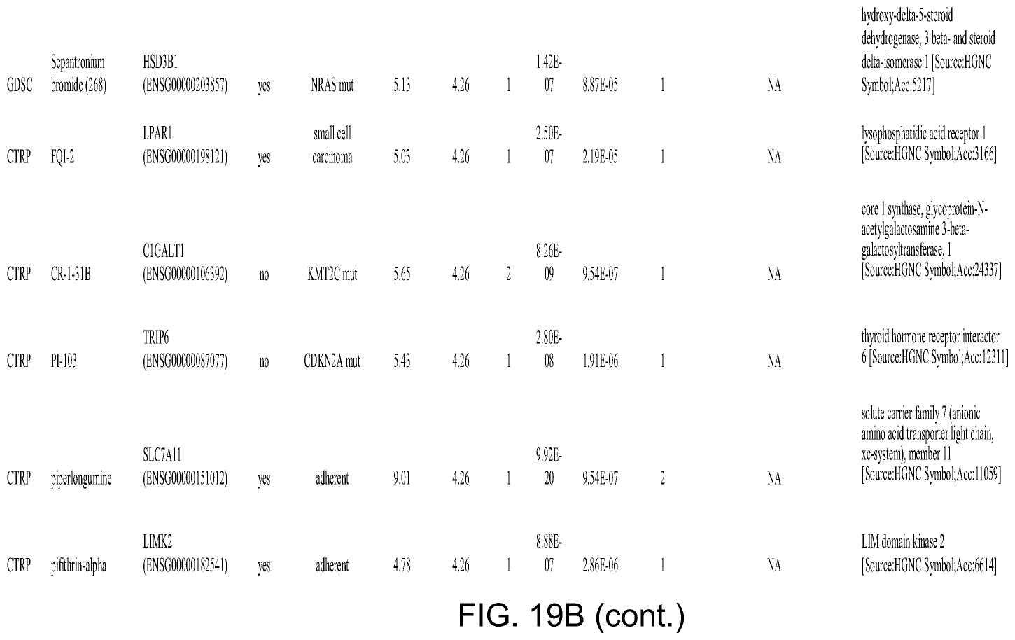

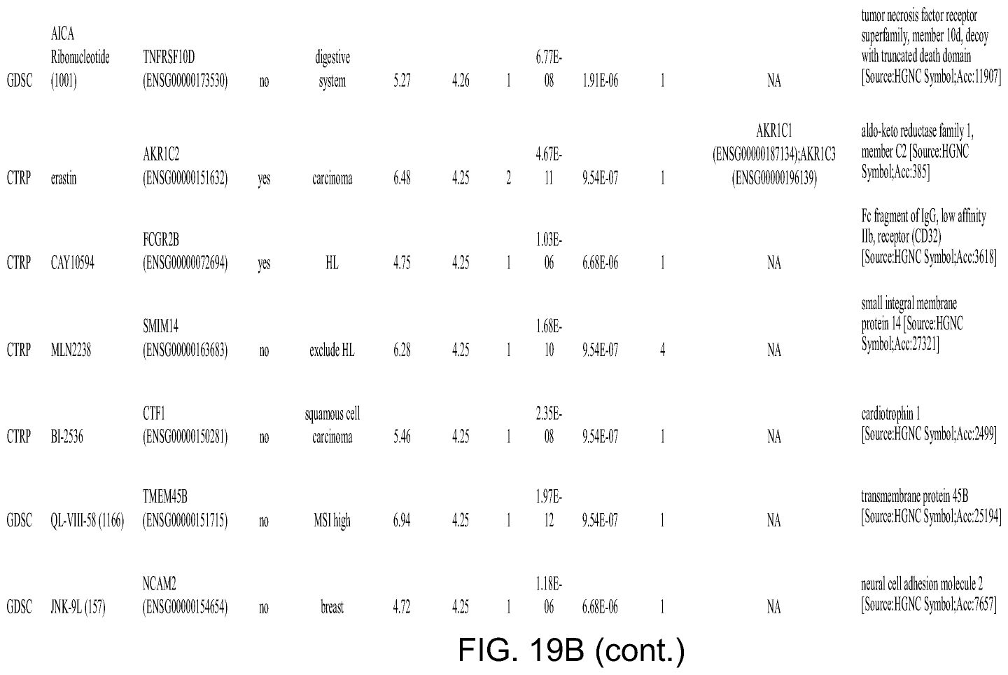

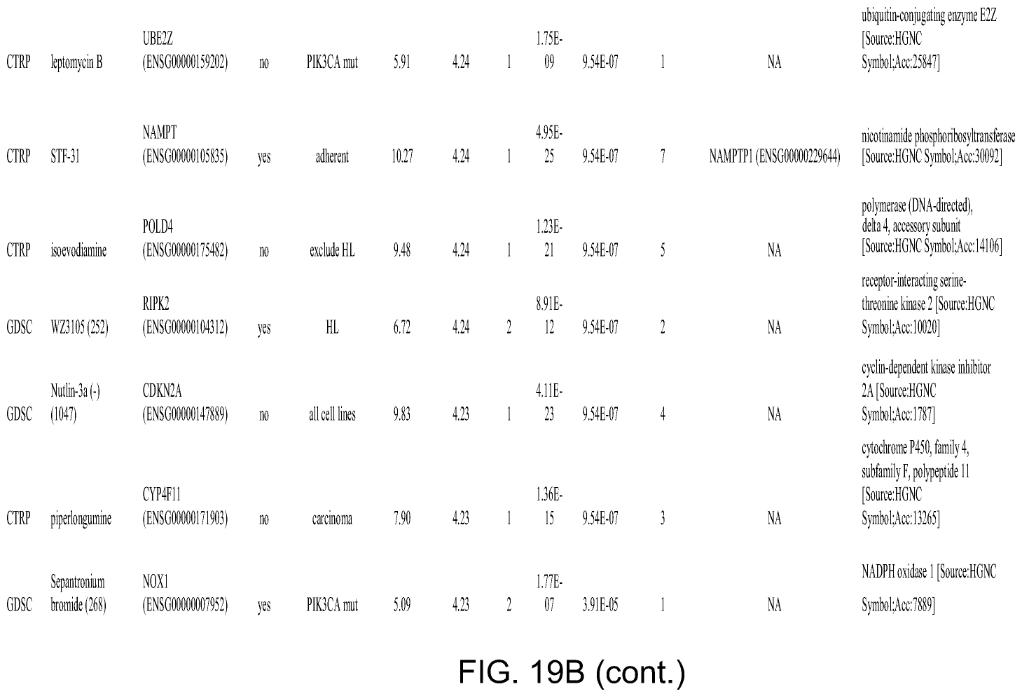

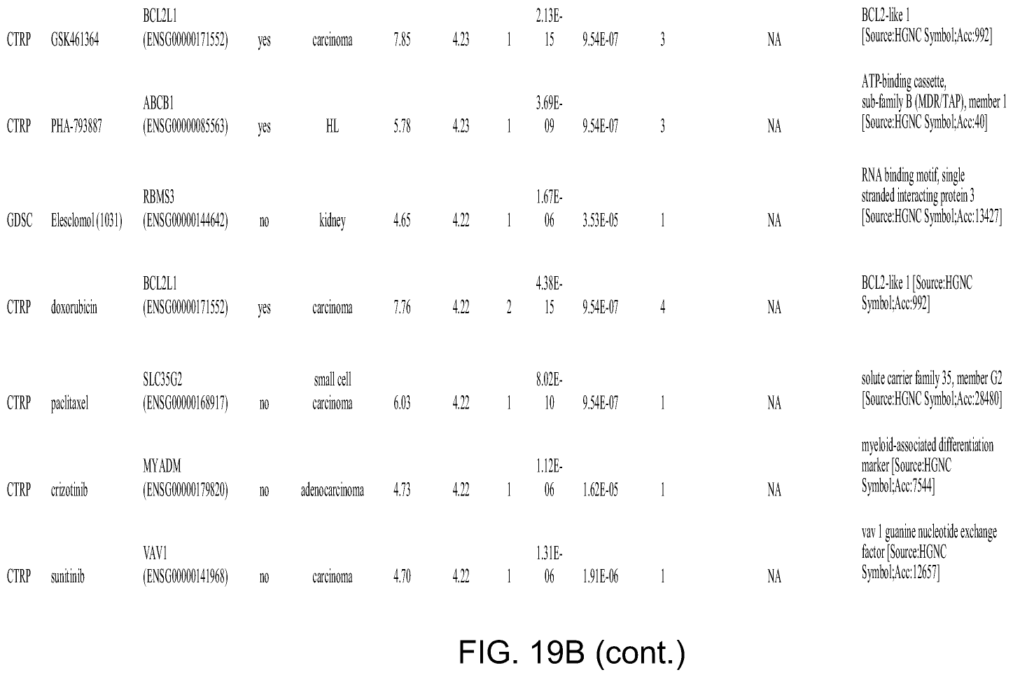

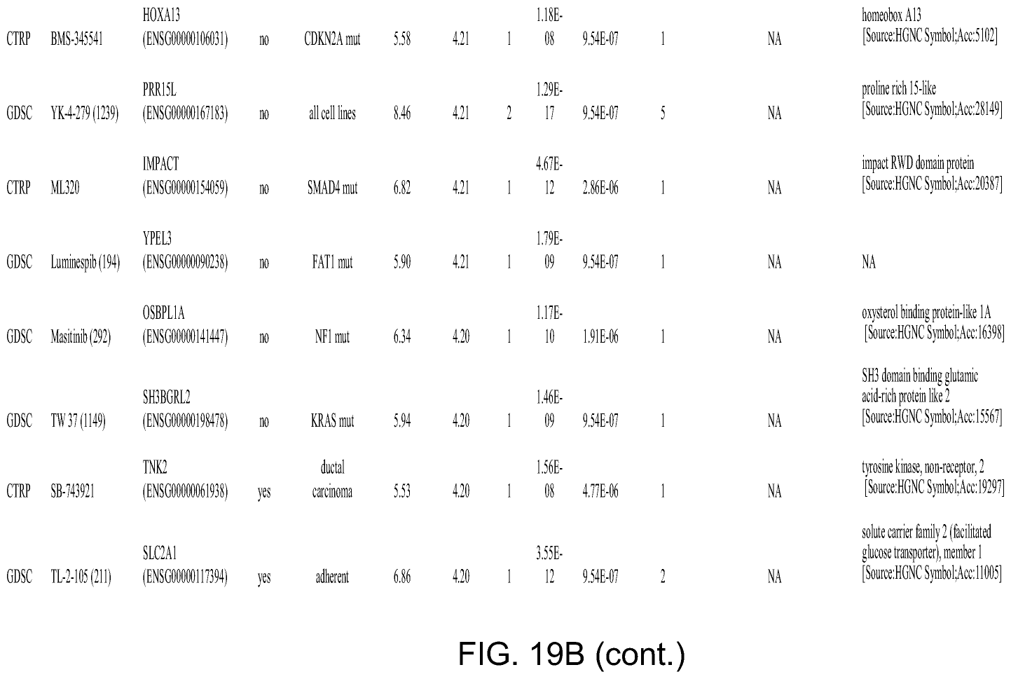

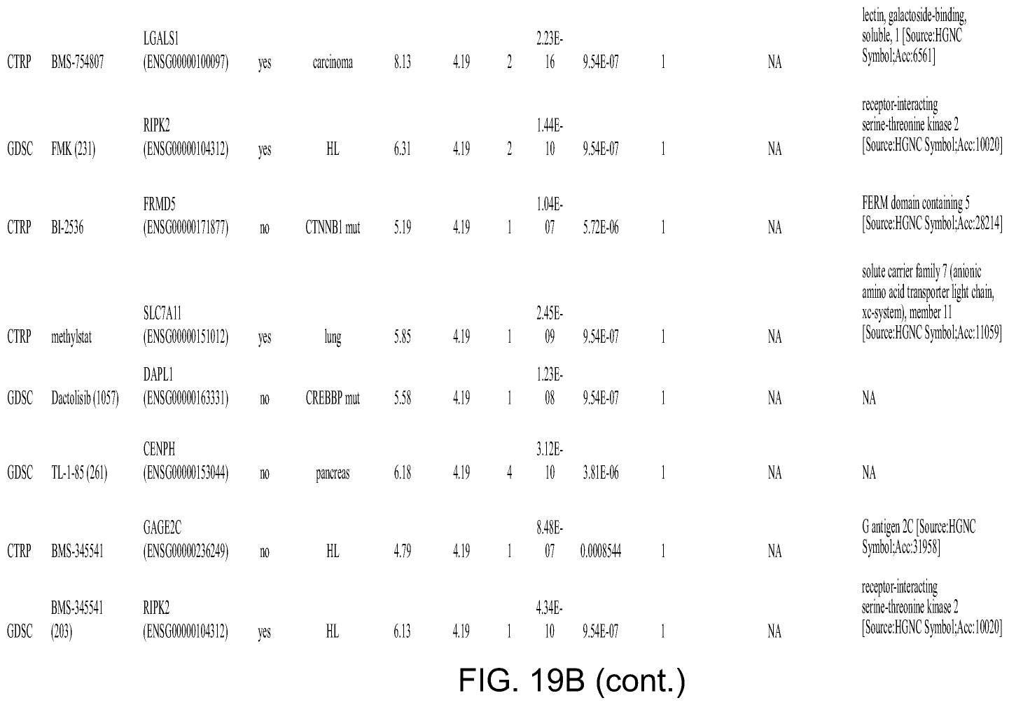

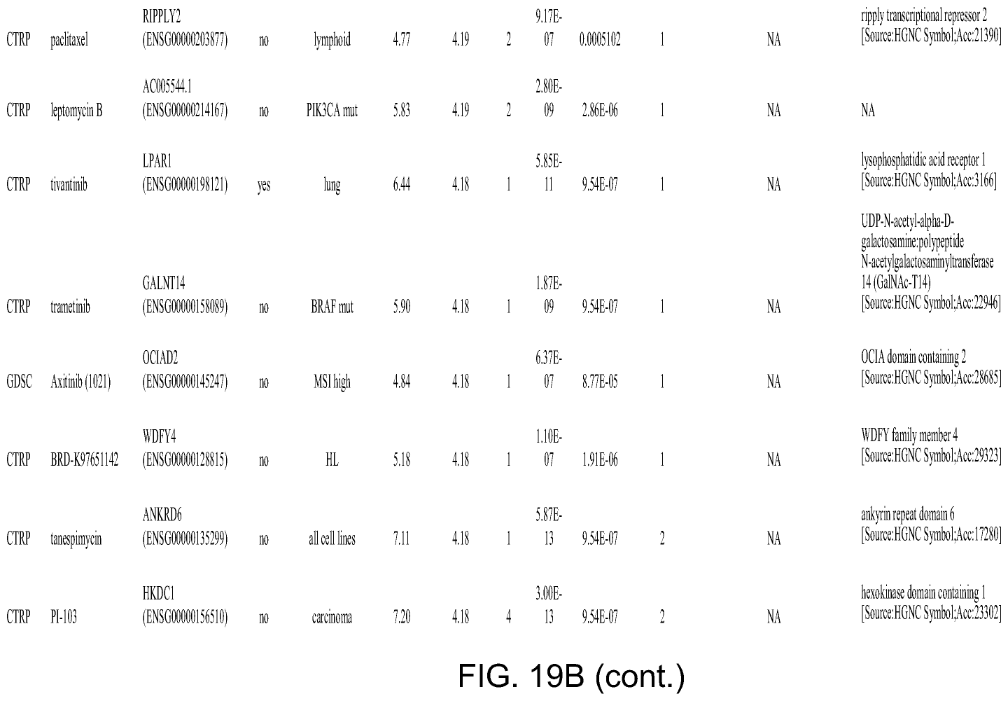

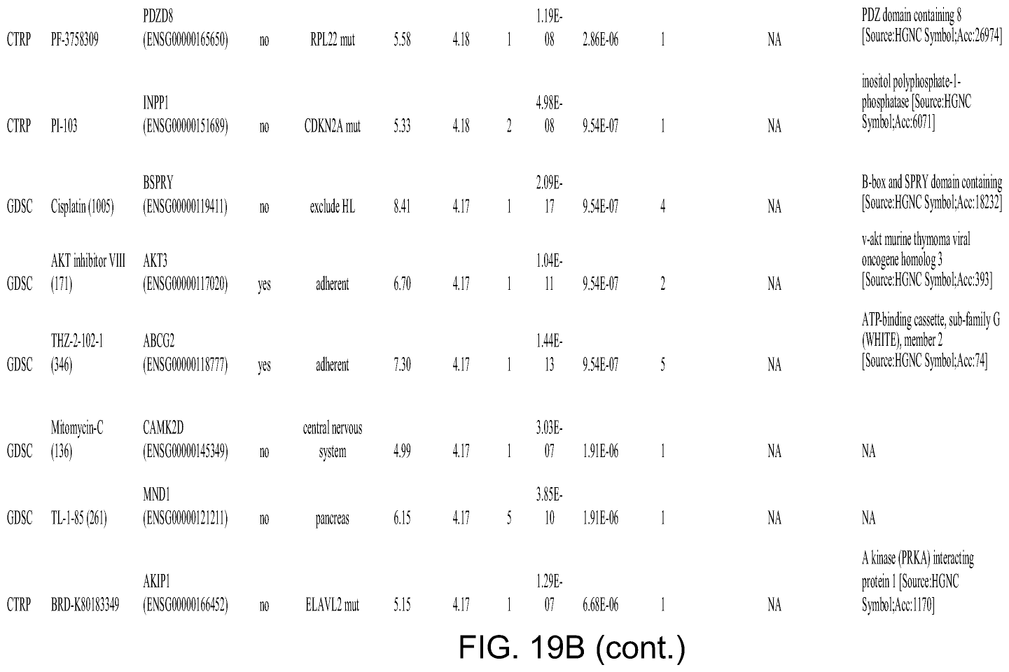

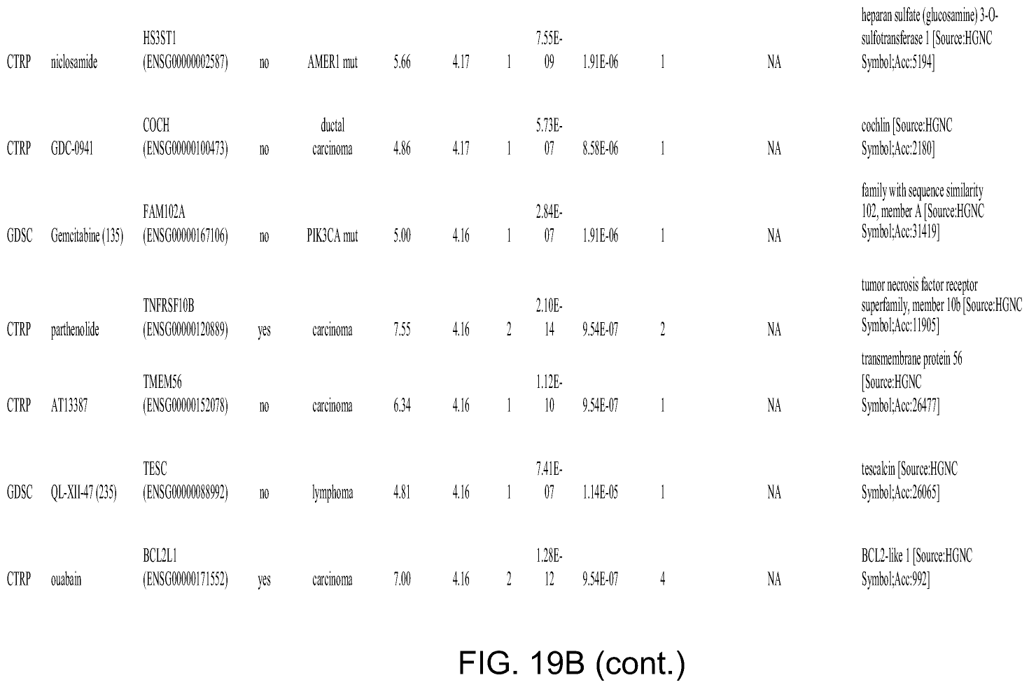

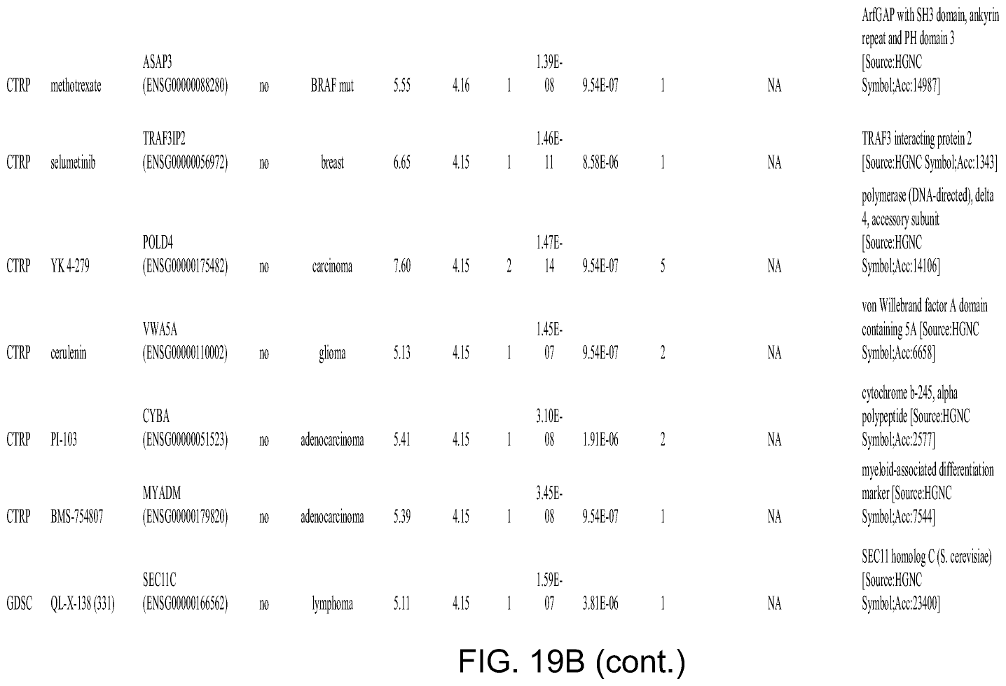

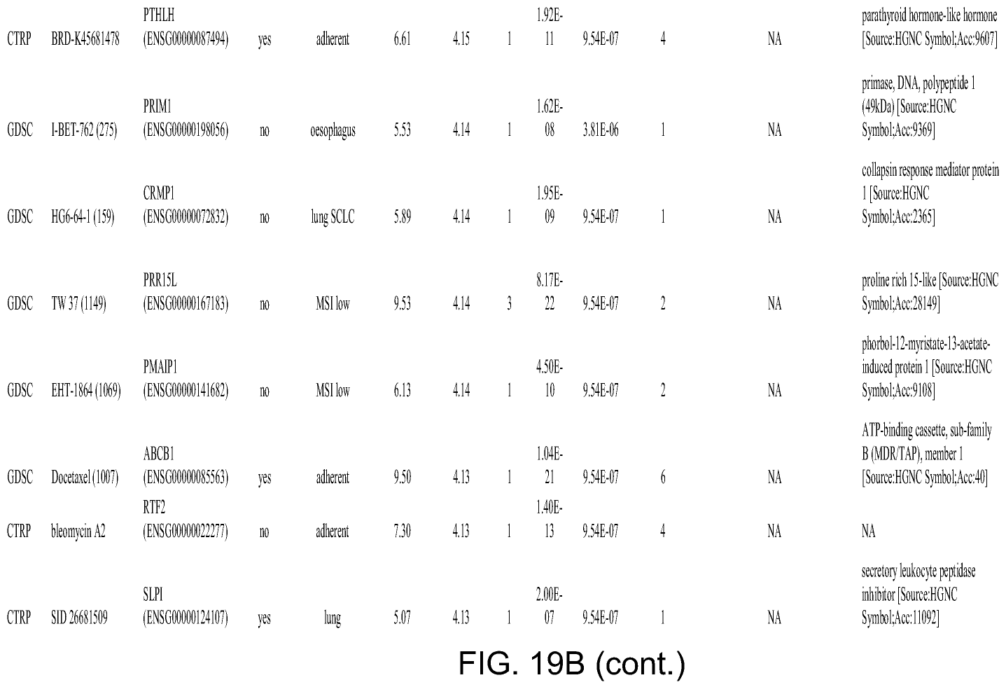

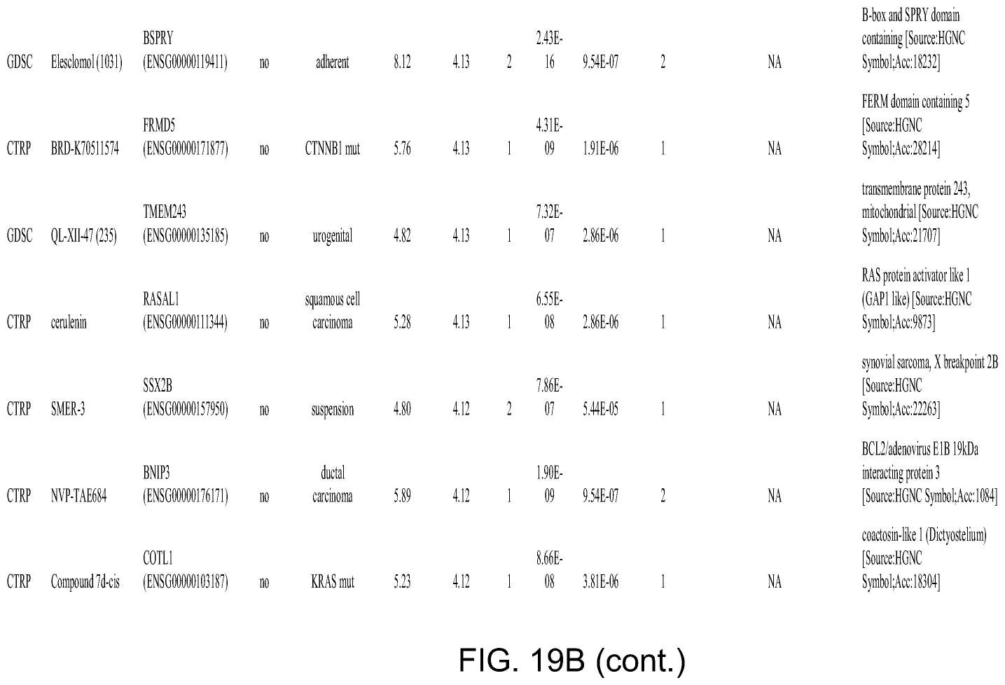

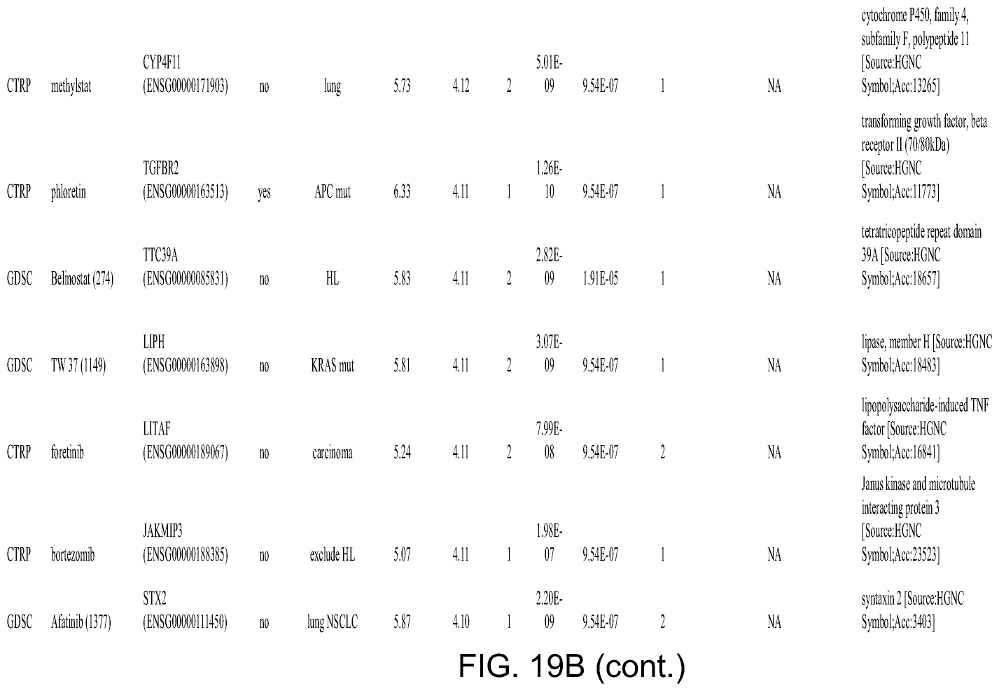

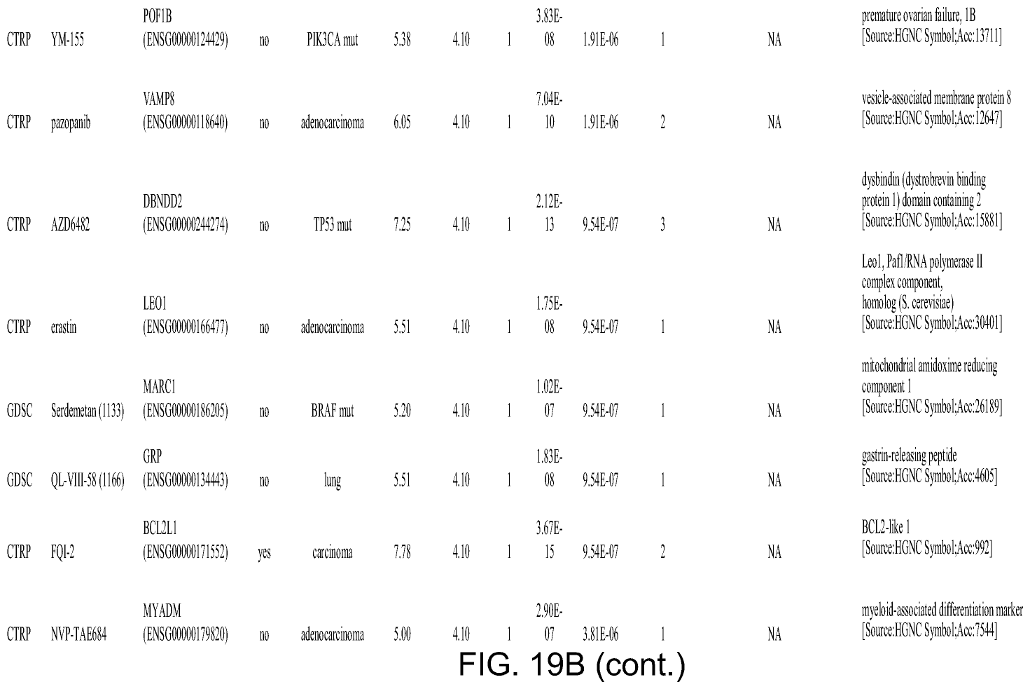

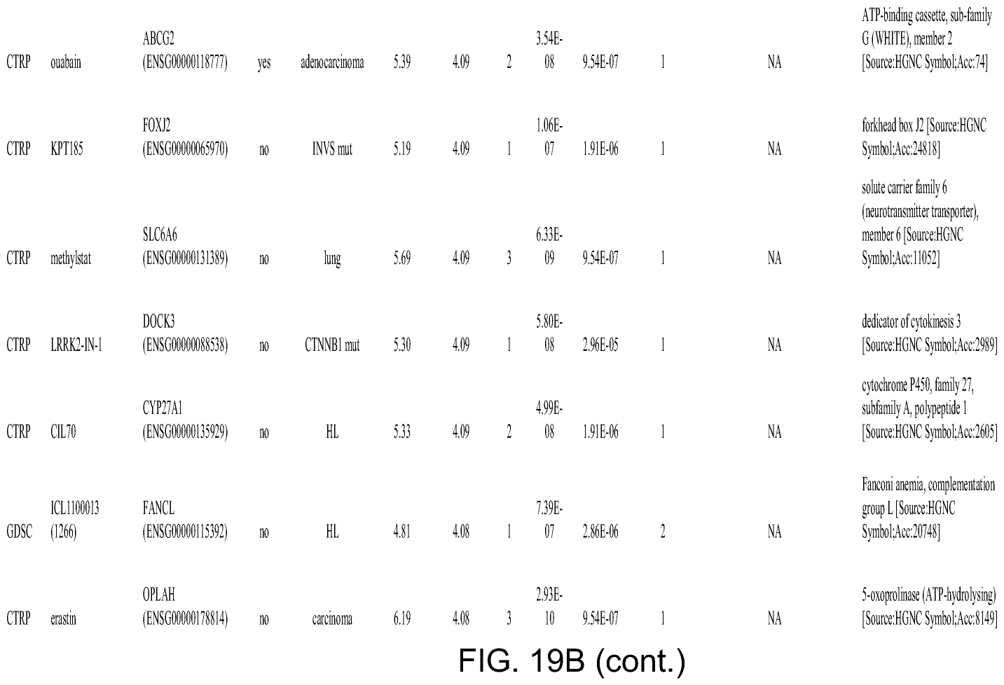

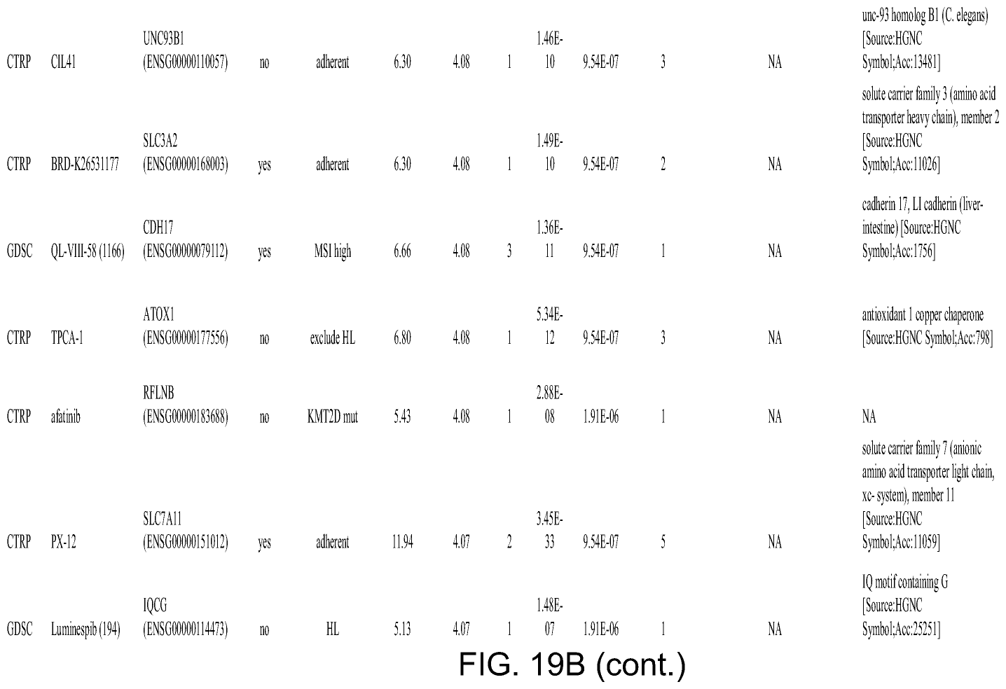

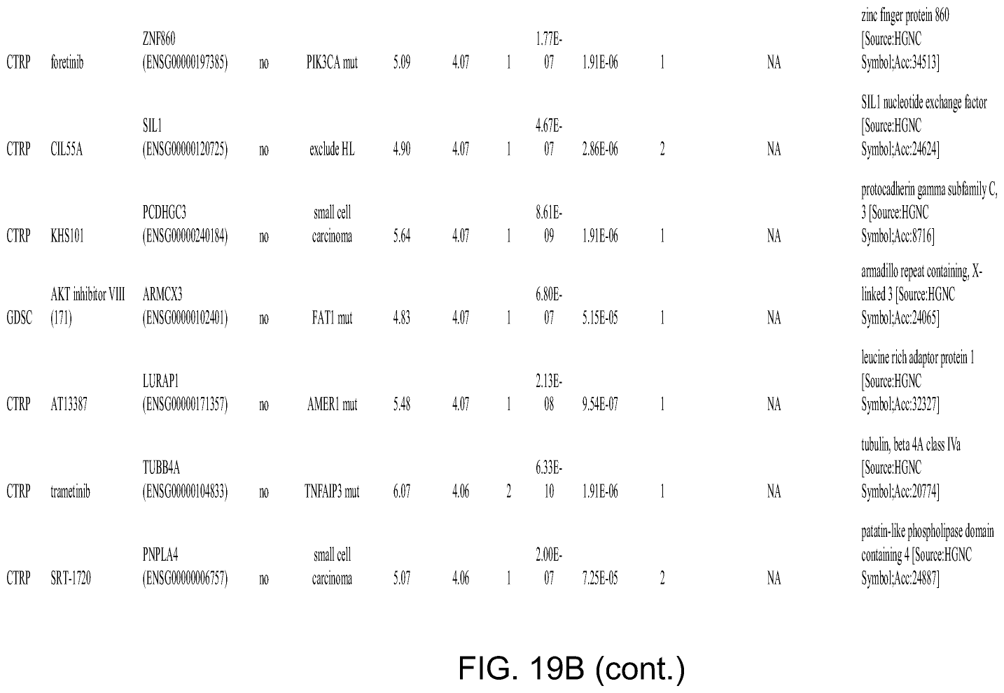

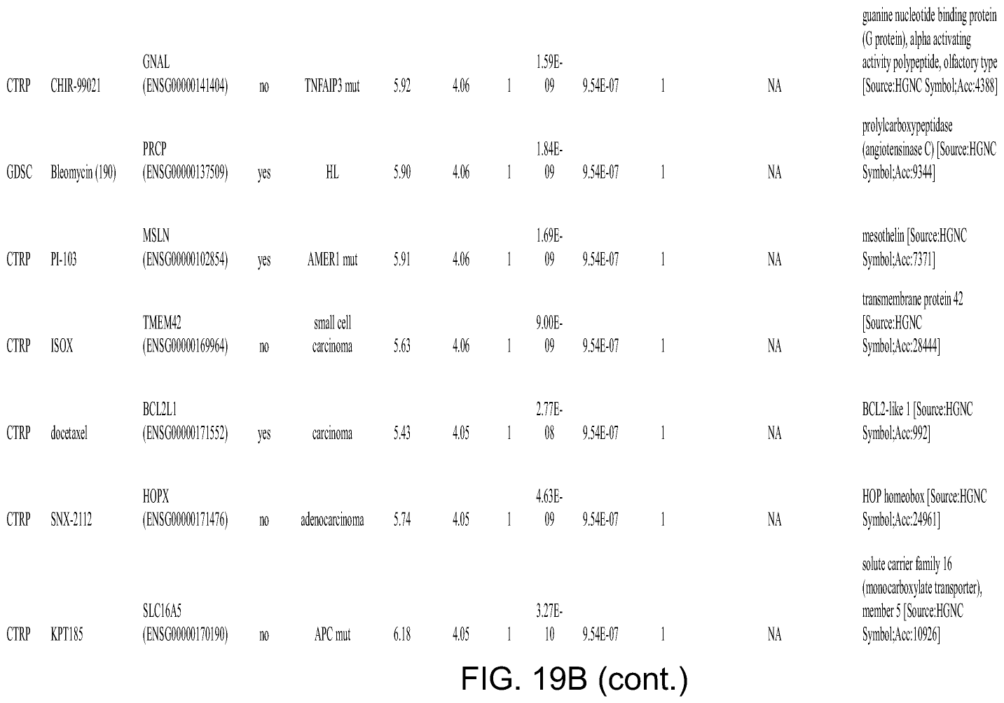

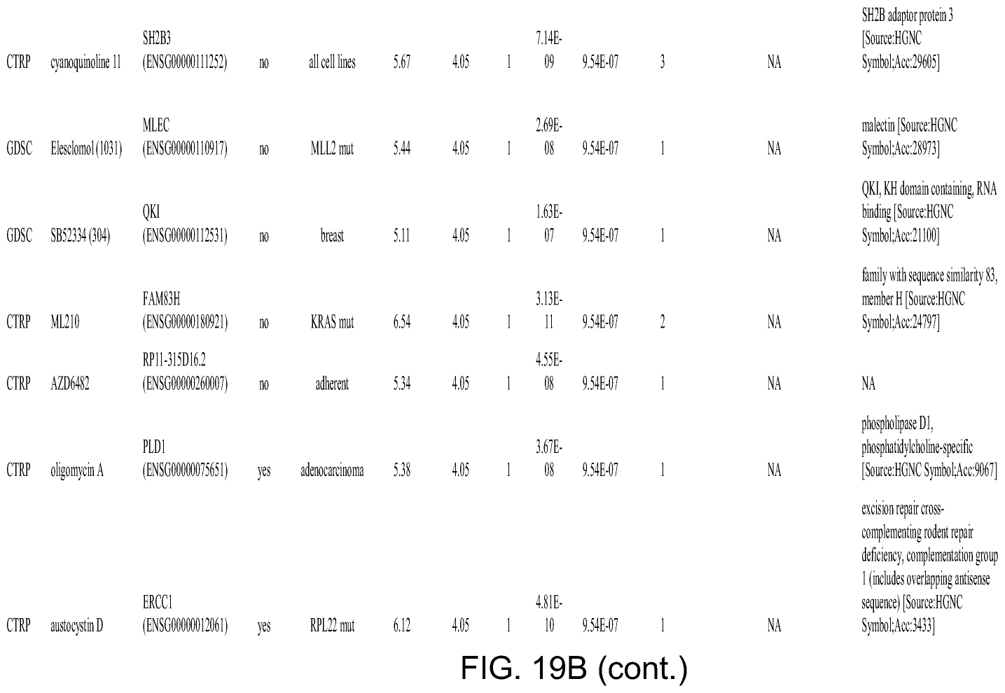

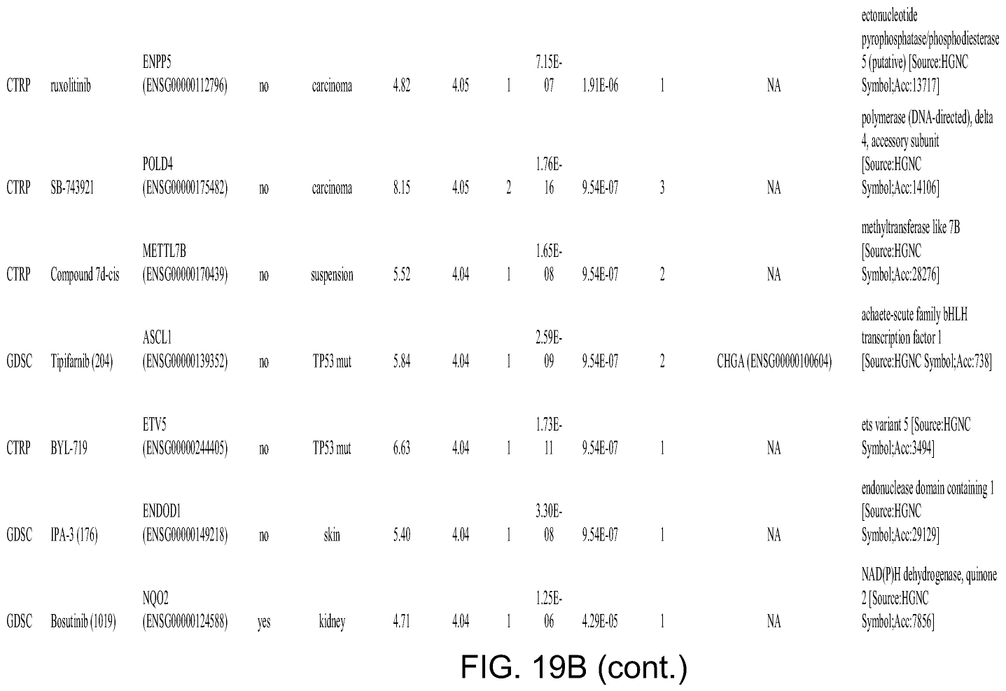

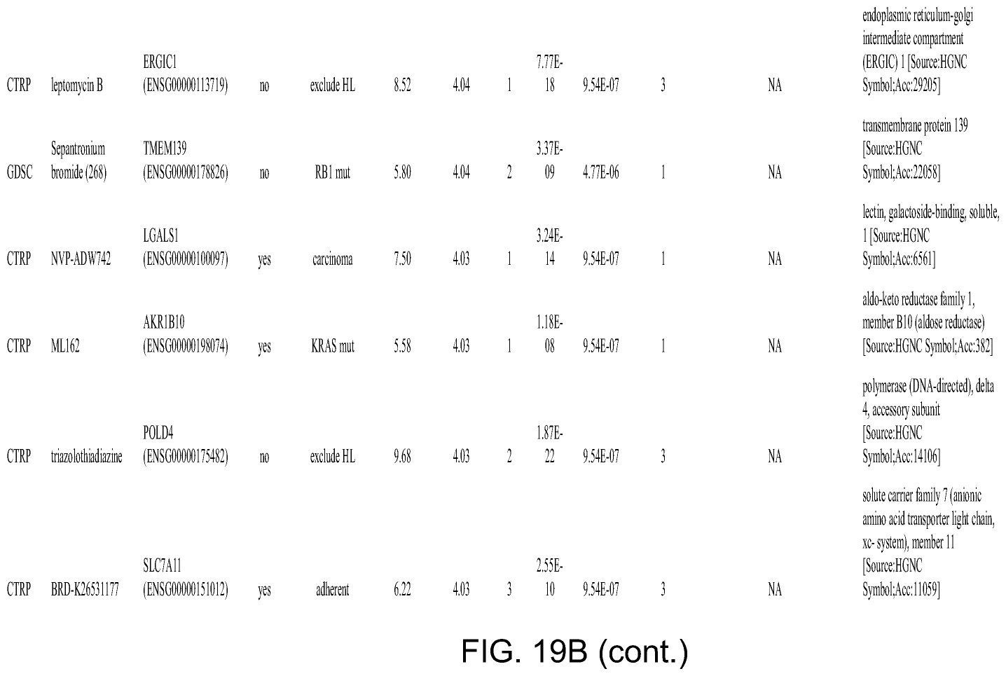

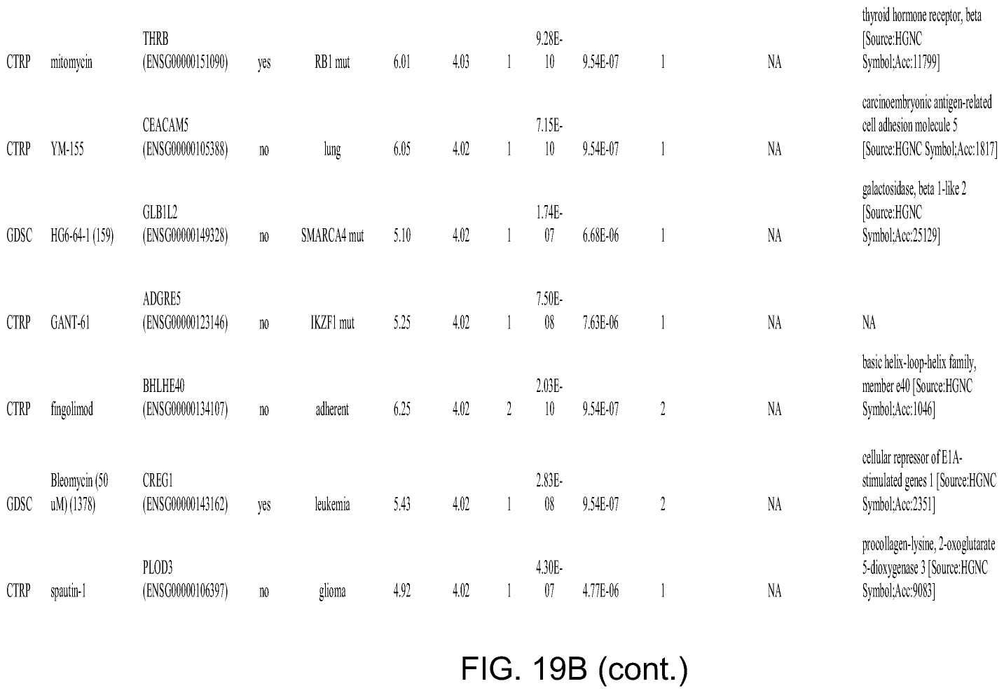

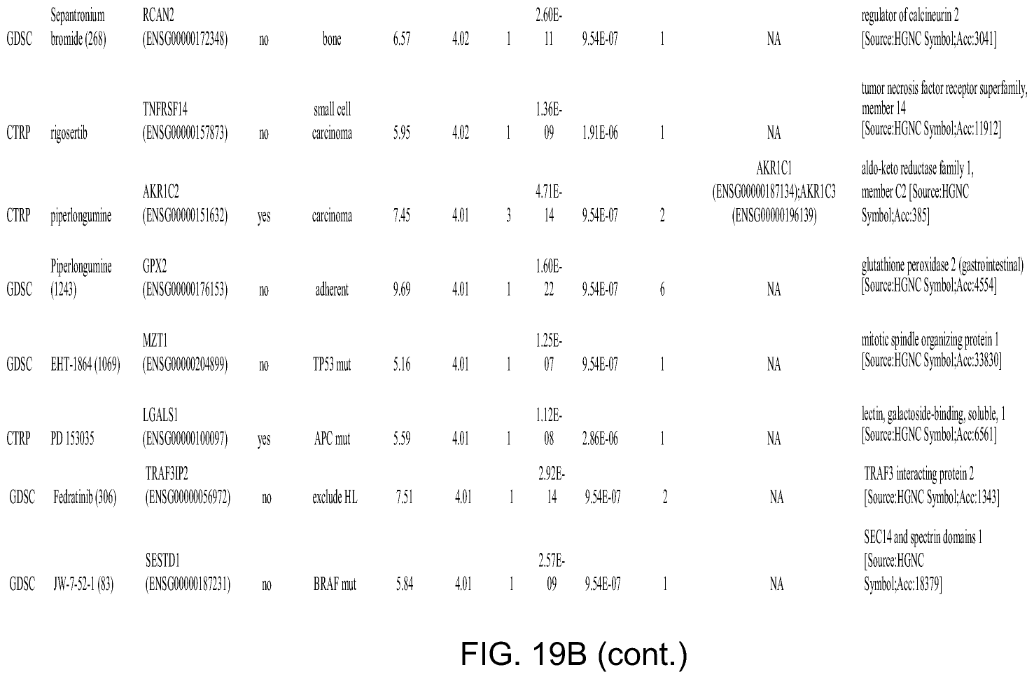

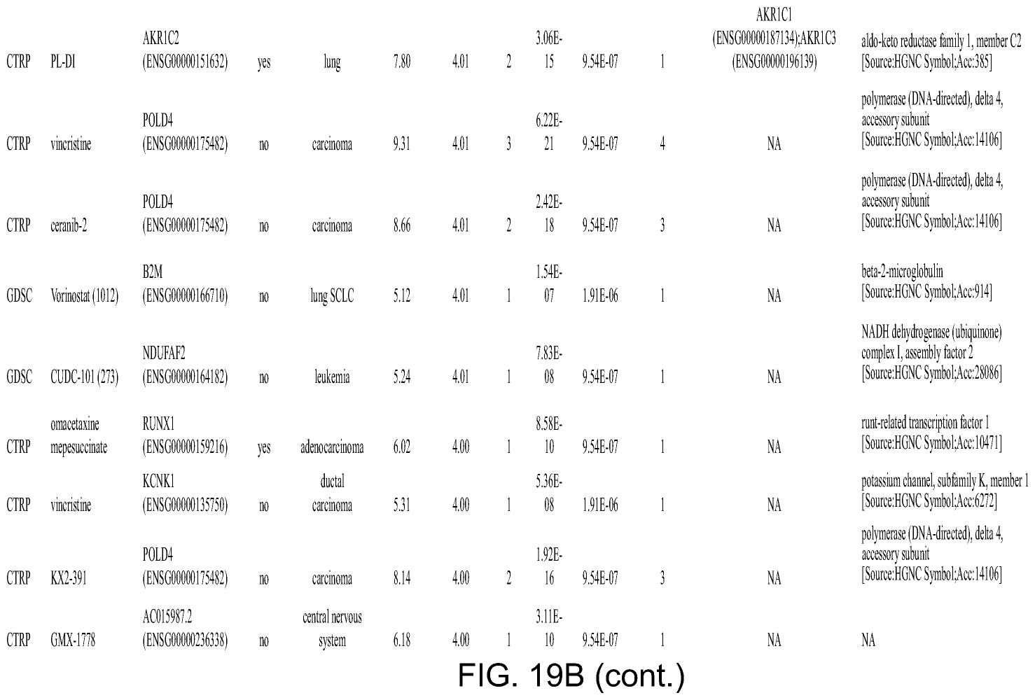

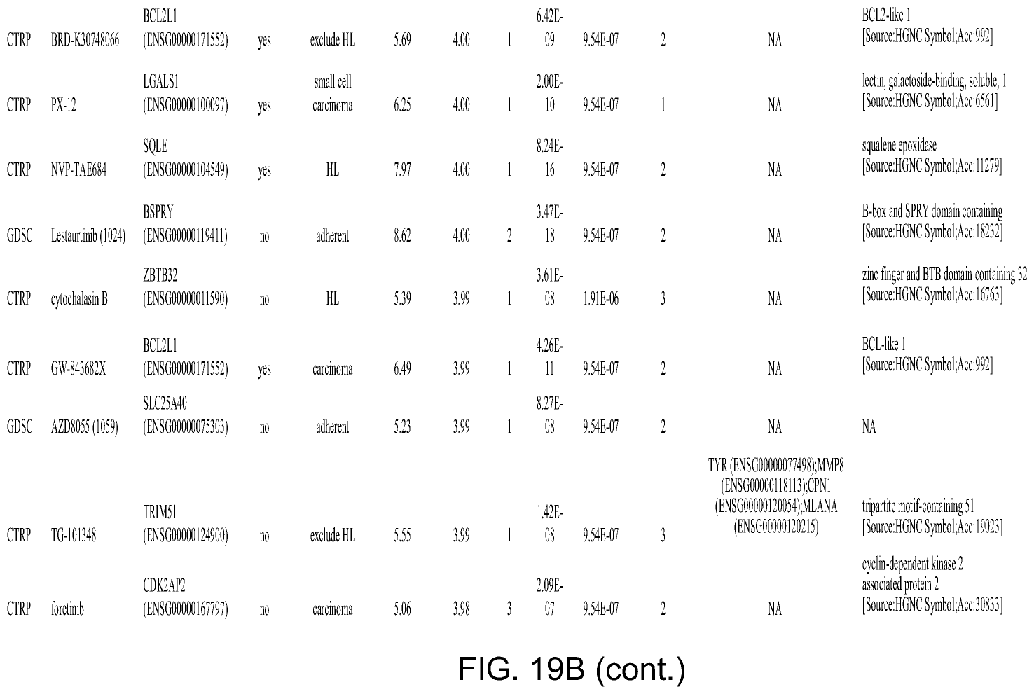

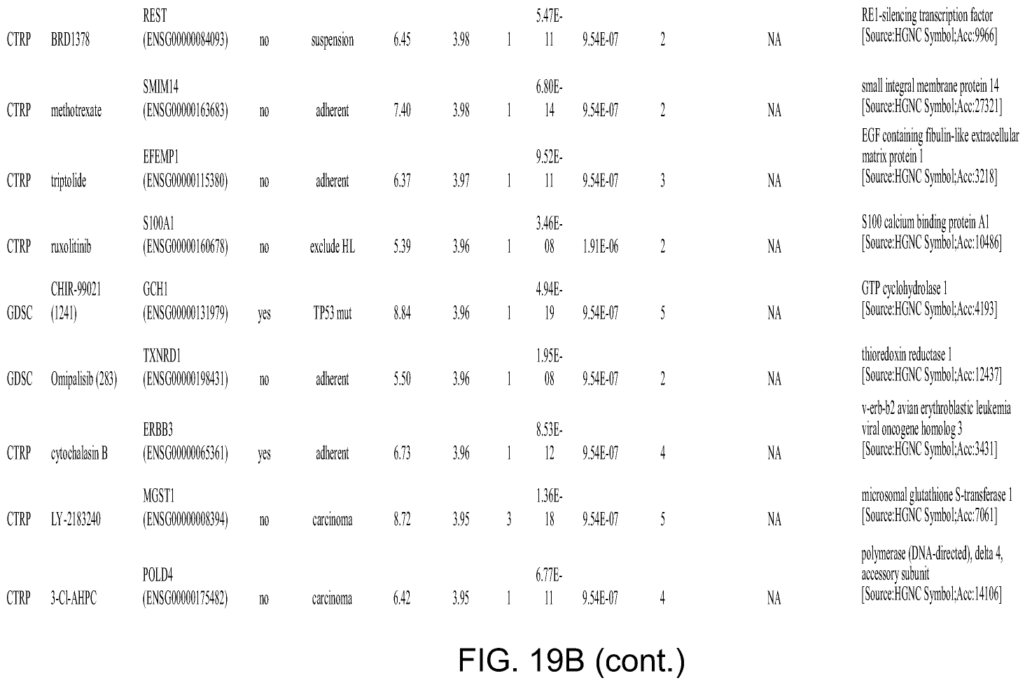

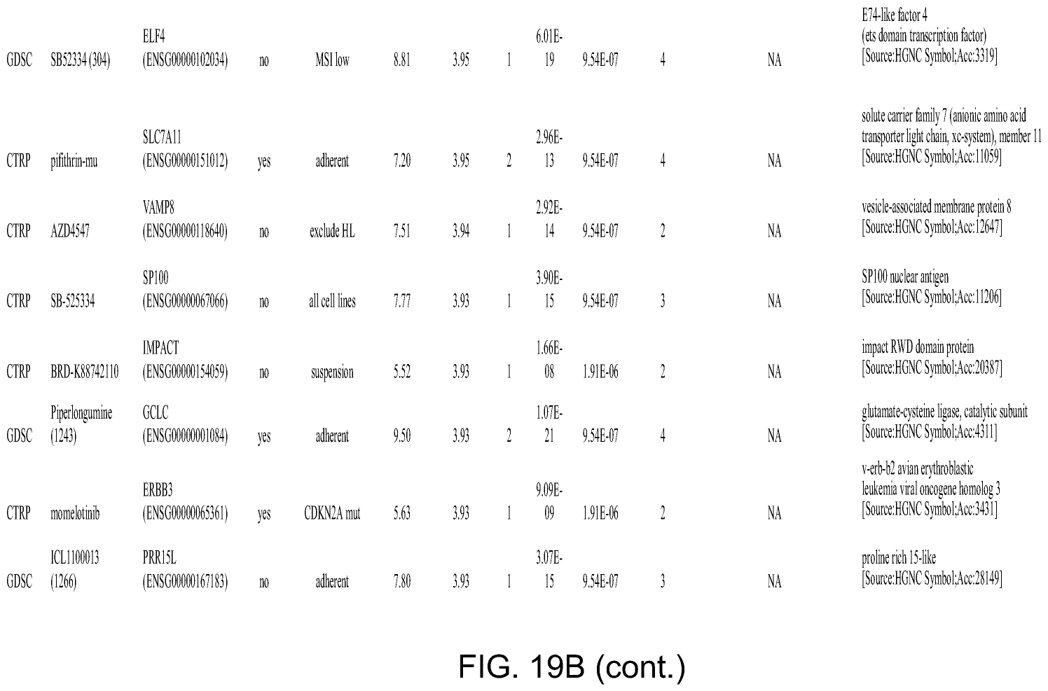

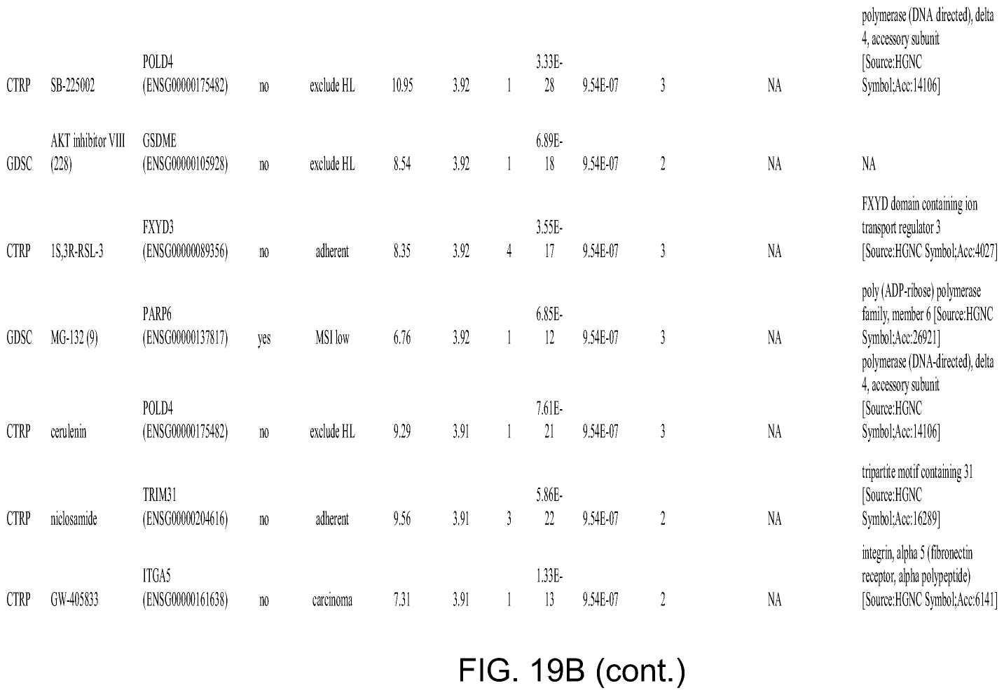

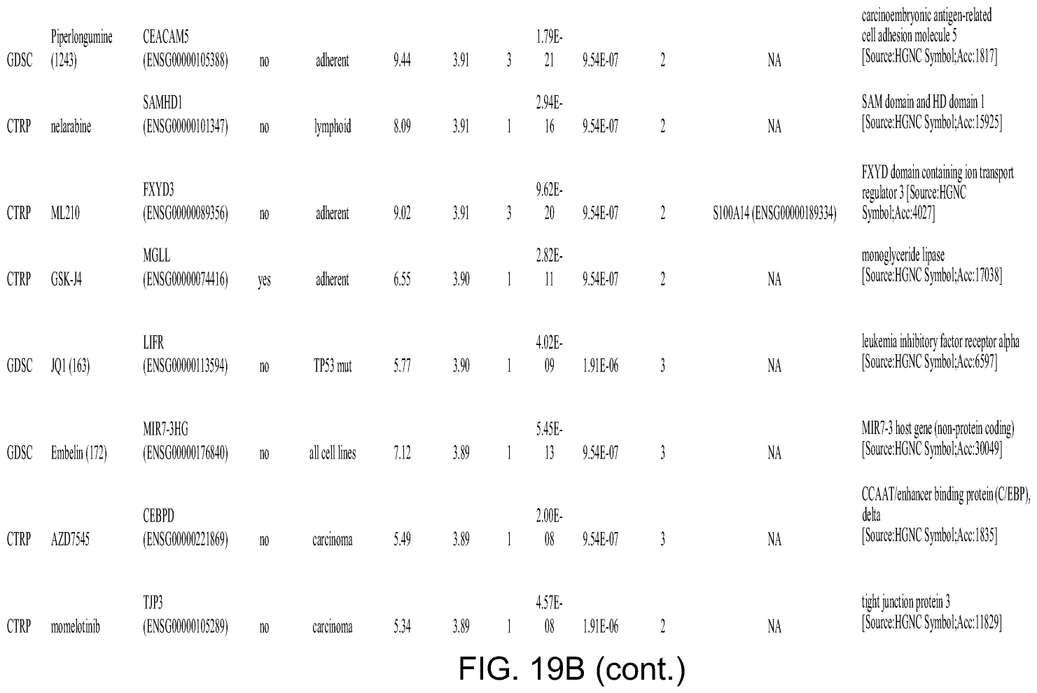

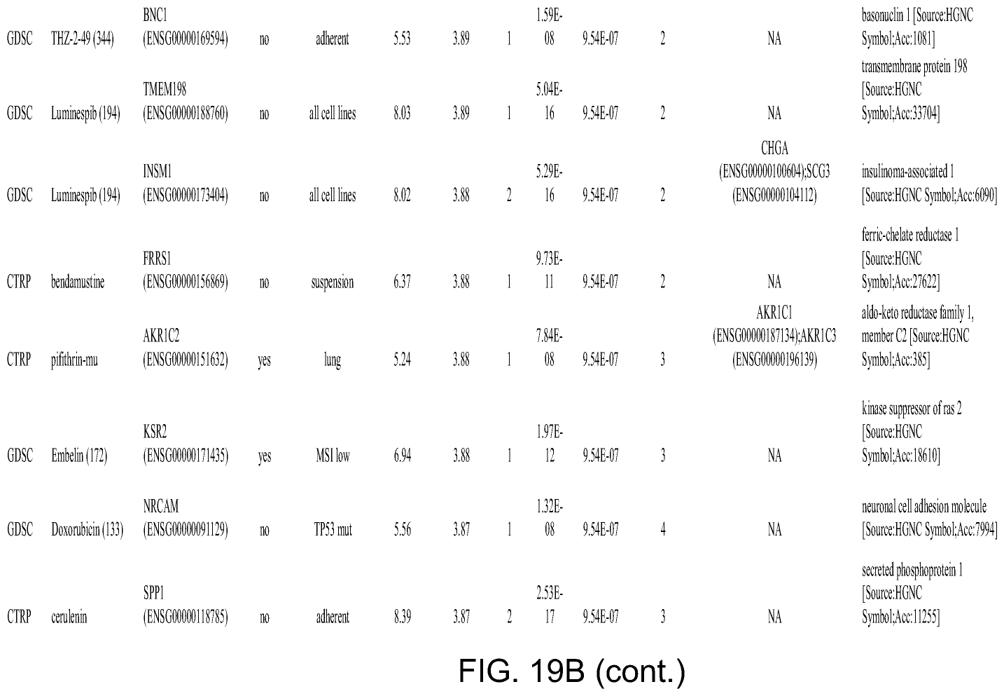

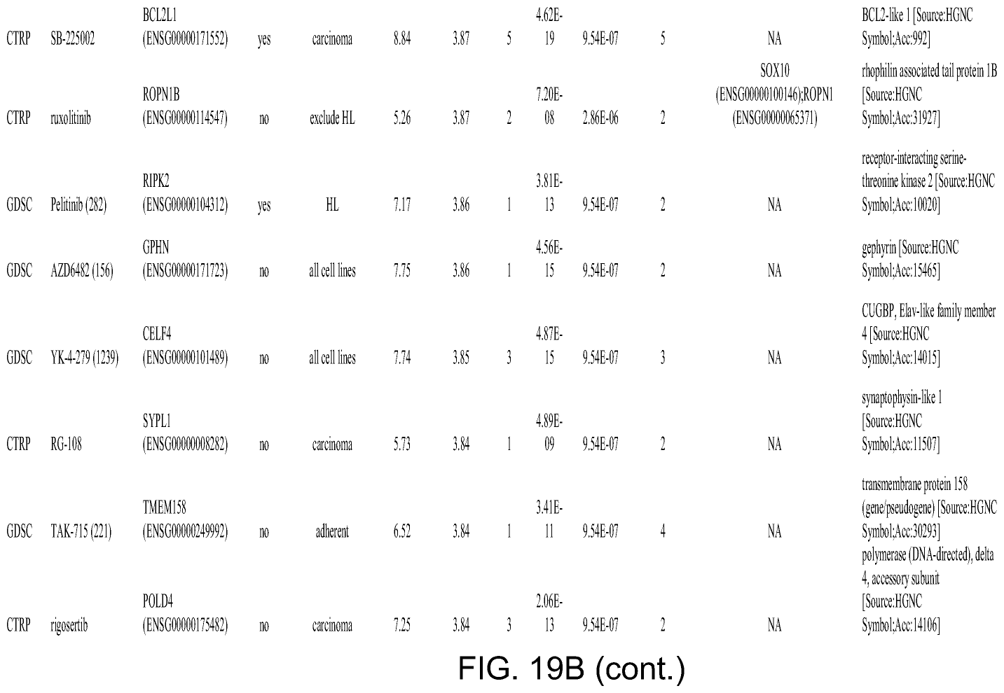

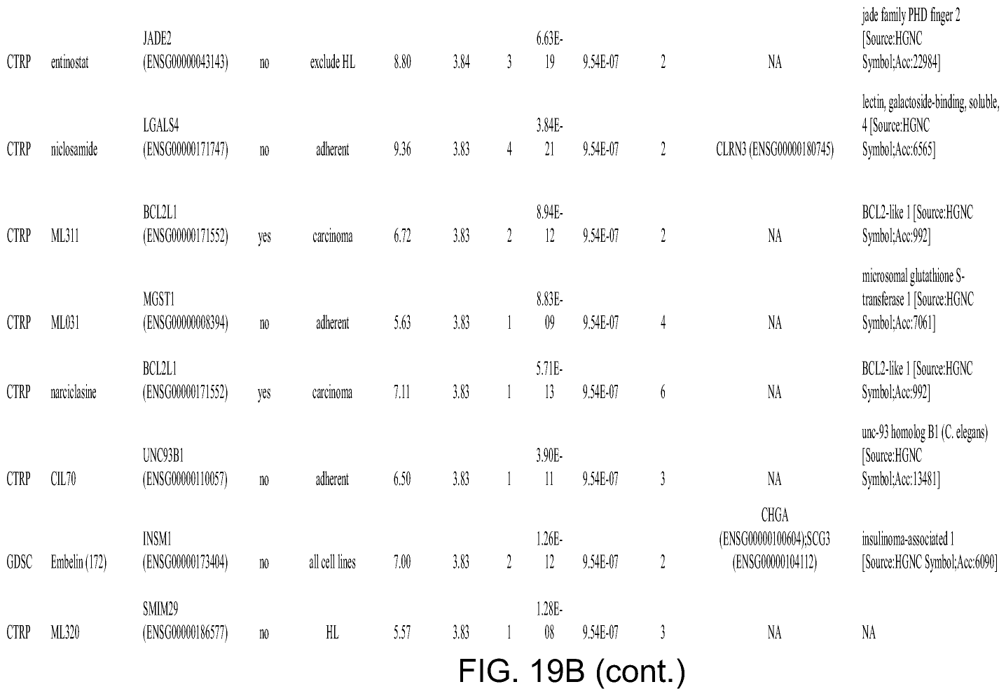

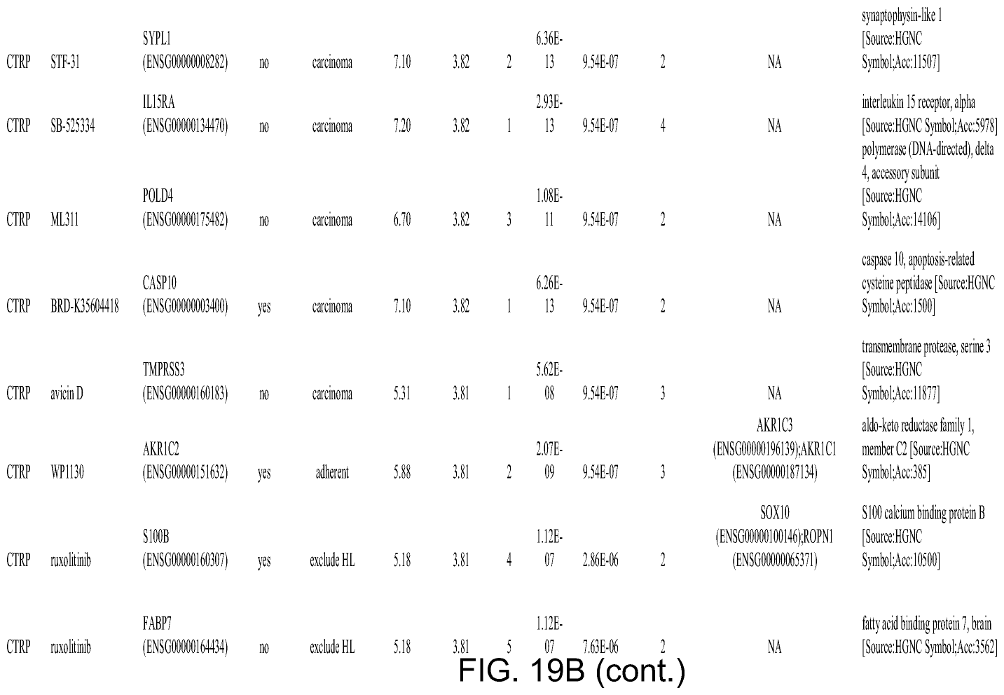

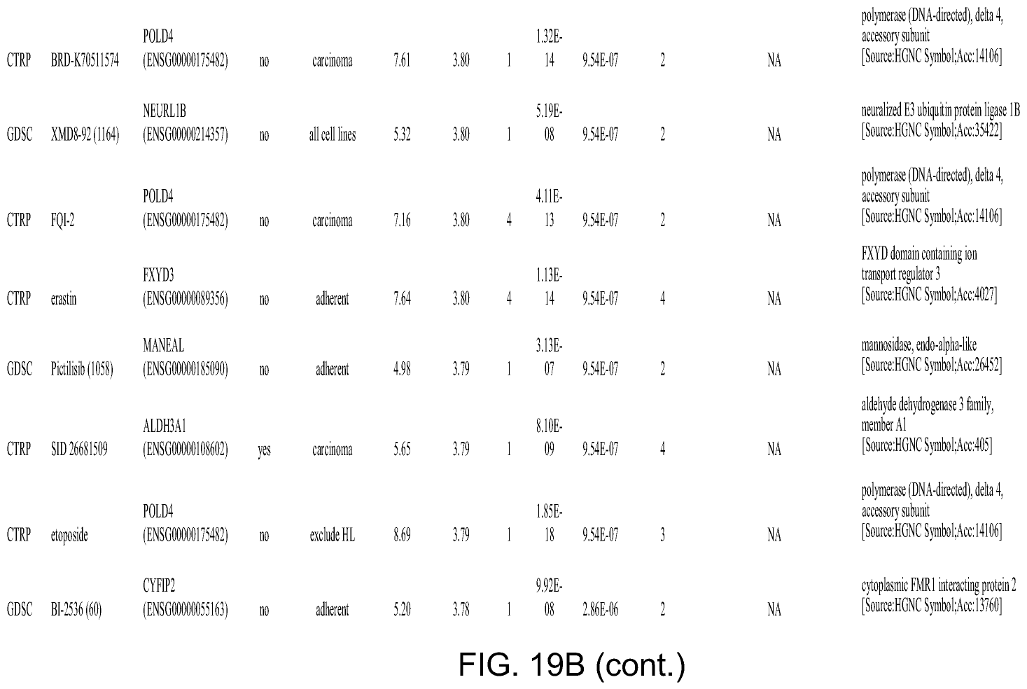

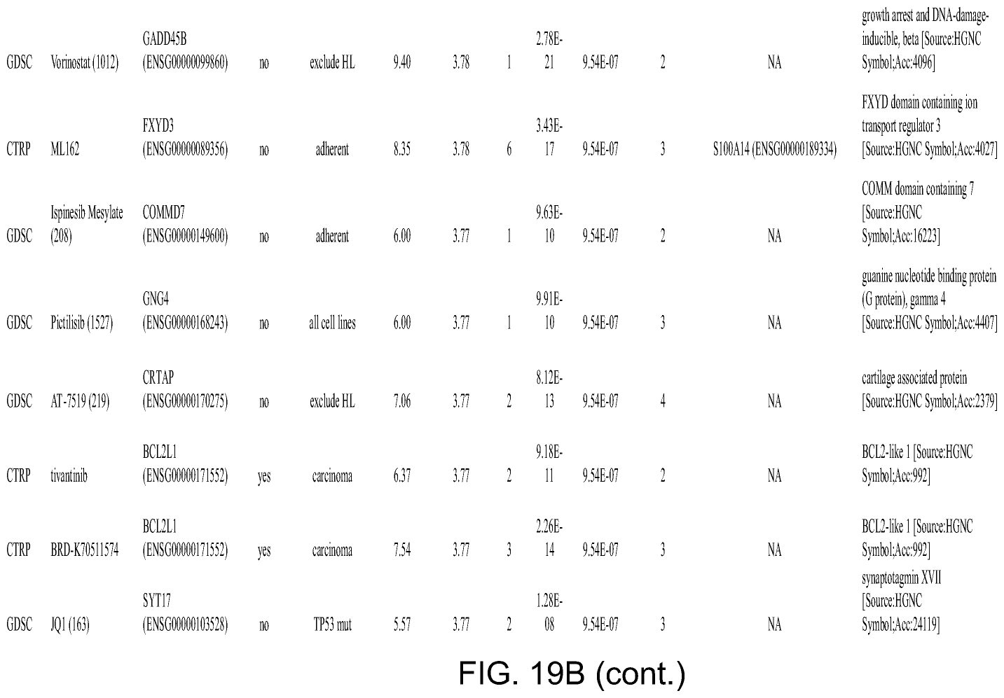

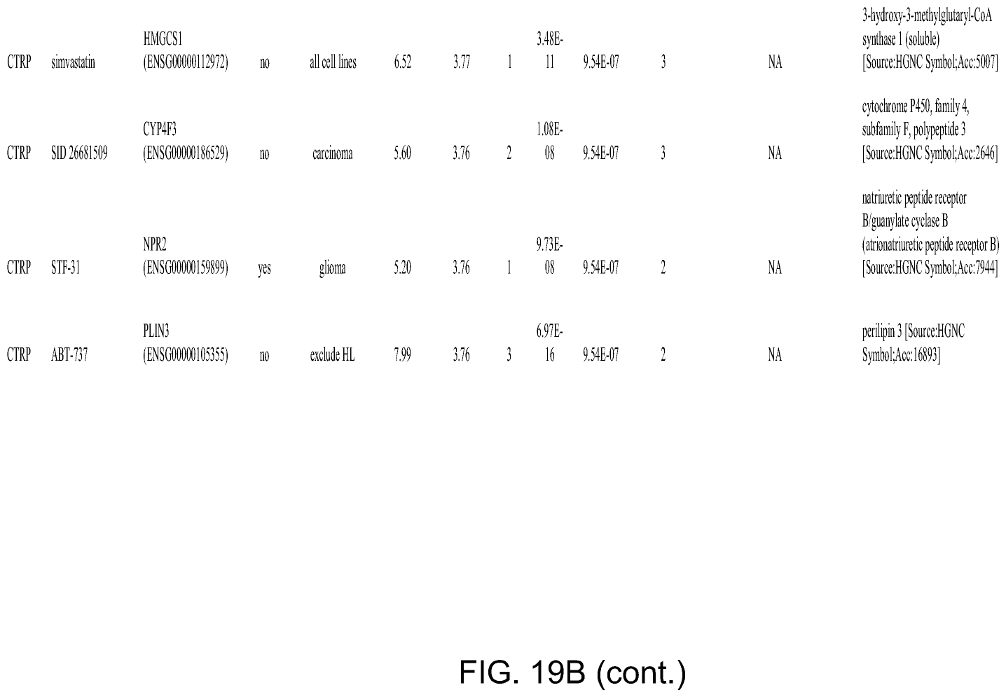

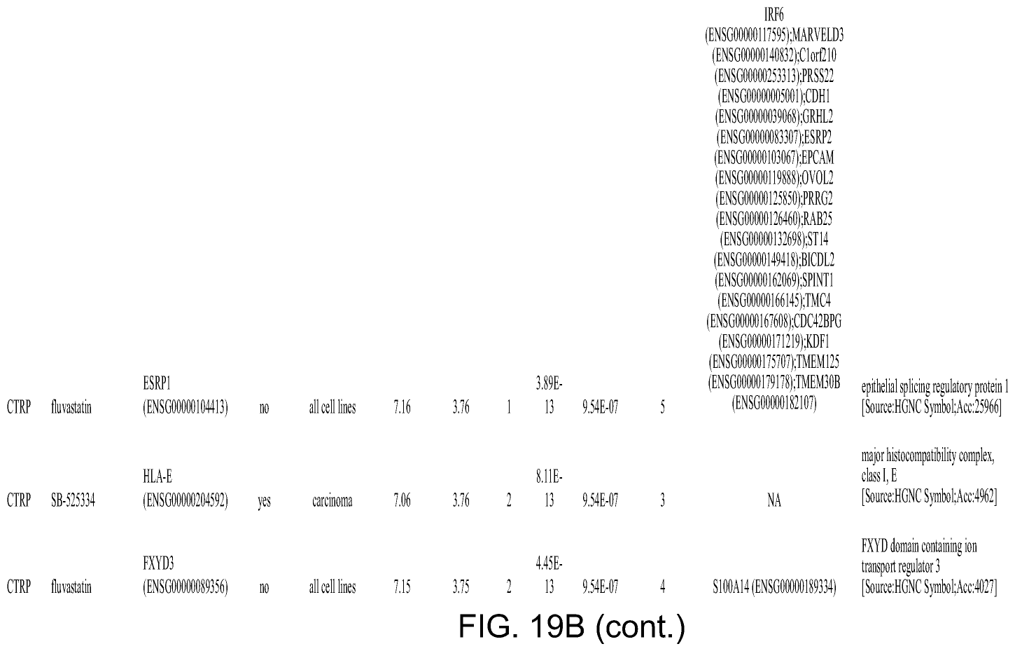

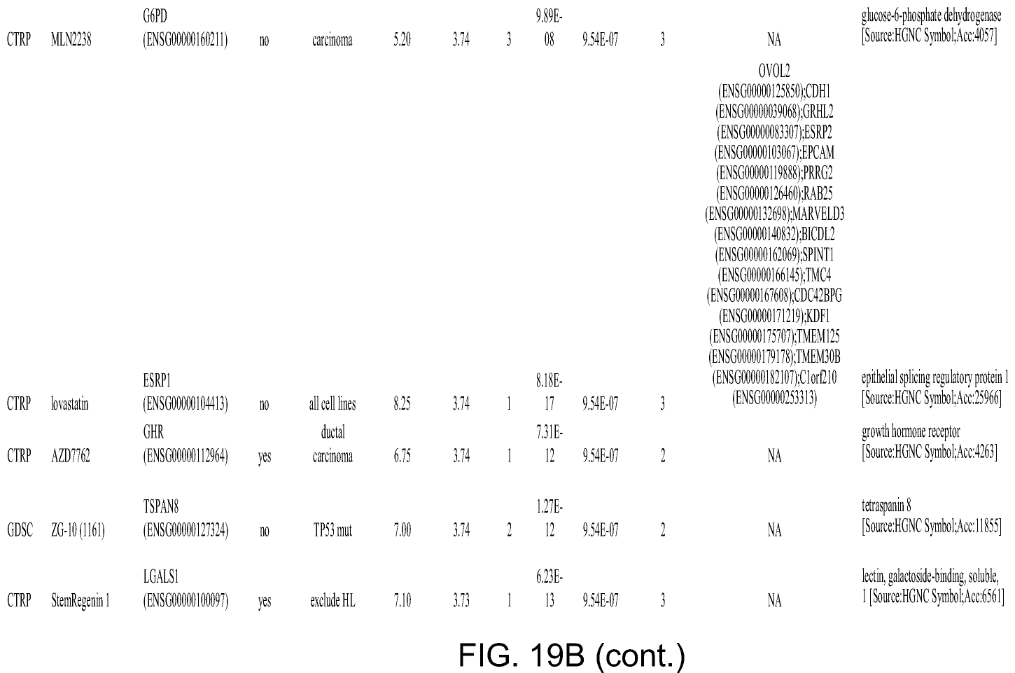

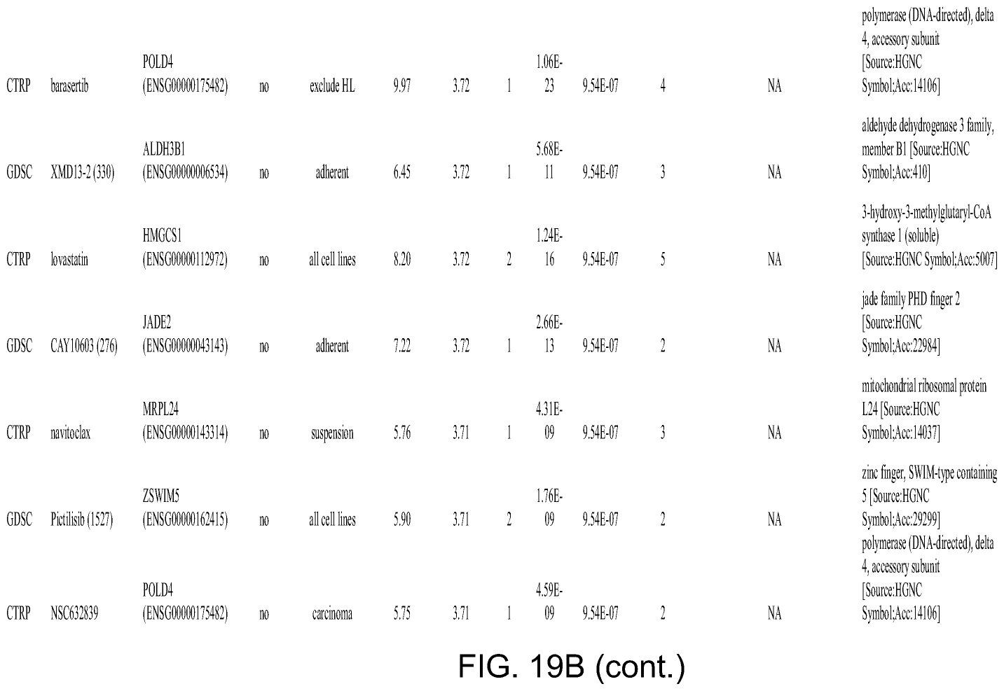

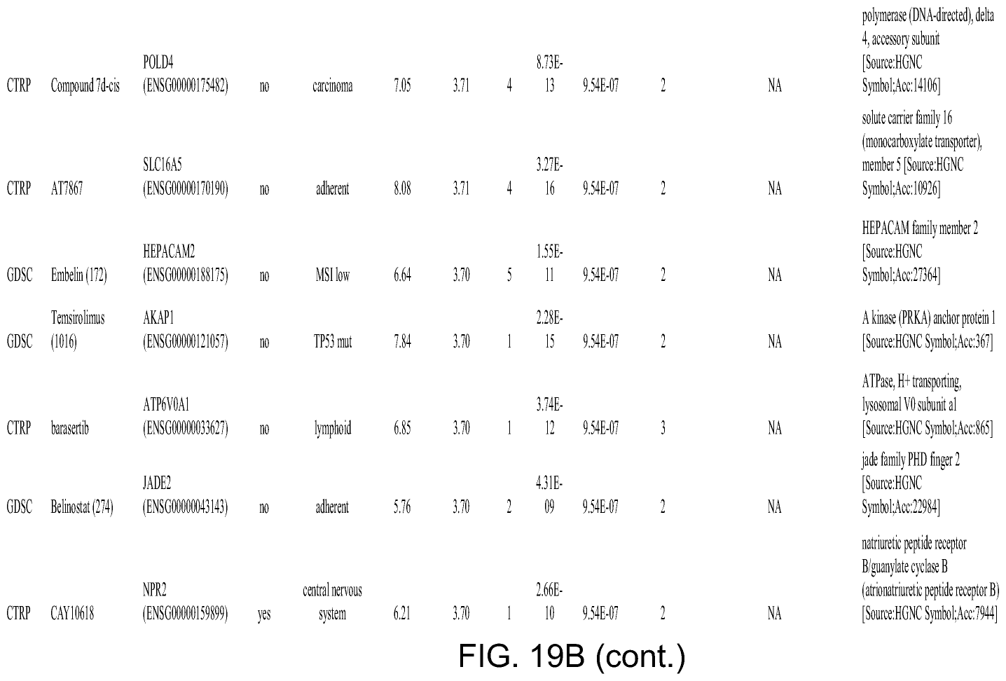

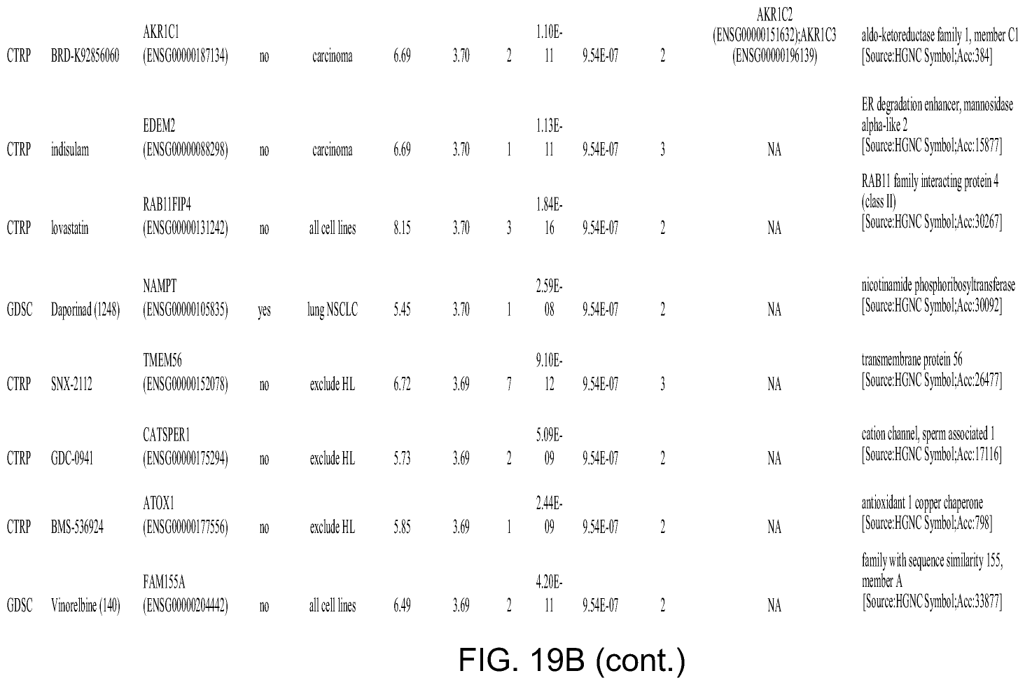

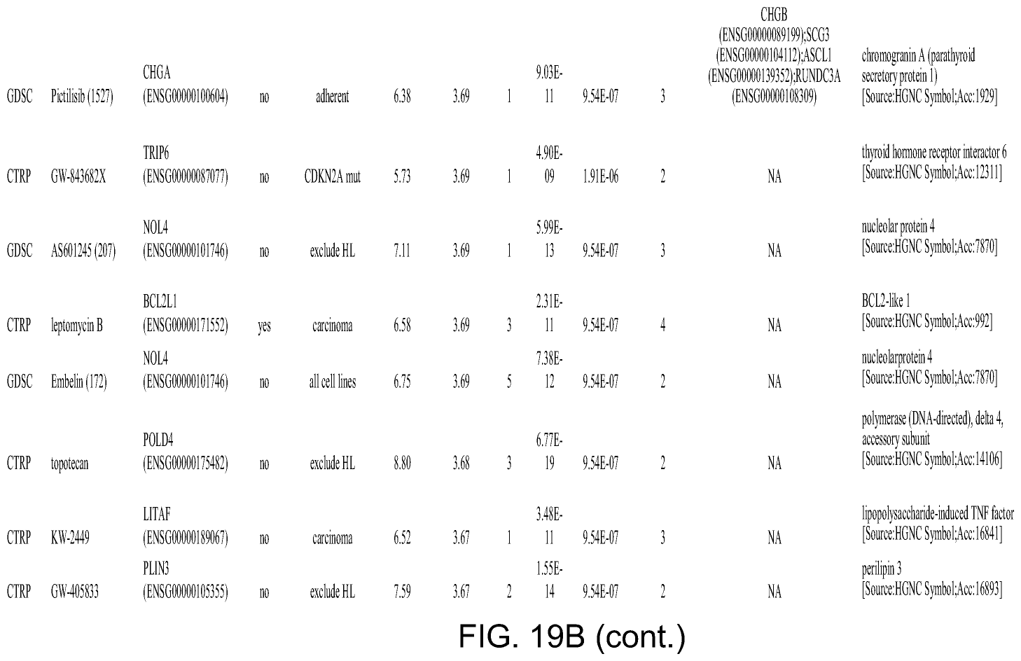

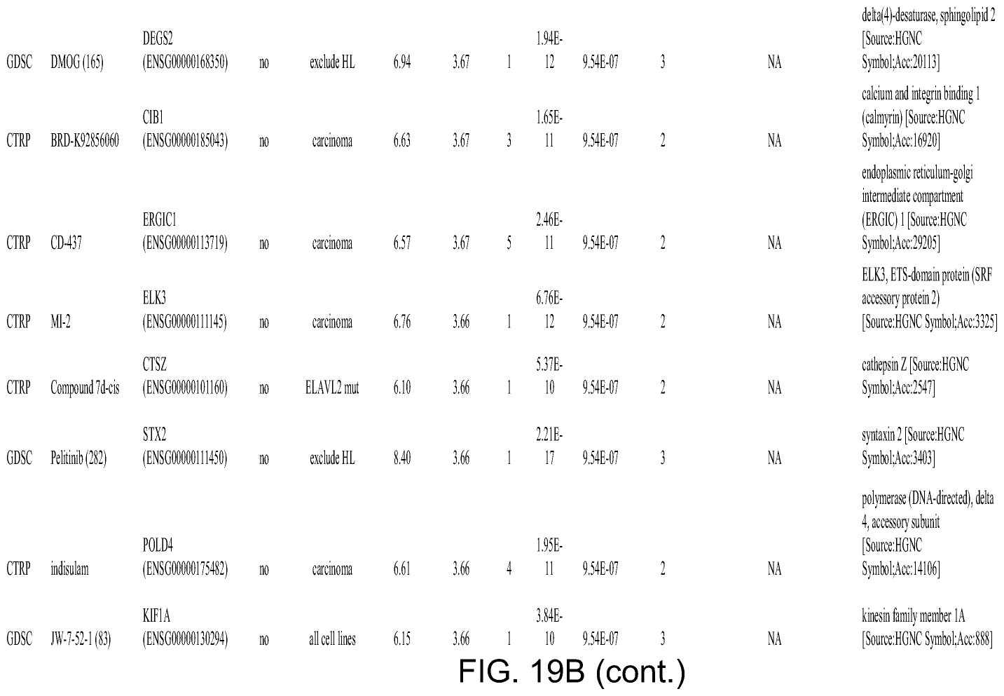

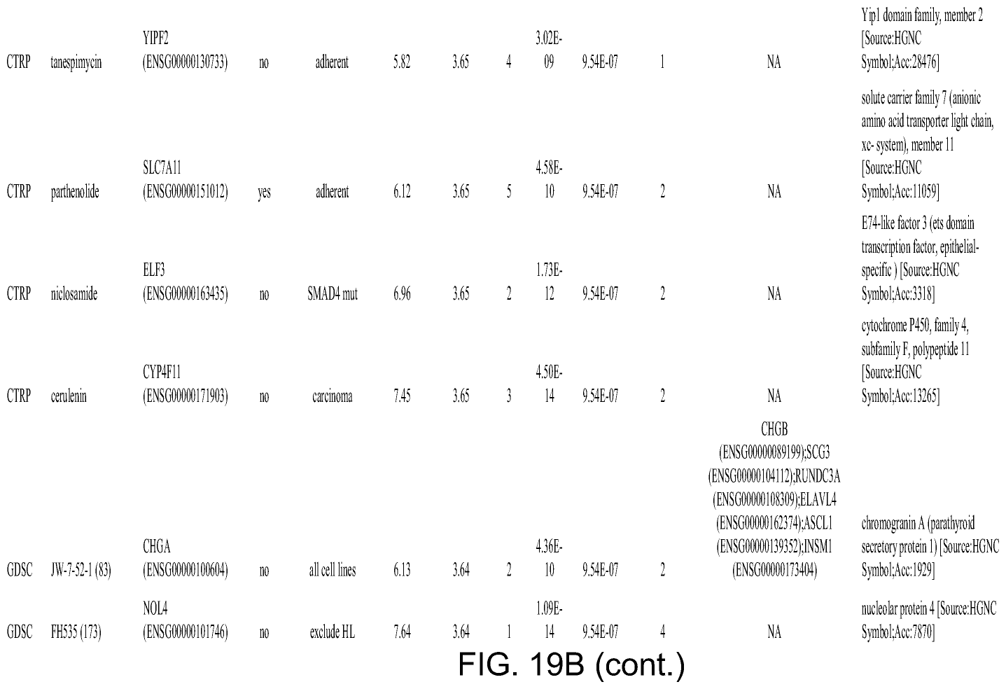

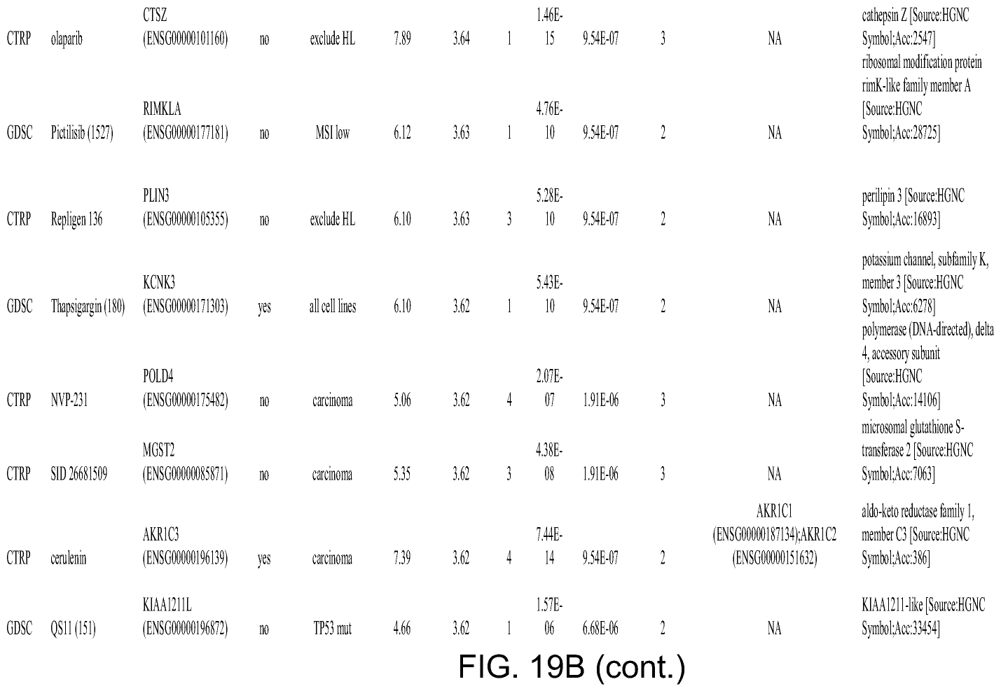

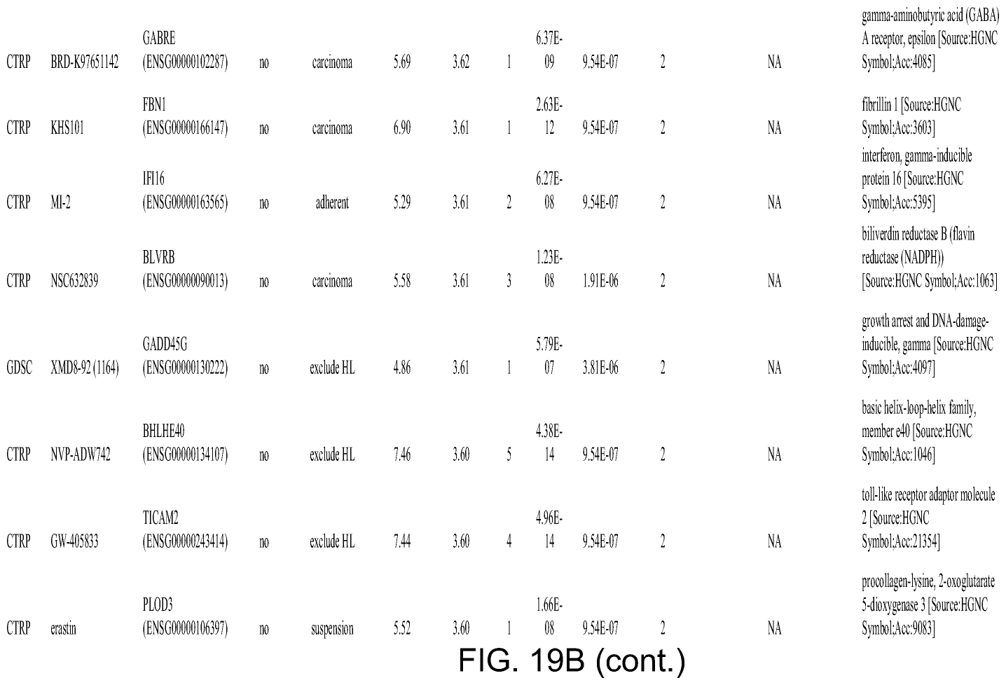

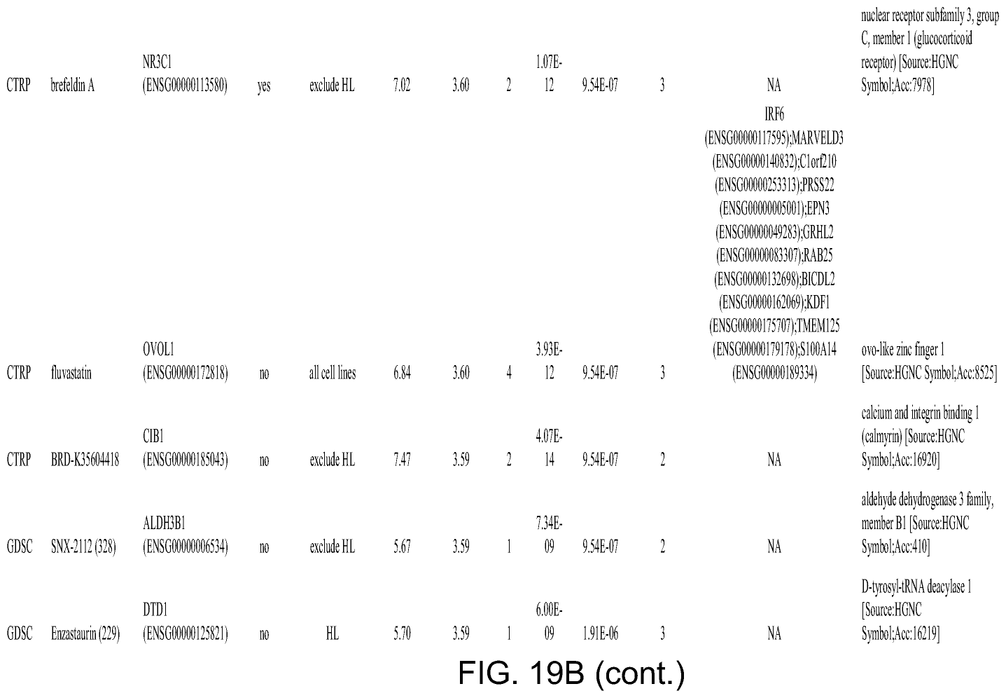

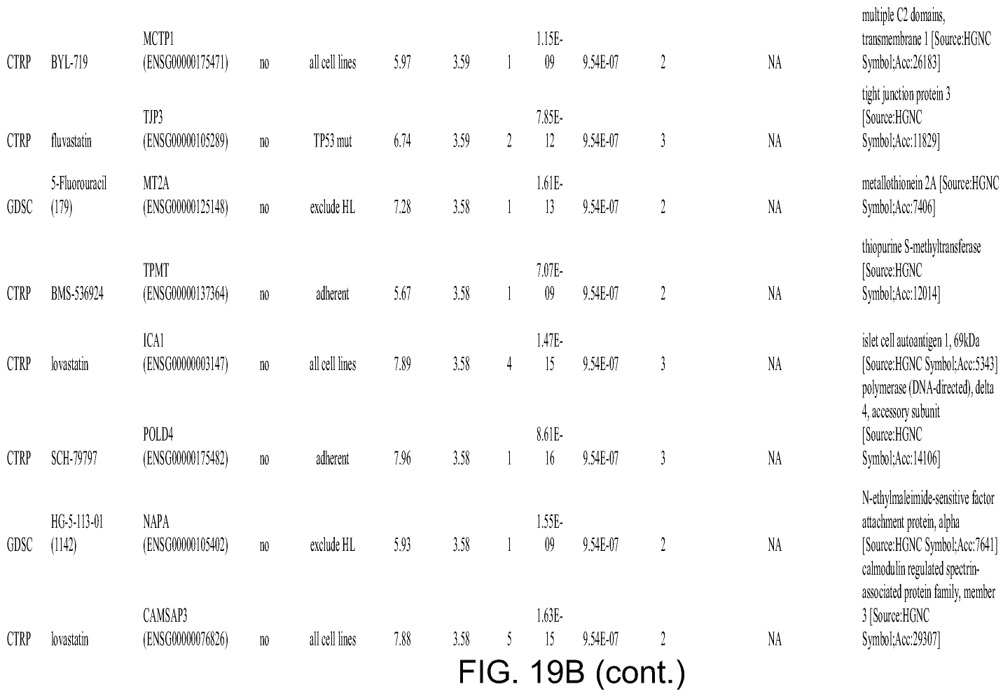

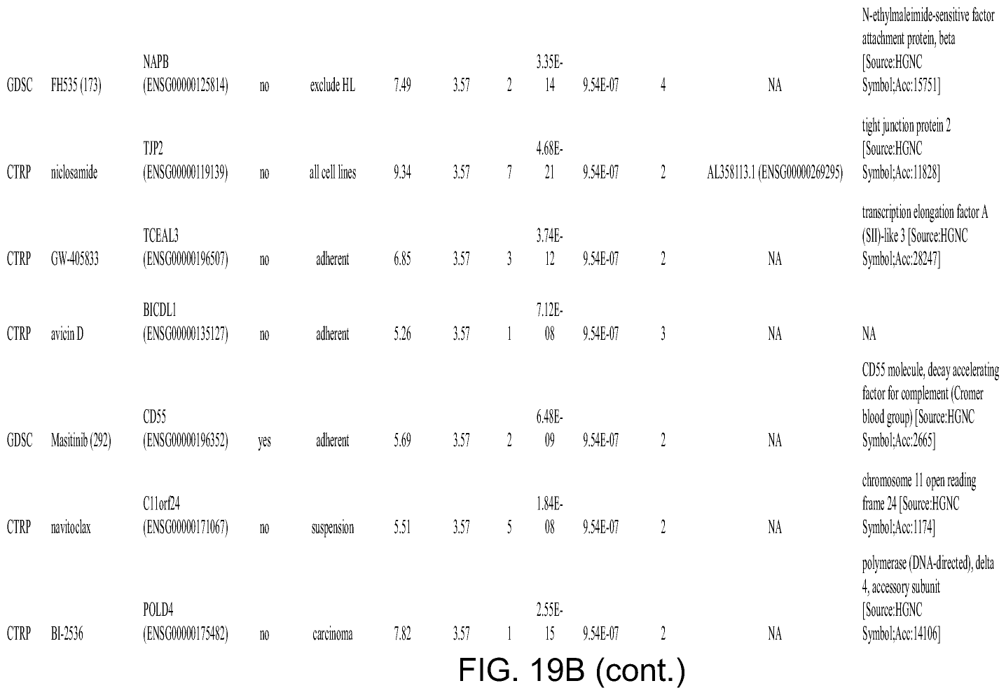

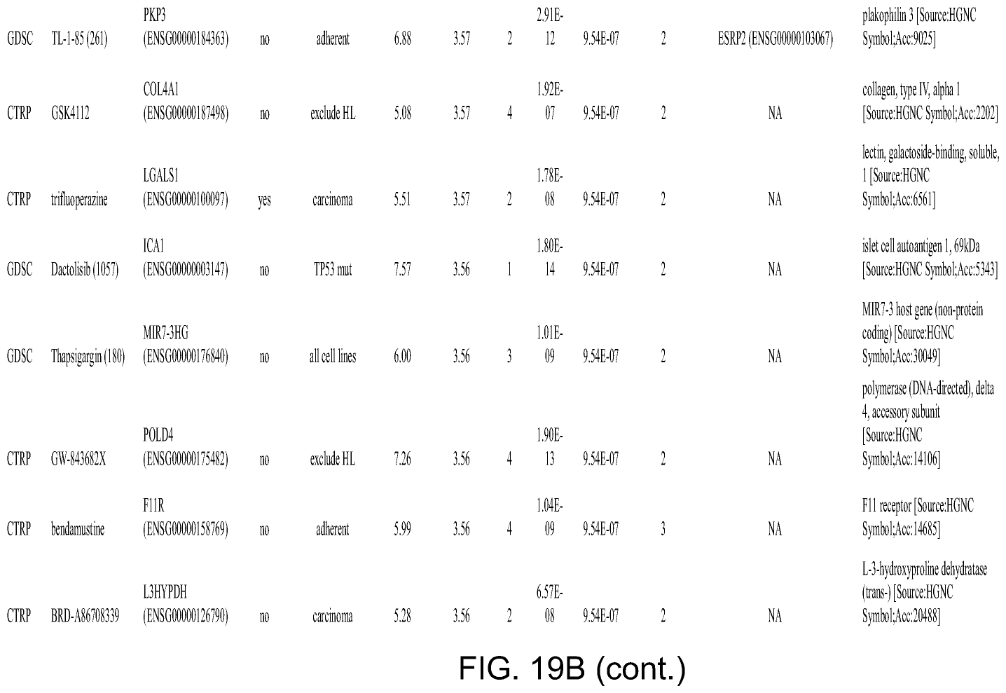

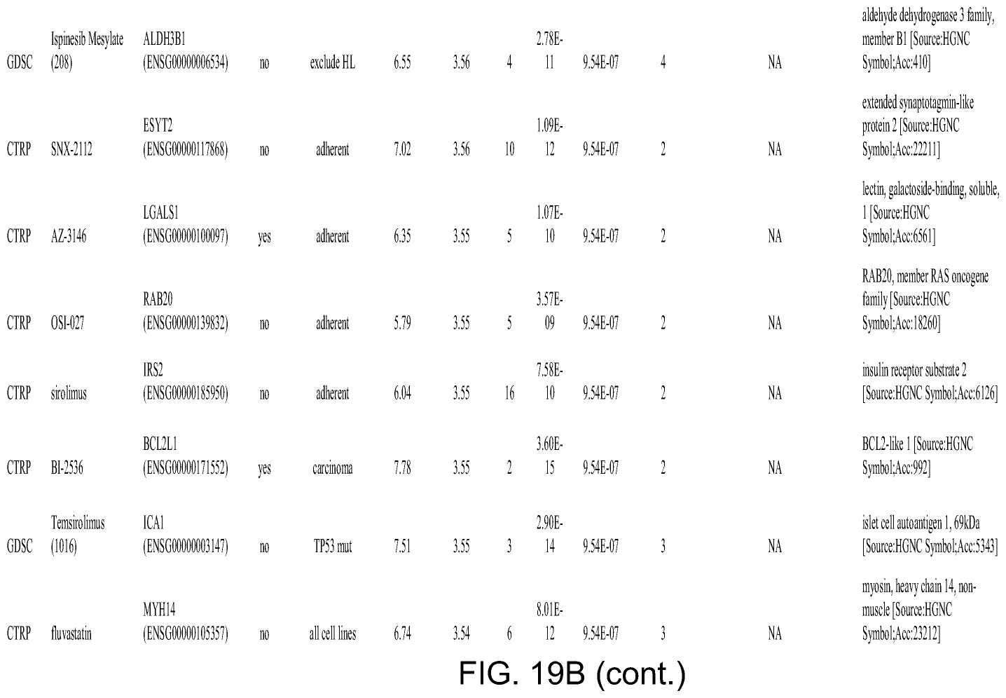

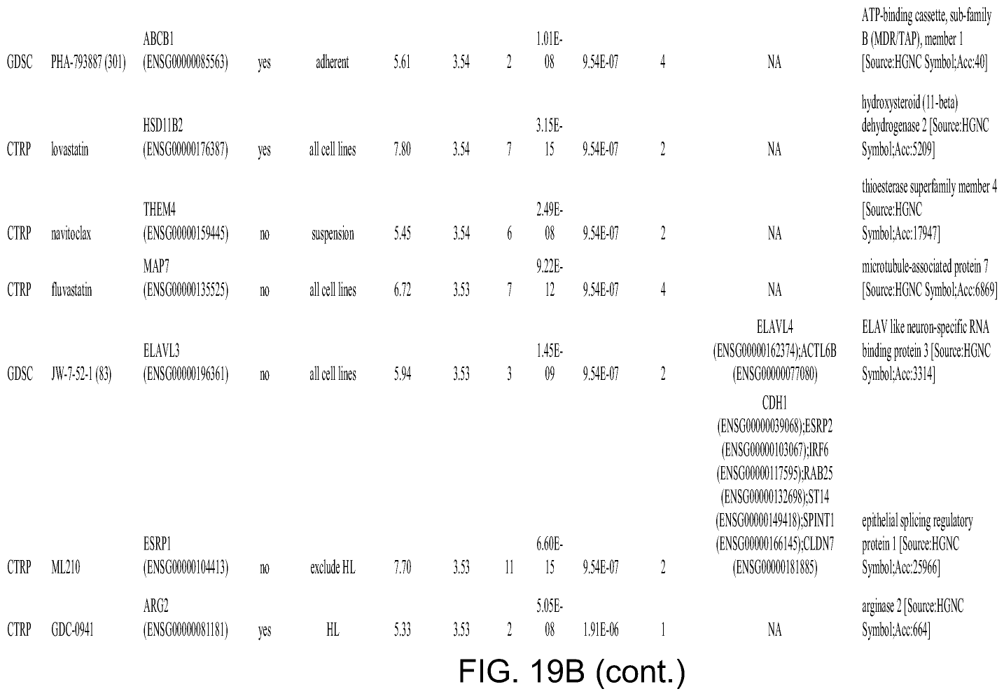

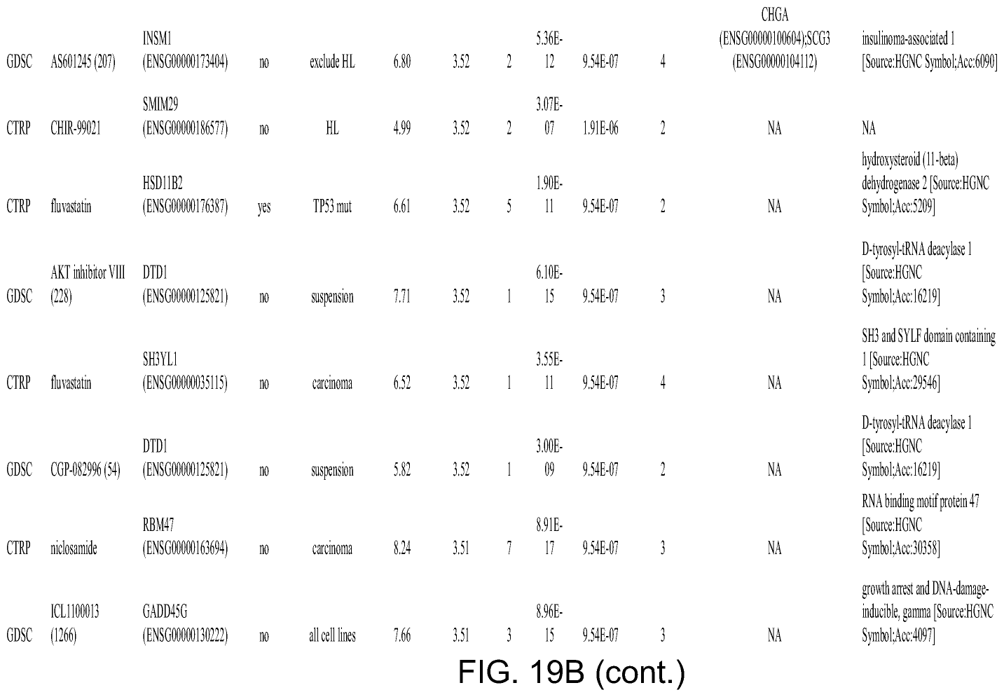

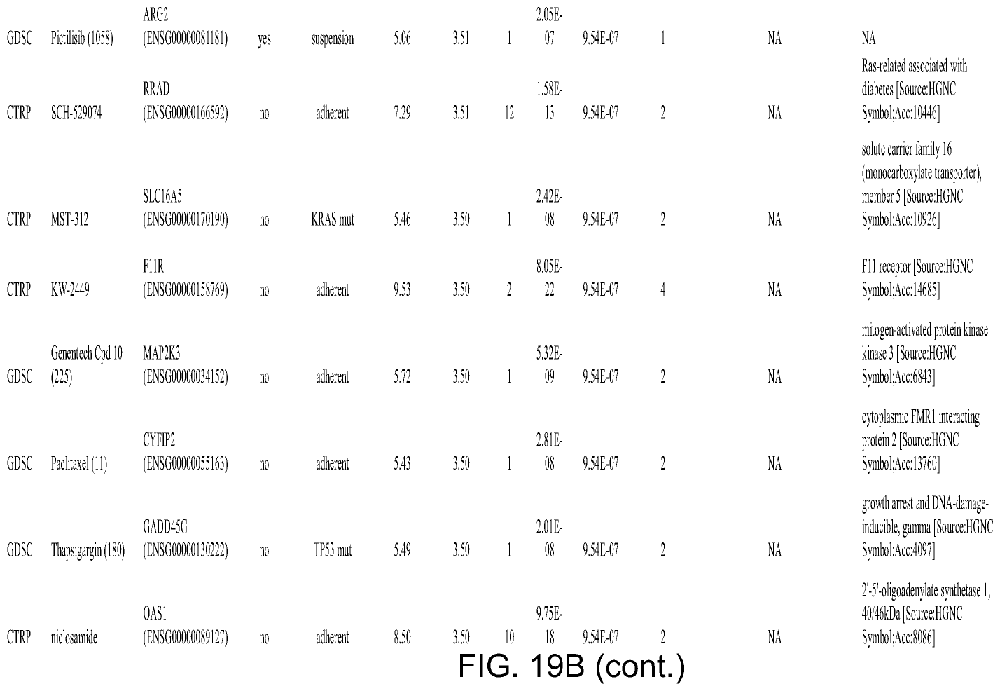

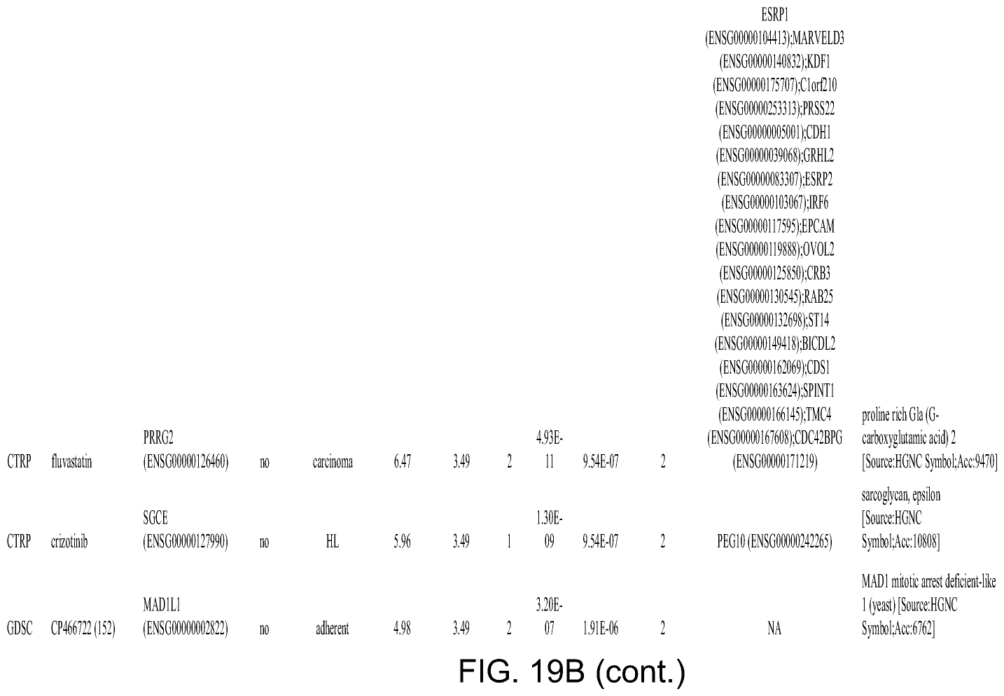

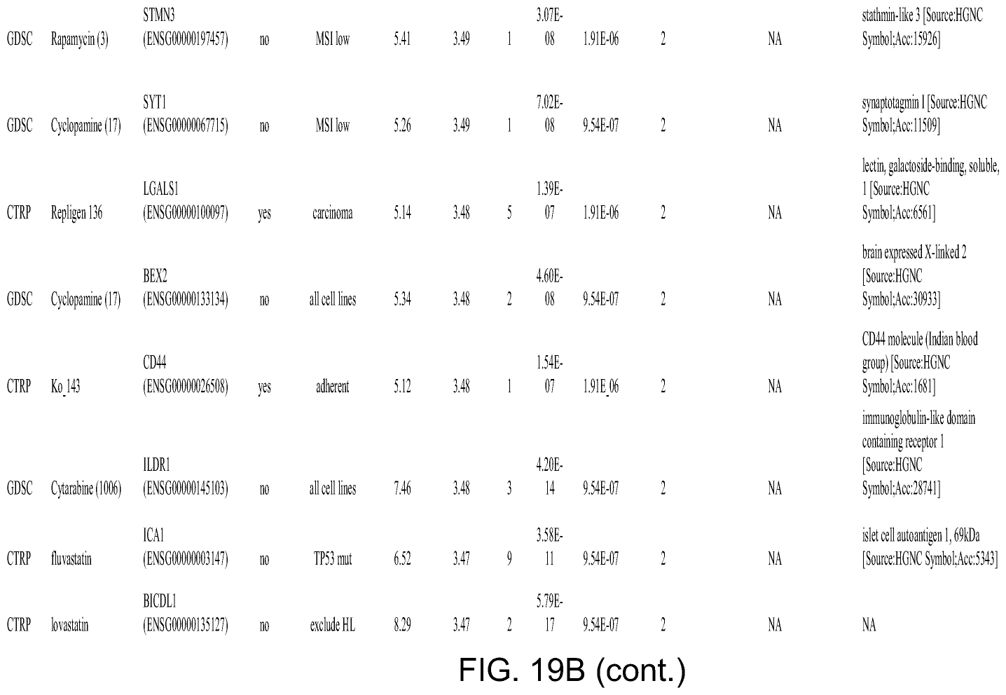

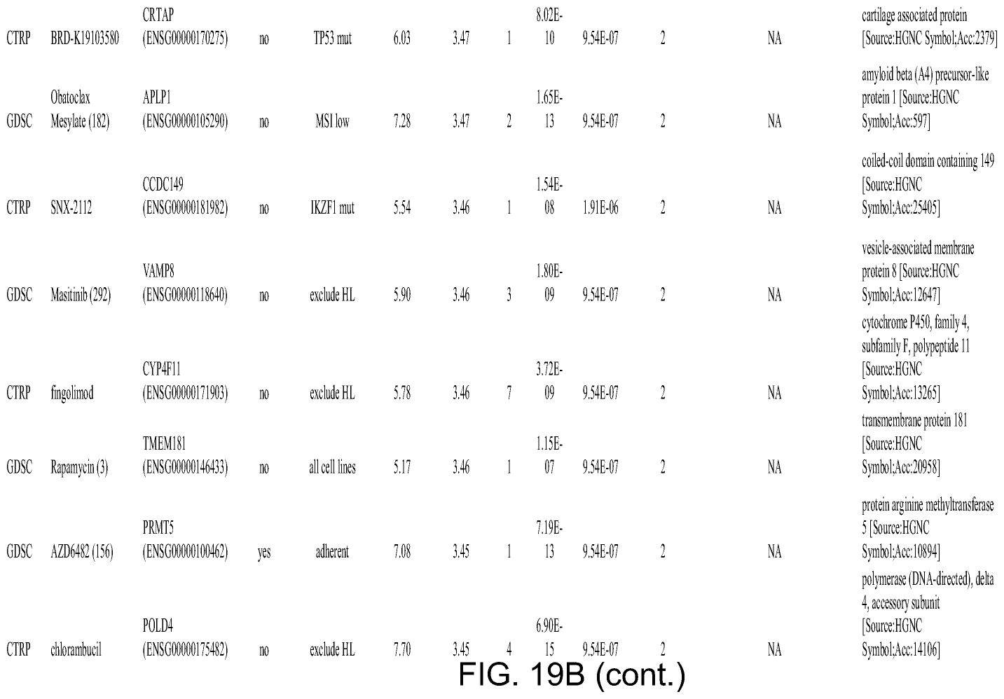

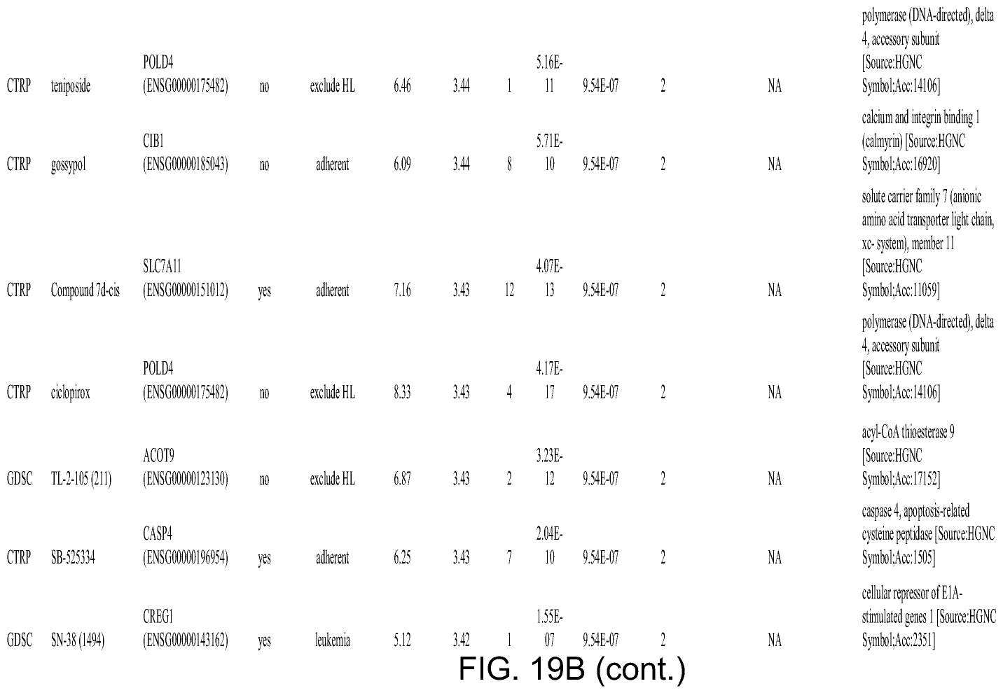

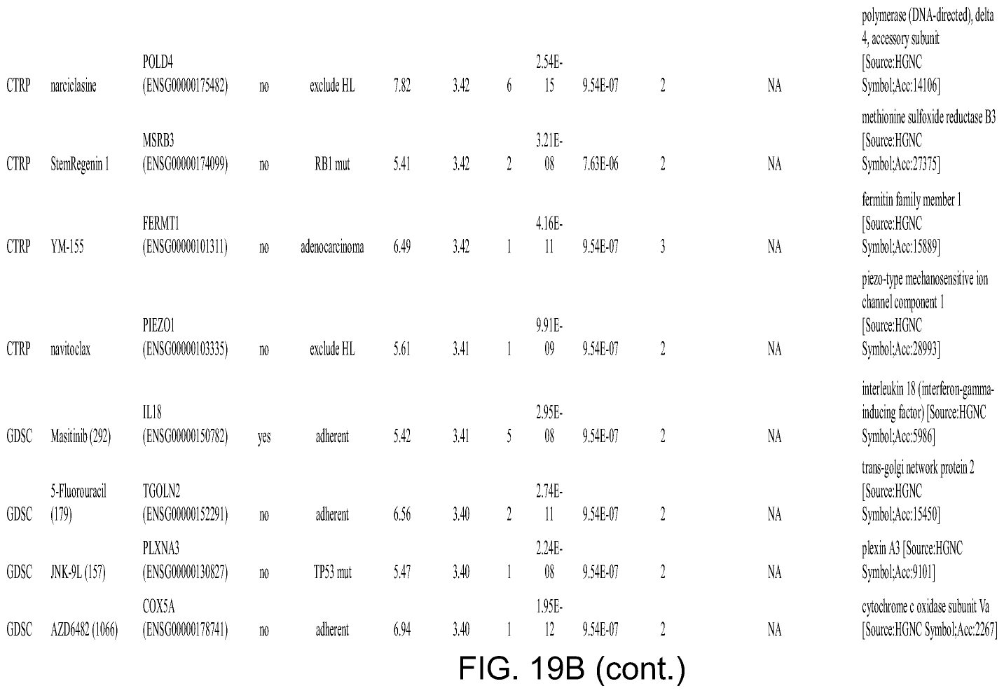

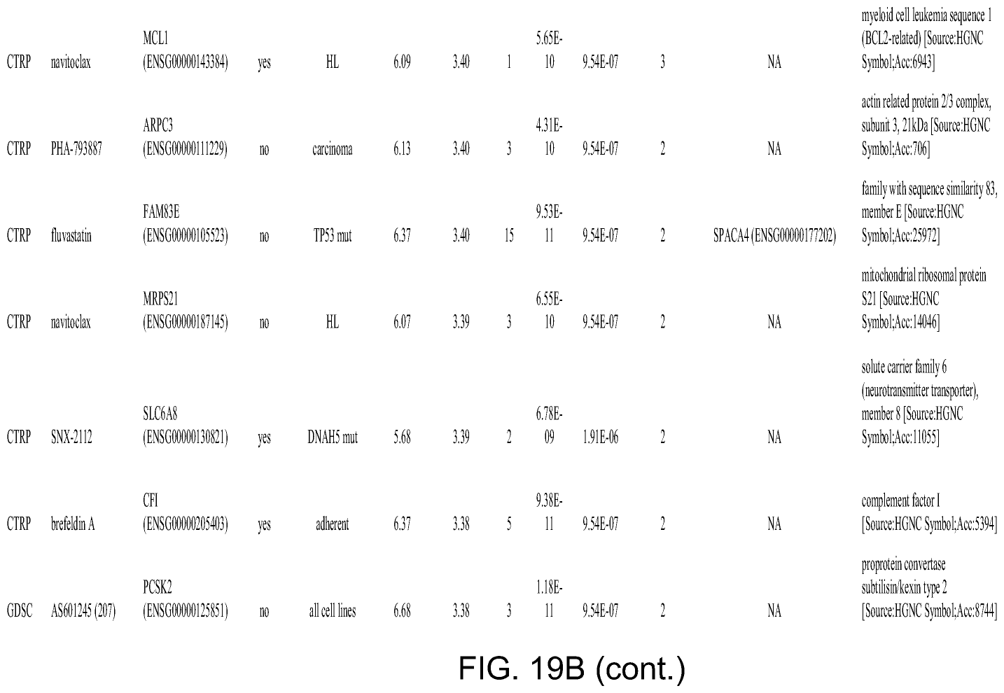

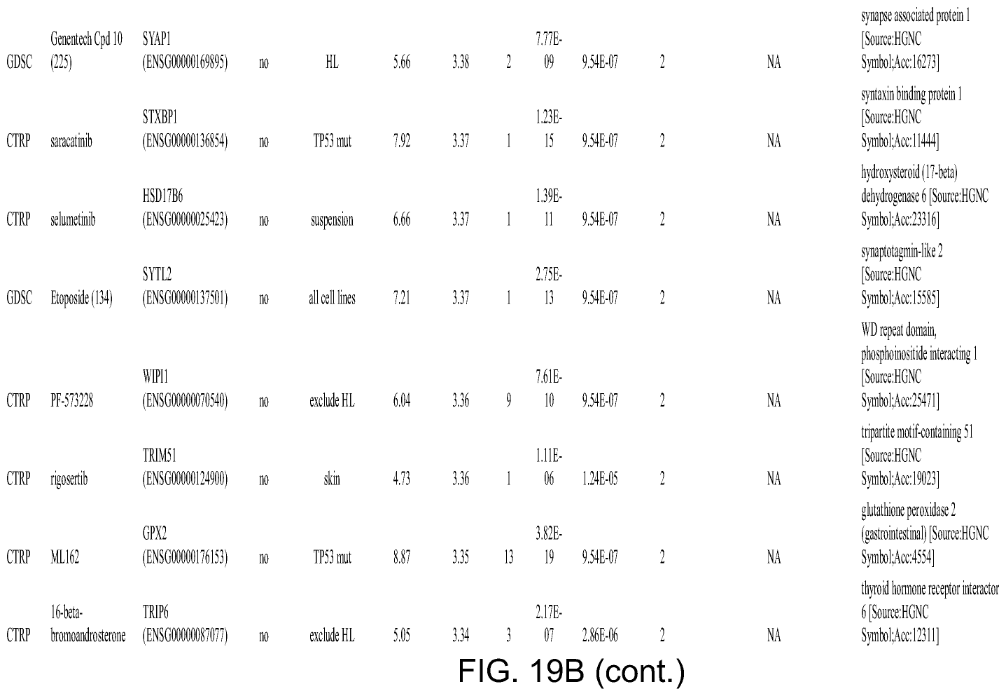

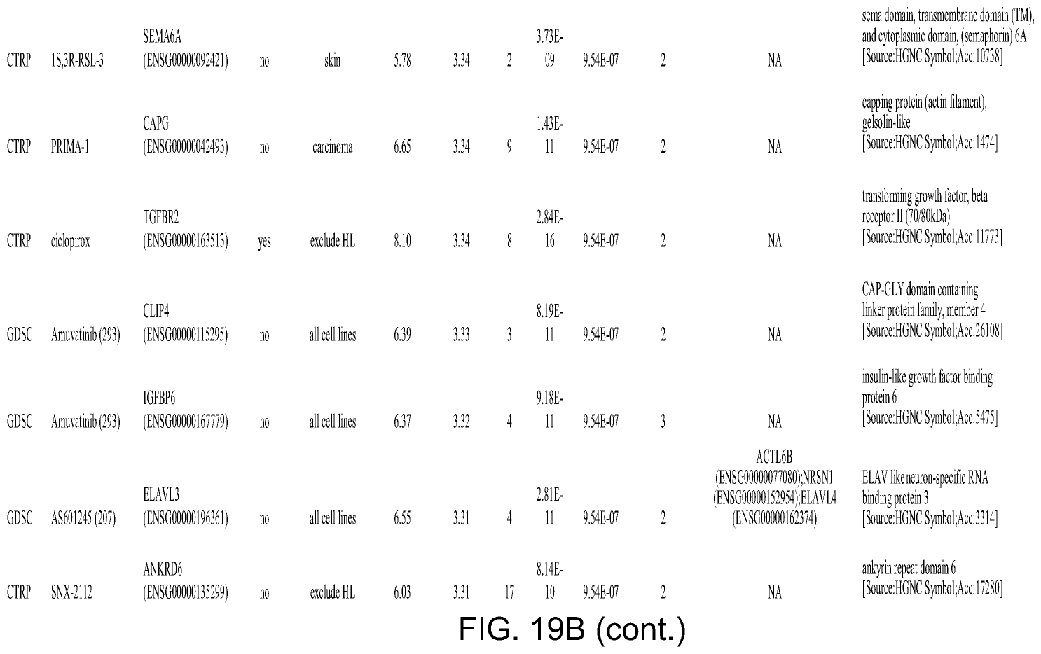

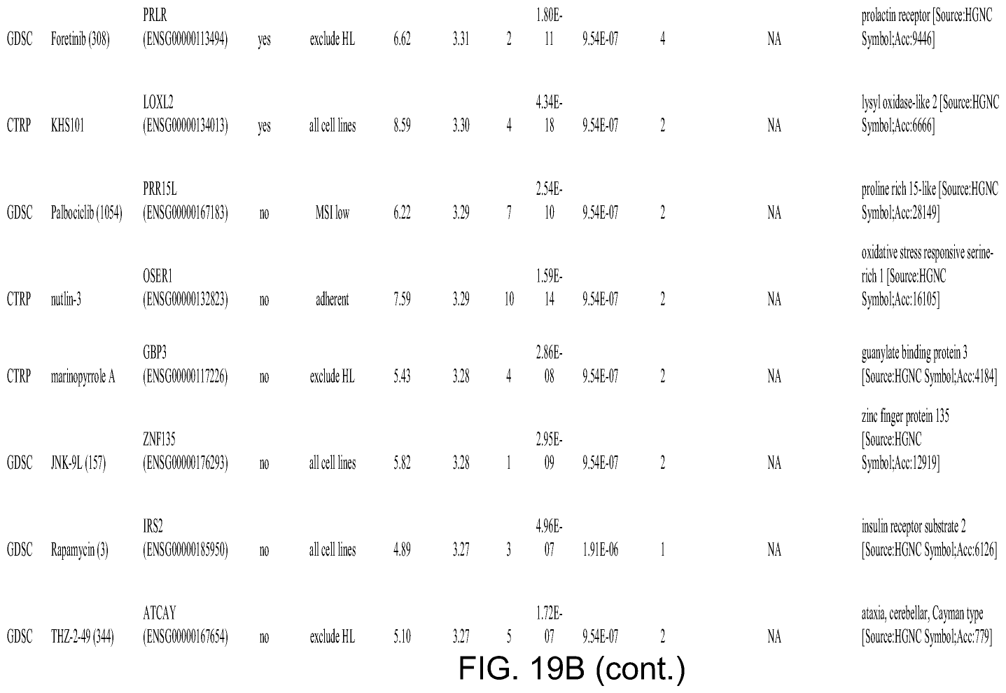

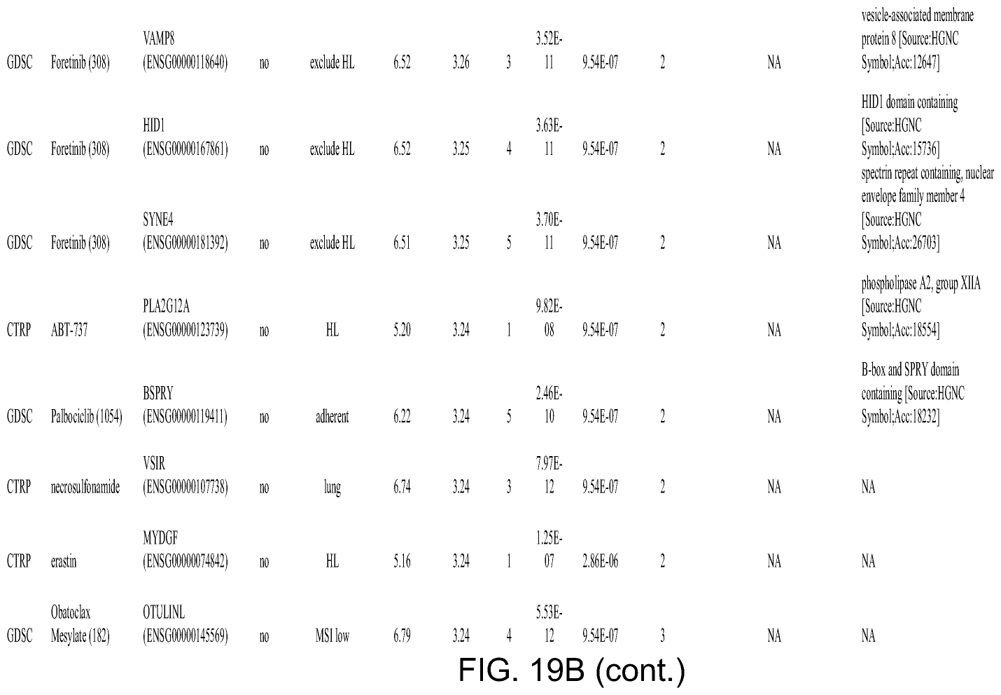

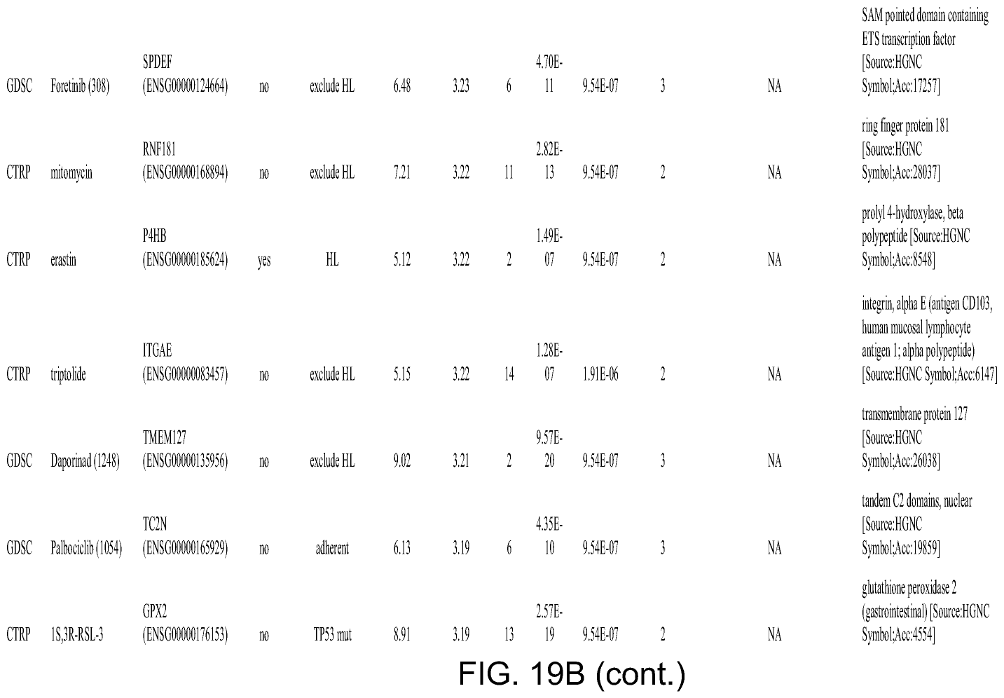

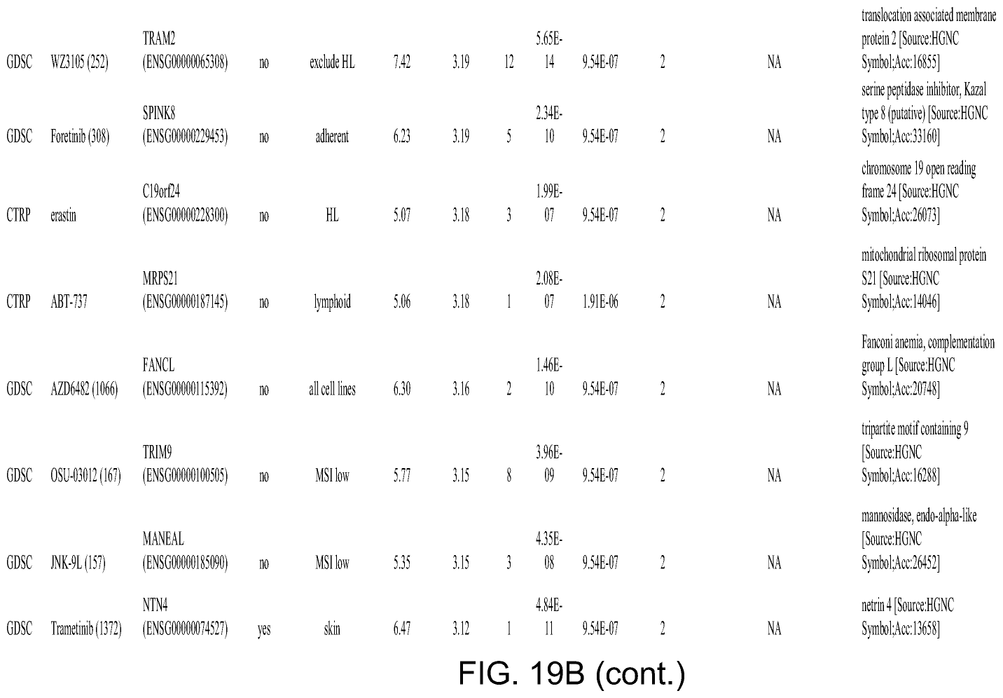

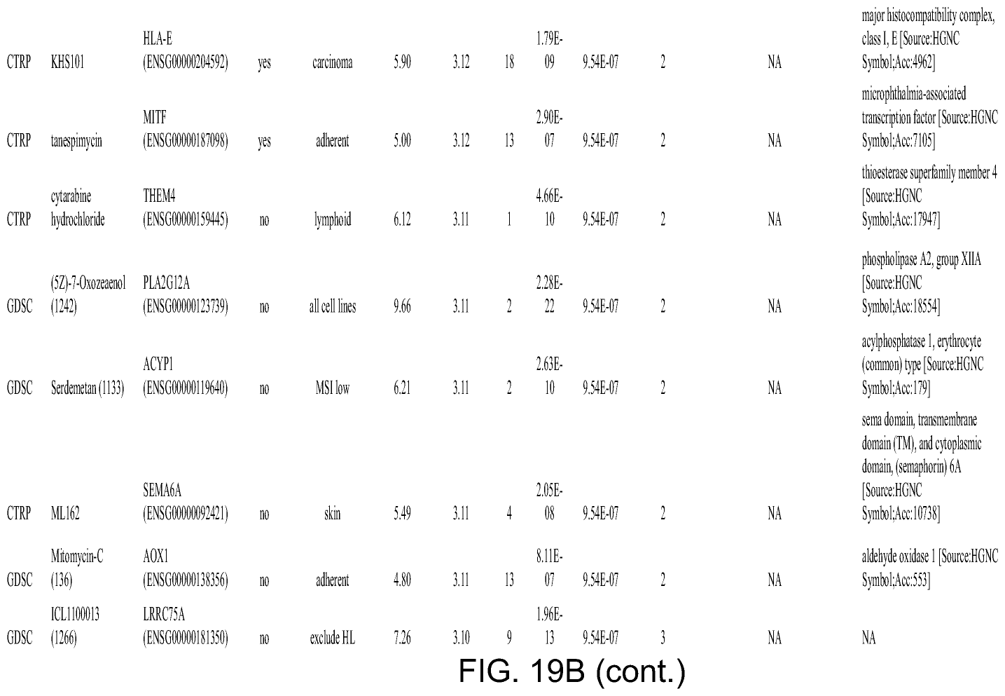

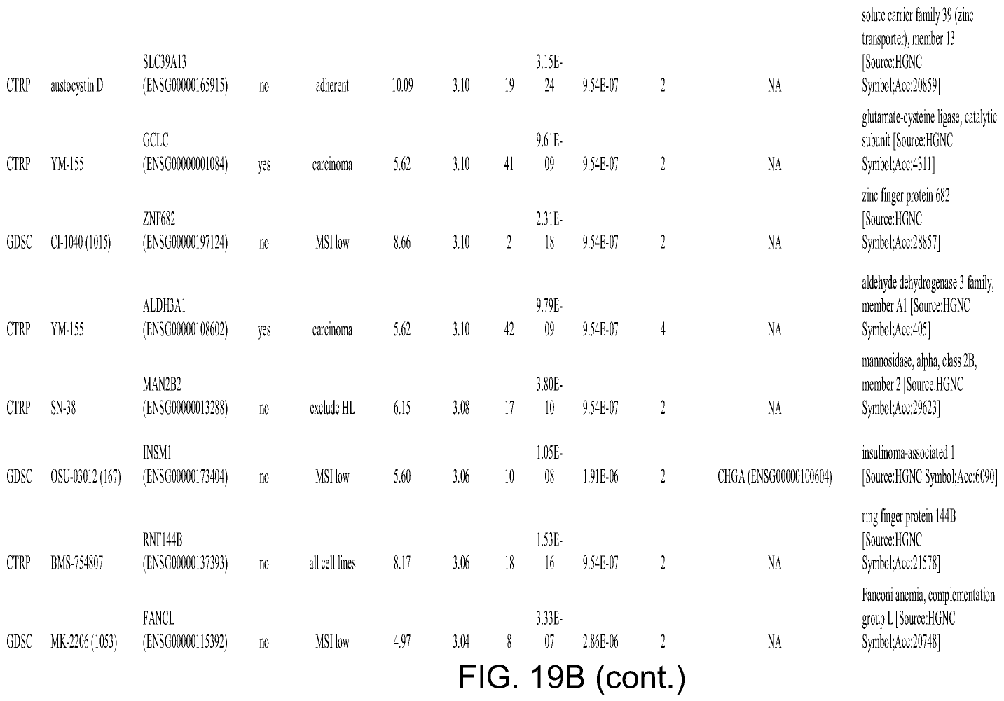

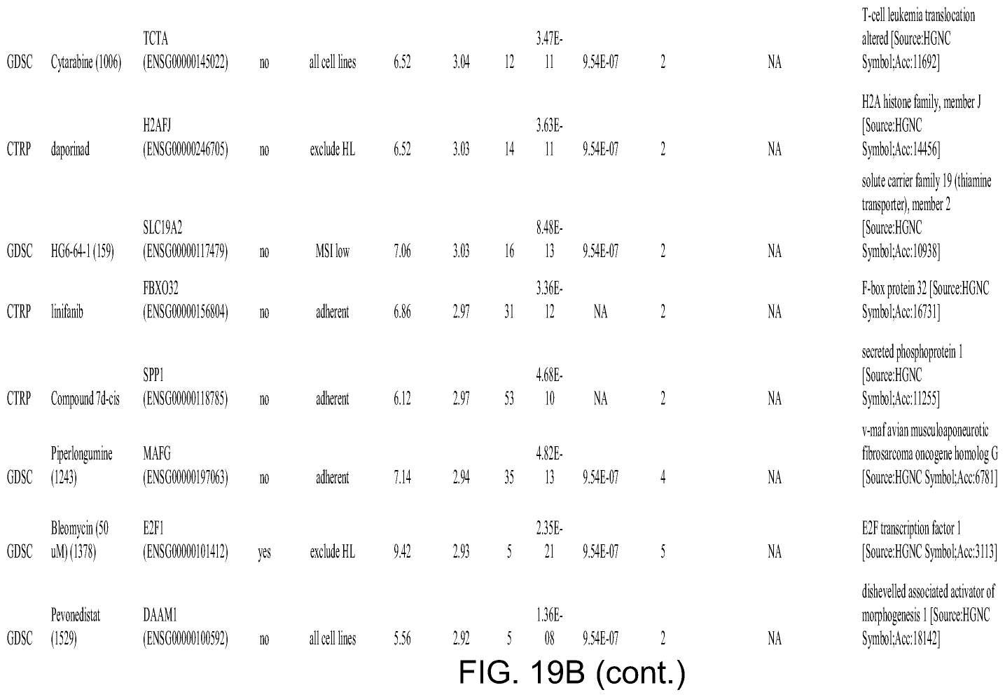

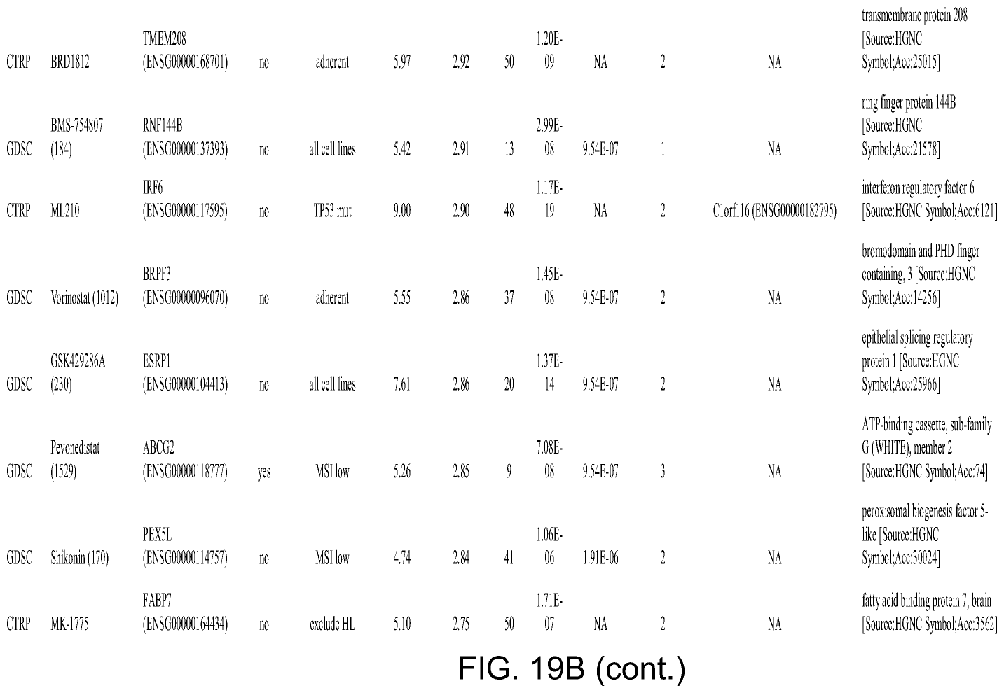

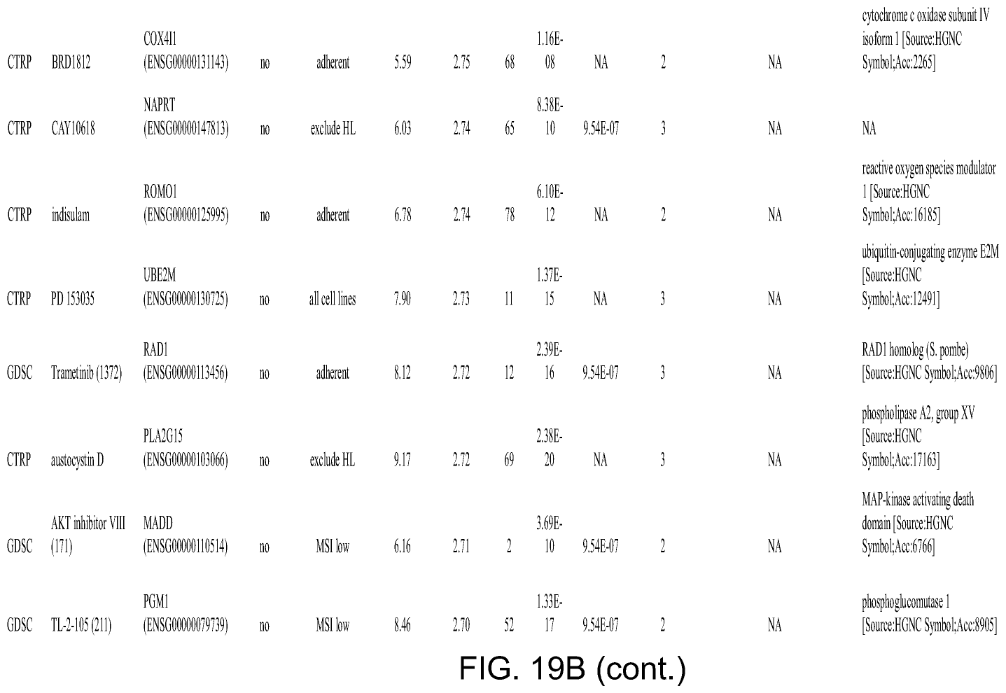

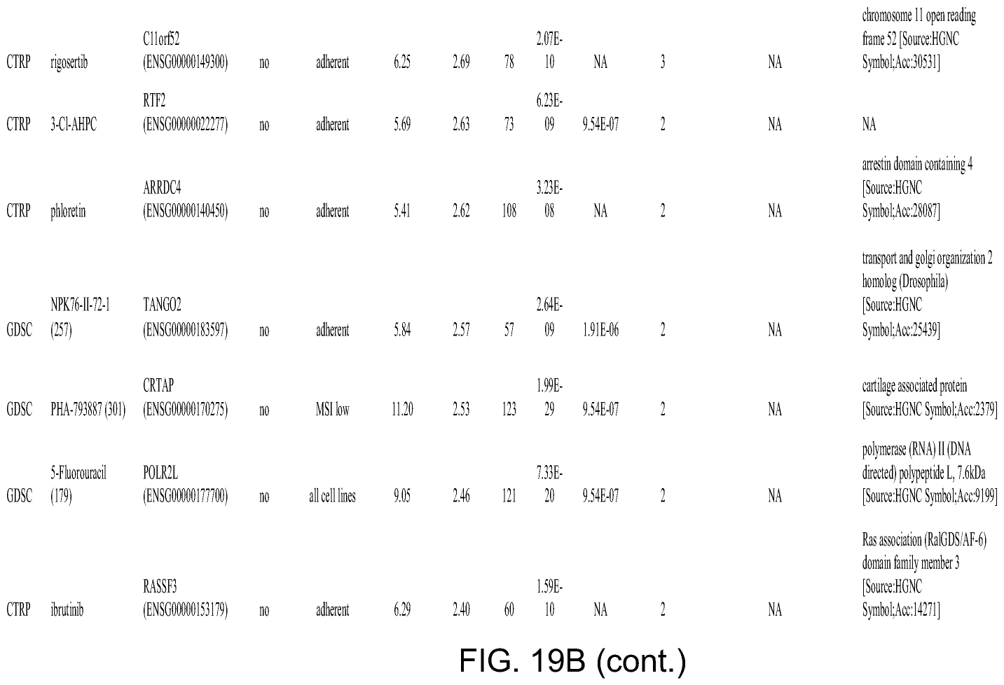

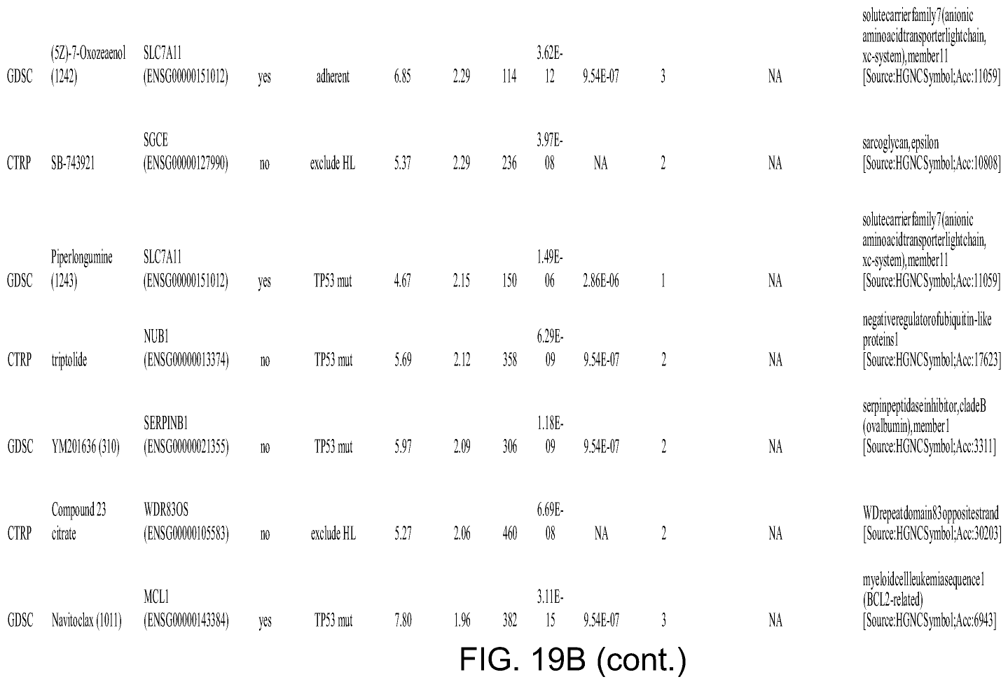

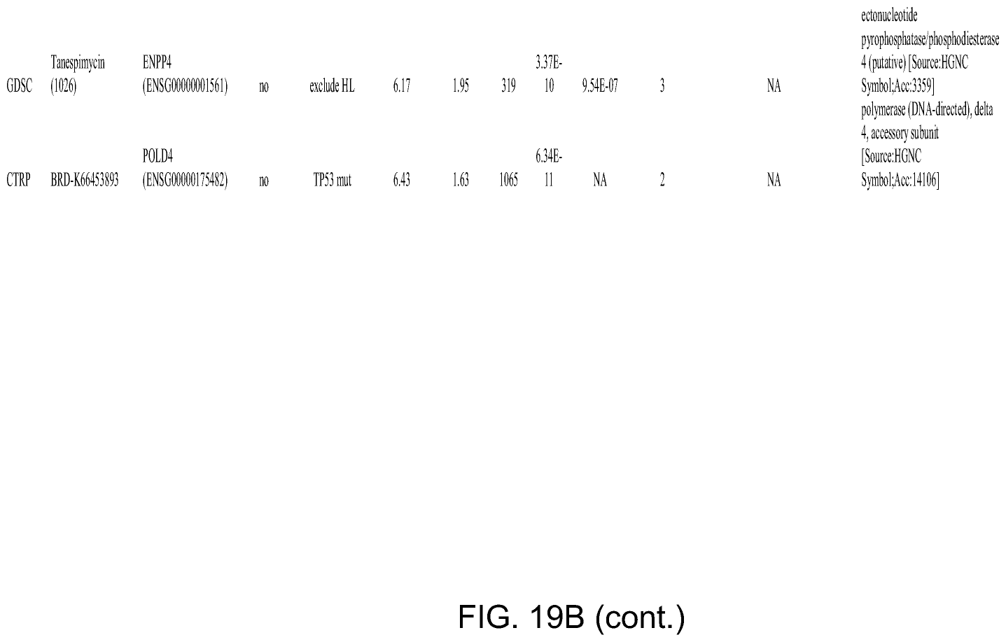

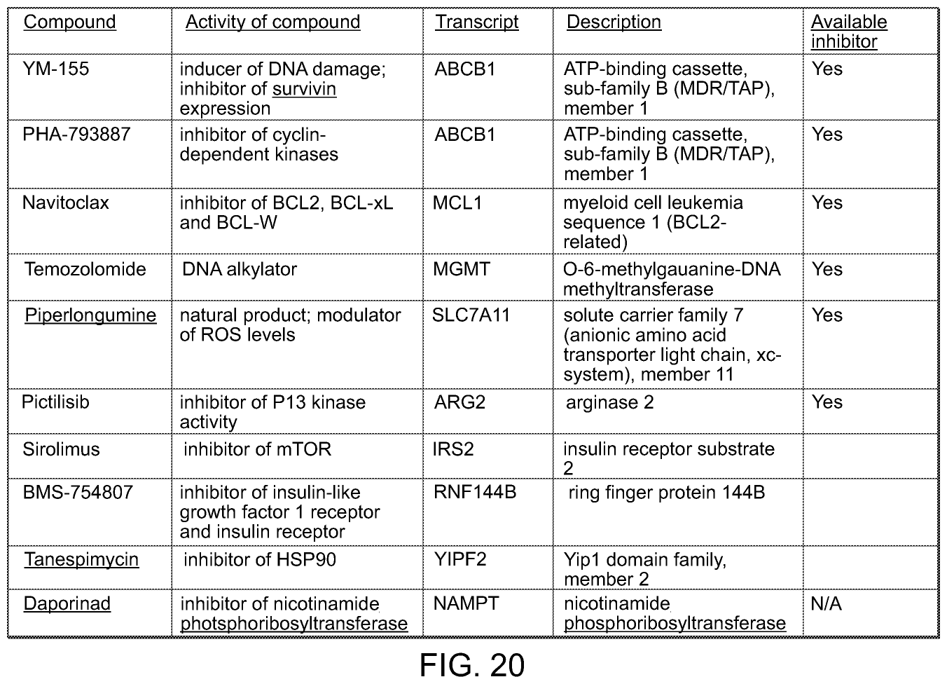

Using this approach, 768 drug/mRNA relationships across 348 small molecules and 488 genes were identified (median: 2 genes associated per compound, maximum: 15; median: 1 compound associated per gene, maximum: 33), including 10 instances where high expression of the same transcript was significantly correlated with a lack of response to the same small molecule in both datasets (FIGS. 1E, 8C, 19, and 20). For 82 of the associated transcripts, annotated small-molecule inhibitors existed for the protein product. These relationships included some that were clinically appreciated (e.g., MGMT, encoding O-6-Methylguanine-DNA Methyltransferase, and the alkylating agent temozolomide), others for which prior experimental evidence has been reported (e.g., the apoptosis regulator MCL1 and the BCL2-family inhibitor navitoclax), and some for which the underlying biology was not clear and the therapeutic potential was undetermined, including MGLL and the lysine demethylase inhibitor GSK-J4, AIFM2 and inhibitors of GPX4 such as ML210, and SLC7A11 and a subset of small molecules including PRIMA-1, shikonin, and piperlongumine (15, 16).

### Example 3: Prioritization of Resistance-Associated Transcripts

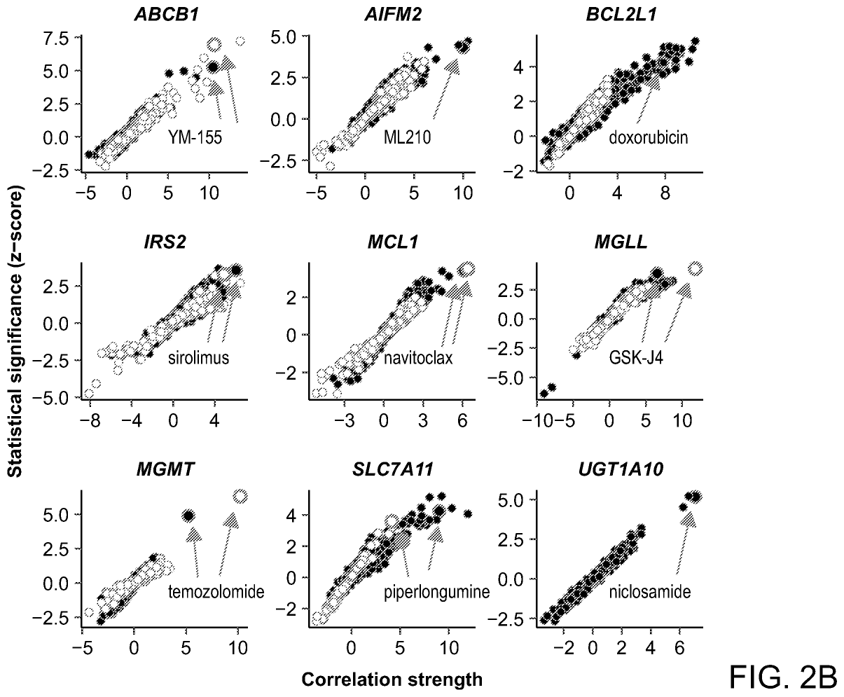

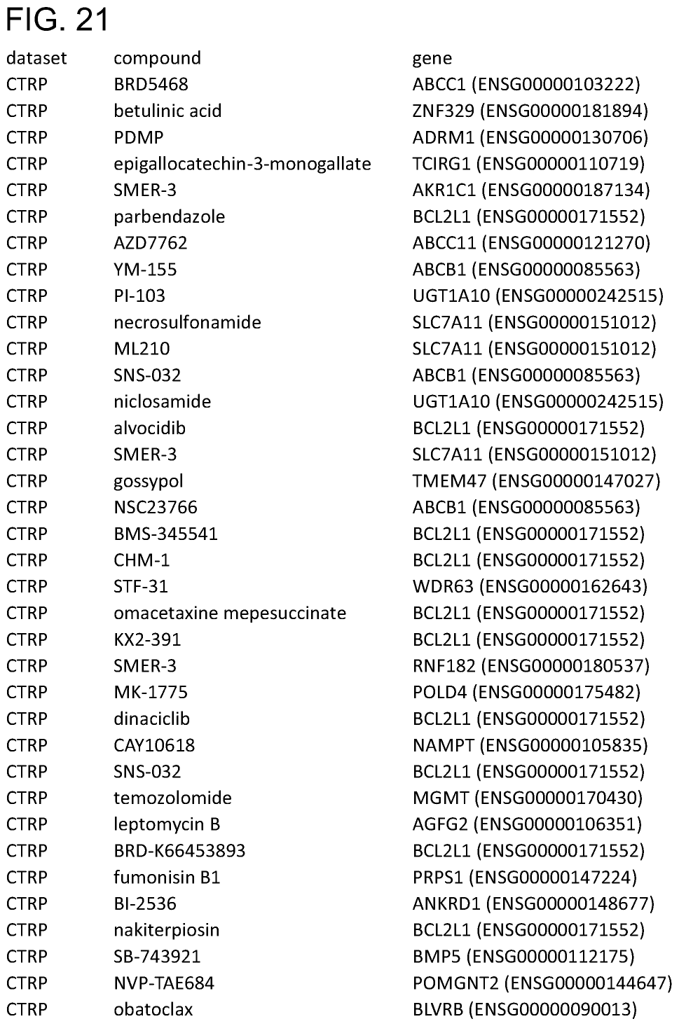

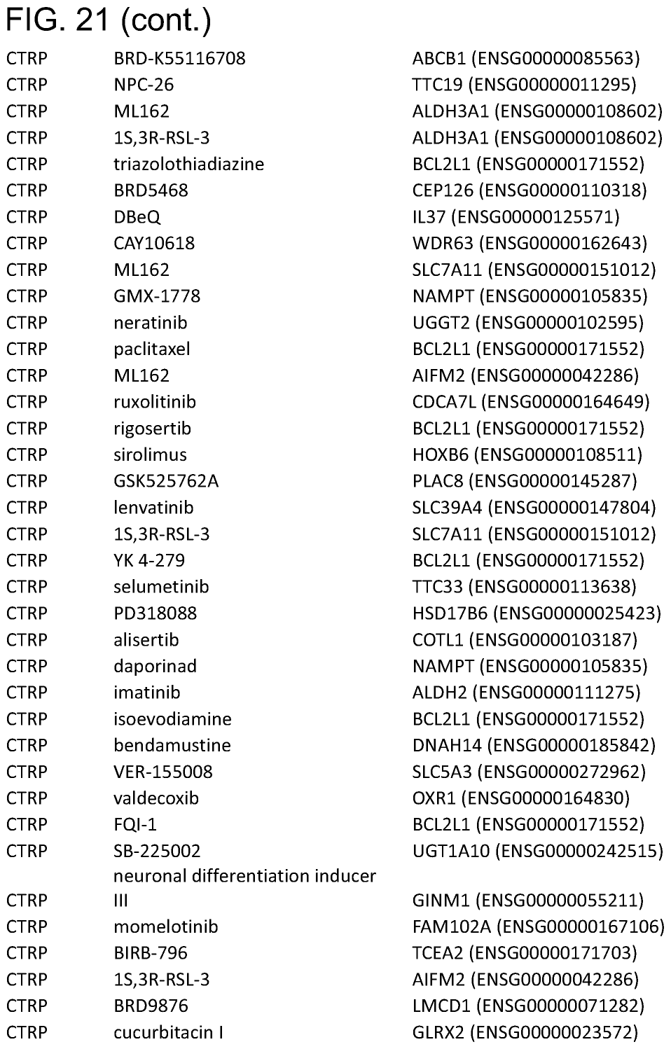

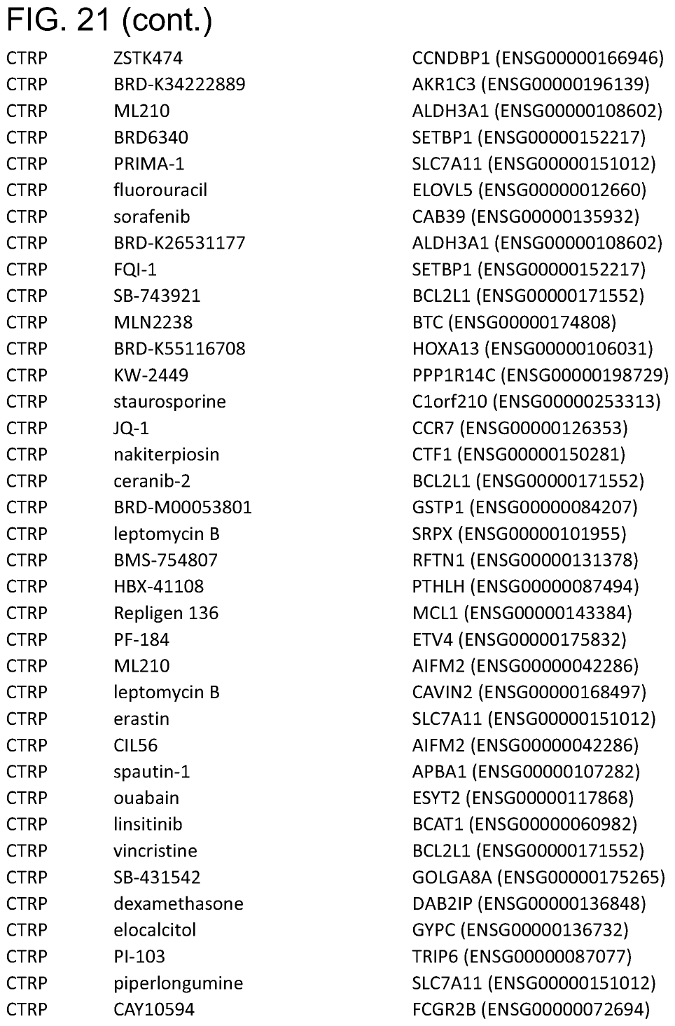

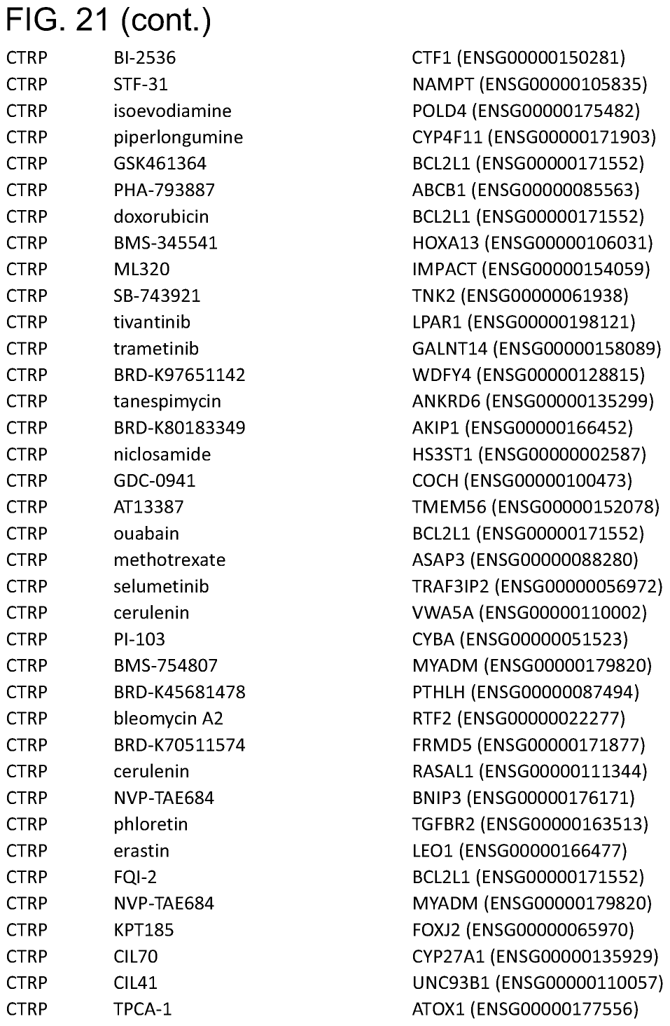

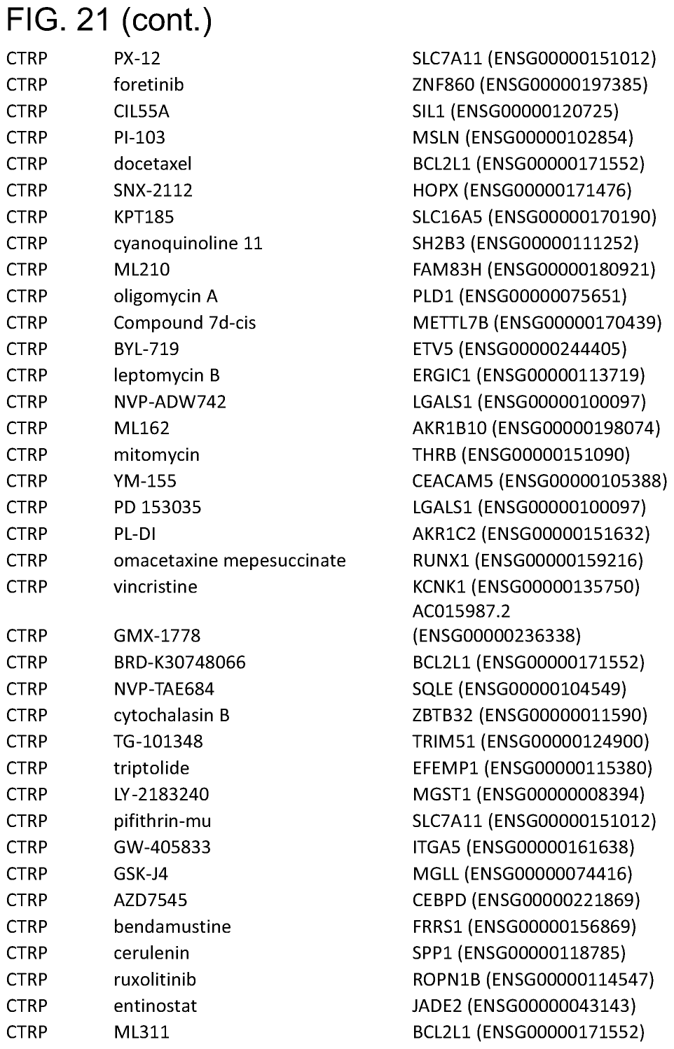

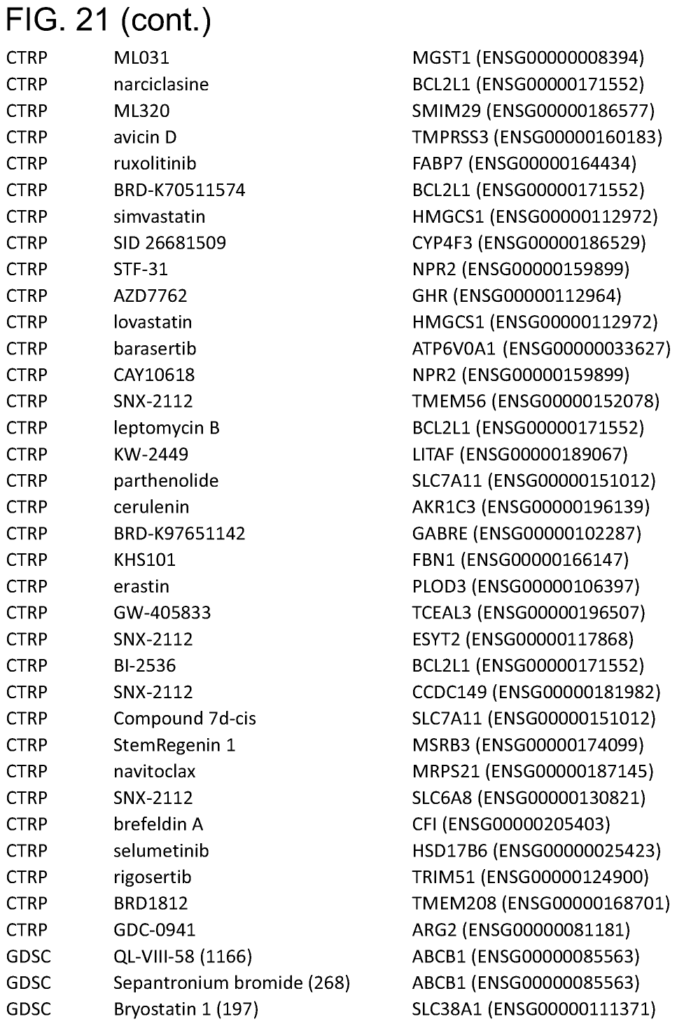

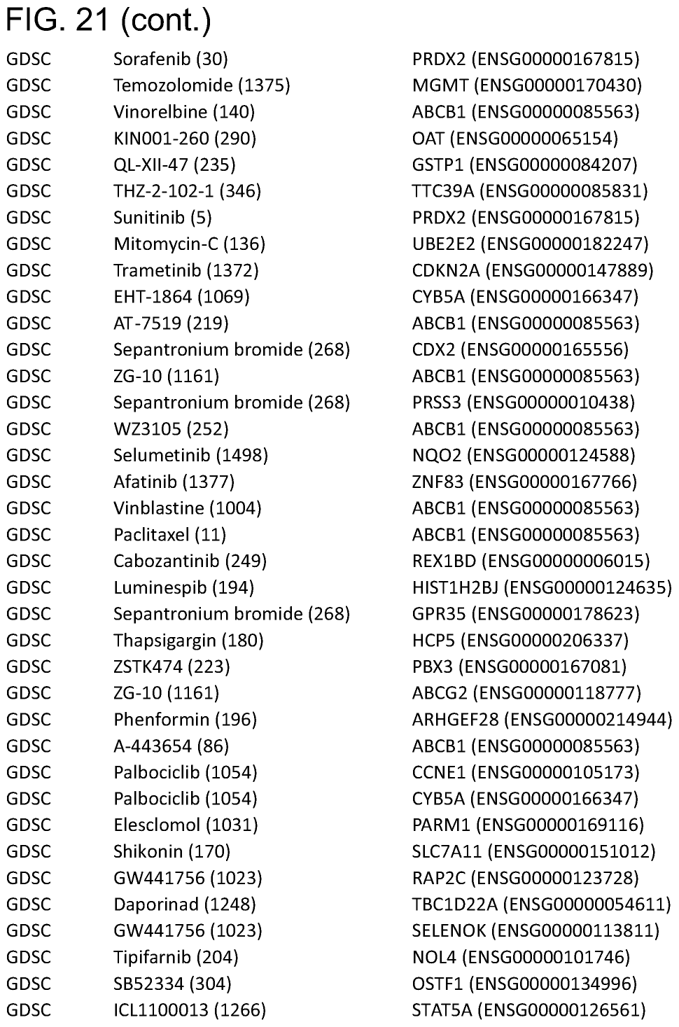

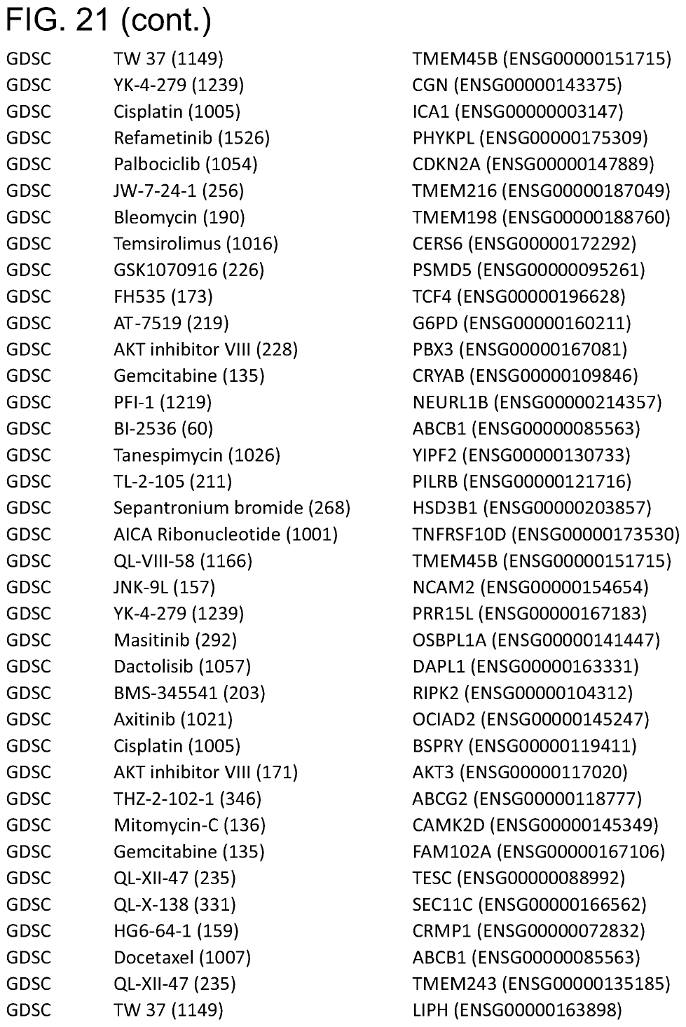

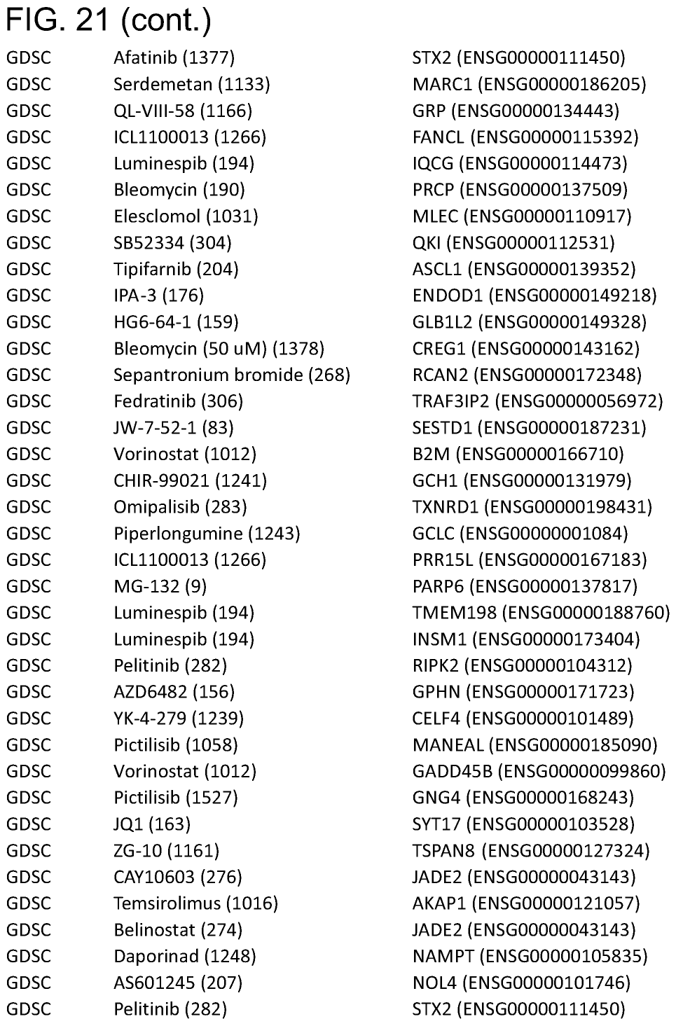

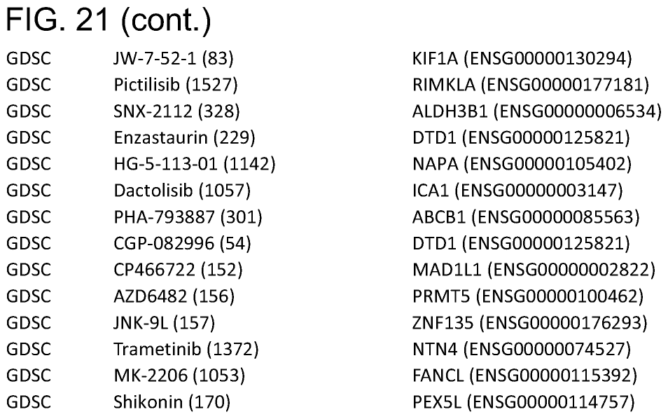

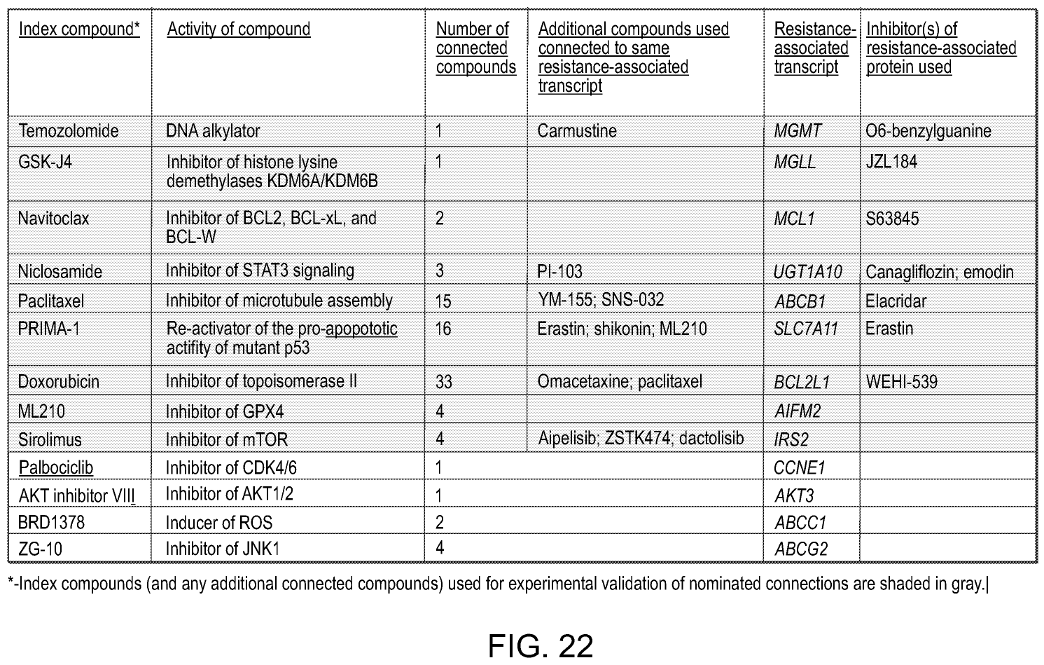

A subset of the highest-confidence relationships was nominated based on significance and behavior across compounds, contexts, and datasets, and this subset of connections was also tested using an orthogonal decision-tree-based analytical approach that combines multiple feature types (e.g., mutation, copy number, reverse-phase protein array) with expression to ensure robustness (FIG. 21) (17). A subset of 23 small molecules with a range of mechanisms and annotated targets that were correlated with 13 putative resistance-associated transcripts was prioritized, selecting nine for in-depth characterization (FIGS. 2A, 2B, 9A, 9B, and 22). Connections that were observed in both the CTRP and Sanger resources were prioritized, as well as those for which the encoded protein had a commercially-available annotated small-molecule inhibitor(s). Prioritized transcripts were additionally selected to represent a range of cellular functions, including regulation of apoptosis (BCL2L1, MCL1), DNA damage repair (MGMT), lipid metabolism (MGLL), putative mitochondrial metabolism (AIFM2), amino acid transport (SLC7A11), insulin signaling (IRS2), and drug processing and metabolism (UGT1A10, ABCB1). These transcripts were also selected to represent the diversity of associations identified: some connections were specific to several compounds with distinct mechanisms and patterns of toxicity (BCL2L1, SLC7A11, and ABCB1), while remaining connections were highly selective for a single small molecule or groups of mechanistically-related small molecules with shared patterns of toxicity.

### Example 4: Validation that mRNA Expression Levels of Resistance-Associated Transcripts are Correlated to Single-Agent Drug Sensitivity

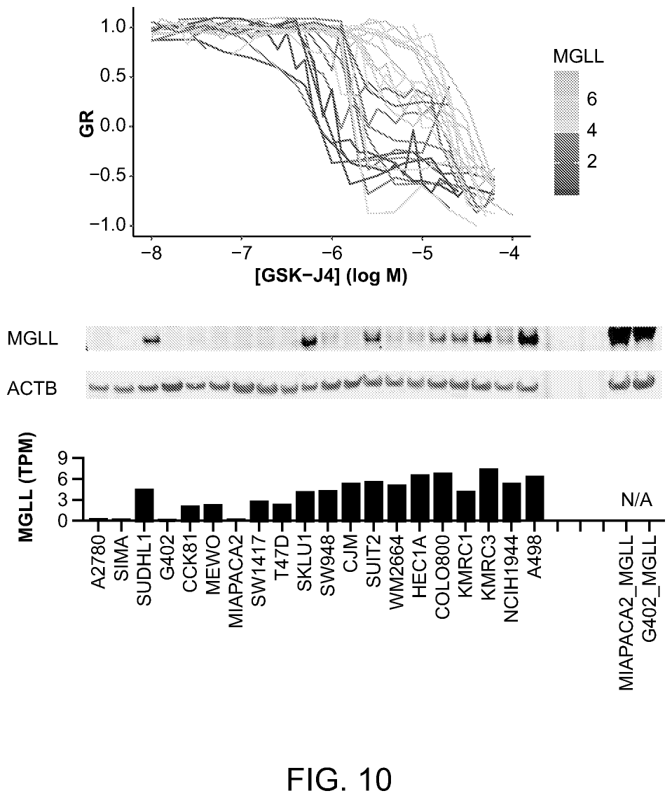

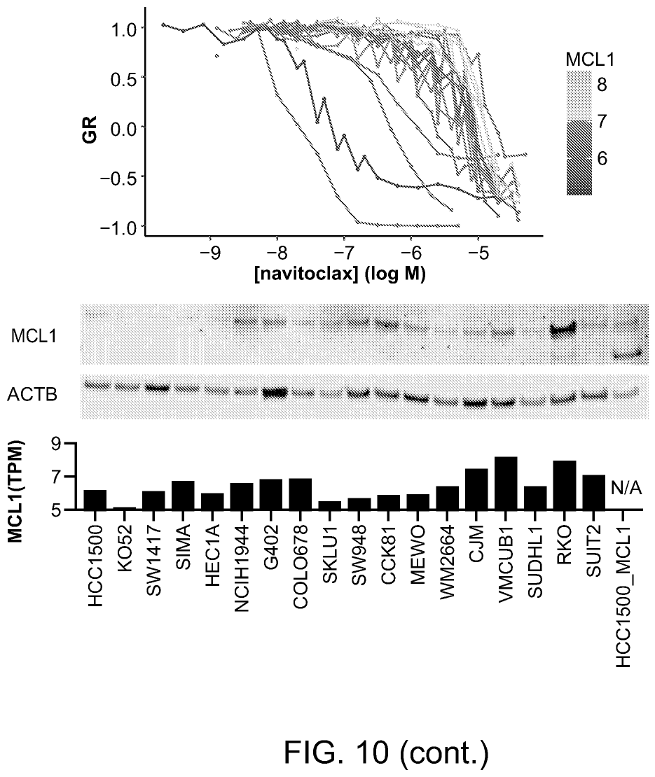

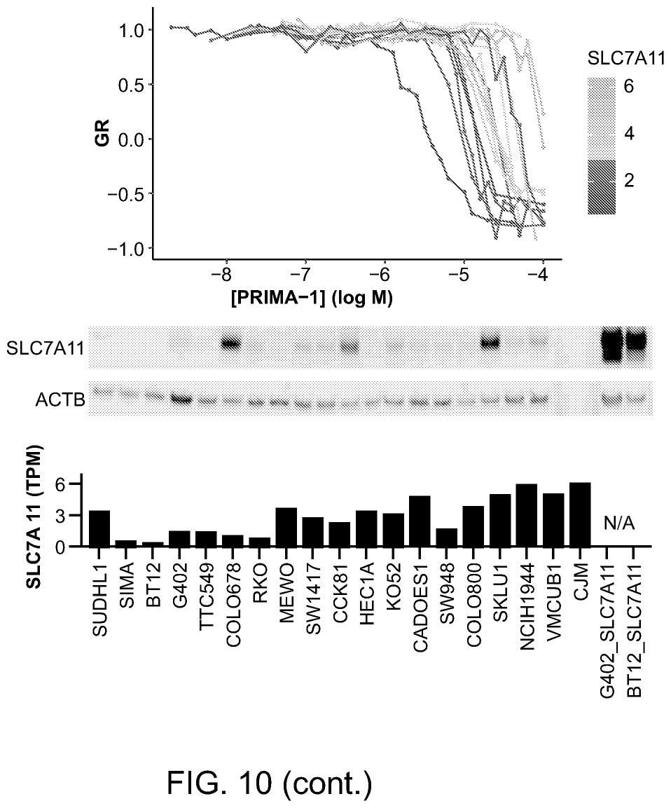

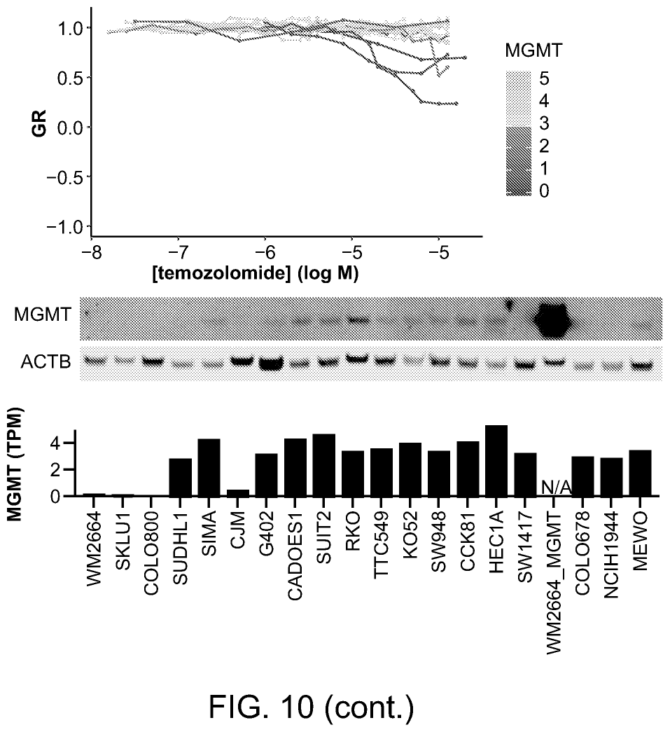

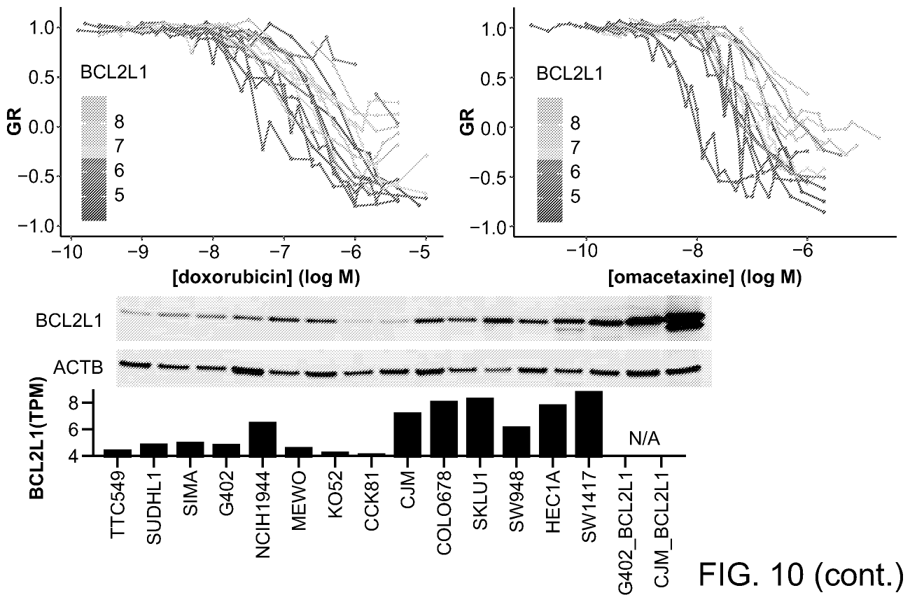

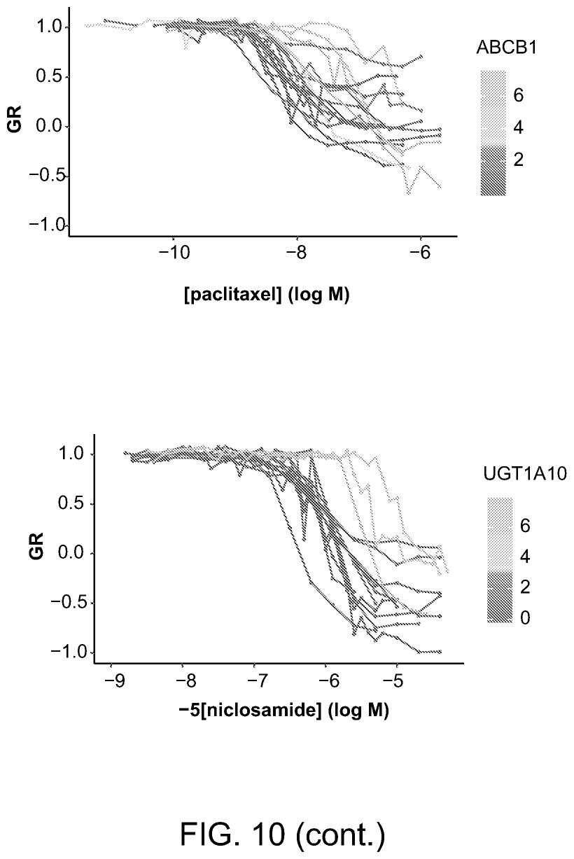

To confirm that specific mRNAs were indeed associated with decreased drug response, a panel of cell lines was first obtained, selected to represent the range of basal mRNA expression of resistance-associated transcripts, and cell line identity was confirmed using SNP-based DNA fingerprinting. Concentration-response curves were then established for index compounds and inhibitors (where available) using CellTiter-Glo, which measures ATP levels, as a surrogate for cell viability (FIG. 10). In parallel, protein levels of resistance-associated transcripts were evaluated by western blotting to orthogonally confirm the relative mRNA expression levels used as a foundation for this approach (FIGS. 10A to 10E). As predicted by CCLE/CTRP data, protein expression was well correlated with drug sensitivity across all cell lines, drugs, and proteins. Overall, these results were highly consistent with and broadly confirmed the original predictions and gene/drug relationships uncovered in the CCLE/CTRP data sets.

### Example 5: Ectopic Expression of Candidate Resistance-Associated Transcripts is Sufficient to Drive Drug Resistance

Having established that the relationships observed in the large-scale datasets were apparent in focused experiments, the next goal was to determine whether these transcripts played a causal role in resistance or were merely markers associated with response. To do this, it was assessed whether the ectopic overexpression of candidate resistance transcripts predicted by drug/mRNA relationships in deficient (or lowly-expressing), drug-sensitive cell lines would be sufficient to confer resistance.

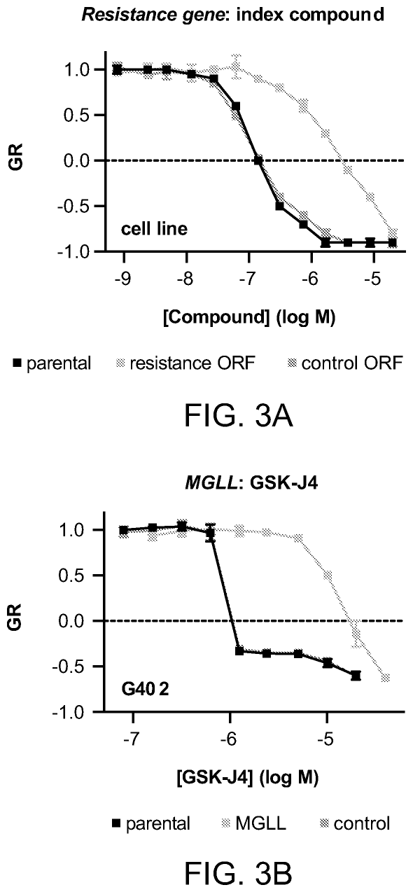

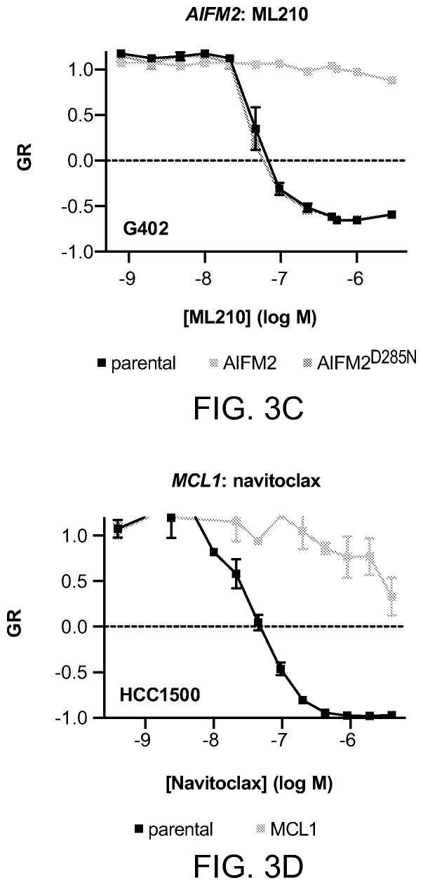

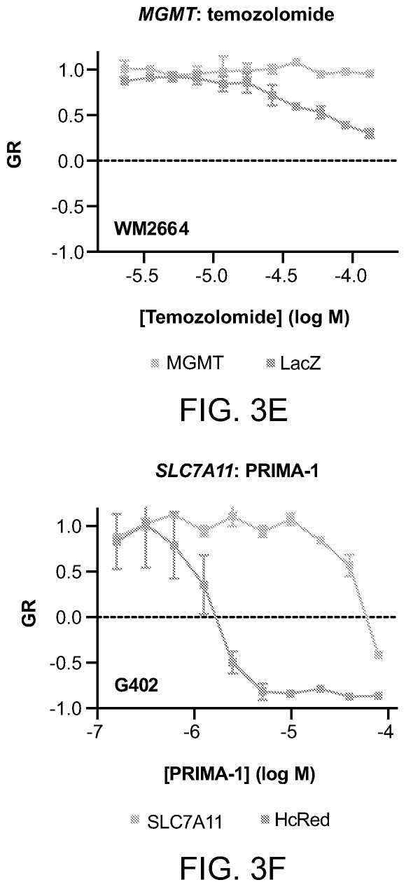

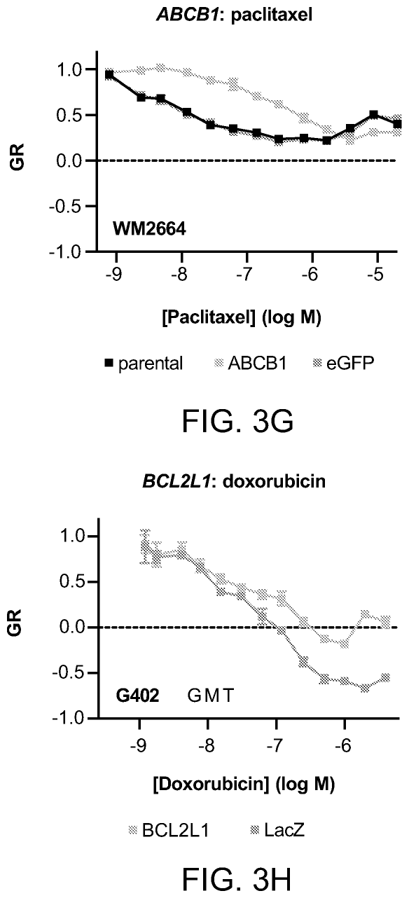

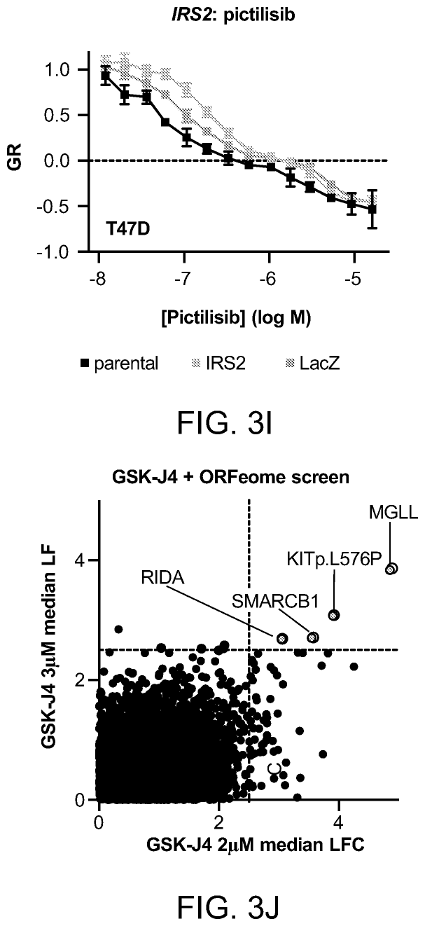

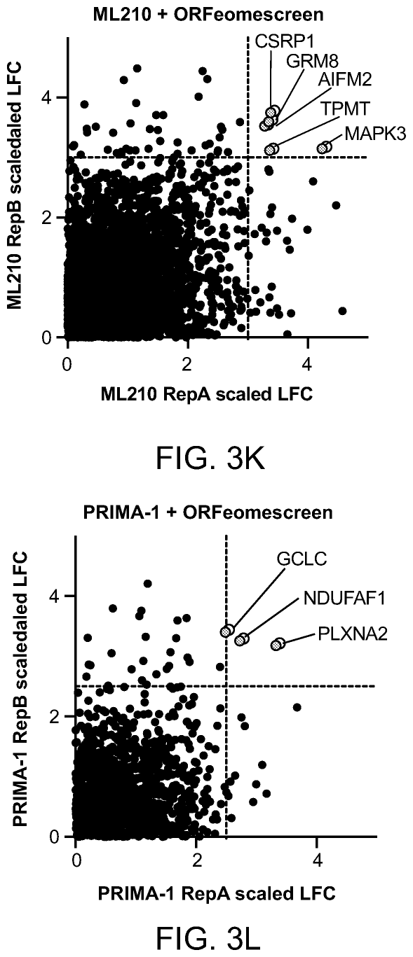

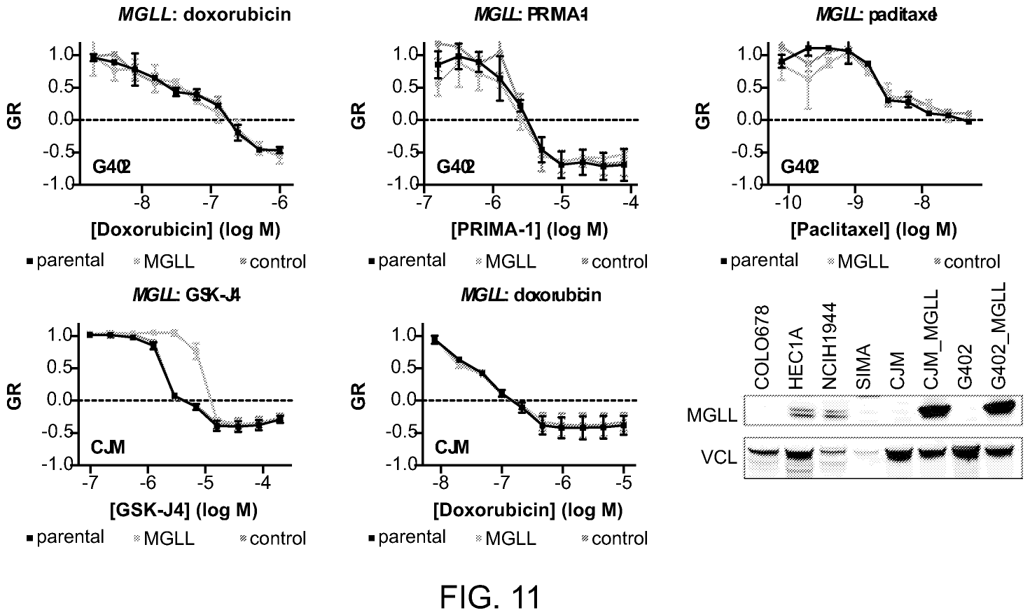

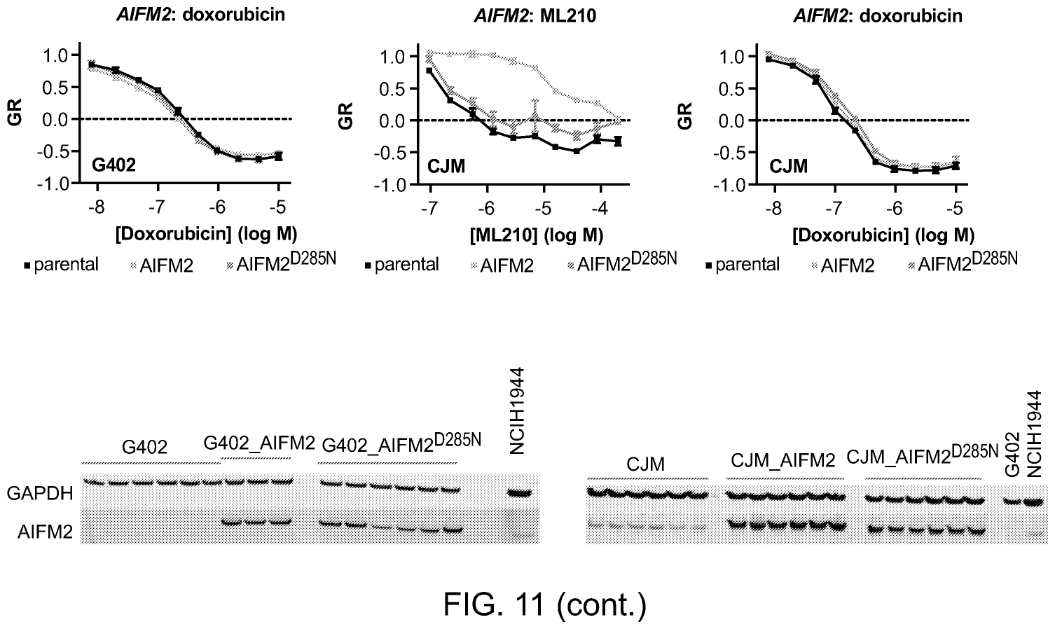

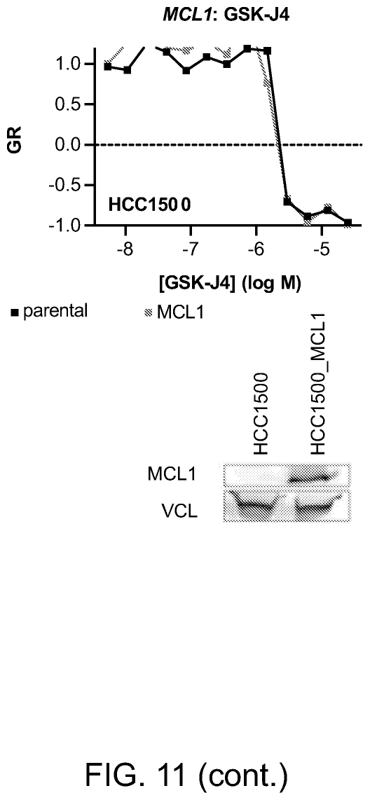

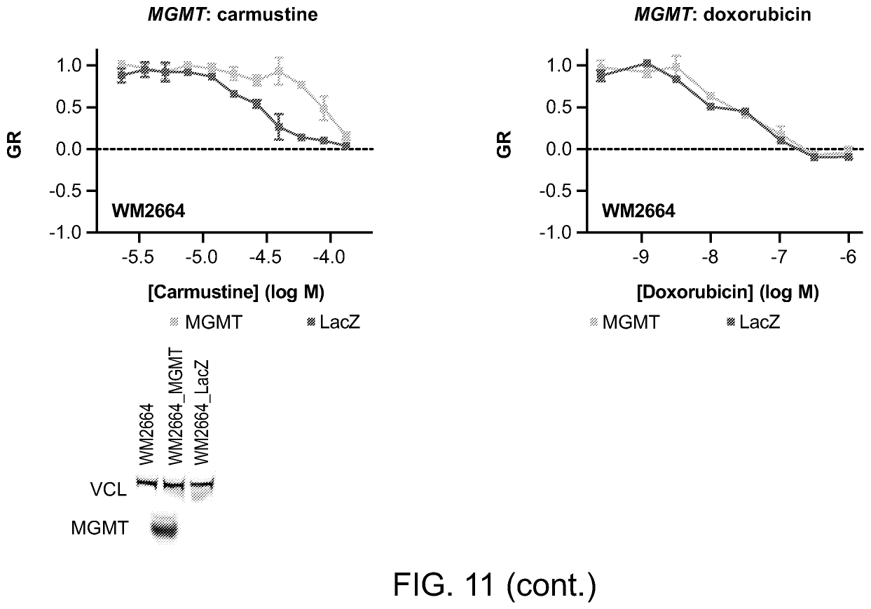

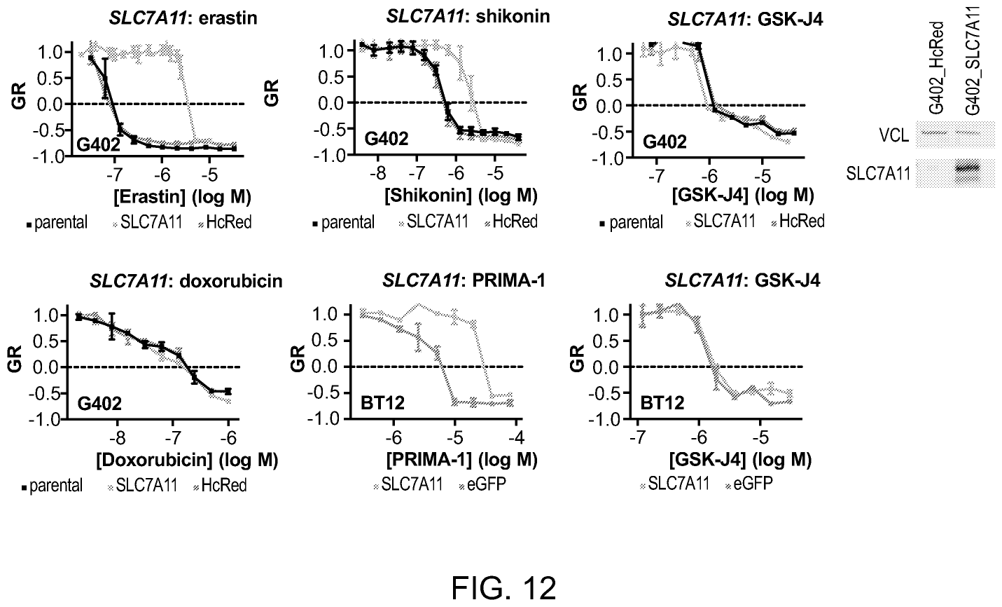

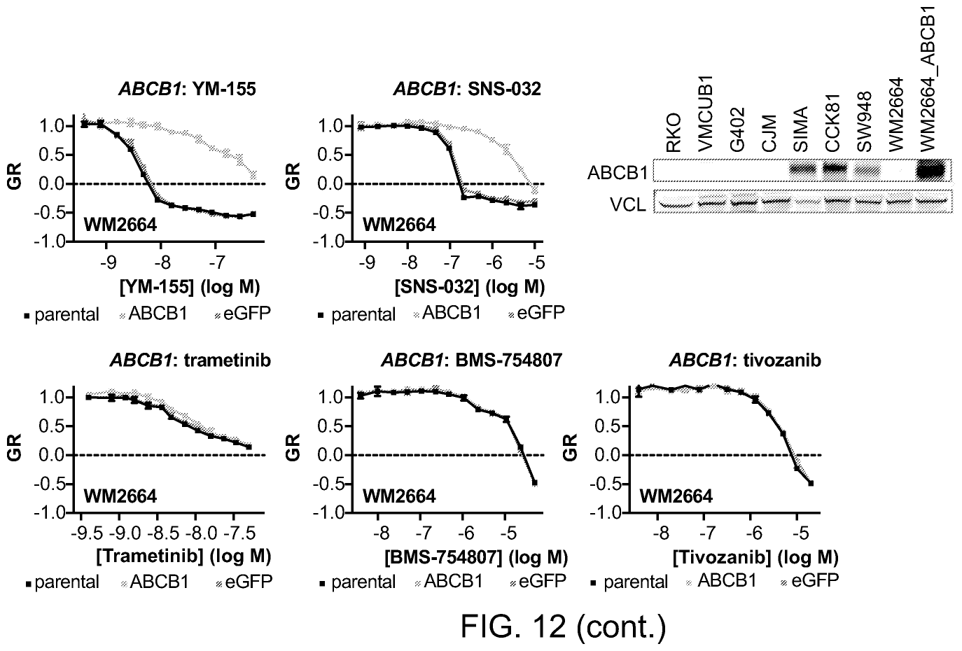

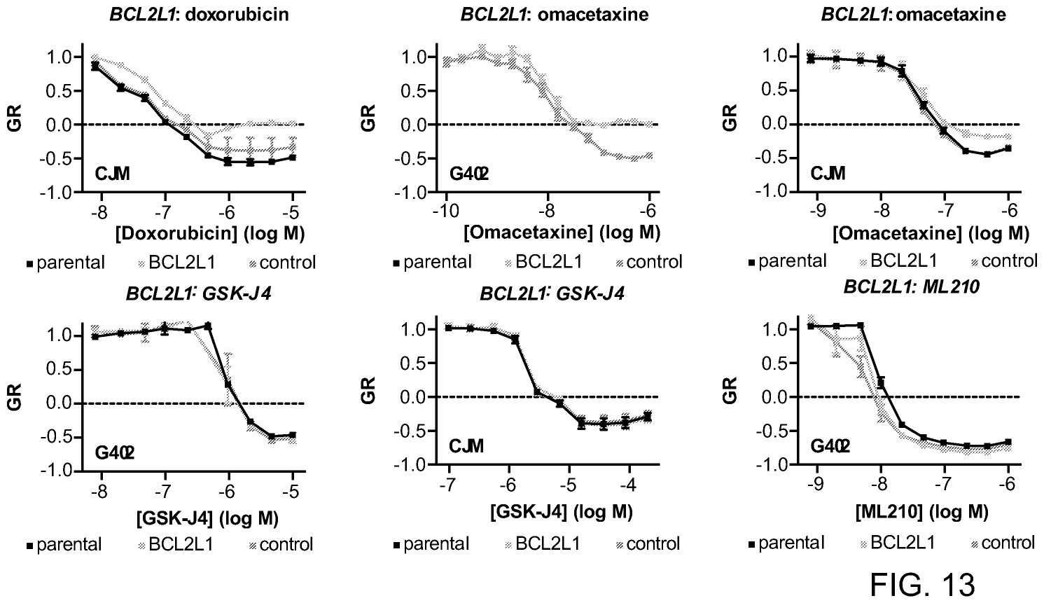

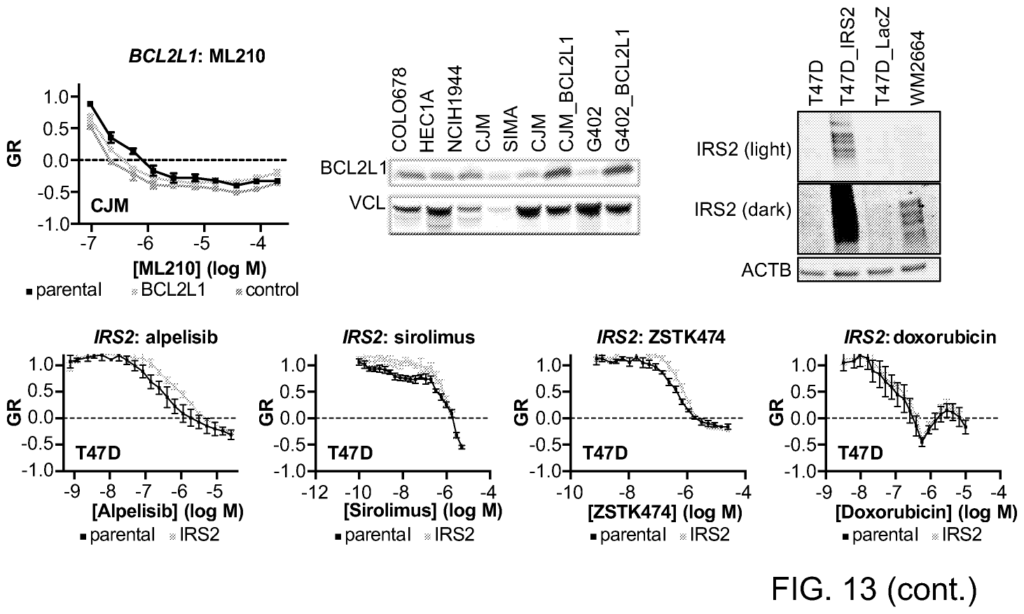

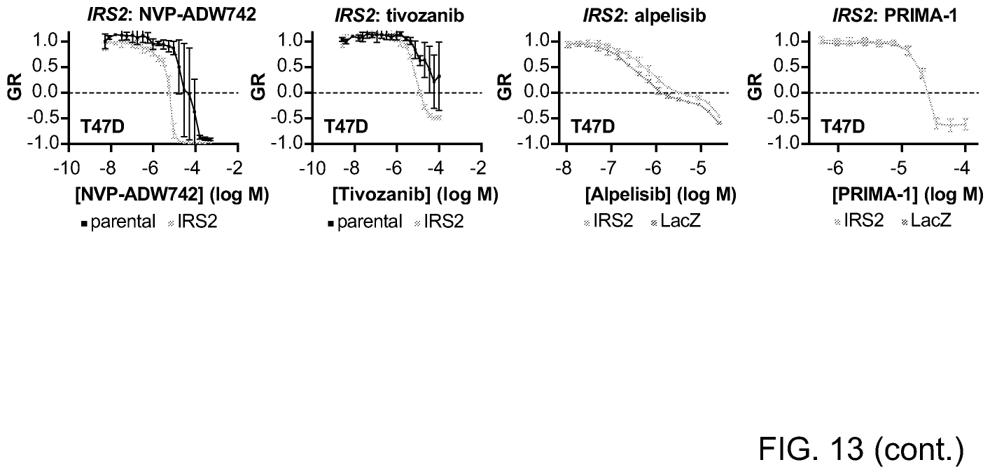

Parental cell lines were virally transduced with cDNAs that constitutively overexpressed BCL2L1, AIFM2, or MGLL (in G402 and CJM cells); ABCB1 or MGMT (in WM2664 cells); SLC7A11 (in BT12 and G402 cells); MCL1 (in HCC1500 cells); or IRS2 (in T47D cells) and subsequently drug sensitivity was measured. To account for potential differences in growth rates and to distinguish between cytostatic and cytotoxic drug responses, the normalized growth rate inhibition (GR) metric was used to assess cell viability (FIG. 3A) (18). Overexpression of MGLL conferred resistance to GSK-J4, resulting in a 20-fold shift in GR₅₀ (the value where GR=0.5) (FIG. 3B). Significantly, MGLL expression was not sufficient to confer resistance to a series of other small molecules that are unrelated to GSK-J4 or its associated biology (FIG. 11A). AIFM2, but not a kinase- and DNA-binding-dead mutant (AIFM2^{D285N}) of AIFM2, conferred selective resistance to ML210 (>500-fold) and more modest resistance to PRIMA-1 (approximately threefold) (FIGS. 3C and 11B). MCL1 conferred selective resistance (100-fold) to navitoclax (FIGS. 3D and 11C). MGMT conferred selective resistance to temozolomide (no detectable toxicity) and carmustine (4-fold) (FIGS. 3E and 11D). SLC7A11 conferred selective resistance to PRIMA-1 (20-fold), erastin (30-fold), shikonin (10-fold), and ML210 (8-fold) (FIGS. 3F and 12A). ABCB1 conferred selective resistance to paclitaxel (50-fold), SNS-032 (25-fold), and BI-2536 (40-fold) (FIGS. 3G and 12B). BCL2L1 conferred selective resistance to doxorubicin and omacetaxine, with modest (2-4-fold) changes in GR₅₀ accompanied by a shift from cytotoxic to cytostatic responses at high concentrations (FIGS. 3H and 13A). IRS2 conferred selective, yet modest and reproducible, resistance to pictilisib, sirolimus, alpelisib, and ZSTK474 (2-4-fold) (FIGS. 3I and 13B). Collectively, these results indicated that transcripts identified as correlates to drug resistance using this approach were not simply passive markers of a drug-resistant state, but were in fact sufficient to confer drug resistance.

### Example 6: Drug-Rescue Screens Performed with a Near-Genome-Scale ORF Library Highlighted the Specificity of Resistance-Associated Transcripts to Small Molecule Inhibitors

While the resistance-associated transcripts uncovered using this approach were nearly all sufficient to induce drug resistance when ectopically expressed, it was next examined if there were other genes that were sufficient to induce resistance, but that were not identified as correlated with resistance in the analysis that was carried out. Thus, to explore a broad landscape of potential resistance mechanisms in an unbiased fashion, G402 cells were infected with a near-genome-scale open-reading frame (ORF) library containing 17,255 ORFs at a low MOI to generate a population of cells, each expressing a single unique ORF (19). After infection and selection, cells were split into vehicle (DMSO) and drug-treatment arms, with remaining cells harvested for sequencing for baseline relative library abundance (early timepoint). The infected cells were treated with three compounds that were highly potent at suppressing growth in G402 cells and where novel connections had been implicated by the analyses performed above: GSK-J4 (at two different concentrations), ML210, or PRIMA-1 (FIGS. 3J-3L). After a 9-day treatment with GSK-J4, MGLL was the top resistance-conferring ORF across replicates and concentrations, which indicated that this gene is uniquely sufficient to augment GSK-J4 sensitivity (FIG. 3J). Similarly, for ML210, AIFM2 was the third-most enriched gene across the genome-scale collection (FIG. 3K). Unfortunately, SLC7A11 was not detectable in the screening library at the early timepoint or under any drug condition; however, in PRIMA-1-treated cells, GCLC, which encodes glutamate-cysteine ligase catalytic subunit and acts immediately downstream of SLC7A11 (a cysteine/glutamate transporter), was the third most enriched ORF (FIG. 3L) (20). Collectively, these results indicated the relative potential of nominated genes to confer resistance meets or exceeds that of the vast majority of other transcripts.

### Example 7: Combinatorial Inhibition of Resistance-Associated Proteins and Indexed Compounds Engendered Synergistic Responses

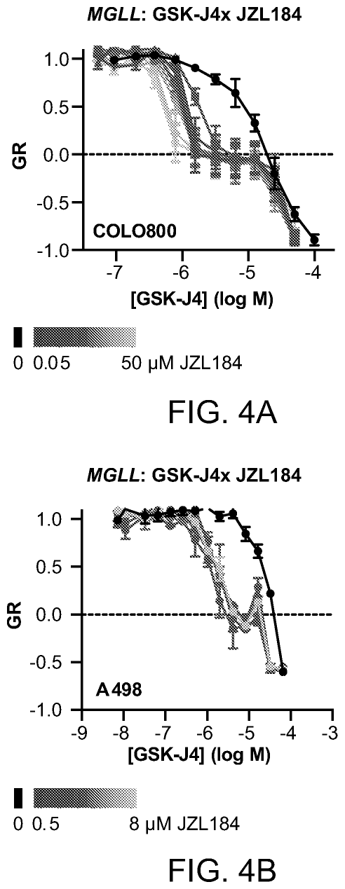

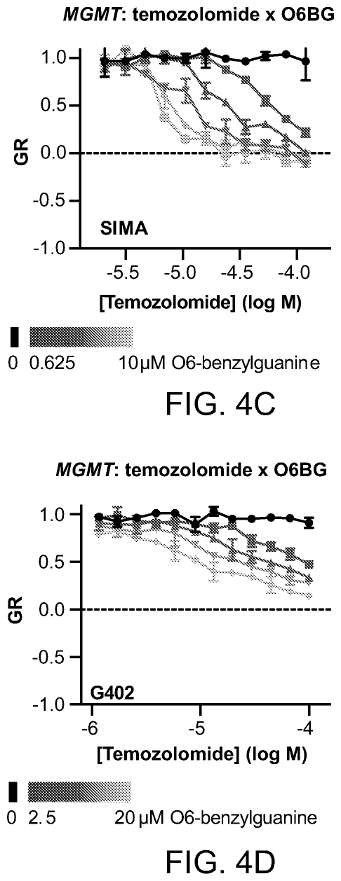

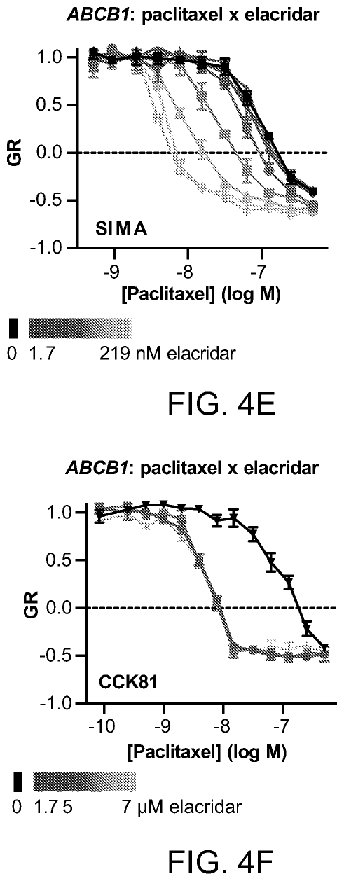

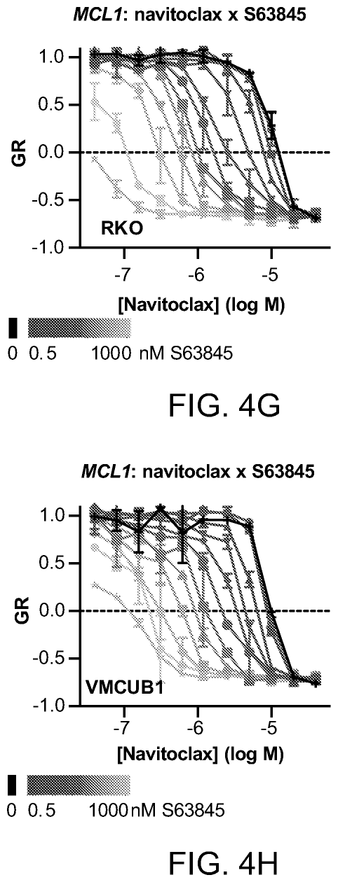

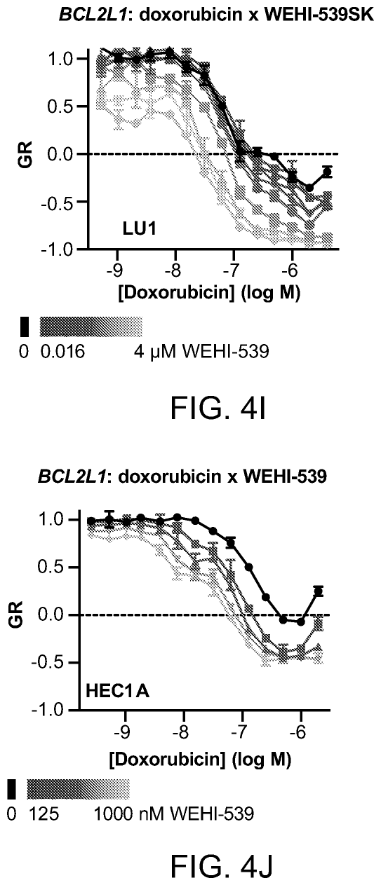

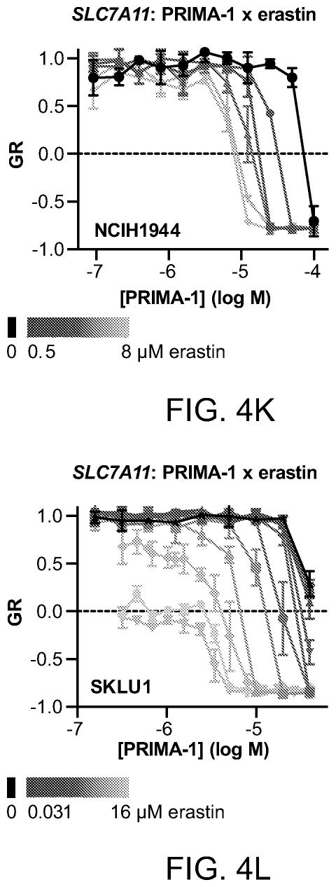

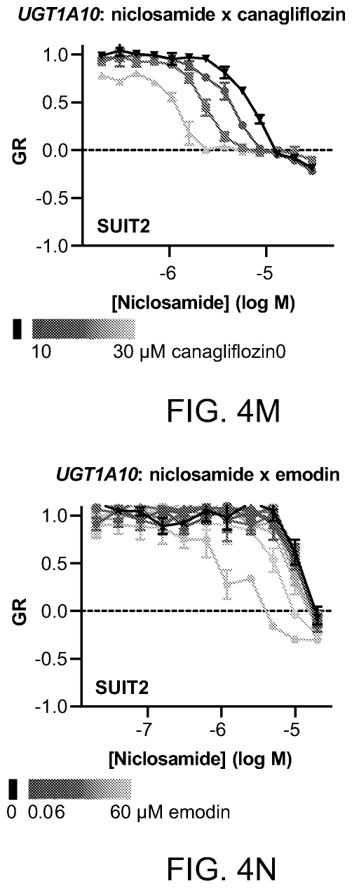

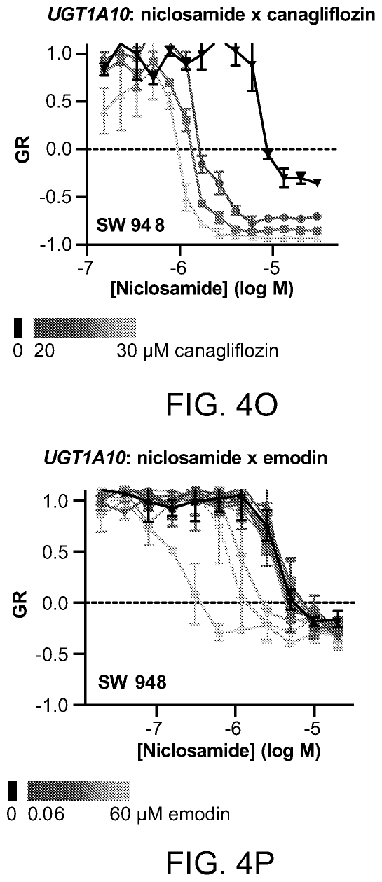

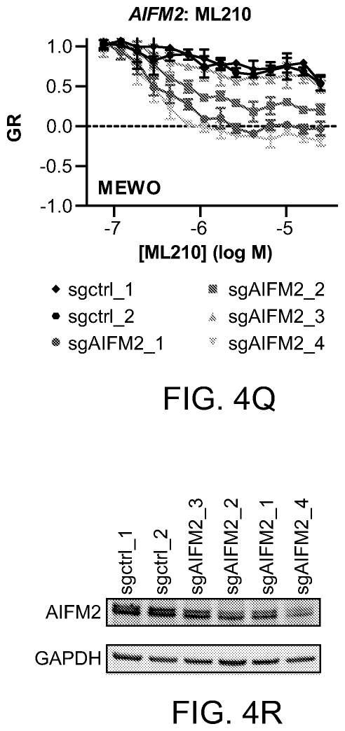

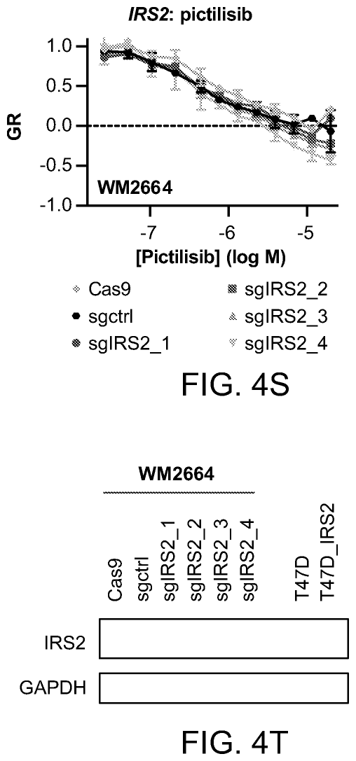

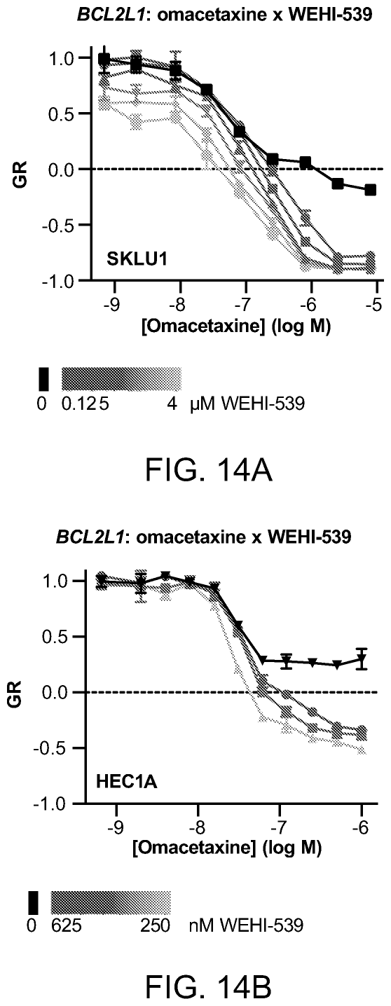

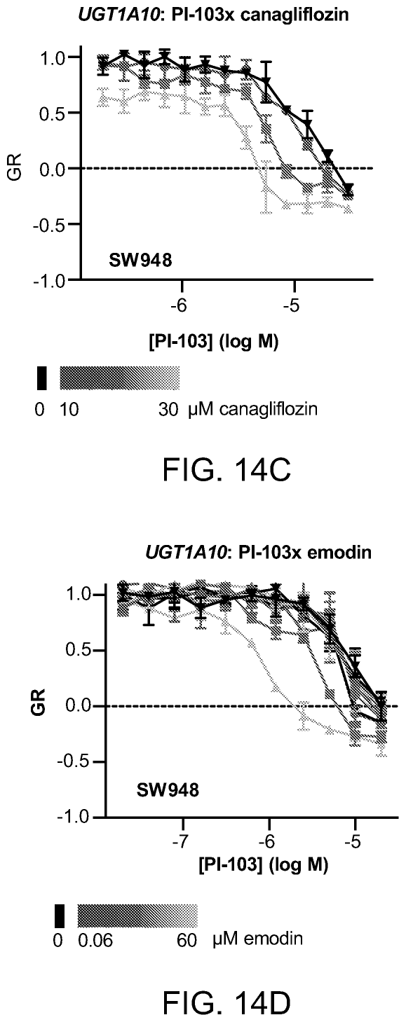

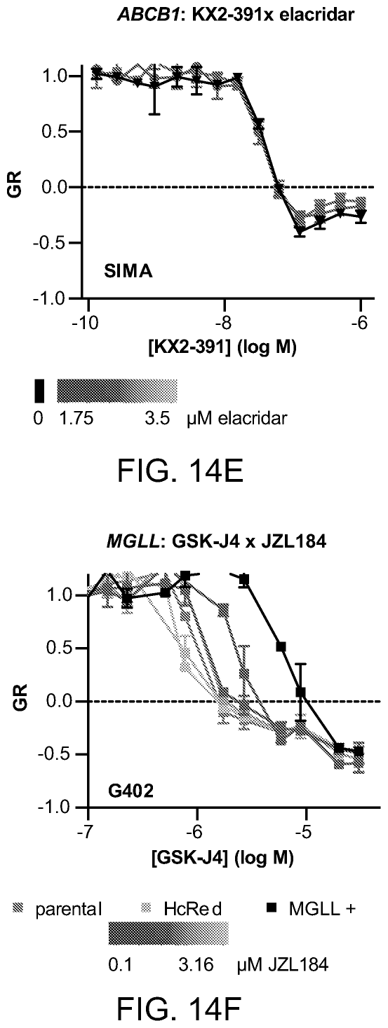

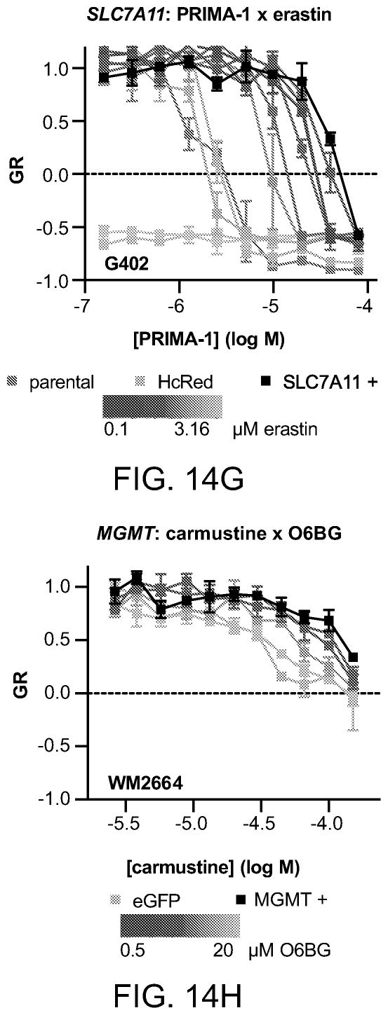

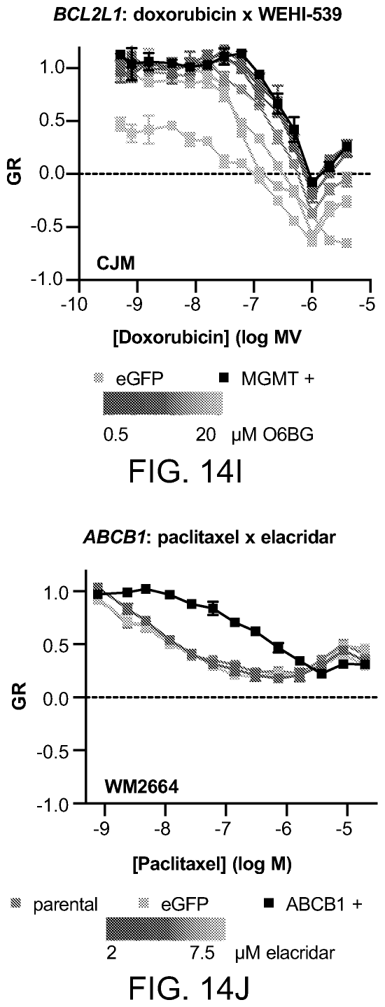

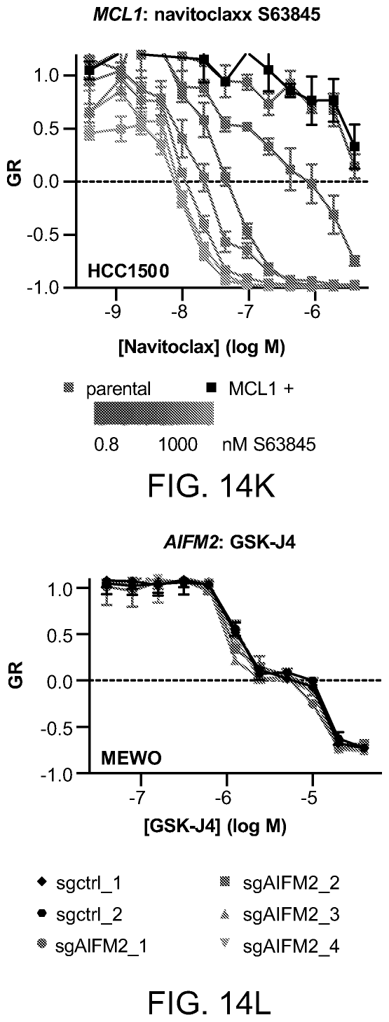

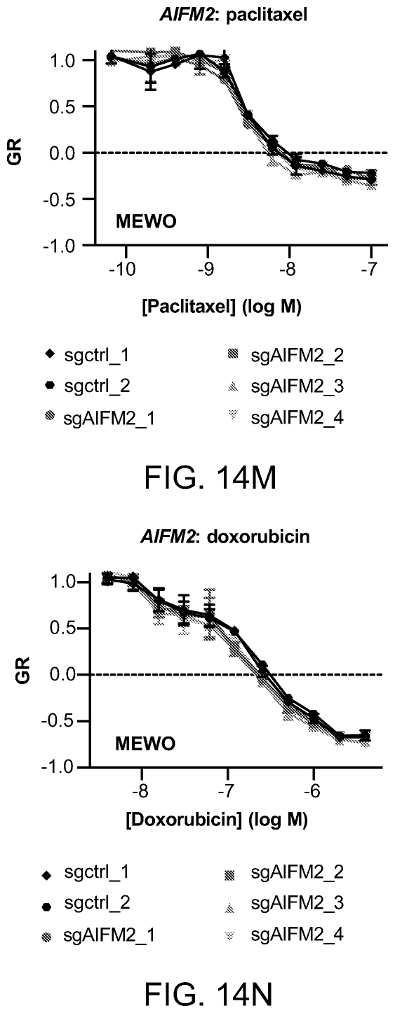

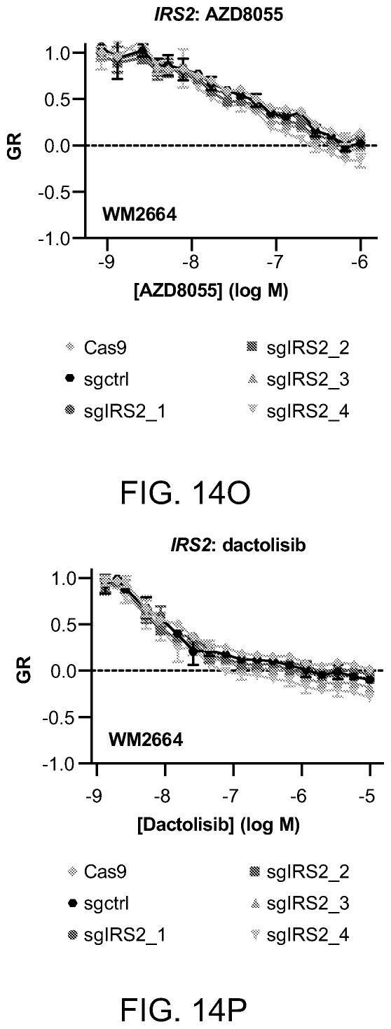

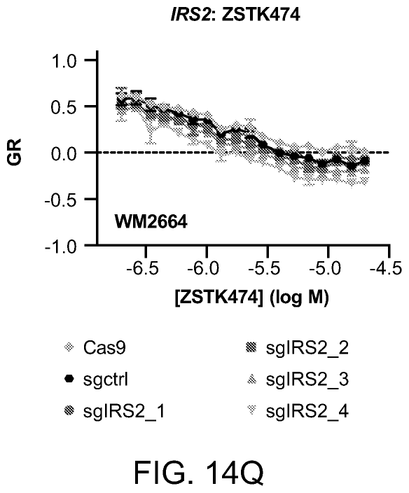

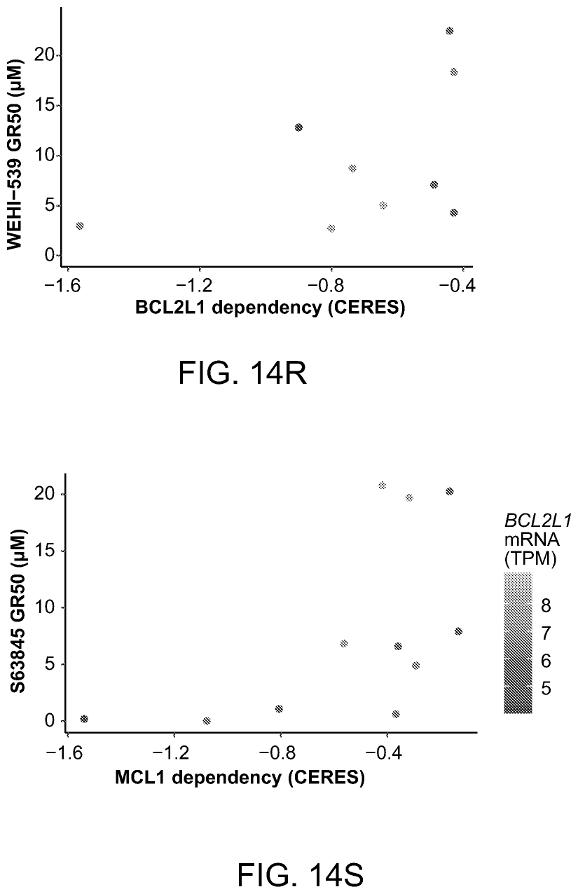

Having demonstrated that ectopic expression of nominated transcripts was sufficient to confer resistance, the necessity of these transcripts was then tested via use of pharmacological inhibitors targeting their protein products. To do this, cell lines were chosen that had detectable expression of the mentioned nominated resistance transcripts and displayed reduced drug sensitivity compared to cells with low transcript expression. In these cell lines, the effects of co-treatment of index compounds with inhibitors of nominated proteins were tested. Published inhibitors of MGLL (JZL184), MGMT (O6-benzylguanine), ABCB1 (elacridar), MCL1 (S63845), BCL2L1 (WEHI-539), and SLC7A11 (erastin) were acquired. While no selective inhibitors of UGT1A10 have been described, the established substrates emodin and canagliflozin were used as competitive inhibitors (21, 22). To ensure a complete evaluation of these relationships, multiple concentrations of inhibitors with a full concentration-response of index compounds in cell lines with high expression of the nominated protein were combined. Robust sensitization was observed, most notably at concentrations where each inhibitor showed no effects on viability alone, for GSK-J4+JZL184 (FIGS. 4A and 4B), temozolomide+06-benzylguanine (FIGS. 4C and 4D), paclitaxel+elacridar (FIGS. 4E and 4F), navitoclax+S63845 (FIGS. 4G and 4H), doxorubicin (or omacetaxine)+WEHI-539 (FIGS. 41, 4J, 14A, and 14B), PRIMA-1+erastin (FIGS. 4K and 4L), and niclosamide (or PI-103)+canagliflozin (or emodin) (FIGS. 4M, 4P, 14C, and 14D). Similarly, using the overexpression models described herein, resistance conferred by ectopic overexpression could be reversed by co-treatment with these inhibitors in all tested contexts (FIGS. 14F to 14K).

As small-molecule inhibitors were not available for AIFM2 or IRS2, CRISPR-Cas9 was used to knock out these genes in proficient cell lines. Targeting AIFM2 with four different sgRNAs resulted in sensitization to ML210 in the MEWO cell line, with the degree of sensitization consistent with level of protein loss (FIGS. 4Q, 4R, 14L and 14N, and 23). Consistent with the modest phenotype observed with IRS2 overexpression, knockdown of IRS2 in WM2664 cells resulted in modest but consistent sensitization to pictilisib and other mTOR/PI3K inhibitors (FIGS. 4S, 4T, 14O-14Q, and 23).

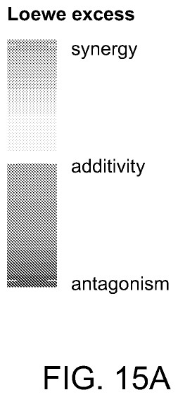

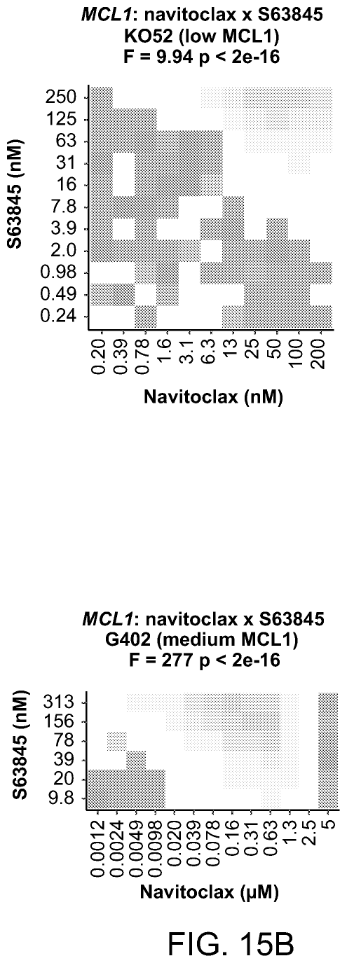

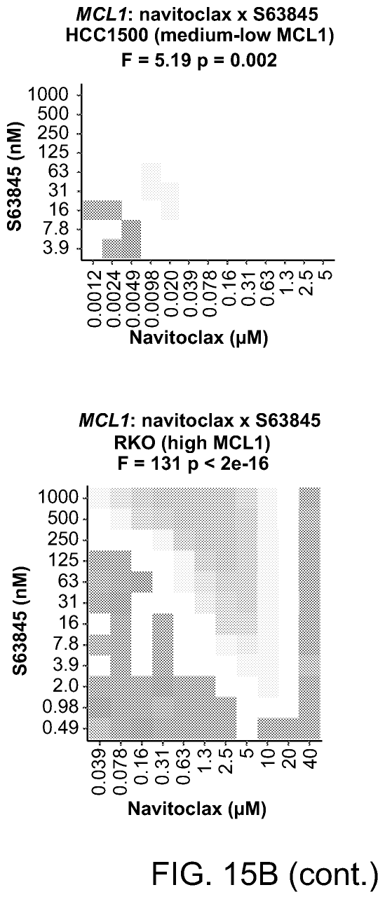

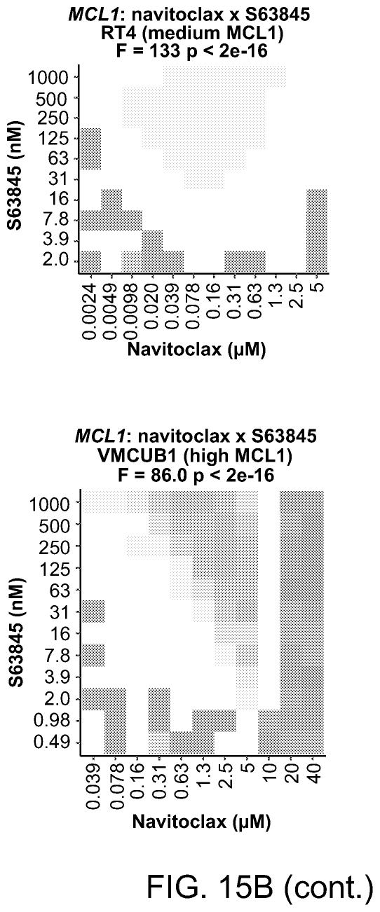

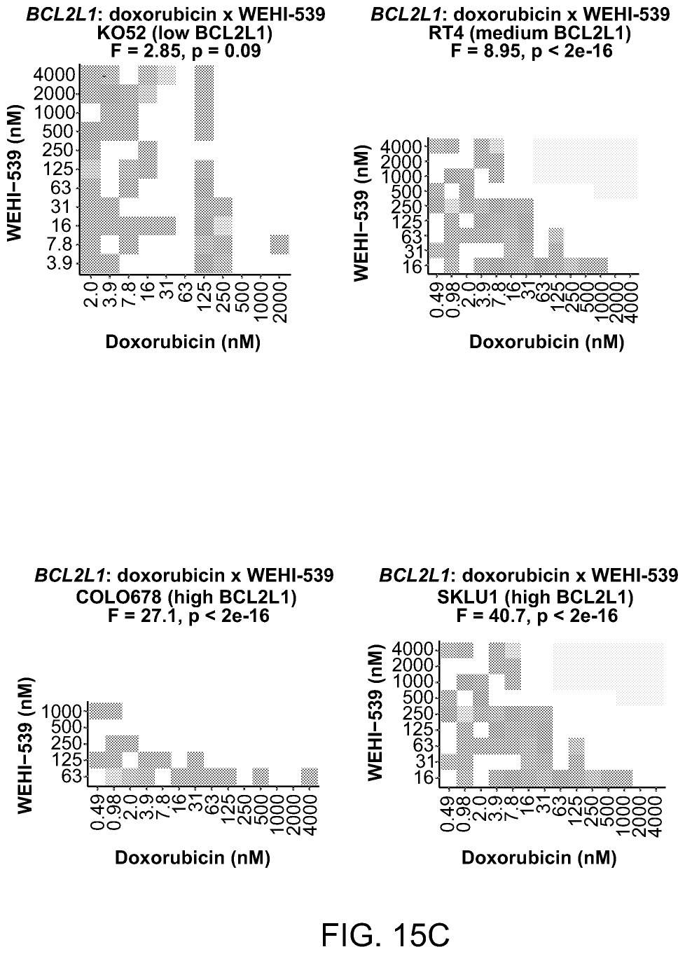

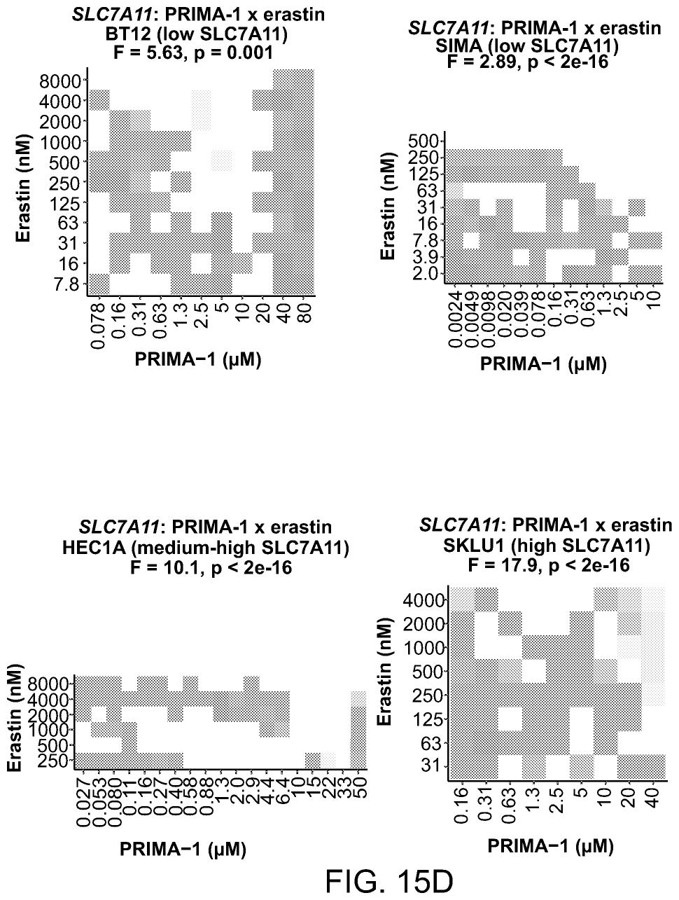

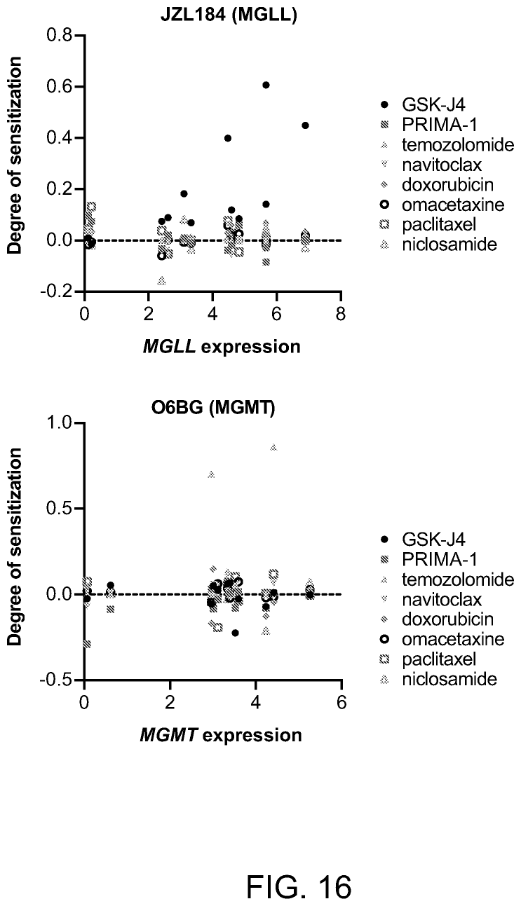

In all cases, sensitization was observed at concentrations at which each of these drug (or gene) combination partners produced no growth decrement on their own. In a number of cases, such as JZL184, only modest growth impact was observed across all concentrations, even at the highest concentration tested (FIG. 4A). The data obtained indicated two things. First, it indicated that the co-targets that cooperate to re-sensitize cell lines resistant to the indexed agent are themselves not dependencies in cancer cell lines. When the genetic dependency profiles of resistance-associated transcripts using DepMap dependency scores for both CRISPR and RNAi-mediated inhibition (www.depmap.org) was assessed, it was found that only BCL2L1 and MCL1 represented robust dependencies across multiple cell lines, whereas suppression of all other transcripts was generally well tolerated (consistent with single-agent pharmacological response profiles) (FIGS. 14R and 14S). Secondly, it indicated that co-treatment will be highly synergistic (FIG. 15). Thus, the discovery of these highly synergistic drug combinations would in principle have been difficult to uncover using empiric combination testing or using single-agent/single-gene approaches.

### Example 8: Synergy is Specific to Cell Lines with Outlier Co-Target Expression

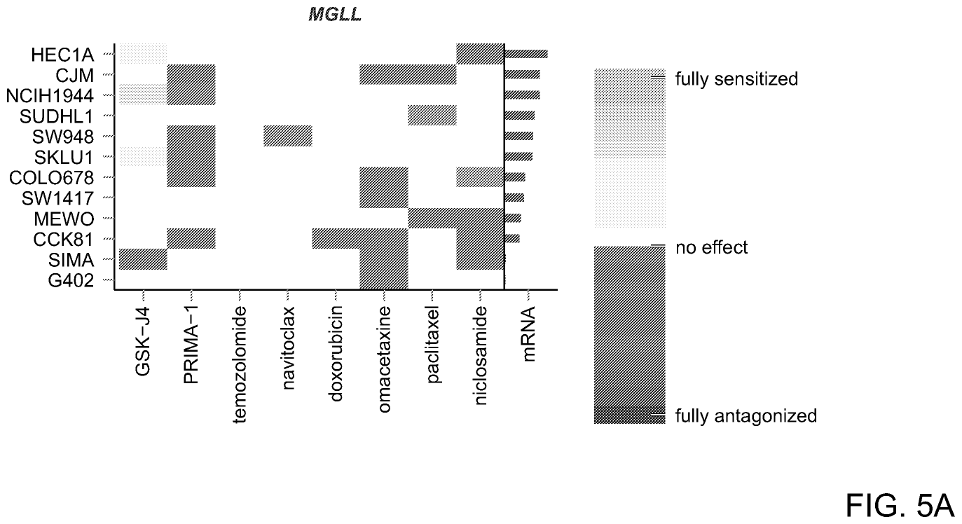

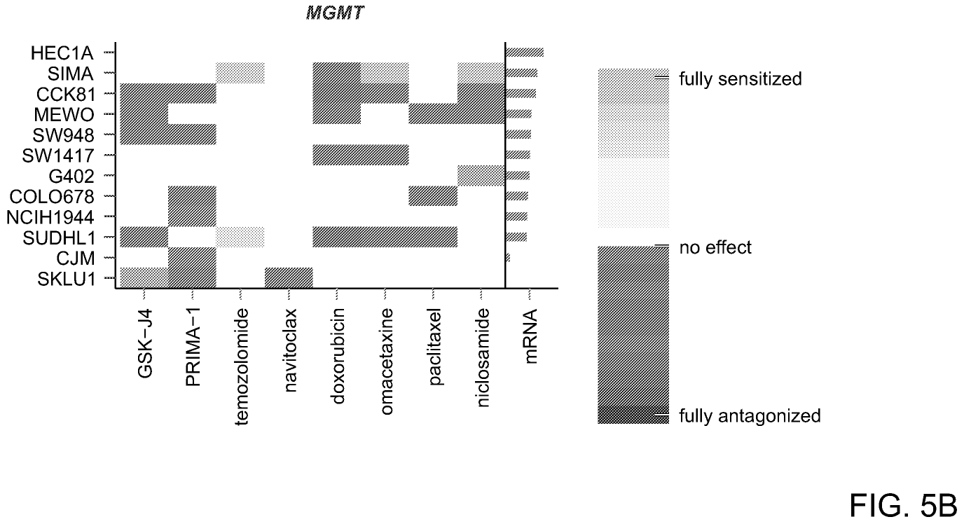

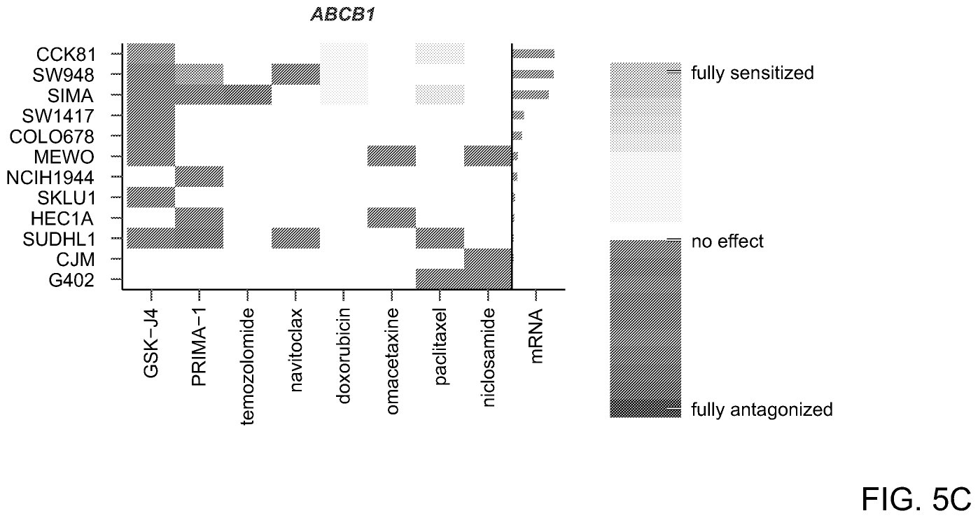

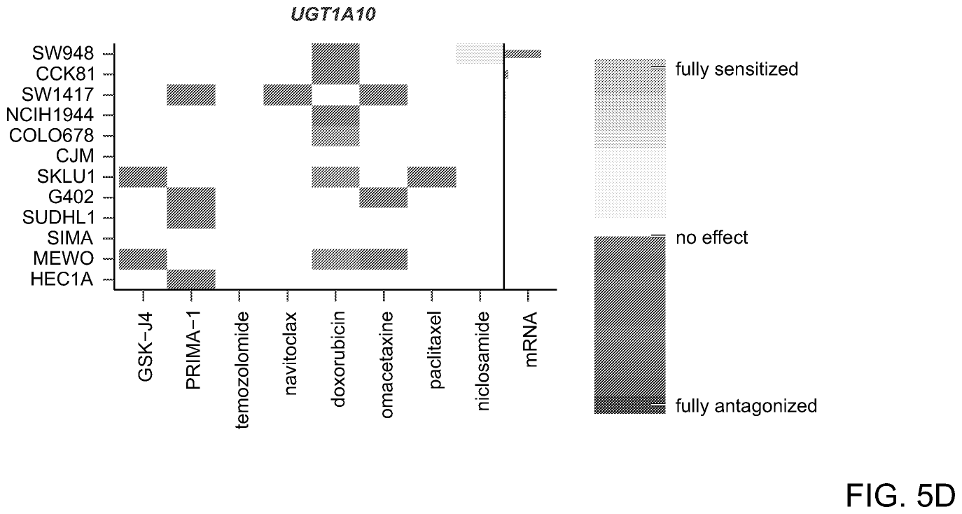

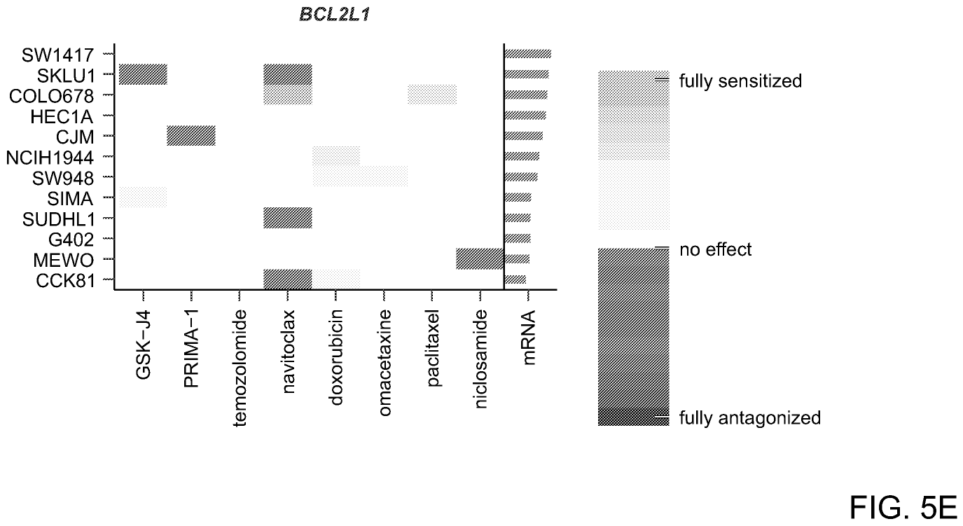

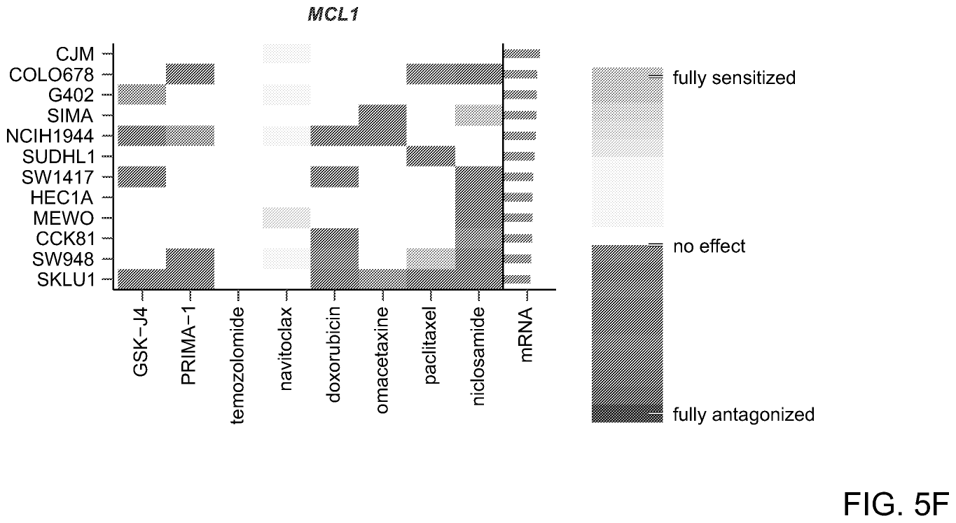

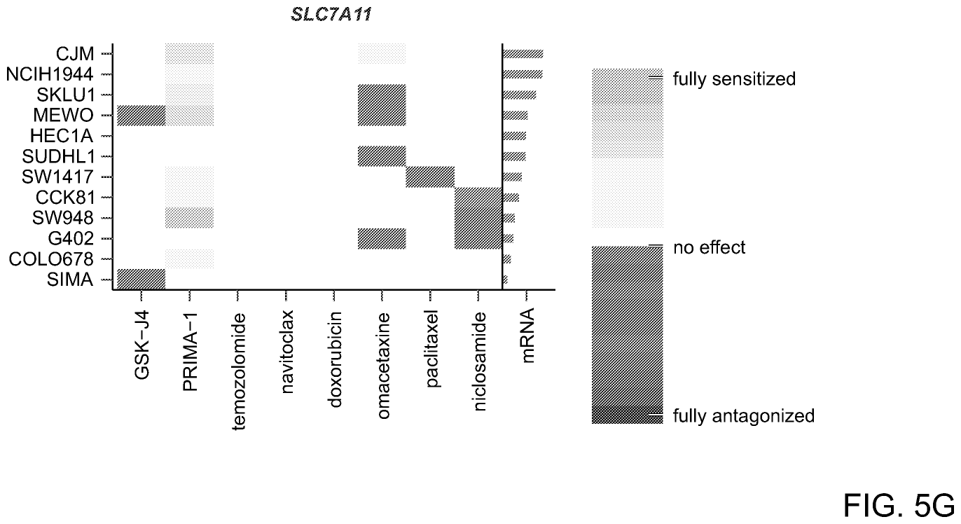

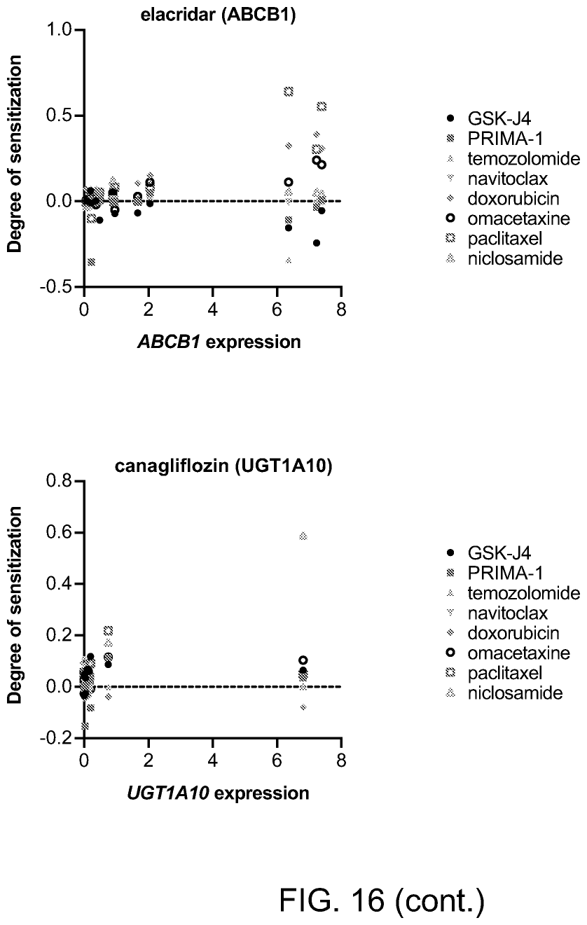

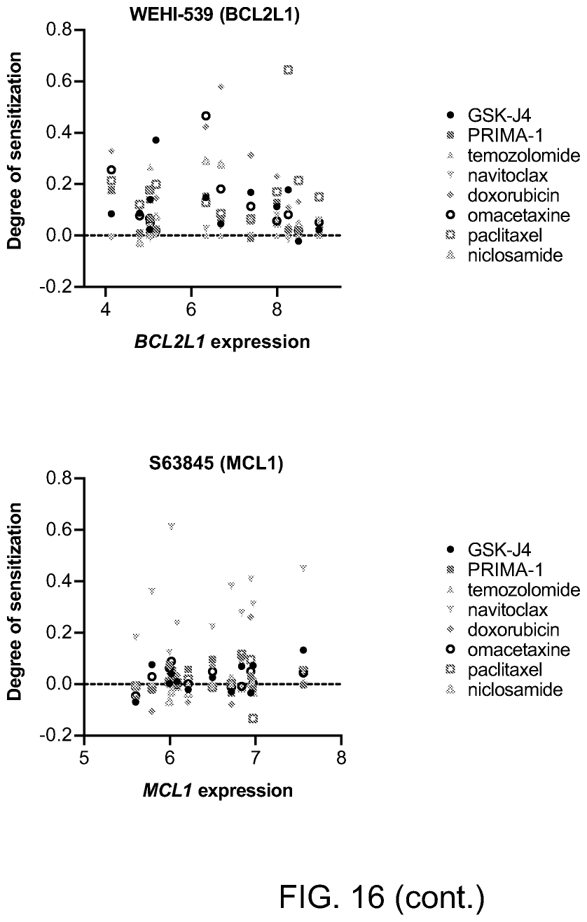

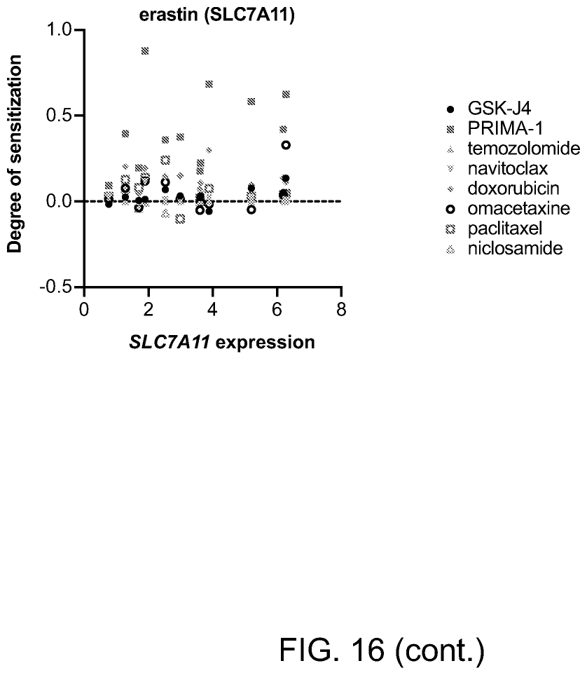

The generalizability across models and specificity of co-treatments were both tested by combining eight inhibitors of nominated co-targets with a full concentration-response of all index compounds, for a total of 56 drug combinations tested. Significantly, these experiments were conducted across a panel of 12 cell lines that represented a diversity of levels of target mRNA expression. The shift in response (change in GR₅₀) observed was calculated for each combination relative to single-agent response, and these responses were normalized across cell lines by the single-agent response identified in the most intrinsically sensitive and resistant cell line in the panel (FIGS. 5 and 16).

Of the nominated relationships tested, all showed sensitization (i.e., synergy) on combination treatment across multiple cell lines, as compared to control relationships that were not nominated by the approach. For all seven synergistic relationships, there was a clear association between the degree of sensitization and expression of the resistance-associated protein (FIGS. 5A to 5G and 16A to 16G). As expected, for four combinations (MGLL: GSK-J4+JZL184; MGMT: temozolomide+06-benzylguanine; ABCBJ: paclitaxel or YM-155+elacridar; UGT1A10: niclosamide+canagliflozin or emodin), no synergy was observed in endogenously deficient cell lines, and the degree of sensitization in proficient cell lines resulted in sensitivity at concentrations similar to that observed for sensitive, deficient cell lines (FIGS. 5A to 5D and 16A to 16D). No available cell lines were endogenously deficient in BCL2L1 or MCL1; nonetheless, there was a clear difference in the degree of sensitization between low- and high-expressing cell lines (FIGS. 5E, 5F, 16E, and 16F). However, for PRIMA-1, sensitization was observed on combination with the purported SLC7A11 inhibitor erastin even in cell lines lacking endogenous expression of SLC7A11 (FIGS. 5G and 16G). For 6/7 synergistic relationships tested, measurable synergy in all proficient cell lines was observed. However, no synergy was observed between temozolomide and 06-benzylguanine in all MGMT-proficient cell lines (FIGS. 5B and 16B).

### Example 9: Discovery of Drug/Gene Correlations Reveals a Diversity of Underlying Biology

After validation of the relationships between indexed compounds and their associated synergistic targets, the biology associated with these relationships was examined in detail. Within the prioritized list of correlated drug/gene pairs, a number of relationships that were highly consistent with the underlying, well-appreciated biology of both the indexed drug and the resistance-associated transcript were identified.

For example, the relationship between the alkylating compound temozolomide and the methyltransferase MGMT is quite clear; MGMT resolved the DNA damage caused by temozolomide, effectively antagonizing its mechanism of action. Other classes of relationships were also noted. The relationship between the primary target of navitoclax, BCL2, and its associated resistance gene MCL1 is likely explained by functional redundancy between these two family members, as is resistance to AKT1/AKT2 inhibitors to expression of AKT3. Similarly, drug/gene pairs that could be explained by pathway redundancies were uncovered, including CDK4/6 inhibition and cyclin E1 (CCNE1) and PIK3CA and IRS2. In addition, a number of relationships between seemingly unrelated drugs and efflux pumps were noted, including ABCB1 (paclitaxel), ABCC1 (BRD1378), ABCG2 (ZG-10), and UGT1A10 (niclosamide). However, for a number of drug/gene pairs, the biology or mechanisms underlying the relationship remained unclear even after this deeper assessment, including GSK-J4 (MGLL), ML210 (AIFM2), and PRIMAL (SLC7A11). Among these, the relationship between GSK-J4 and MGLL was particularly intriguing, as the relationship was quite robust and both targets had well-credentialed biology associated with them that had heretofore shown no obvious connection. Accordingly, this relationship, and the mechanism underlying it, was examined in greater detail.

### Example 10: Broad Validation of MGLL Inhibition and GSK-J4 as a Synergistic Drug Combination

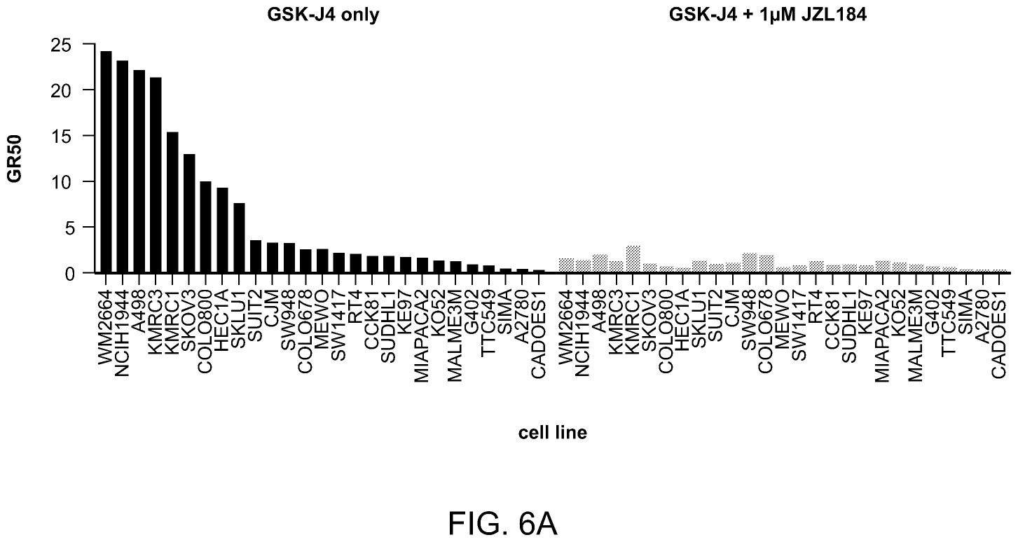

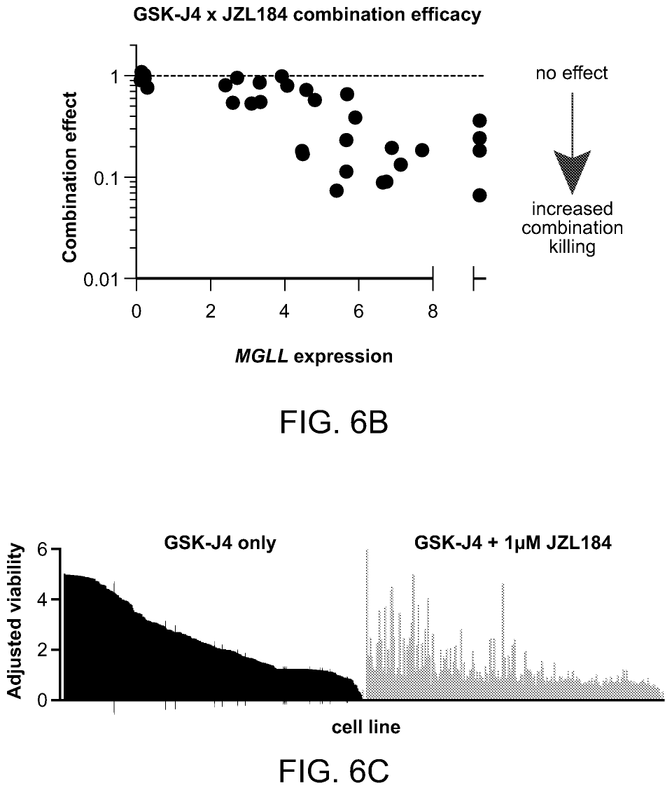

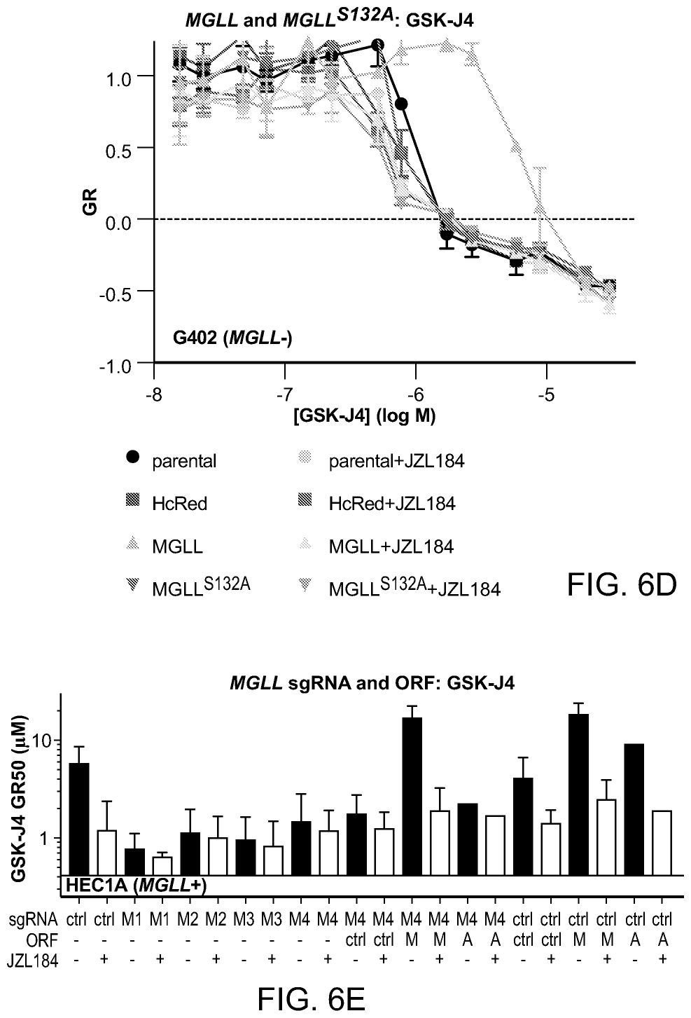

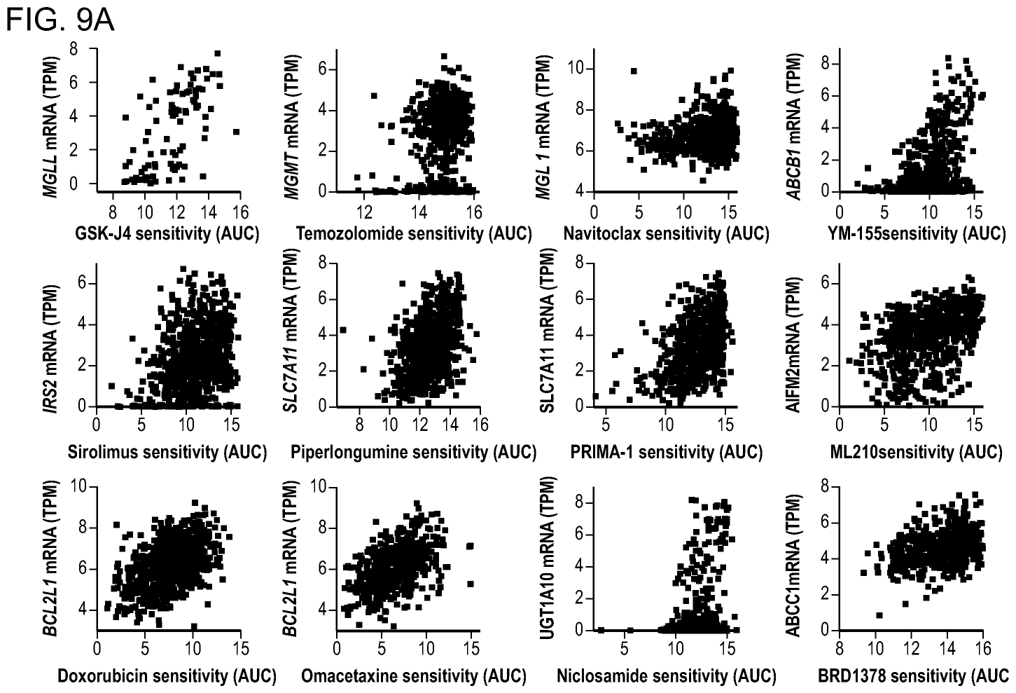

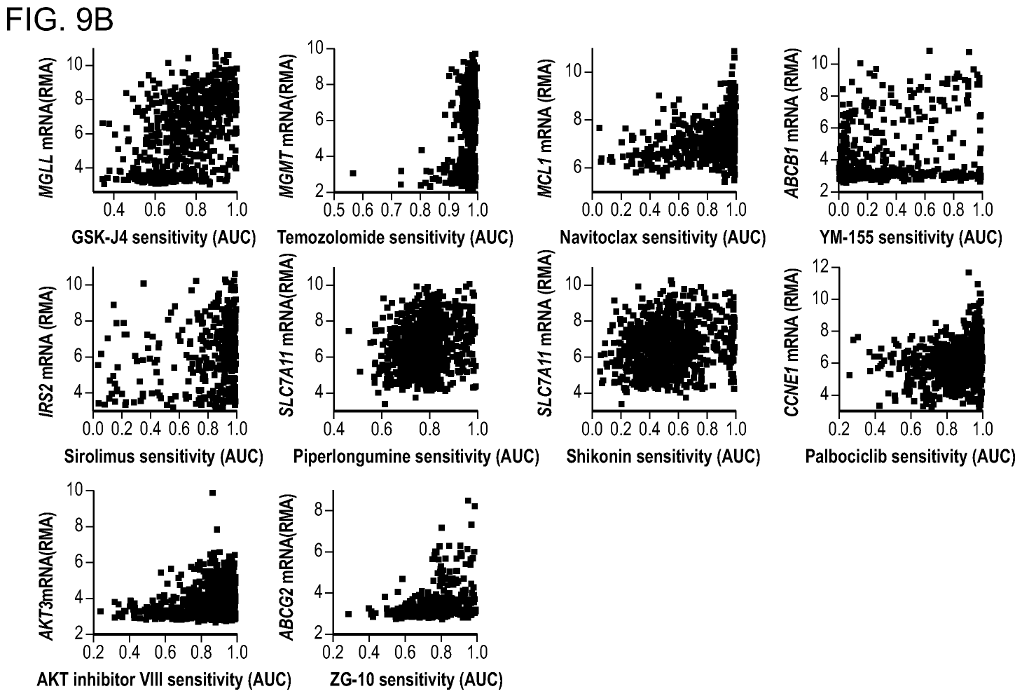

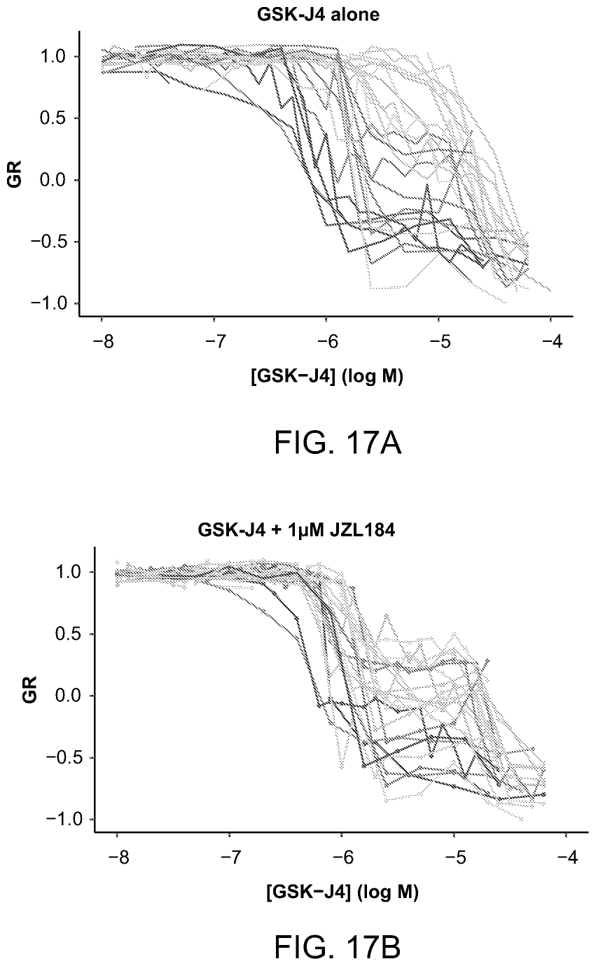

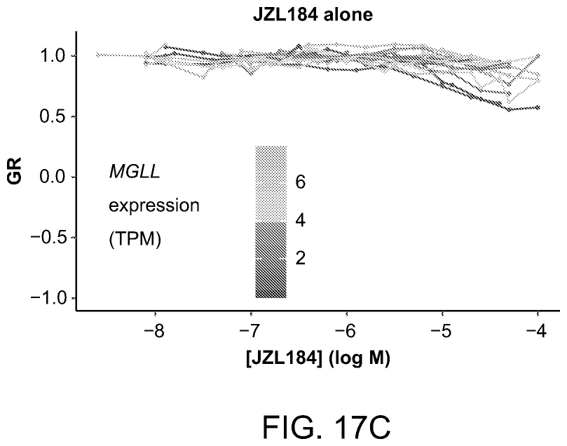

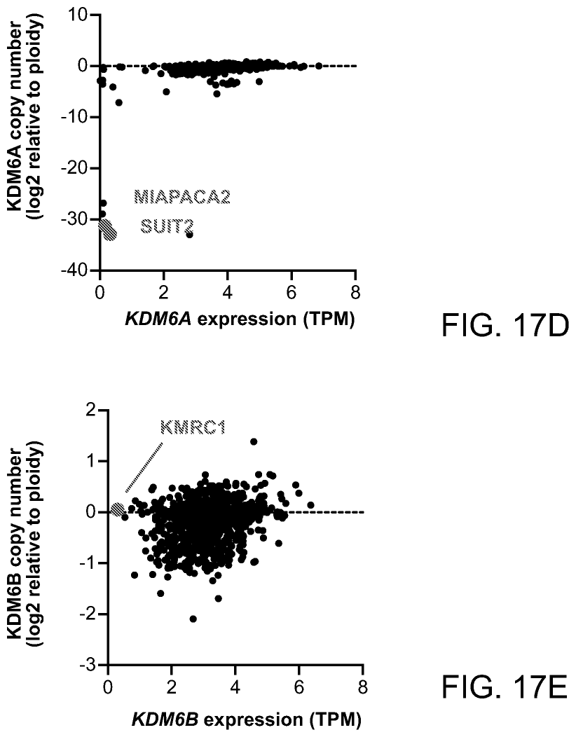

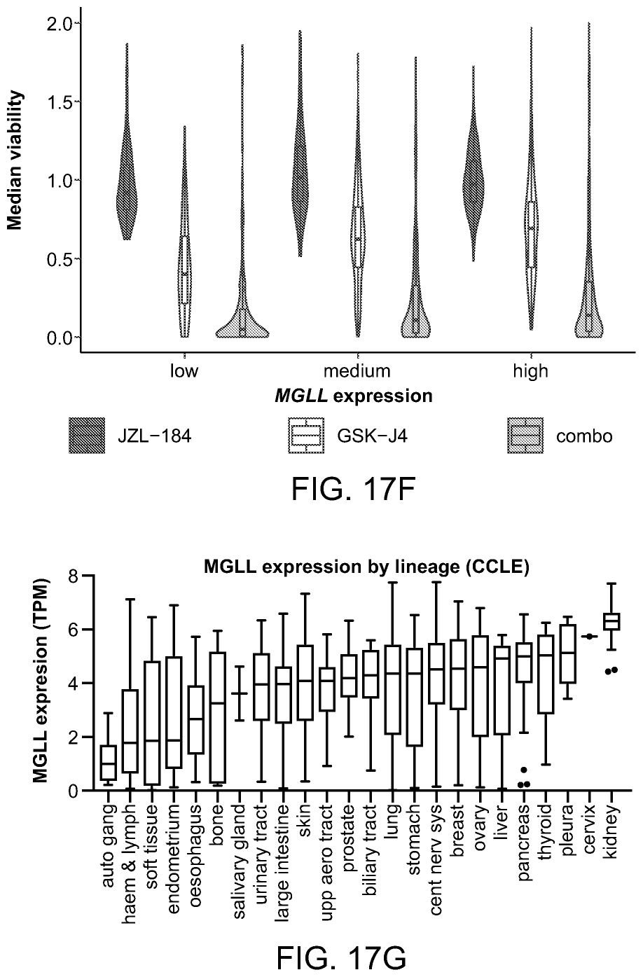

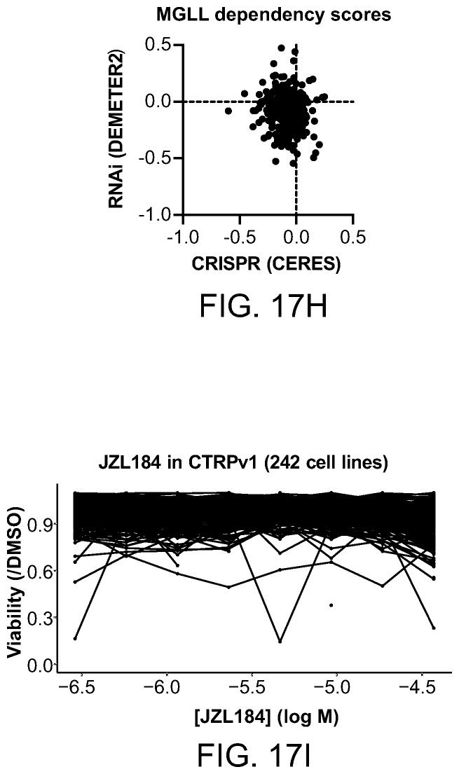

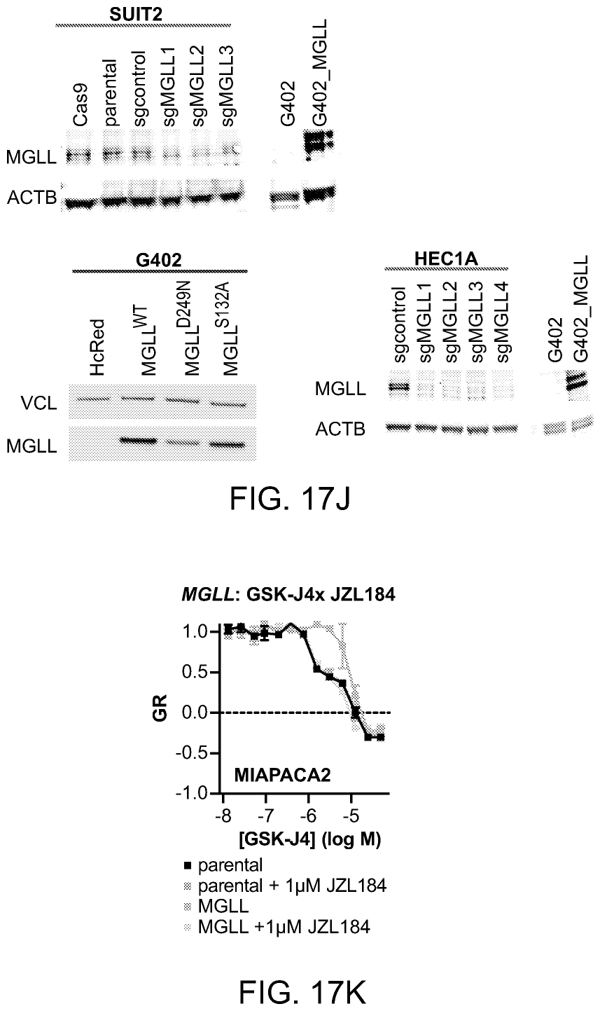

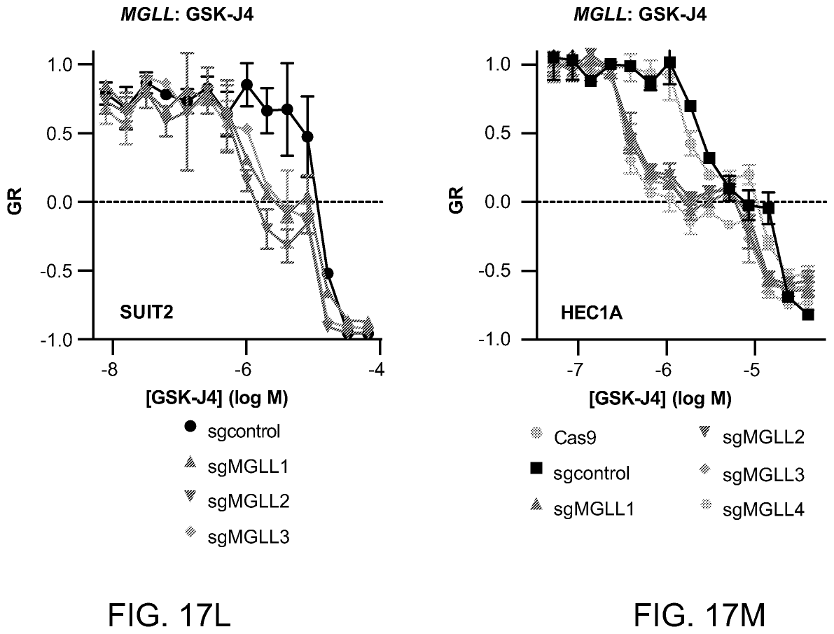

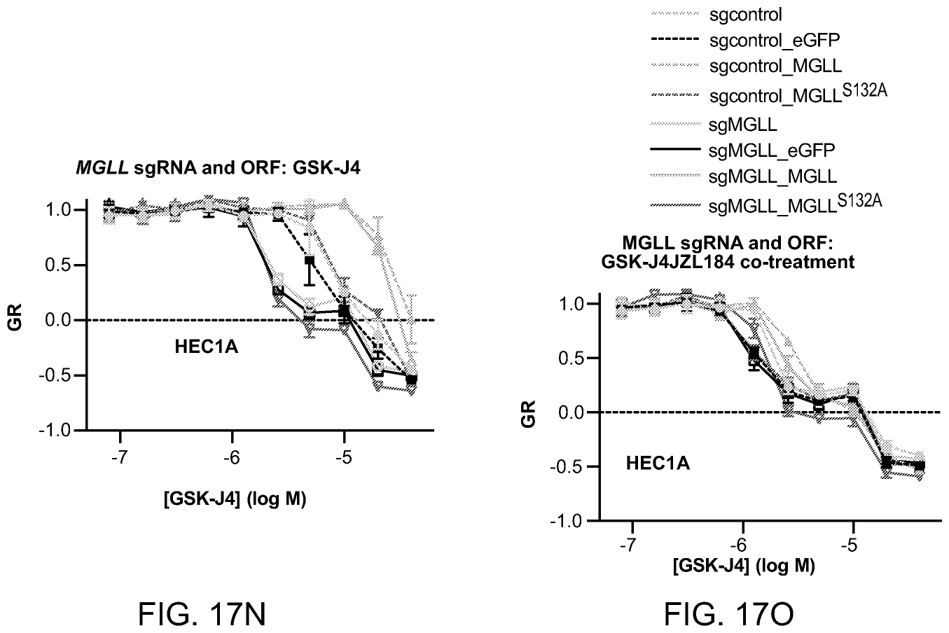

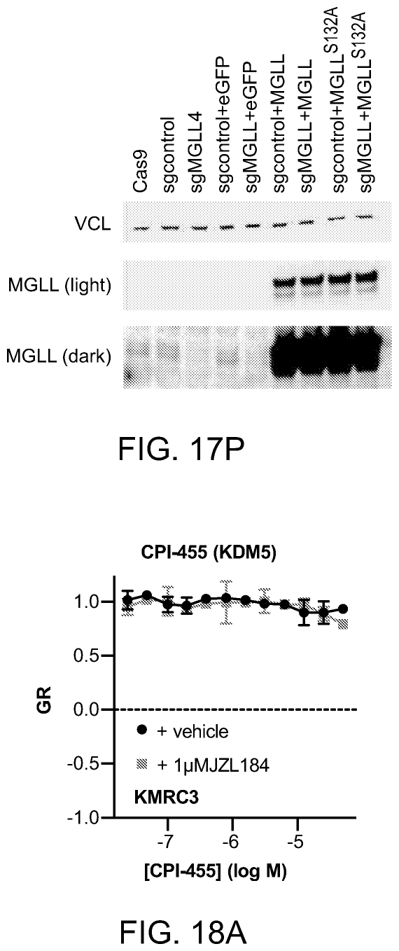

The following was undertaken with the goal of understanding the biological underpinnings of the relationship between GSK-J4, an annotated inhibitor of histone lysine demethylases (in particular, KDM6A/B), and MGLL, which encodes the serine hydrolase monoglyceride lipase, that converts monoglycerides such as 2-arachidonylglycerol to arachidonic acid in endocannabinoid signaling. Supporting the robustness of this relationship, a recent study had reported the relative toxicity of GSK-J4 across 638 cell lines (23). MGLL mRNA expression again emerges as the top predictive feature when combining this study with GDSC expression data (FIGS. 2A, 2B, and 9B). Overall, the effects of MGLL inhibition by JZL184 on GSK-J4 sensitivity in a total of 27 cell lines (FIGS. 6A, 6B, and 17A to 17C) were validated. In all cell lines tested for which MGLL protein was detectable, sensitization of GSK-J4 was observed on co-treatment with JZL184 (FIGS. 6A, 6B, and 17A to 17C). This included cell lines annotated as deficient in one or the other of the targets of GSK-JA 4 (KDM6A: SUIT2, MIAPACA2; KDM6B: KMRC1) (FIGS. 17D and 17E). The combination of GSK-J4 and JZL184 was tested in >500 cell lines using PRISM, a high-throughput pooled-screening assay. In the vast majority (93.5%), it was observed that the cell lines were more sensitive to the combination than to GSK-J4 alone, an effect that was dependent upon their level of MGLL expression (FIGS. 6C and 17F) (5).

### Example 11: Confirmation that MGLL is Not a Cancer-Cell-Line Dependency

Expression of MGLL varied within and across cancer cell-line types, with particularly high expression observed in pancreatic and renal cell lines and low expression observed in neuroblastoma cell lines (FIG. 17G). As noted above, to explore the role of MGLL across cancer cell lines, public shRNA and CRISPR gene dependency data in the DepMap portal were queried, and no evidence that any cell lines were strongly dependent on MGLL was identified (FIG. 17H). Consistent with this absence of evidence, JZL184 did not demonstrate dose-dependent toxicity across 242 cell lines in CTRPv1 and exhibited very modest effects in individual validation experiments or the pooled PRISM assay (FIGS. 17C, 17F, and 17I), which indicated that genetic or pharmacological suppression of MGLL alone is largely inert across cancer cell-line models (24).

### Example 12: MGLL Activity and Expression is Sufficient to Confer Resistance to GSK-J4

To better understand whether the catalytic activity of MGLL was required for effects on GSK-J4 sensitivity, an enzyme-dead mutant (MGLL^{S132A}) was overexpressed in G402 cells. It was observed that MGLL^{S132A} overexpressing G402 cells were equally sensitive to GSK-J4 as parental or control-infected cells, while MGLL^{WT} again conferred significant resistance (FIGS. 6D and 17J). Additionally, it was observed that sensitivity could be restored to MGLL^{WT}-overexpressing cells with JZL184, but JZL184 had no effect on parental, control-infected, or MGLL^{S132A} expressing cells (FIG. 6D), which confirmed that MGLL enzymatic activity is necessary for driving resistance to GSK-J4.

### Example 13: MGLL Activity and Expression is Necessary for Resistance to GSK-J4

Finally, whether knockout of MGLL could phenocopy co-treatment with the MGLL inhibitor JZL184 was evaluated (FIGS. 6E and 17J to 17P). Knockout of MGLL with 4 different sgRNAs was observed to sensitize HEC1A and SUIT2 cells to GSK-J4, and, for guides that achieved near-complete knockout, JZL184 co-treatment did not further sensitize cells to GSK-J4 (FIGS. 6E and 17J to 17M). Further, MGLL knockout in HEC1A cells was rescued by overexpression of MGLL when using an sgRNA that targeted MGLL across an intron-exon boundary and that therefore could not cut the MGLL ORF. This overexpression could again be re-sensitized to GSK-J4 by treatment with the JZL184 (FIGS. 6E and 17N to 17P), which indicated that MGLL inhibition, whether pharmacological or by protein depletion or genetic ablation of enzymatic activity, was sufficient to restore sensitivity to GSK-J4.

### Example 14: MGLL Inhibition Synergizes with GSK-J4, but not with Other Epigenetic Modulatory Compounds

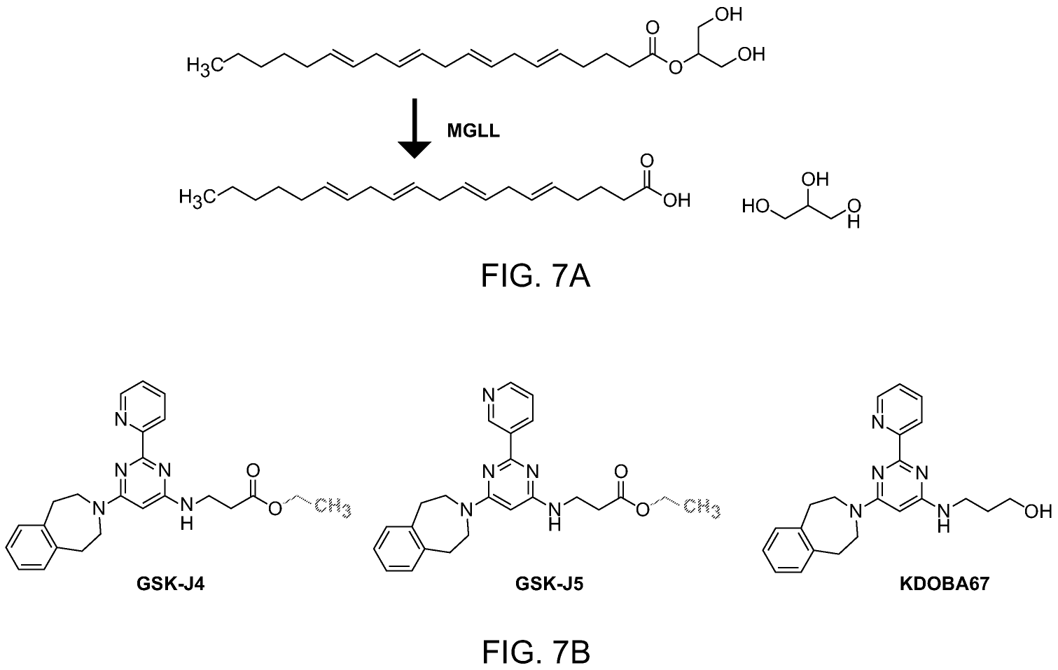

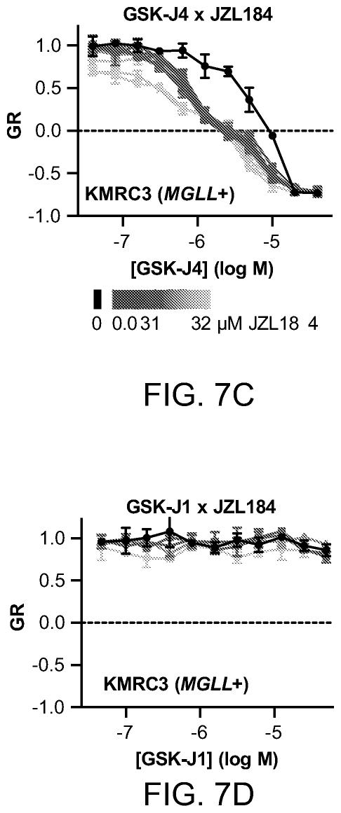

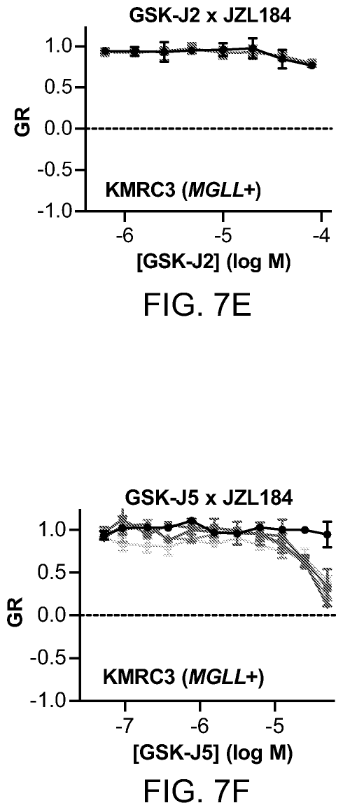

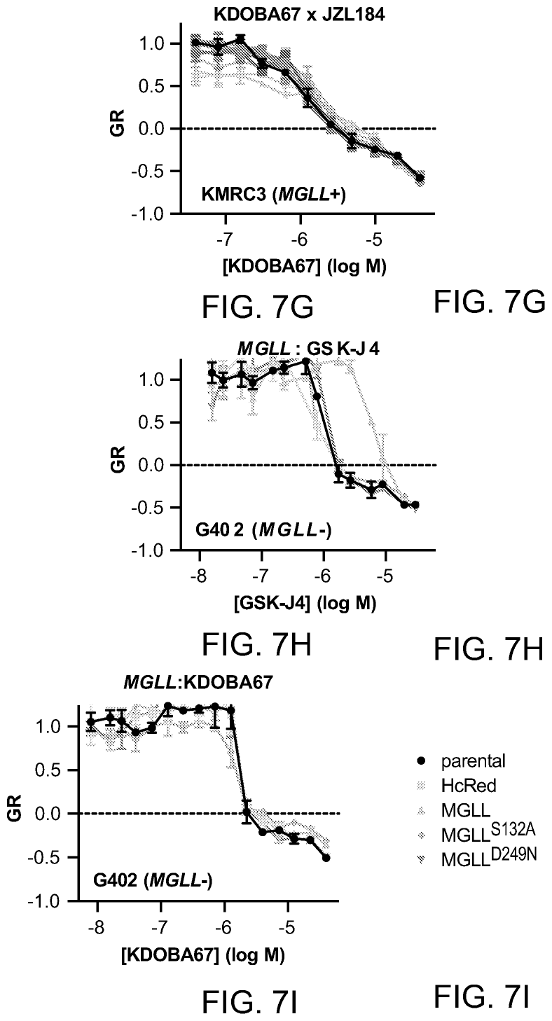

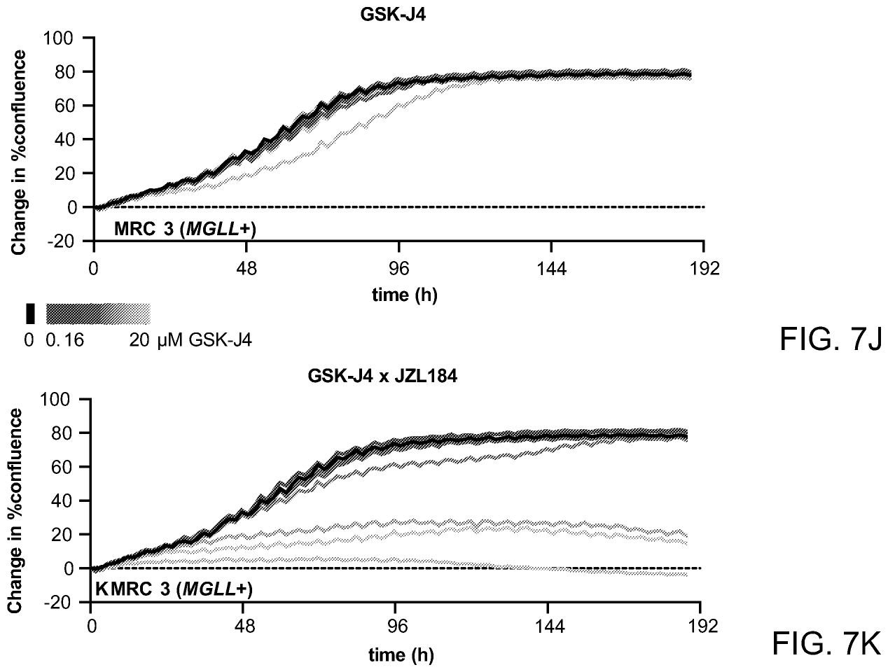

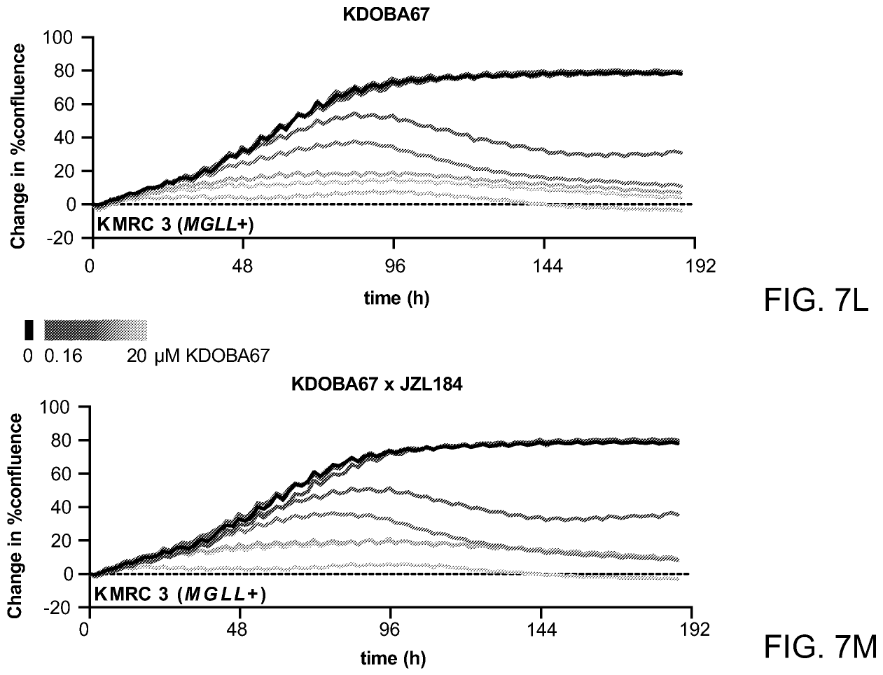

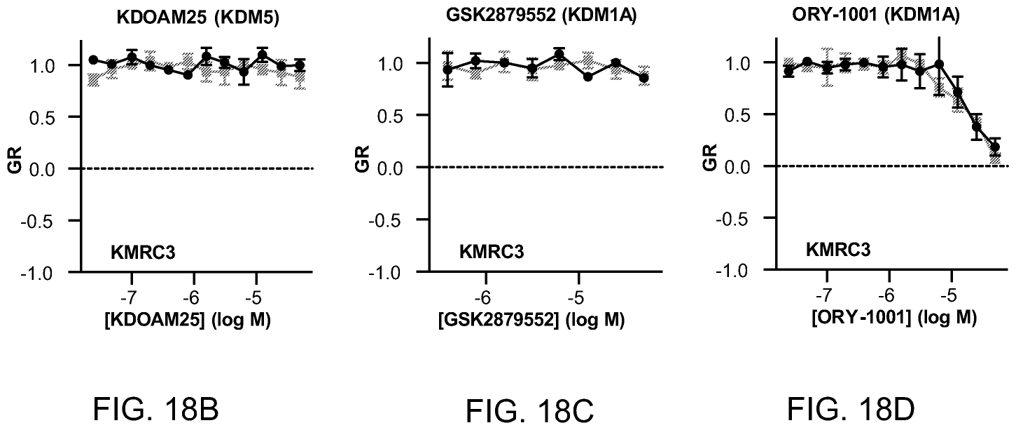

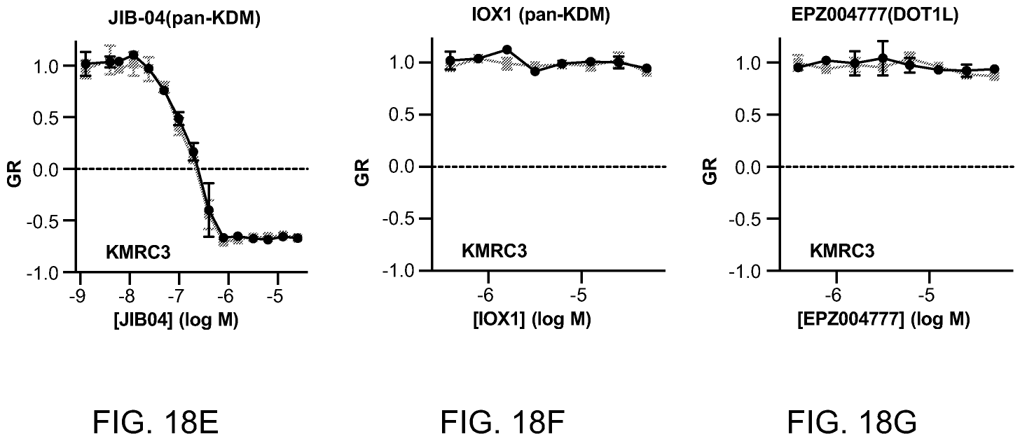

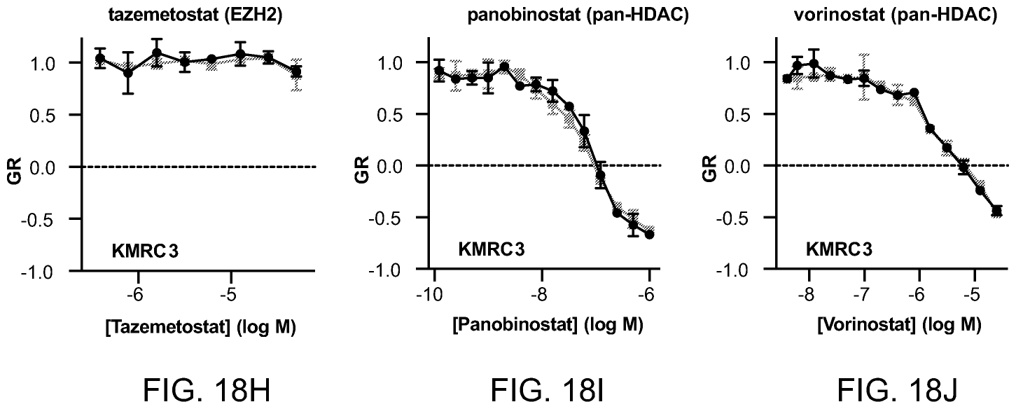

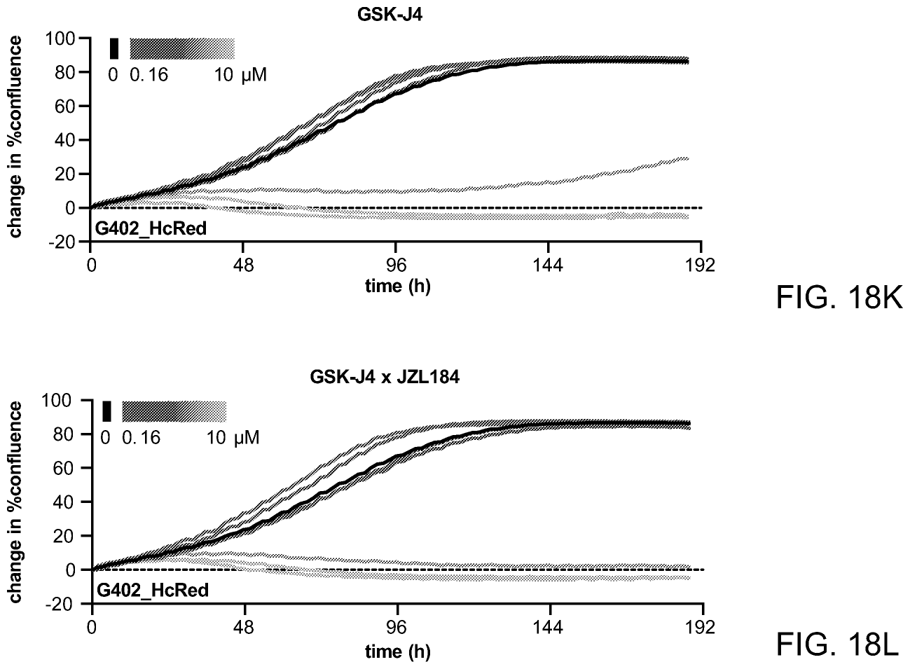

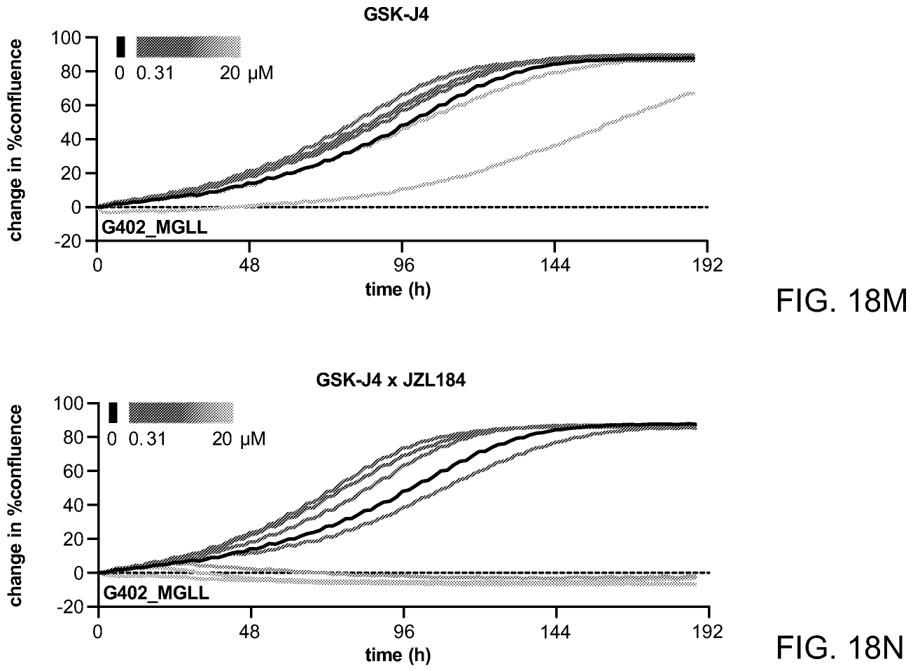

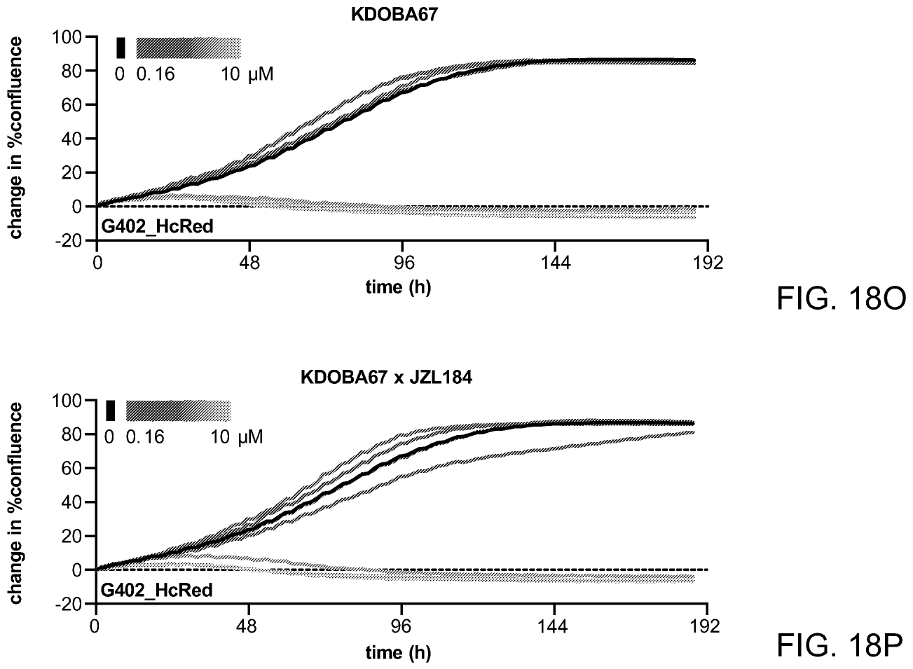

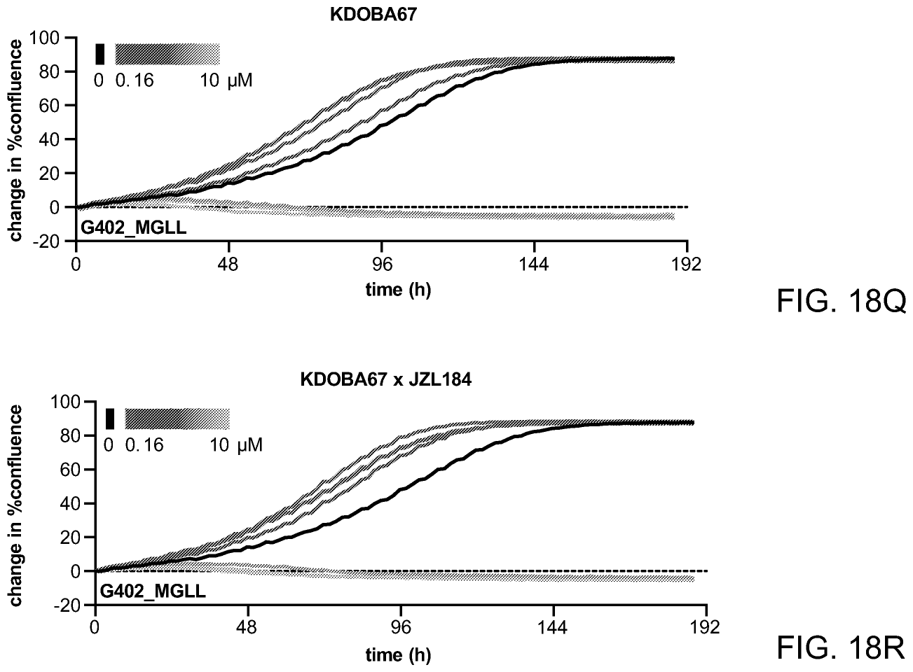

To assess whether MGLL could augment sensitivity to other inhibitors of chromatin modifying enzymes, a panel of other small molecules annotated to inhibit chromatin-related processes, targeting other KDM proteins, histone deacetylases (HDACs), and histone methyltransferases (e.g., EZH2, DOT1L) was tested. None of these compounds exhibited differential activity in G402 versus G402-MGLL cells, nor did any show synergy with JZL184 in G402-MGLL cells or KMRC3 cells which express high endogenous levels of MGLL (FIGS. 7A and 18A to 18J). The effects of a number of GSK-J4 analogues (FIG. 7B) were also tested. GSK-J4 is an ethyl ester with high cellular permeability that is ostensibly acted upon by cellular esterases to generate GSK-J1, the bioactive, but cell-impermeable, inhibitor of KDM6 family members (25). GSK-J1 only showed toxicity at high concentrations (>20 μM), but this toxicity was not affected by MGLL overexpression or JZL184 co-treatment (FIGS. 7C and 7D). GSK-J2, an isomer of GSK-J1 that is inactive against KDM6 proteins, showed minimal toxicity (<30% at 80 μM) that was again not affected by MGLL overexpression or inhibition (FIG. 7E). However, as expected in view of the above results, GSK-J5, the ethyl ester of GSK-J2, was sensitized by JZL184 and showed reduced toxicity on MGLL overexpression, although it was much less potent than GSK-J4 (FIG. 7F).

### Example 15: Sensitivity to KDOBA67, a GSK-J4 Analogue, was Unaffected by MGLL

KDOBA67, a recently-described cell-permeable GSK-J1 derivative that does not require esterase activation and has similar on-target KDM6 activity, showed equivalent toxicity to GSK-J4 in MGLL-deficient cell lines, but was more potent in resistant lines that expressed MGLL either endogenously or ectopically (FIGS. 7C and 7G to 7I) (26, 27). Additionally, cells that expressed MGLL either endogenously or ectopically could not be further sensitized to KDOBA67 by JZL184. Using the IncuCyte live-cell microscopy system, temporal effects were observed on cellular proliferation on treatment with KDOBA67, GSK-J4 or GSK-J4+JZL184 as early as 24 hours in KMRC3 cells (FIGS. 7J to 7M and 18K to 18R). In summary, the behavior of KDOBA67 was functionally similar to the combination of GSK-J4+JZL184.

### Example 16: GSK-J4, but not KDOBA67, Resembles an MGLL Substrate

In view of the above observation that MGLL was unable to mediate resistance to KDOBA67, the structures of respective GSK-J4 analogues were examined more closely. Such comparison revealed that J4, but not KDOBA67, resembled an MGLL substrate. Accordingly, without wishing to be bound by theory, it is likely that MGLL is capable of modifying J4, but not KDOBA67, thereby reducing J4 function but not KDOBA67 function.

### Example 17: MGLL Modifies GSK-J4, but not KDOBA67

The GSK-J4-modifying impact of MGLL is examined further via a recombinant protein/mass spec assay. MGLL-mediated modification of GSK-J4 is thereby confirmed, while no modification of KDOBA67 is expected.

The expansion of public cancer cell-line datasets with robust genomic characterization and small-molecule sensitivity measurements has been leveraged herein to generate a comprehensive list of potential resistance-associated transcripts. The focus of the instant disclosure has been upon small-molecule/transcript relationships where cell lines deficient in a transcript (or exhibiting low relative expression levels) were identified as sensitive to a given compound relative to those exhibiting high expression, while accounting for a number of potential confounding features including specificity across compounds and gene co-expression.

From this list of potential “co-targets”, a set of known relationships or those with clear biological connections were selected to validate the approach. The latter (those with clear biological connections) was also investigated for a number of novel connections. The causal necessity for these relationships was tested by using ectopic overexpression to confer resistance in deficient cells, and their sufficiency was explored by inhibiting and/or knocking out the nominated co-target in proficient cells. Importantly, by leveraging data across many cellular models, the degree of resistance (and sensitization) conferred by comparison with intrinsically sensitive (or resistant) cell lines (FIG. 5) could be benchmarked. This enabled relative quantitation of the degree of synergy and resistance observed; for several of the relationships tested, the presence (or absence) of the co-target was observed to render the most resistant cell line as responsive as the most sensitive (and vice versa). Additionally, specificity was tested by measuring effects of these perturbations across a series of additional compounds that either also shared or did not show a strong predicted co-target relationship.

As a consequence of focusing on a number of drug combination partners that are inert as single agents, the combinations investigated herein were deeply synergistic. The potential for synergistic relationships to be translated clinically has recently been called into question (28, 29), and at least a subset of drug combinations have been suggested to achieve cure by virtue of combining mechanistically distinct agents that do not share overlapping resistance mechanisms (28). Accordingly, understanding the mechanisms of resistance to individual therapies (and groups of therapies), as was performed here, may be of utility in identifying particular regimens that are effective in combating resistance (29). It is also contemplated that the instant approach can be employed to identify drug combinations that optimize both synergy and non-overlapping resistance mechanisms, thereby harnessing such combinations to achieve maximal long-term efficacy.

The instant approach can therefore be applied and extended in a number of ways. First, it can be used to identify potential resistance mechanisms that may contribute to relapse in initially responsive settings. Second, it can be used to prioritize combinations for increased initial efficacy if the co-target biomarker is already present, or to de-prioritize single-agent treatment where it is likely to be less efficacious, particularly where alternatives might exist. For example, drug efflux pump expression (e.g., of ABCB1, encoding P-glycoprotein) has been observed as a clinical mechanism of resistance to various chemotherapeutics (30). ABCB1 inhibitors were discontinued from clinical development, largely due to challenges with selectivity and on-target toxicity (31). However, the approach disclosed herein is capable of identifying not only compounds for which ABCB1 is a potential mechanism of resistance, but also mechanistically-related compounds that do not have this liability. For example, both paclitaxel and the clinical candidate KX2-391 have been described to inhibit microtubule polymerization and possess broadly similar toxicity profiles across cancer cell lines (as evidenced by their co-clustering) (13). However, it was herein observed that KX2-391 response was not correlated with ABCB1 expression or affected by the presence of active ABCB1 protein (FIG. 14E). Thus, in situations where ABCB1 expression limits therapeutic efficacy, KX2-391, or similar molecules, could provide a more effective alternative. Third, data generated herein indicated that combinations can achieve synergy by augmenting or impacting overlapping biology. For example, via antagonizing mechanism of action (MGLL, MGMT), redundant family members (MCL1), or redundant pathway members (IRS2, CCNE1, AKT3); perturbing downstream consequences (BCL2L1); or pumping out the drug (ABC family members). Thus, it is conceivable that one could discover the mechanism or pathways of drug action by systematically identifying drug/gene and drug/drug interactions, as has been disclosed and performed herein.

Despite the selective focus herein on examples that exhibit a specific directionality (high expression associated with resistance), it has also been observed herein that the general approach of the instant disclosure appears equally capable of identifying transcripts associated with the opposite directionality, whereby inhibition (or knockdown) of the co-target would be expected to confer resistance (or, conversely, where overexpression or activation may cause sensitization). A number of validated examples of this directionality have been described, such as for the compounds YM-155 (biomarker: SLC35F2), DNDMP (PDE3A), ML239 (FADS2), or birinapant (TNF) (12, 32-34). The instant general approach, as disclosed herein, may therefore more broadly represent a means to identify non-neutral (strongly synergistic or antagonistic) combinations.

Overall, the approach listed in the instant disclosure was successful in identifying several novel resistance-associated mechanisms, the relative impact of which was supported by near-genome-scale ORFeome screening for PRIMA-1, ML210, and GSK-J4 (FIG. 3). Perhaps the most unexpected relationship identified was that observed herein between MGLL and GSK-J4, as no obvious biological connection has been previously described between histone lysine methylation and monoglyceride metabolism. Consistent with previous studies demonstrating rapid effects of GSK-J4 on transcriptomic- and protein-based cellular stress responses, effects of GSK-J4 on cell proliferation were observed within 12 hours (23, 26, 27), and it was observed that these effects could be durably rescued by MGLL (FIGS. 7J, 7K, and 18K to 18N). Noting that a subset of pediatric cancers, including diffuse intrinsic pontine glioma and neuroblastoma, have been identified as indications for GSK-J4, the instant findings are expected to help inform initial responses in these diseases (23, 35, 36). Published GSK-J4 cellular data have also observed a rapid (within 6 hours) induction of ATF4/metallothionein/ER stress. Further exploration of these and other newly identified relationships is expected to enhance the understanding of drug resistance mechanisms and provide insight into potentially effective single-agent and combination treatments for disease.

### Example 18: Application of Newly Identified Combination Therapies

A mammalian subject having cancer—optionally a subject already having received a KDM6A/KDM6B inhibitor such as GSK-J4—is administered a MGLL inhibitory agent at a sufficient dose to render the cancer of the subject sensitive to GSK-J4, thereby effectively treating the cancer in the subject. Optionally, GSK-J4 and the MGLL inhibitory agent (e.g., JZL 184) is co-administered to the subject, or is administered with dosage timing sufficiently contemporaneous to achieve an anti-cancer result in the mammalian subject. A parallel approach can be used for the other combination therapies newly identified herein.

### REFERENCES

1. Konieczkowski, D. J., C. M. Johannessen, and L. A. Garraway, A Convergence-Based Framework for Cancer Drug Resistance. Cancer Cell, 2018. 33(5): p. 801-815.
2. Chapman, P. B., et al., Improved survival with vemurafenib in melanoma with BRAF V600E mutation. N Engl J Med, 2011. 364(26): p. 2507-16.
3. Robert, C., et al., Improved overall survival in melanoma with combined dabrafenib and trametinib. N Engl J Med, 2015. 372(1): p. 30-9.
4. Konieczkowski, D. J., et al., A melanoma cell state distinction influences sensitivity to MAPK pathway inhibitors. Cancer Discov, 2014. 4(7): p. 816-27.
5. Yu, C., et al., High-throughput identification of genotype-specific cancer vulnerabilities in mixtures of barcoded tumor cell lines. Nat Biotechnol, 2016. 34(4): p. 419-23.
6. Muellner, M. K., et al., A chemical-genetic screen reveals a mechanism of resistance to PI3K inhibitors in cancer. Nat Chem Biol, 2011. 7(11): p. 787-93.
7. Lehár, J., et al., Synergistic drug combinations tend to improve therapeutically relevant selectivity. Nat Biotechnol, 2009. 27(7): p. 659-66.
8. Mathews Griner, L. A., et al., High-throughput combinatorial screening identifies drugs that cooperate with ibrutinib to kill activated B-cell-like diffuse large B-cell lymphoma cells. Proc Natl Acad Sci USA, 2014. 111(6): p. 2349-54.
9. Greco, W. R., G. Bravo, and J. C. Parsons, The search for synergy: a critical review from a response surface perspective. Pharmacol Rev, 1995. 47(2): p. 331-85.
10. Foucquier, J. and M. Guedj, Analysis of drug combinations: current methodological landscape. Pharmacol Res Perspect, 2015. 3(3): p. e00149.
11. Iorio, F., et al., A Landscape of Pharmacogenomic Interactions in Cancer. Cell, 2016. 166(3): p. 740-754.
12. Rees, M. G., et al., Correlating chemical sensitivity and basal gene expression reveals mechanism of action. Nat Chem Biol, 2016. 12(2): p. 109-16.
13. Seashore-Ludlow, B., et al., Harnessing Connectivity in a Large-Scale Small-Molecule Sensitivity Dataset. Cancer Discov, 2015. 5(11): p. 1210-23.
14. Barretina, J., et al., The Cancer Cell Line Encyclopedia enables predictive modelling of anticancer drug sensitivity. Nature, 2012. 483(7391): p. 603-7.
15. Hegi, M. E., et al., MGMT gene silencing and benefit from temozolomide in glioblastoma. N Engl J Med, 2005. 352(10): p. 997-1003.
16. Tahir, S. K., et al., Identification of expression signatures predictive of sensitivity to the Bcl-2 family member inhibitor ABT-263 in small cell lung carcinoma and leukemia/lymphoma cell lines. Mol Cancer Ther, 2010. 9(3): p. 545-57.
17. Tsherniak, A., et al., Defining a Cancer Dependency Map. Cell, 2017. 170(3): p. 564-576.e16.
18. Hafner, M., et al., Growth rate inhibition metrics correct for confounders in measuring sensitivity to cancer drugs. Nat Methods, 2016. 13(6): p. 521-7.
19. Piccioni, F., S. T. Younger, and D. E. Root, Pooled Lentiviral-Delivery Genetic Screens. Curr Protoc Mol Biol, 2018. 121: p. 32.1.1-32.1.21.
20. Conrad, M. and H. Sato, The oxidative stress-inducible cystine/glutamate antiporter, system x (c) (−): cystine supplier and beyond. Amino Acids, 2012. 42(1): p. 231-46.
21. Ramirez, J., et al., Glucuronidation of OTS167 in Humans Is Catalyzed by UDP-Glucuronosyltransferases UGT1A1, UGT1A3, UGT1A8, and UGT1A10. Drug Metab Dispos, 2015. 43(7): p. 928-35.
22. Pattanawongsa, A., et al., Inhibition of Human UDP-Glucuronosyltransferase Enzymes by Canagliflozin and Dapagliflozin: Implications for Drug-Drug Interactions. Drug Metab Dispos, 2015. 43(10): p. 1468-76.
23. Lochmann, T. L., et al., Targeted inhibition of histone H3K27 demethylation is effective in high-risk neuroblastoma. Sci Transl Med, 2018. 10(441).
24. Basu, A., et al., An interactive resource to identify cancer genetic and lineage dependencies targeted by small molecules. Cell, 2013. 154(5): p. 1151-61.
25. Heinemann, B., et al., Inhibition of demethylases by GSK-J1/J4. Nature, 2014. 514(7520): p. E1-2.
26. Cottone, L., et al., Epigenetic inactivation of oncogenic brachyury (TBXT) by H3K27 histone demethylase controls chordoma cell survival. bioRxiv, 2018: p. 432005.
27. Hookway, E. S., The Role of the Lysine Demethylases KDM5 and KDM6 in Bone Malignancies, in Nuffield Department of Orthopaedics, Rheumatology and Musculoskeletal Sciences. 2016, University of Oxford. p. 245.
28. Palmer, A. C. and P. K. Sorger, Combination Cancer Therapy Can Confer Benefit via Patient-to-Patient Variability without Drug Additivity or Synergy. Cell, 2017. 171(7): p. 1678-1691.e13.
29. Menden, M. P., et al., A cancer pharmacogenomic screen powering crowd-sourced advancement of drug combination prediction. bioRxiv, 2018: p. 200451.
30. Patch, A. M., et al., Whole-genome characterization of chemoresistant ovarian cancer. Nature, 2015. 521(7553): p. 489-94.
31. Crowley, E., C. A. McDevitt, and R. Callaghan, Generating inhibitors of P-glycoprotein: where to, now? Methods Mol Biol, 2010. 596: p. 405-32.
32. Krepler, C., et al., The novel SMAC mimetic birinapant exhibits potent activity against human melanoma cells. Clin Cancer Res, 2013. 19(7): p. 1784-94.
33. de Waal, L., et al., Identification of cancer-cytotoxic modulators of PDE3A by predictive chemogenomics. Nat Chem Biol, 2016. 12(2): p. 102-8.
34. Winter, G. E., et al., The solute carrier SLC35F2 enables YM155-mediated DNA damage toxicity. Nat Chem Biol, 2014.
35. Grasso, C. S., et al., Functionally defined therapeutic targets in diffuse intrinsic pontine glioma. Nat Med, 2015. 21(6): p. 555-9.
36. Hashizume, R., et al., Pharmacologic inhibition of histone demethylation as a therapy for pediatric brainstem glioma. Nat Med, 2014. 20(12): p. 1394-6.
37. Durinck, S., et al., Mapping identifiers for the integration of genomic datasets with the R/Bioconductor package biomaRt. Nat Protoc, 2009. 4(8): p. 1184-91.
38. Kim, S., ppcor: An R Package for a Fast Calculation to Semi-partial Correlation Coefficients. Commun Stat Appl Methods, 2015. 22(6): p. 665-674.
39. Cowley, G. S., et al., Parallel genome-scale loss of function screens in 216 cancer cell lines for the identification of context-specific genetic dependencies. Sci Data, 2014. 1: p. 140035.
40. Ritz, C., et al., Dose-Response Analysis Using R. PLoS One, 2015. 10(12): p. e0146021.
41. Van der Borght, K., et al., BIGL: Biochemically Intuitive Generalized Loewe null model for prediction of the expected combined effect compatible with partial agonism and antagonism. Sci Rep, 2017. 7(1): p. 17935.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A pharmaceutical composition for treating a cancer in a subject comprising a KDM6A/KDM6B inhibitor and a MGLL inhibitory agent, and a pharmaceutically acceptable carrier.

2. The pharmaceutical composition of claim 1, wherein the KDM6A/B inhibitor is selected from the group consisting of GSK-J4, IOX1, GSK-J1 and caffeic acid.

3. The pharmaceutical composition of claim 1, wherein the MGLL inhibitory agent is selected from the group consisting of JZL 184, URB602, pristimerin, an 0-hexafluorosiopropyl carbamate, and an oligonucleotide inhibitor ofMGLL.

4. The pharmaceutical composition of claim 1,
wherein the cancer is selected from the group consisting of AML, ALL and prostate cancer.

5. A pharmaceutical composition for treating a cancer in a subject selected from the group consisting of:
A pharmaceutical composition comprising a STAT3 signaling inhibitor and a UGTJAJO inhibitory agent, and a pharmaceutically acceptable carrier;
A pharmaceutical composition comprising a CDK4/6 inhibitor and a CCNE1 inhibitory agent, and a pharmaceutically acceptable carrier;
A pharmaceutical composition comprising an AKT1/2 inhibitor and an AKT3 inhibitory agent, and a pharmaceutically acceptable carrier;
A pharmaceutical composition comprising a topoisomerase II inhibitor and a BCL2L1 inhibitory agent, and a pharmaceutically acceptable carrier;
A pharmaceutical composition comprising a PI3 kinase inhibitor and an IRS2 inhibitory agent, and a pharmaceutically acceptable carrier;
A pharmaceutical composition comprising a GPX4 inhibitor and an AIFM2 inhibitory agent, and a pharmaceutically acceptable carrier;
A pharmaceutical composition comprising an inducer of reactive oxygen species (ROS) and an ABCC1 inhibitory agent, and a pharmaceutically acceptable carrier;
A pharmaceutical composition comprising a JNK1 inhibitor and an ABCG2 inhibitory agent, and a pharmaceutically acceptable carrier; and
A pharmaceutical composition comprising an E3-ubiquitin ligase inhibitor and a UGT1A6 inhibitory agent, and a pharmaceutically acceptable carrier.

6. The pharmaceutical composition of claim 5, wherein the STAT3 signaling inhibitor is selected from the group consisting of niclosamide (5-Chloro-N-(2-chloro-4-nitrophenyl)-2-hydroxybenzamide), S31-201, stattic, nifuroxazide, C188-9, SH-4-54, napabucasin, artesunate, BP-1-102, cryptotanshinone, SH5-07 (SH-5-07), ochromycinone (STA-21), APTSTAT3-9R and HO-3867.

7. The pharmaceutical composition of claim 5, wherein the UGT1A10 inhibitory agent is an oligonucleotide inhibitor of UGT1A10.

8. The pharmaceutical composition of claim 5, wherein the cancer is selected from the group consisting of breast cancer, head and neck squamous cell carcinoma (HNSCC), non-small cell lung cancer (NSCLC), hepatocellular carcinoma (HCC), colorectal cancer (CRC), gastric adenocarcinoma and melanoma.

9. (canceled)

10. The pharmaceutical composition of claim 5, wherein the CDK4/6 inhibitor is selected from the group consisting of flavopiridol, abemaciclib, ribociclib and palbociclib.

11. The pharmaceutical composition of claim 5, wherein the CCNE1 inhibitory agent is an oligonucleotide inhibitor of CCNE1.

12. The pharmaceutical composition of claim 5, wherein the cancer is selected from the group consisting of breast cancer, non-small cell lung cancer (NSCLC), mantle cell lymphoma, liposarcoma, melanoma, glioblastoma, pancreatic cancer, and colorectal cancer.

13. (canceled)

14. The pharmaceutical composition of claim 5, wherein:
the AKT1/2 inhibitor is selected from the group consisting of BAY1125976 and AKT inhibitor VIII;
the AKT3 inhibitory agent is an oligonucleotide inhibitor of AKT3, and/or
the cancer is selected from the group consisting of breast cancer, head and neck cancer, squamous cell carcinoma, endometrial cancer, non-small cell lung cancer (NSCLC), renal cancer, gastric cancer, ovarian cancer, pancreatic cancer, colon cancer, oesophageal cancer and thyroid cancer.

15-17. (canceled)

18. The pharmaceutical composition of claim 5, wherein:
the topoisomerase II inhibitor is selected from the group consisting of amsacrine, etoposide, etoposide phosphate, teniposide, ICRF-193, genistein and doxorubicin;
the BCL2L1 inhibitory agent is selected from the group consisting of Z36, 2,3-DCPE hydrochloride, arctigenin, (±)-gossypol, gossypol-acetic acid, R(−)-gossypol and an oligonucleotide inhibitor of BCL2L; and/or
the cancer is selected from the group consisting of breast cancer, bladder cancer, Kaposi's sarcoma, lymphoma, acute lymphocytic leukemia and colorectal cancer.

19-21. (canceled)

22. The pharmaceutical composition of claim 5, wherein:
the PI3 kinase inhibitor is selected from the group consisting of Pictilisib, Idelalisib, Copanlisib, Taselisib, Perifosine, Buparlisib (BKM120), Duvelisib (IPI-145), Alpelisib (BYL719), Umbralisib, (TGR 1202), Copanlisib (BAY 80-6946), PX-866, Dactolisib, CUDC-907, Voxtalisib, CUDC-907, ME-401, IPI-549, SF1126, RP6530, INK1117, XL147 (also known as SAR245408), Palomid 529, GSK1059615, ZSTK474, PWT33597, IC87114, TG100-115, CAL263, RP6503, PI-103, GNE-477 and AEZS-136;
the IRS2 inhibitory agent is an oligonucleotide inhibitor of IRS2, and/or
the cancer is selected from the group consisting of leukemia, breast cancer, lung cancer, colorectal cancer, hematologic malignancies, thyroid cancer, inflammatory conditions, multiple myeloma, and lymphoma, optionally B-cell lymphomas, optionally CLL or follicular lymphoma.

23-25. (canceled)

26. The pharmaceutical composition of claim 5, wherein:
the GPX4 inhibitor is selected from the group consisting of ML210, racemic RSL3, (1S,3R)-RSL3, ML162, CIL56, DPI19, DPI18, DPI17, DPI13, DPI12, altretamine and FIN56;
the AIFM2 inhibitory agent is an oligonucleotide inhibitor of AIFM2, and/or
the cancer is selected from the group consisting of a diffuse large B cell lymphoma (DLBCL) and a renal cell carcinoma.

27-29. (canceled)

30. The pharmaceutical composition of claim 5, wherein:
the inducer of reactive oxygen species (ROS) is selected from the group consisting of BRD1378, BRD5459, BRD56491 and BRD9092;
the ABCC1 inhibitory agent is selected from the group consisting of MK-571, Reversan and an oligonucleotide inhibitor of ABCC1; and/or
the cancer is selected from the group consisting of colon, pancreatic, breast, glioma, glioblastoma, non-small cell lung cancer, multiple myeloma, prostate cancer, hepatoma and leukemia.

31-33. (canceled)

34. The pharmaceutical composition of claim 5, wherein:
the JNK1 inhibitor is selected from the group consisting of ZG-10, tanzisertib (CC-930), SP600125, JNK Inhibitor V, INK Inhibitor XIV, L-JNKi 1 trifluoroacetate salt, INK Inhibitor XV, JNK Inhibitor XI and JNK-IN-8;
the ABCG2 inhibitory agent is selected from the group consisting of elacridar, vismodegib, fumitremorgin C, Ko 143, novobiocin sodium salt and an oligonucleotide inhibitor of ABCG2, and/or
the cancer is selected from the group consisting of liver cancer, breast cancer, skin cancer, brain cancer, leukemia, multiple myeloma and lymphoma.

35-37. (canceled)

38. The pharmaceutical composition of claim 5, wherein:
the E3-ubiquitin ligase inhibitor is selected from the group consisting of SMER-3, Heclin and SZL P1-41;
the UGT1A6 inhibitory agent is an oligonucleotide inhibitor of UGT1A6; and/or
the cancer is selected from the group consisting of malignant melanoma and multiple myeloma.

39-40. (canceled)

41. A method selected from the group consisting of:
A method for reducing resistance of a cell, tissue and/or subject to a KDM6A/KDM6B inhibitor comprising administering a MGLL inhibitory agent to the cell, tissue and/or subject, thereby reducing resistance of the cell, tissue and/or subject to the KDM6A/KDM6B inhibitor;
A method for reducing resistance of a cell, tissue and/or subject to a STAT3 signaling inhibitor comprising administering an UGT1A10 inhibitory agent to the cell, tissue and/or subject, thereby reducing resistance of the cell, tissue and/or subject to the STAT3 signaling inhibitor;
A method for reducing resistance of a cell, tissue and/or subject to a CDK4/6 inhibitor comprising administering a CCNE1 inhibitory agent to the cell, tissue and/or subject, thereby reducing resistance of the cell, tissue and/or subject to the CDK4/6 inhibitor;
A method for reducing resistance of a cell, tissue and/or subject to an AKT1/2 inhibitor comprising administering an AKT3 inhibitory agent to the cell, tissue and/or subject, thereby reducing resistance of the cell, tissue and/or subject to the AKT1/2 inhibitor;
A method for reducing resistance of a cell, tissue and/or subject to a topoisomerase II inhibitor comprising administering a BCL2L1 inhibitory agent to the cell, tissue and/or subject, thereby reducing resistance of the cell, tissue and/or subject to the topoisomerase II inhibitor;
A method for reducing resistance of a cell, tissue and/or subject to a PI3 kinase inhibitor comprising administering an IRS2 inhibitory agent to the cell, tissue and/or subject, thereby reducing resistance of the cell, tissue and/or subject to the PI3 kinase inhibitor;
A method for reducing resistance of a cell, tissue and/or subject to a GPX4 inhibitor comprising administering an AIFM2 inhibitory agent to the cell, tissue and/or subject, thereby reducing resistance of the cell, tissue and/or subject to the GPX4 inhibitor;
A method for reducing resistance of a cell, tissue and/or subject to an inducer of reactive oxygen species (ROS) comprising administering an ABCC1 inhibitory agent to the cell, tissue and/or subject, thereby reducing resistance of the cell, tissue and/or subject to the ROS inducer;
A method for reducing resistance of a cell, tissue and/or subject to a JNK1 inhibitor comprising administering an ABCG2 inhibitory agent to the cell, tissue and/or subject, thereby reducing resistance of the cell, tissue and/or subject to the JNK1 inhibitor; and
A method for reducing resistance of a cell, tissue and/or subject to an E3-ubiquitin ligase inhibitor comprising administering an UGT1A6 inhibitory agent to the cell, tissue and/or subject, thereby reducing resistance of the cell, tissue and/or subject to the E3-ubiquitin ligase inhibitor.

42-50. (canceled)


SEQUENCE LISTING (14 sequences)

SEQ ID NO: 1 - DNA, 20 bp, Artificial Sequence
  Description of Artificial Sequence Synthetic DNA
acagtgggtacatgcgcatg

SEQ ID NO: 2 - DNA, 20 bp, Artificial Sequence
  Description of Artificial Sequence Synthetic DNA
cagggtgtattcatccagcg

SEQ ID NO: 3 - DNA, 20 bp, Artificial Sequence
  Description of Artificial Sequence Synthetic DNA
ccagagcaagaacctgacgg

SEQ ID NO: 4 - DNA, 20 bp, Artificial Sequence
  Description of Artificial Sequence Synthetic DNA
gcagtaagagccaatcgtcg

SEQ ID NO: 5 - DNA, 20 bp, Artificial Sequence
  Description of Artificial Sequence Synthetic DNA
tcaggattggcaagaaccag

SEQ ID NO: 6 - DNA, 20 bp, Artificial Sequence
  Description of Artificial Sequence Synthetic DNA
gctctctcggaataagacag

SEQ ID NO: 7 - DNA, 20 bp, Artificial Sequence
  Description of Artificial Sequence Synthetic DNA
acacatccctgacgaaaacg

SEQ ID NO: 8 - DNA, 20 bp, Artificial Sequence
  Description of Artificial Sequence Synthetic DNA
ctggtgttcgcccacgacca

SEQ ID NO: 9 - DNA, 20 bp, Artificial Sequence
  Description of Artificial Sequence Synthetic DNA
ccagcgctcacggttcatcg

SEQ ID NO: 10 - DNA, 20 bp, Artificial Sequence
  Description of Artificial Sequence Synthetic DNA
cactctcattcactcccaag

SEQ ID NO: 11 - DNA, 20 bp, Artificial Sequence
  Description of Artificial Sequence Synthetic DNA
agagtacatcaaagtgcaga

SEQ ID NO: 12 - DNA, 20 bp, Artificial Sequence
  Description of Artificial Sequence Synthetic DNA
tgagcgggtgagcaatctgg

SEQ ID NO: 13 - DNA, 20 bp, Artificial Sequence
  Description of Artificial Sequence Synthetic DNA
ccgcgcatttcagagcacaa

SEQ ID NO: 14 - DNA, 20 bp, Artificial Sequence
  Description of Artificial Sequence Synthetic DNA
cagagccttgcgcaattttg